(19) United States
(12) Patent Application Publication
(10) Pub. No.: US 2024/0131011 A1
(43) Pub. Date: Apr. 25, 2024

(54) PYRUVATE KINASE R (PKR) ACTIVATING COMPOSITIONS

(71) Applicant: FORMA Therapeutics, Inc., Watertown, MA (US)

(72) Inventors: George P. Luke, Clinton, CT (US); Suresh Babu, Ypsilanti, MI (US)

(21) Appl. No.: 18/125,932

(22) Filed: Mar. 24, 2023

Related U.S. Application Data
(60) Provisional application No. 63/323,808, filed on Mar. 25, 2022.

(51) Int. Cl. A61K 31/436; A61K 9/00; A61K 9/20

(52) U.S. Cl. CPC A61K 31/436; A61K 9/0053; A61K 9/2009; A61K 9/2013; A61K 9/2018; A61K 9/2027; A61K 9/2054

(57) ABSTRACT

The present disclosure provides crystalline solid forms, spray-dried dispersions and pharmaceutical compositions, including solid oral dosage forms, of (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one (“Compound 1”), and preparation methods thereof.

### CROSS-REFERENCE TO RELATED APPLICATIONS

This application claims the benefit of U.S. Provisional Application No. 63/323,808, filed Mar. 25, 2022, which is incorporated by reference in its entirety.

### TECHNICAL FIELD

The present disclosure is directed to solid forms, dispersions and pharmaceutical compositions of a pyruvate kinase R (PKR) activating compound. More specifically, the present disclosure is directed to crystalline solid forms, spray-dried dispersions and pharmaceutical compositions of (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one, and preparation methods thereof.

### BACKGROUND

Chemical compounds can form one or more different pharmaceutically acceptable solid forms, including amorphous and crystalline forms. Amorphous solid forms include dispersions, such as spray-dried dispersions, of amorphous and crystalline chemical compounds. Individual solid forms of bioactive chemical compounds can have different properties. There is a need for the identification and selection of appropriate solid forms of bioactive chemical compounds (including appropriate crystalline forms, where applicable) for the development of pharmaceutically acceptable dosage forms for the treatment of various diseases or conditions.

The compound (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one (“Compound 1”),

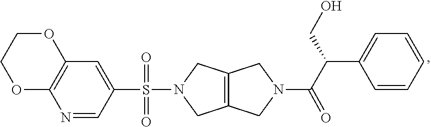 [SMILES: O=C([C@H](CO)c1ccccc1)N1CC2=C(C1)CN(S(=O)(=O)c1cnc3c(c1)OCCO3)C2]

is a small molecule PKR activator which modulates pyruvate kinase activity. Compound 1 is described in International Publication No. WO 2018/175474 as one of many compounds suitable as small molecule modulators of pyruvate kinase activity. There remains a need for identifying solid forms of Compound 1 useful for various therapeutic applications.

### SUMMARY

One aspect of the disclosure relates to solid oral dosage forms comprising a stabilized amorphous pharmaceutical composition of the compound (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one (also referred to as “stabilized amorphous Compound 1”). As used herein, the term “stabilized amorphous Compound 1” refers to an amorphous solid form of Compound 1 that is stabilized (e.g., by combination with certain stabilizing polymers and/or other manufacturing processes) to prevent the formation of crystalline forms of Compound 1 or solid phase separation of Compound 1 under certain storage conditions described herein (e.g., stabilized amorphous pharmaceutical compositions comprising Compound 1 and one or more additional components that do not show crystalline diffraction peaks by XRPD analysis (Method D) after 2 weeks of storage at 60° C./75% RH (exposed), and/or show a single glass transition temperature (T_{G}) with no melt endotherm by DSC analysis (Method B) after 2 weeks of storage at 60° C./75% RH (exposed)).

In some embodiments, the stabilized amorphous Compound 1 is obtained by spray drying a solution of Compound 1 with a stabilizing polymer. The inventors discovered that amorphous Compound 1 has higher oral bioavailability than certain crystalline forms of Compound 1, including crystalline form Type A. Accordingly, in some embodiments, solid oral dosage forms comprising stabilized amorphous Compound 1 advantageously provide superior oral bioavailability of Compound 1 in comparison to solid oral dosage forms comprising certain crystalline forms of Compound 1.

Also disclosed herein are an amorphous spray-dried dispersion (SDD) of Compound 1, preparation methods thereof, and pharmaceutical compositions containing the same. The present disclosure provides various solid forms of Compound 1, including one or more pharmaceutically acceptable crystalline and amorphous forms for Compound 1, useful for the therapeutic oral administration of Compound 1. The various solid forms of Compound 1 can be identified by certain characteristic properties. For example, certain crystalline forms of Compound 1 have distinct characteristic XRPD peaks.

Another aspect of the disclosure relates to solid forms of Compound 1. Solid forms of Compound 1 disclosed herein include various crystalline forms (including Type A, Type B, Type C, Type D, Type E, Type F, Type G, Type H, Type I, Type J, Type K, Type L, and Type M) of Compound 1, preparation methods thereof, and pharmaceutical compositions containing the same.

One aspect of the present disclosure relates to novel crystalline solid forms of Compound 1:

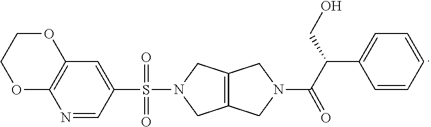 [SMILES: O=C([C@H](CO)c1ccccc1)N1CC2=C(C1)CN(S(=O)(=O)c1cnc3c(c1)OCCO3)C2]

A novel Compound 1 crystalline form Type A can be identified by X-ray Powder Diffraction (XRPD) pattern having one or more characteristic diffractions at angles (2 theta±0.2) of 4.61, 15.66, 23.19, and 24.76. A novel Compound 1 crystalline form Type A can be identified by X-ray Powder Diffraction (XRPD) pattern having one or more characteristic diffractions at angles (2 theta±0.2) of 4.6, 15.7, 23.2, and 24.8. A novel Compound 1 crystalline form Type A can be identified by X-ray Powder Diffraction (XRPD) pattern having one or more characteristic diffractions at angles (2 theta±0.2) of 4.6, 7.2, 15.7, 21.3, 23.2, and 24.8.

A novel Compound 1 crystalline form Type B can be identified by X-ray Powder Diffraction (XRPD) pattern having one or more characteristic diffractions at angles (2 theta±0.2) of 4.52, 15.57, 22.89, 23.34, and 25.13. A novel Compound 1 crystalline form Type B can be identified by X-ray Powder Diffraction (XRPD) pattern having one or more characteristic diffractions at angles (2 theta±0.2) of 4.5, 15.6, 22.9, 23.3, and 25.1. A novel Compound 1 crystalline form Type B can be identified by X-ray Powder Diffraction (XRPD) pattern having one or more characteristic diffractions at angles (2 theta±0.2) of 4.5, 15.6, 22.2, 22.9, 23.3, and 25.1.

A novel Compound 1 crystalline form Type C can be identified by X-ray Powder Diffraction (XRPD) pattern having one or more characteristic diffractions at angles (2 theta±0.2) of 4.55, 18.85, 23.02, and 24.65. A novel Compound 1 crystalline form Type C can be identified by X-ray Powder Diffraction (XRPD) pattern having one or more characteristic diffractions at angles (2 theta±0.2) of 4.5, 18.9, 23.0, and 24.7. A novel Compound 1 crystalline form Type C can be identified by X-ray Powder Diffraction (XRPD) pattern having one or more characteristic diffractions at angles (2 theta±0.2) of 4.5, 7.3, 11.2, 18.9, 23.0, and 24.7.

A novel Compound 1 crystalline form Type D can be identified by X-ray Powder Diffraction (XRPD) pattern having one or more characteristic diffractions at angles (2 theta±0.2) of 9.72, 13.08, 15.74, 21.90, and 23.59. A novel Compound 1 crystalline form Type D can be identified by X-ray Powder Diffraction (XRPD) pattern having one or more characteristic diffractions at angles (2 theta±0.2) of 9.7, 13.1, 15.7, 21.9, and 23.6. A novel Compound 1 crystalline form Type D can be identified by X-ray Powder Diffraction (XRPD) pattern having one or more characteristic diffractions at angles (2 theta±0.2) of 6.2, 9.7, 13.1, 15.7, 21.9, and 23.6 and not having a diffraction at an angle (2 theta±0.2) of 23.3.

A novel Compound 1 crystalline form Type E can be identified by X-ray Powder Diffraction (XRPD) pattern having one or more characteristic diffractions at angles (2 theta±0.2) of 15.12, 15.75, 17.48, 20.05, 21.93, and 26.72. A novel Compound 1 crystalline form Type E can be identified by X-ray Powder Diffraction (XRPD) pattern having one or more characteristic diffractions at angles (2 theta±0.2) of 15.1, 15.8, 17.5, 20.1, 21.9, and 26.7. A novel Compound 1 crystalline form Type E can be identified by X-ray Powder Diffraction (XRPD) pattern having one or more characteristic diffractions at angles (2 theta±0.2) of 15.1, 15.8, 17.5, 20.1, 21.9, and 26.7.

A novel Compound 1 crystalline form Type F can be identified by X-ray Powder Diffraction (XRPD) pattern having one or more characteristic diffractions at angles (2 theta±0.2) of 5.45, 14.66, 16.00, 16.79, 20.01, 21.36, and 22.45. A novel Compound 1 crystalline form Type F can be identified by X-ray Powder Diffraction (XRPD) pattern having one or more characteristic diffractions at angles (2 theta±0.2) of 5.4, 14.7, 16.0, 16.8, 20.0, 21.4, and 22.5. A novel Compound 1 crystalline form Type F can be identified by X-ray Powder Diffraction (XRPD) pattern having one or more characteristic diffractions at angles (2 theta±0.2) of 5.4, 14.7, 16.0, 16.8, and 21.4.

A novel Compound 1 crystalline form Type G can be identified by X-ray Powder Diffraction (XRPD) pattern having one or more characteristic diffractions at angles (2 theta±0.2) of 5.36, 14.34, 16.58, and 21.35. A novel Compound 1 crystalline form Type G can be identified by X-ray Powder Diffraction (XRPD) pattern having one or more characteristic diffractions at angles (2 theta±0.2) of 5.4, 14.3, 16.6, and 21.4. A novel Compound 1 crystalline form Type G can be identified by X-ray Powder Diffraction (XRPD) pattern having one or more characteristic diffractions at angles (2 theta±0.2) of 5.4, 14.3, 16.6, 21.3, and 22.3.

A novel Compound 1 crystalline form Type H can be identified by X-ray Powder Diffraction (XRPD) pattern having one or more characteristic diffractions at angles (2 theta±0.2) of 5.8, 14.7, 16.6, 20.0, 21.3, and 25.4.

A novel Compound 1 crystalline form Type I can be identified by X-ray Powder Diffraction (XRPD) pattern having one or more characteristic diffractions at angles (2 theta±0.2) of 5.2, 14.6, 15.5, 20.2, and 21.1.

A novel Compound 1 crystalline form Type J can be identified by X-ray Powder Diffraction (XRPD) pattern having one or more characteristic diffractions at angles (2 theta±0.2) of 4.5, 5.7, 22.8, 23.1, and 24.5.

A novel Compound 1 crystalline form Type K can be identified by X-ray Powder Diffraction (XRPD) pattern having one or more characteristic diffractions at angles (2 theta±0.2) of 4.6, 15.4, 15.6, 16.1, 23.2, and 27.4.

A novel Compound 1 crystalline form Type L can be identified by X-ray Powder Diffraction (XRPD) pattern having one or more characteristic diffractions at angles (2 theta±0.2) of 5.9, 11.9, 17.8, 21.6, 23.9, and 36.1.

A novel Compound 1 crystalline form Type M can be identified by X-ray Powder Diffraction (XRPD) pattern having one or more characteristic diffractions at angles (2 theta±0.2) of 4.5, 5.8, 9.7, 15.6, 21.9, and 26.7.

Another aspect of the present disclosure relates to a pharmaceutical composition comprising a therapeutically effective amount of any of the crystalline solid forms of Compound 1 described above, and one or more pharmaceutically acceptable excipients.

Yet another aspect of the present disclosure relates to a novel amorphous solid dispersion of Compound 1. The novel amorphous solid form of Compound 1 can be prepared by spray-drying a mixture comprising Compound 1 and a polymer.

Still another aspect of the present disclosure relates to a pharmaceutical composition comprising the novel amorphous solid form of Compound 1 described above. The pharmaceutical composition may be in an oral dosage form, such as tablets.

Another aspect of the present disclosure relates to tablet dosage forms comprising Compound 1.

### BRIEF DESCRIPTION OF THE DRAWINGS

FIG. 1 depicts a reaction scheme to prepare Compound 1.

FIG. 2 depicts an alternative reaction scheme to prepare Compound 1.

FIG. 3 depicts an XRPD pattern of Compound 1 crystalline form Type A.

FIG. 4 depicts a thermogravimetric analysis (TGA) curve (upper curve) and a differential scanning calorimetry (DSC) thermogram (lower curve) for Compound 1 crystalline form Type A.

FIG. 5 depicts a DSC cycling thermogram for Compound 1 crystalline form Type A.

FIG. 6 depicts a dynamic vapor sorption (DVS) isotherm for Compound 1 crystalline form Type A.

FIG. 7 depicts an XRPD pattern of Compound 1 crystalline form Type B.

FIG. 8 depicts a thermogravimetric analysis (TGA) curve (upper curve) and a differential scanning calorimetry (DSC) thermogram (lower curve) for Compound 1 crystalline form Type B.

FIG. 9 depicts a DSC cycling thermogram for Compound 1 crystalline form Type B.

FIG. 10 depicts two thermogravimetric analysis (TGA) curves for Compound 1 crystalline form Type B.

FIG. 11 depicts a dynamic vapor sorption (DVS) isotherm for Compound 1 crystalline form Type B.

FIG. 12 depicts an XRPD pattern of Compound 1 crystalline form Type C.

FIG. 13 depicts a thermogravimetric analysis (TGA) curve (upper curve) and a differential scanning calorimetry (DSC) thermogram (lower curve) for Compound 1 crystalline form Type C.

FIG. 14 depicts a DSC cycling thermogram for Compound 1 crystalline form Type C.

FIG. 15 depicts thermogravimetric analysis (TGA) curves for Compound 1 crystalline form Type C.

FIG. 16 depicts a dynamic vapor sorption (DVS) isotherm for Compound 1 crystalline form Type C.

FIG. 17 depicts an XRPD pattern of Compound 1 crystalline form Type D.

FIG. 18 depicts a thermogravimetric analysis (TGA) curve (upper curve) and a differential scanning calorimetry (DSC) thermogram (lower curve) for Compound 1 crystalline form Type D.

FIG. 19 depicts a ¹H NMR spectrum of Type A (upper curve) and Type D (lower curve) crystalline forms of Compound 1.

FIG. 20 depicts an XRPD pattern of Compound 1 crystalline form Type E.

FIG. 21 depicts an XRPD pattern of Compound 1 crystalline form Type F.

FIG. 22 is a thermogravimetric analysis (TGA) curve (upper curve) and a differential scanning calorimetry (DSC) thermogram (lower curve) for Compound 1 crystalline form Type F.

FIG. 23 depicts an XRPD pattern of Compound 1 crystalline form Type G.

FIG. 24 depicts an XRPD pattern of Compound 1 crystalline form Type H.

FIG. 25 depicts an XRPD pattern of Compound 1 crystalline form Type I.

FIG. 26 depicts an XRPD pattern of Compound 1 crystalline form Type J.

FIG. 27 depicts an XRPD pattern of Compound 1 crystalline form Type K.

FIG. 28 depicts an XRPD pattern of Compound 1 crystalline form Type L.

FIG. 29 depicts an XRPD pattern of Compound 1 crystalline form Type M.

FIG. 30 depicts an XRPD pattern of a spray-dried dispersion (SDD) of Compound 1.

FIG. 31 depicts a differential scanning calorimetry (DSC) thermogram for a spray-dried dispersion (SDD) of Compound 1.

FIG. 32 depicts a graph of the plasma concentration over time following administration of three formulations of Compound 1 in rats.

FIG. 33 depicts a graph of the plasma concentration over time following administration of four formulations of Compound 1 in monkeys.

FIG. 34 depicts a graph of time-dependent solubility of Type A of Compound 1 in biorelevant media.

FIG. 35 depicts a graph of time-dependent solubility of a spray-dried dispersion (SDD) of Compound 1 in biorelevant media.

FIG. 36 depicts overlayed XRPD patterns of five spray-dried dispersions (SDDs) of Compound 1, overlayed with the XRPD pattern of crystalline Compound 1 (Type A).

FIG. 37 depicts overlayed differential scanning calorimetry (DSC) thermograms of five spray-dried dispersions (SDDs) of Compound 1.

FIG. 38 depicts a graph of the kinetic solubility profiles of five SDDs of Compound 1 at different drug loadings.

FIG. 39 depicts overlayed XRPD patterns of a spray dried dispersion of Compound 1 (SDD 0) after storage (a) in a sealed vial for 2 weeks at 60° C., (b) in an unsealed vial for 2 weeks at 40° C. and 75% relative humidity, and (c) in an unsealed vial for 2 weeks at 60° C. and 75% relative humidity, overlayed with the XRPD pattern of crystalline Compound 1 (Type A).

FIG. 40 depicts overlayed XRPD patterns of a spray dried dispersion of Compound 1 (SDD 1) after storage (a) in a sealed vial for 2 weeks at 60° C., (b) in an unsealed vial for 2 weeks at 40° C. and 75% relative humidity, and (c) in an unsealed vial for 2 weeks at 60° C. and 75% relative humidity, overlayed with the XRPD pattern of crystalline Compound 1 (Type A).

FIG. 41 depicts overlayed XRPD patterns of a spray dried dispersion of Compound 1 (SDD 2) after storage (a) in a sealed vial for 2 weeks at 60° C., (b) in an unsealed vial for 2 weeks at 40° C. and 75% relative humidity, and (c) in an unsealed vial for 2 weeks at 60° C. and 75% relative humidity, overlayed with the XRPD pattern of crystalline Compound 1 (Type A).

FIG. 42 depicts overlayed XRPD patterns of a spray dried dispersion of Compound 1 (SDD 3) after storage (a) in a sealed vial for 2 weeks at 60° C., (b) in an unsealed vial for 2 weeks at 40° C. and 75% relative humidity, and (c) in an unsealed vial for 2 weeks at 60° C. and 75% relative humidity, overlayed with the XRPD pattern of crystalline Compound 1 (Type A).

FIG. 43 depicts overlayed XRPD patterns of a spray dried dispersion of Compound 1 (SDD 4) after storage (a) in a sealed vial for 2 weeks at 60° C., (b) in an unsealed vial for 2 weeks at 40° C. and 75% relative humidity, and (c) in an unsealed vial for 2 weeks at 60° C. and 75% relative humidity, overlayed with the XRPD pattern of crystalline Compound 1 (Type A).

FIG. 44 depicts overlayed XRPD patterns of two spray dried dispersions of Compound 1 (SDDs 5 and 6), overlayed with the XRPD pattern of crystalline Compound 1 (Type A).

FIG. 45 depicts overlayed DSC thermograms of two spray dried dispersions of Compound 1 (SDDs 5 and 6).

FIG. 46 depicts overlayed XRPD patterns of a spray dried dispersion of Compound 1 (SDD 5) after storage (a) in a sealed vial for 1 week at 60° C., (b) in an unsealed vial for 1 week at 25° C. and 60% relative humidity, and (c) in an unsealed vial for 1 week at 40° C. and 75% relative humidity.

FIG. 47 depicts overlayed DSC thermograms of a spray dried dispersion of Compound 1 (SDD 5) after storage (a) in a sealed vial for 1 week at 60° C., (b) in an unsealed vial for 1 week at 25° C. and 60% relative humidity, and (c) in an unsealed vial for 1 week at 40° C. and 75% relative humidity.

FIG. 48 depicts overlayed DSC thermograms of a spray dried dispersion of Compound 1 (SDD 5) after storage (a) in a sealed vial for 2 weeks at 60° C., (b) in an unsealed vial for 2 weeks at 25° C. and 60% relative humidity, and (c) in an unsealed vial for 2 weeks at 40° C. and 75% relative humidity.

FIG. 49 depicts overlayed XRPD patterns of a spray dried dispersion of Compound 1 (SDD 6) after storage (a) in a sealed vial for 1 week at 60° C., (b) in an unsealed vial for 1 week at 25° C. and 60% relative humidity, and (c) in an unsealed vial for 1 week at 40° C. and 75% relative humidity.

FIG. 50 depicts overlayed DSC thermograms of a spray dried dispersion of Compound 1 (SDD 6) after storage (a) in a sealed vial for 1 week at 60° C., (b) in an unsealed vial for 1 week at 25° C. and 60% relative humidity, and (c) in an unsealed vial for 1 week at 40° C. and 75% relative humidity.

FIG. 51 depicts overlayed DSC thermograms of a spray dried dispersion of Compound 1 (SDD 6) after storage (a) in a sealed vial for 2 weeks at 60° C., (b) in an unsealed vial for 2 weeks at 25° C. and 60% relative humidity, and (c) in an unsealed vial for 2 weeks at 40° C. and 75% relative humidity.

FIG. 52 depicts a graph of the dissolution profile of a tablet formulation of Compound 1.

### DETAILED DESCRIPTION

The chemical compound (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one (“Compound 1”),

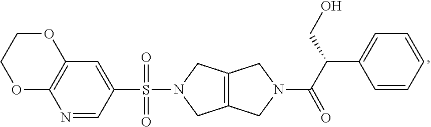 [SMILES: O=C([C@H](CO)c1ccccc1)N1CC2=C(C1)CN(S(=O)(=O)c1cnc3c(c1)OCCO3)C2]

is a small molecule modulator of pyruvate kinase. The present disclosure provides various solid forms of Compound 1, pharmaceutical compositions thereof, and methods of preparing those novel solid forms of Compound 1. The solid forms described herein (e.g., crystalline solid forms and amorphous solid forms) are associated with favorable characteristics such as favorable or improved solubility, dissolution, bioavailability, stability, and ease of formulation relative to other forms of Compound 1. For example, certain amorphous solid dispersions described herein advantageously have high drug loads (e.g., ≥25%, ≥40%, ≥50%, etc.), are free or substantially free of crystalline Compound 1, are physically stable (i.e., remain free or substantially free of crystalline Compound 1 over time in accelerated stability studies), are highly soluble, and/or do not require extensive drying to remove residual solvents. Further, certain tablet dosage forms described herein advantageously have high drug loads (e.g., ≥10 weight % of the tablet core, ≥15 weight % of the tablet core, ≥30 weight % of the tablet core), small tablet sizes (e.g., tablet core weight≤1200 mg, ≤1000 mg, ≤800 mg, ≤700 mg, etc. per tablet), are free or substantially free of crystalline Compound 1, and/or are physically stable (i.e., remain free or substantially free of crystalline Compound 1 over time in accelerated stability studies).

In some embodiments, Compound 1 is in a crystalline solid form (e.g., Type A, Type B, Type C, Type D, Type E, Type F, or Type G). In some embodiments, Compound 1 is in a crystalline solid form (e.g., Type A, Type B, Type C, Type D, Type E, Type F, Type G, Type H, Type I, Type J, Type K, Type L, or Type M). In some embodiments, the crystalline solid form is Type A. In some embodiments, the crystalline solid form is Type B. In some embodiments, the crystalline solid form is Type C. In some embodiments, the crystalline solid form is Type D. In some embodiments, the crystalline solid form is Type E. In some embodiments, the crystalline solid form is Type F. In some embodiments, the crystalline solid form is Type G. In some embodiments, the crystalline solid form is Type H. In some embodiments, the crystalline solid form is Type I. In some embodiments, the crystalline solid form is Type J. In some embodiments, the crystalline solid form is Type K. In some embodiments, the crystalline solid form is Type L. In some embodiments, the crystalline solid form is Type M.

In some embodiments, Compound 1 is in amorphous form (e.g., an amorphous solid dispersion). In some embodiments, the amorphous solid dispersion comprises Compound 1 and a polymer.

### Compound 1 Crystalline Form Type A

A novel Compound 1 crystalline form Type A can be identified by an X-ray Powder Diffraction (XRPD) pattern having one or more characteristic diffractions at angles (2 theta±0.2) of 4.61, 15.66, 23.19, and 24.76. A novel Compound 1 crystalline form Type A can be identified by an X-ray Powder Diffraction (XRPD) pattern having one or more characteristic diffractions at angles (2 theta±0.2) of 4.6, 15.7, 23.2, and 24.8. In some embodiments, Compound 1 crystalline form Type A can be identified by X-ray Powder Diffraction (XRPD), having one or more characteristic diffractions at angles (2 theta±0.2) of 4.61, 15.66, 23.19, and 24.76, corresponding to d-spacing (angstroms±0.2) of 19.19, 5.66, 3.84, and 3.60, respectively. In some embodiments, Compound 1 crystalline form Type A can be identified by X-ray Powder Diffraction (XRPD), having one or more characteristic diffractions at angles (2 theta±0.2) of 4.6, 15.7, 23.2, and 24.8, corresponding to d-spacing (angstroms±0.2) of 19.2, 5.7, 3.8, and 3.6, respectively.

In some embodiments, Compound 1 crystalline form Type A can be identified by an XRPD pattern having one or more characteristic diffractions at angles (2 theta±0.2) of 4.6, 7.2, 15.7, 21.3, 23.2, and 24.8. In some embodiments, Compound 1 crystalline form Type A can be identified by XRPD, having one or more characteristic diffractions at angles (2 theta±0.2) of 4.6, 7.2, 15.7, 21.3, 23.2, and 24.8, corresponding to d-spacing (angstroms±0.2) of 19.2, 12.3, 5.7, 4.2, 3.8, and 3.6, respectively.

In some embodiments, Compound 1 crystalline form Type A can be identified by an XRPD pattern having one or more characteristic diffractions at angles (2 theta±0.2) of 4.61, 7.22, 15.66, 20.48, 21.35, 21.66, 22.47, 23.19, 24.76, and 26.73. In some embodiments, Compound 1 crystalline form Type A can be identified by an XRPD pattern having one or more characteristic diffractions at angles (2 theta±0.2) of 4.6, 7.2, 15.7, 20.5, 21.4, 21.7, 22.5, 23.2, 24.8, and 26.7. In some embodiments, Compound 1 crystalline form Type A can be identified by XRPD, having one or more characteristic diffractions at angles (2 theta±0.2) of 4.61, 7.22, 15.66, 20.48, 21.35, 21.66, 22.47, 23.19, 24.76, and 26.73, corresponding to d-spacing (angstroms±0.2) of 19.19, 12.25, 5.66, 4.34, 4.16, 4.10, 3.96, 3.84, 3.60, and 3.34, respectively. In some embodiments, Compound 1 crystalline form Type A can be identified by XRPD, having one or more characteristic diffractions at angles (2 theta±0.2) of 4.6, 7.2, 15.7, 20.5, 21.4, 21.7, 22.5, 23.2, 24.8, and 26.7, corresponding to d-spacing (angstroms±0.2) of 19.2, 12.2, 5.7, 4.3, 4.2, 4.1, 4.0, 3.8, 3.6, and 3.3, respectively.

In some embodiments, Compound 1 crystalline form Type A is characterized by an X-ray Power Diffraction having one or more characteristic diffractions at angles (2 theta±0.2) of:
4.61
5.80
7.22
7.68
11.21
12.31
14.44
15.66
16.95
18.02
19.20
20.48
21.35
21.66
22.47
23.19
24.76
26.73
28.01
28.49
29.35
30.25
32.14
34.12
36.46

In some embodiments, Compound 1 crystalline form Type A is characterized by an X-ray Power Diffraction having one or more characteristic diffractions at angles (2 theta±0.2) of:
4.6
5.8
7.2
7.7
11.2
12.3
14.4
15.7
16.9
18.0
19.2
20.5
21.3
21.7
22.5
23.2
24.8
26.7
28.0
28.5
29.4
30.3
32.1
34.1
36.5

In some embodiments, Compound 1 crystalline form Type A is characterized by an X-ray Power Diffraction pattern having one or more characteristic diffractions at angles (2 theta 0.2) and corresponding d-spacing (angstroms±0.2) of:

| 2 theta | d-spacing |
|---|---|
| 4.61 | 19.19 |
| 5.80 | 15.24 |
| 7.22 | 12.25 |
| 7.68 | 11.50 |
| 11.21 | 7.89 |
| 12.31 | 7.19 |
| 14.44 | 6.13 |
| 15.66 | 5.66 |
| 16.95 | 5.23 |
| 18.02 | 4.92 |
| 19.20 | 4.62 |
| 20.48 | 4.34 |
| 21.35 | 4.16 |
| 21.66 | 4.10 |
| 22.47 | 3.96 |
| 23.19 | 3.84 |
| 24.76 | 3.60 |
| 26.73 | 3.34 |
| 28.01 | 3.19 |
| 28.49 | 3.13 |
| 29.35 | 3.04 |
| 30.25 | 2.95 |
| 32.14 | 2.79 |
| 34.12 | 2.63 |
| 36.46 | 2.46 |

In some embodiments, Compound 1 crystalline form Type A is characterized by an X-ray Power Diffraction pattern having one or more characteristic diffractions at angles (2 theta 0.2) and corresponding d-spacing (angstroms±0.2) of:

| 2 theta | d-spacing |
|---|---|
| 4.6 | 19.2 |
| 5.8 | 15.2 |
| 7.2 | 12.2 |
| 7.7 | 11.5 |
| 11.2 | 7.9 |
| 12.3 | 7.2 |
| 14.4 | 6.1 |
| 15.7 | 5.7 |
| 16.9 | 5.2 |
| 18.0 | 4.9 |
| 19.2 | 4.6 |
| 20.5 | 4.3 |
| 21.3 | 4.2 |
| 21.7 | 4.1 |
| 22.5 | 4.0 |
| 23.2 | 3.8 |
| 24.8 | 3.6 |
| 26.7 | 3.3 |
| 28.0 | 3.2 |
| 28.5 | 3.1 |
| 29.4 | 3.0 |
| 30.3 | 3.0 |
| 32.1 | 2.8 |
| 34.1 | 2.6 |
| 36.5 | 2.5 |

In some embodiments, Compound 1 crystalline form Type A is characterized by a thermogravimetric analysis (TGA) thermogram with a weight loss of about 1.9% up to 100° C. In some embodiments, Compound 1 crystalline form Type A is characterized by a differential scanning calorimetry (DSC) endotherm having a peak temperature of about 85.9° C. and an onset temperature of about 146.0° C. In some embodiments, Compound 1 crystalline form Type A is characterized by a dynamic vapor sorption (DVS) of about 3.4% water uptake by weight up to 40% relative humidity. In some embodiments, Compound 1 crystalline form Type A is characterized by a dynamic vapor sorption (DVS) of about 1.0% water uptake by weight from 40% to 80% relative humidity.

### Compound 1 Crystalline Form Type B

A novel Compound 1 crystalline form Type B can be identified by an X-ray Powder Diffraction (XRPD) pattern having one or more characteristic diffractions at angles (2 theta±0.2) of 4.52, 15.57, 22.89, 23.34, and 25.13. A novel Compound 1 crystalline form Type B can be identified by an X-ray Powder Diffraction (XRPD) pattern having one or more characteristic diffractions at angles (2 theta±0.2) of 4.5, 15.6, 22.9, 23.3, and 25.1. In some embodiments, Compound 1 crystalline form Type B can be identified by X-ray Powder Diffraction (XRPD), having one or more characteristic diffractions at angles (2 theta±0.2) of 4.52, 15.57, 22.89, 23.34, and 25.13, corresponding to d-spacing (angstroms±0.2) of 19.53, 5.69, 3.89, 3.81, and 3.54, respectively. In some embodiments, Compound 1 crystalline form Type B can be identified by X-ray Powder Diffraction (XRPD), having one or more characteristic diffractions at angles (2 theta±0.2) of 4.5, 15.6, 22.9, 23.3, and 25.1, corresponding to d-spacing (angstroms±0.2) of 19.5, 5.7, 3.9, 3.8, and 3.5, respectively.

In some embodiments, Compound 1 crystalline form Type B can be identified by an XRPD pattern having one or more characteristic diffractions at angles (2 theta±0.2) of 4.5, 15.6, 22.2, 22.9, 23.3, and 25.1. In some embodiments, Compound 1 crystalline form Type B can be identified by XRPD, having one or more characteristic diffractions at angles (2 theta±0.2) of 4.5, 15.6, 22.2, 22.9, 23.3, and 25.1, corresponding to d-spacing (angstroms±0.2) of 19.5, 5.7, 4.0, 3.9, 3.8, and 3.5, respectively.

In some embodiments, Compound 1 crystalline form Type B can be identified by an XRPD pattern having one or more characteristic diffractions at angles (2 theta±0.2) of 4.52, 9.86, 15.57, 19.93, 22.19, 22.89, 23.34, 25.13, and 28.30. In some embodiments, Compound 1 crystalline form Type B can be identified by an XRPD pattern having one or more characteristic diffractions at angles (2 theta±0.2) of 4.5, 9.9, 15.6, 19.9, 22.2, 22.9, 23.3, 25.1, and 28.3. In some embodiments, Compound 1 crystalline form Type B can be identified by XRPD, having one or more characteristic diffractions at angles (2 theta±0.2) of 4.52, 9.86, 15.57, 19.93, 22.19, 22.89, 23.34, 25.13, and 28.30, corresponding to d-spacing (angstroms±0.2) of 19.53, 8.97, 5.69, 4.45, 4.00, 3.89, 3.81, 3.54, and 3.15, respectively. In some embodiments, Compound 1 crystalline form Type B can be identified by XRPD, having one or more characteristic diffractions at angles (2 theta±0.2) of 4.5, 9.9, 15.6, 19.9, 22.2, 22.9, 23.3, 25.1, and 28.3, corresponding to d-spacing (angstroms±0.2) of 19.5, 9.0, 5.7, 4.5, 4.0, 3.9, 3.8, 3.5, and 3.2, respectively.

In some embodiments, Compound 1 crystalline form Type B is characterized by an X-ray Power Diffraction having one or more characteristic diffractions at angles (2 theta±0.2) of:
4.52
8.98
9.86
12.37
13.18
15.57
16.86
18.21
19.11
19.93
20.92
22.19
22.89
23.34
25.13
25.80
26.71
28.30
29.39

In some embodiments, Compound 1 crystalline form Type B is characterized by an X-ray Power Diffraction having one or more characteristic diffractions at angles (2 theta±0.2) of:
4.5
9.0
9.9
12.4
13.2
15.6
16.9
18.2
19.1
19.9
20.9
22.2
22.9
23.3
25.1
25.8
26.7
28.3
29.4

In some embodiments, Compound 1 crystalline form Type B is characterized by an X-ray Power Diffraction pattern having one or more characteristic diffractions at angles (2 theta±0.2) and corresponding d-spacing (angstroms±0.2) of:

| 2 theta | d-spacing |
|---|---|
| 4.52 | 19.53 |
| 8.98 | 9.85 |
| 9.86 | 8.97 |
| 12.37 | 7.15 |
| 13.18 | 6.72 |
| 15.57 | 5.69 |
| 16.86 | 5.26 |
| 18.21 | 4.87 |
| 19.11 | 4.64 |
| 19.93 | 4.45 |
| 20.92 | 4.25 |
| 22.19 | 4.00 |
| 22.89 | 3.89 |
| 23.34 | 3.81 |
| 25.13 | 3.54 |
| 25.80 | 3.45 |
| 26.71 | 3.34 |
| 28.30 | 3.15 |
| 29.39 | 3.04 |

In some embodiments, Compound 1 crystalline form Type B is characterized by an X-ray Power Diffraction pattern having one or more characteristic diffractions at angles (2 theta±0.2) and corresponding d-spacing (angstroms±0.2) of:

| 2 theta | d-spacing |
|---|---|
| 4.5 | 19.5 |
| 9.0 | 9.9 |
| 9.9 | 9.0 |
| 12.4 | 7.2 |
| 13.2 | 6.7 |
| 15.6 | 5.7 |
| 16.9 | 5.3 |
| 18.2 | 4.9 |
| 19.1 | 4.6 |
| 19.9 | 4.5 |
| 20.9 | 4.2 |
| 22.2 | 4.0 |
| 22.9 | 3.9 |
| 23.3 | 3.8 |
| 25.1 | 3.5 |
| 25.8 | 3.5 |
| 26.7 | 3.3 |
| 28.3 | 3.2 |
| 29.4 | 3.0 |

In some embodiments, Compound 1 crystalline form Type B is characterized by a thermogravimetric analysis (TGA) thermogram with a weight loss of about 1.8% up to 100° C., and/or a thermogravimetric analysis (TGA) thermogram with a weight loss of about 2.3% up to 120° C. In some embodiments, Compound 1 crystalline form Type B is characterized by a differential scanning calorimetry (DSC) endotherm having an onset temperature of about 138.2-139.2° C. In some embodiments, Compound 1 crystalline form Type B is characterized by a dynamic vapor sorption (DVS) of about 2.9% water uptake by weight up to 60% relative humidity, and a dynamic vapor sorption (DVS) of about 0.4% water uptake by weight from 60% to 80% relative humidity.

### Compound 1 Crystalline Form Type C

A novel Compound 1 crystalline form Type C can be identified by an X-ray Powder Diffraction (XRPD) pattern having one or more characteristic diffractions at angles (2 theta±0.2) of 4.55, 18.85, 23.02, and 24.65. A novel Compound 1 crystalline form Type C can be identified by an X-ray Powder Diffraction (XRPD) pattern having one or more characteristic diffractions at angles (2 theta±0.2) of 4.5, 18.9, 23.0, and 24.7. In some embodiments, Compound 1 crystalline form Type C can be identified by X-ray Powder Diffraction (XRPD), having one or more characteristic diffractions at angles (2 theta±0.2) of 4.55, 18.85, 23.02, and 24.65, corresponding to d-spacing (angstroms±0.2) of 19.43, 4.71, 3.86, and 3.61, respectively. In some embodiments, Compound 1 crystalline form Type C can be identified by X-ray Powder Diffraction (XRPD), having one or more characteristic diffractions at angles (2 theta±0.2) of 4.5, 18.9, 23.0, and 24.7, corresponding to d-spacing (angstroms±0.2) of 19.4, 4.7, 3.9, and 3.6, respectively.

In some embodiments, Compound 1 crystalline form Type C can be identified by an XRPD pattern having one or more characteristic diffractions at angles (2 theta±0.2) of 4.5, 7.3, 11.2, 18.9, 23.0, and 24.7. In some embodiments, Compound 1 crystalline form Type C can be identified by XRPD, having one or more characteristic diffractions at angles (2 theta±0.2) of 4.5, 7.3, 11.2, 18.9, 23.0, and 24.7, corresponding to d-spacing (angstroms±0.2) of 19.4, 12.0, 7.9, 4.7, 3.9, and 3.6, respectively.

In some embodiments, Compound 1 crystalline form Type C can be identified by an XRPD pattern having one or more characteristic diffractions at angles (2 theta±0.2) of 4.55, 7.34, 9.07, 11.17, 18.34, 18.85, 19.57, 21.66, 23.02, and 24.65. In some embodiments, Compound 1 crystalline form Type C can be identified by an XRPD pattern having one or more characteristic diffractions at angles (2 theta±0.2) of 4.5, 7.3, 9.1, 11.2, 18.34, 18.9, 19.6, 21.7, 23.0, and 24.7. In some embodiments, Compound 1 crystalline form Type C can be identified by XRPD, having one or more characteristic diffractions at angles (2 theta±0.2) of 4.55, 7.34, 9.07, 11.17, 18.34, 18.85, 19.57, 21.66, 23.02, and 24.65, corresponding to d-spacing (angstroms±0.2) of 19.43, 12.05, 9.75, 7.92, 4.84, 4.71, 4.54, 4.10, 3.86, and 3.61, respectively. In some embodiments, Compound 1 crystalline form Type C can be identified by XRPD, having one or more characteristic diffractions at angles (2 theta±0.2) of 4.5, 7.3, 9.1, 11.2, 18.3, 18.9, 19.6, 21.7, 23.0, and 24.7, corresponding to d-spacing (angstroms±0.2) of 19.4, 12.0, 9.8, 7.9, 4.8, 4.7, 4.5, 4.1, 3.9, and 3.6, respectively.

In some embodiments, Compound 1 crystalline form Type C is characterized by an X-ray Power Diffraction having one or more characteristic diffractions at angles (2 theta±0.2) of:
4.55
7.34
9.07
11.17
12.29
14.51
15.66
18.34
18.85
19.57
20.38
21.66
23.02
24.65
26.39
28.28
30.09
32.31
33.91
37.19

In some embodiments, Compound 1 crystalline form Type C is characterized by an X-ray Power Diffraction having one or more characteristic diffractions at angles (2 theta±0.2) of:
4.5
7.3
9.1
11.2
12.3
14.5
15.7
18.3
18.9
19.6
20.4
21.7
23.0
24.7
26.4
28.3
30.1
32.3
33.9
37.2

In some embodiments, Compound 1 crystalline form Type C is characterized by an X-ray Power Diffraction pattern having one or more characteristic diffractions at angles (2 theta±0.2) and corresponding d-spacing (angstroms±0.2) of:

| 2 theta | d-spacing |
|---|---|
| 4.55 | 19.43 |
| 7.34 | 12.05 |
| 9.07 | 9.75 |
| 11.17 | 7.92 |
| 12.29 | 7.20 |
| 14.51 | 6.11 |
| 15.66 | 5.66 |
| 18.34 | 4.84 |
| 18.85 | 4.71 |
| 19.57 | 4.54 |
| 20.38 | 4.36 |
| 21.66 | 4.10 |
| 23.02 | 3.86 |
| 24.65 | 3.61 |
| 26.39 | 3.38 |
| 28.28 | 3.16 |
| 30.09 | 2.97 |
| 32.31 | 2.77 |
| 33.91 | 2.64 |
| 37.19 | 2.42 |

In some embodiments, Compound 1 crystalline form Type C is characterized by an X-ray Power Diffraction pattern having one or more characteristic diffractions at angles (2 theta 0.2) and corresponding d-spacing (angstroms±0.2) of:

| 2 theta | d-spacing |
|---|---|
| 4.5 | 19.4 |
| 7.3 | 12.0 |
| 9.1 | 9.8 |
| 11.2 | 7.9 |
| 12.3 | 7.2 |
| 14.5 | 6.1 |
| 15.7 | 5.7 |
| 18.3 | 4.8 |
| 18.9 | 4.7 |
| 19.6 | 4.5 |
| 20.4 | 4.4 |
| 21.7 | 4.1 |
| 23.0 | 3.9 |
| 24.7 | 3.6 |
| 26.4 | 3.4 |
| 28.3 | 3.2 |
| 30.1 | 3.0 |
| 32.3 | 2.8 |
| 33.9 | 2.6 |
| 37.2 | 2.4 |

In some embodiments, Compound 1 crystalline form Type C is characterized by a thermogravimetric analysis (TGA) thermogram with a weight loss of about 1.0% up to 100° C., and/or a thermogravimetric analysis (TGA) thermogram with a weight loss of about 2.3% up to 130° C. In some embodiments, Compound 1 crystalline form Type C is characterized by a differential scanning calorimetry (DSC) endotherm having an onset temperature of about 152.2-154.2° C. In some embodiments, Compound 1 crystalline form Type C is characterized by a dynamic vapor sorption (DVS) of about 1.8% water uptake by weight up to 60% relative humidity, and a dynamic vapor sorption (DVS) of about 0.5% water uptake by weight from 60% to 80% relative humidity.

### Compound 1 Crystalline Form Type D

A novel Compound 1 crystalline form Type D can be identified by an X-ray Powder Diffraction (XRPD) pattern having one or more characteristic diffractions at angles (2 theta±0.2) of 9.72, 13.08, 15.74, 21.90, and 23.59. A novel Compound 1 crystalline form Type D can be identified by an X-ray Powder Diffraction (XRPD) pattern having one or more characteristic diffractions at angles (2 theta±0.2) of 9.7, 13.1, 15.7, 21.9, and 23.6. In some embodiments, Compound 1 crystalline form Type D can be identified by X-ray Powder Diffraction (XRPD), having one or more characteristic diffractions at angles (2 theta±0.2) of 9.72, 13.08, 15.74, 21.90, and 23.59, corresponding to d-spacing (angstroms±0.2) of 9.10, 6.77, 5.63, 4.06 and 3.77, respectively. In some embodiments, Compound 1 crystalline form Type D can be identified by X-ray Powder Diffraction (XRPD), having one or more characteristic diffractions at angles (2 theta±0.2) of 9.7, 13.1, 15.7, 21.9, and 23.6, corresponding to d-spacing (angstroms±0.2) of 9.1, 6.8, 5.6, 4.1 and 3.8, respectively.

In some embodiments, Compound 1 crystalline form Type D can be identified by an XRPD pattern having one or more characteristic diffractions at angles (2 theta±0.2) of 6.2, 9.7, 13.1, 15.7, 21.9, and 23.6 and not having a diffraction at an angle (2 theta±0.2) of 23.3. In some embodiments, Compound 1 crystalline form Type D can be identified by XRPD, having one or more characteristic diffractions at angles (2 theta±0.2) of 6.2, 9.7, 13.1, 15.7, 21.9, and 23.6, corresponding to d-spacing (angstroms±0.2) of 14.4, 9.1, 6.8, 5.6, 4.1 and 3.8, respectively, and not having a diffraction at an angle (2 theta±0.2) of 23.3.

In some embodiments, Compound 1 crystalline form Type D can be identified by an XRPD pattern having one or more characteristic diffractions at angles (2 theta±0.2) of 4.27, 6.15, 8.71, 9.72, 12.31, 13.08, 13.76, 15.74, 18.02, 21.90, 23.59, and 26.71. In some embodiments, Compound 1 crystalline form Type D can be identified by an XRPD pattern having one or more characteristic diffractions at angles (2 theta±0.2) of 4.3, 6.2, 8.7, 9.7, 12.3, 13.1, 13.8, 15.7, 18.0, 21.9, 23.6, and 26.7. In some embodiments, Compound 1 crystalline form Type D can be identified by XRPD, having one or more characteristic diffractions at angles (2 theta±0.2) of 4.27, 6.15, 8.71, 9.72, 12.31, 13.08, 13.76, 15.74, 18.02, 21.90, 23.59, and 26.71, corresponding to d-spacing (angstroms±0.2) of 20.68, 14.36, 10.16, 9.10, 7.19, 6.77, 6.44, 5.63, 4.92, 4.06, 3.77, and 3.34, respectively. In some embodiments, Compound 1 crystalline form Type D can be identified by XRPD, having one or more characteristic diffractions at angles (2 theta±0.2) of 4.3, 6.2, 8.7, 9.7, 12.3, 13.1, 13.8, 15.7, 18.0, 21.9, 23.6, and 26.7, corresponding to d-spacing (angstroms 0.2) of 20.7, 14.4, 10.2, 9.1, 7.2, 6.8, 6.4, 5.6, 4.9, 4.1, 3.8, and 3.3, respectively.

In some embodiments, Compound 1 crystalline form Type D is characterized by an X-ray Power Diffraction having one or more characteristic diffractions at angles (2 theta±0.2) of:
4.27
6.15
8.71
9.72
12.31
13.08
13.76
15.74
18.02
19.55
21.90
23.59
24.79
26.71
29.50
30.82
31.74
35.40
37.84
38.61

In some embodiments, Compound 1 crystalline form Type D is characterized by an X-ray Power Diffraction having one or more characteristic diffractions at angles (2 theta±0.2) of:
4.3
6.2
8.7
9.7
12.3
13.1
13.8
15.7
18.0
19.5
21.9
23.6
24.8
26.7
29.5
30.8
31.7
35.4
37.8
38.6

In some embodiments, Compound 1 crystalline form Type D is characterized by an X-ray Power Diffraction pattern having one or more characteristic diffractions at angles (2 theta±0.2) and corresponding d-spacing (angstroms±0.2) of:

| 2 theta | d-spacing |
|---|---|
| 4.27 | 20.68 |
| 6.15 | 14.36 |
| 8.71 | 10.16 |
| 9.72 | 9.10 |
| 12.31 | 7.19 |
| 13.08 | 6.77 |
| 13.76 | 6.44 |
| 15.74 | 5.63 |
| 18.02 | 4.92 |
| 19.55 | 4.54 |
| 21.90 | 4.06 |
| 23.59 | 3.77 |
| 24.79 | 3.59 |
| 26.71 | 3.34 |
| 29.50 | 3.03 |
| 30.82 | 2.90 |
| 31.74 | 2.82 |
| 35.40 | 2.54 |
| 37.84 | 2.38 |
| 38.61 | 2.33 |

In some embodiments, Compound 1 crystalline form Type D is characterized by an X-ray Power Diffraction pattern having one or more characteristic diffractions at angles (2 theta±0.2) and corresponding d-spacing (angstroms±0.2) of:

| 2 theta | d-spacing |
|---|---|
| 4.3 | 20.7 |
| 6.2 | 14.4 |
| 8.7 | 10.2 |
| 9.7 | 9.1 |
| 12.3 | 7.2 |
| 13.1 | 6.8 |
| 13.8 | 6.4 |
| 15.7 | 5.6 |
| 18.0 | 4.9 |
| 19.5 | 4.5 |
| 21.9 | 4.1 |
| 23.6 | 3.8 |
| 24.8 | 3.6 |
| 26.7 | 3.3 |
| 29.5 | 3.0 |
| 30.8 | 2.9 |
| 31.7 | 2.8 |
| 35.4 | 2.5 |
| 37.8 | 2.4 |
| 38.6 | 2.3 |

In some embodiments, Compound 1 crystalline form Type D is characterized by a thermogravimetric analysis (TGA) thermogram with a weight loss of about 9.6% up to 130° C. In some embodiments, Compound 1 crystalline form Type D is characterized by a differential scanning calorimetry (DSC) endotherm having an onset temperature of about 91.9° C.

### Compound 1 Crystalline Form Type E

A novel Compound 1 crystalline form Type E can be identified by an X-ray Powder Diffraction (XRPD) pattern having one or more characteristic diffractions at angles (2 theta±0.2) of 15.12, 15.75, 17.48, 20.05, 21.93, and 26.72. A novel Compound 1 crystalline form Type E can be identified by an X-ray Powder Diffraction (XRPD) pattern having one or more characteristic diffractions at angles (2 theta±0.2) of 15.1, 15.8, 17.5, 20.1, 21.9, and 26.7. In some embodiments, Compound 1 crystalline form Type E can be identified by X-ray Powder Diffraction (XRPD), having one or more characteristic diffractions at angles (2 theta±0.2) of 15.12, 15.75, 17.48, 20.05, 21.93, and 26.72, corresponding to d-spacing (angstroms±0.2) of 5.86, 5.63, 5.07, 4.43, 4.05, and 3.34, respectively. In some embodiments, Compound 1 crystalline form Type E can be identified by X-ray Powder Diffraction (XRPD), having one or more characteristic diffractions at angles (2 theta±0.2) of 15.1, 15.8, 17.5, 20.1, 21.9, and 26.7, corresponding to d-spacing (angstroms±0.2) of 5.9, 5.6, 5.1, 4.4, 4.1, and 3.3, respectively.

In some embodiments, Compound 1 crystalline form Type E can be identified by an XRPD pattern having one or more characteristic diffractions at angles (2 theta±0.2) of 15.1, 15.8, 17.5, 20.1, 21.9, and 26.7. In some embodiments, Compound 1 crystalline form Type E can be identified by XRPD, having one or more characteristic diffractions at angles (2 theta±0.2) of 15.1, 15.8, 17.5, 19.0, 20.1, 21.9, and 26.7, corresponding to d-spacing (angstroms±0.2) of 5.9, 5.6, 5.1, 4.7, 4.4, 4.1, and 3.3, respectively.

In some embodiments, Compound 1 crystalline form Type E can be identified by an XRPD pattern having one or more characteristic diffractions at angles (2 theta±0.2) of 4.59, 15.12, 15.75, 17.48, 20.05, 21.93, 23.18, 23.70, and 26.72. In some embodiments, Compound 1 crystalline form Type E can be identified by an XRPD pattern having one or more characteristic diffractions at angles (2 theta±0.2) of 4.6, 15.1, 15.8, 17.5, 20.1, 21.9, 23.2, 23.7, and 26.7. In some embodiments, Compound 1 crystalline form Type E can be identified by XRPD, having one or more characteristic diffractions at angles (2 theta±0.2) of 4.59, 15.12, 15.75, 17.48, 20.05, 21.93, 23.18, 23.70, and 26.72, corresponding to d-spacing (angstroms±0.2) of 19.27, 5.86, 5.63, 5.07, 4.43, 4.05, 3.84, 3.75, and 3.34, respectively. In some embodiments, Compound 1 crystalline form Type E can be identified by XRPD, having one or more characteristic diffractions at angles (2 theta±0.2) of 4.6, 15.1, 15.8, 17.5, 20.1, 21.9, 23.2, 23.7, and 26.7, corresponding to d-spacing (angstroms±0.2) of 19.3, 5.9, 5.6, 5.1, 4.4, 4.1, 3.8, 3.8, and 3.3, respectively.

In some embodiments, Compound 1 crystalline form Type E can be identified by an XRPD pattern having one or more characteristic diffractions at angles (2 theta±0.2) of 4.59, 9.76, 12.36, 13.12, 15.12, 15.75, 16.84, 17.48, 18.06, 19.02, 20.05, 21.93, 23.18, 23.70, 26.72, and 27.81. In some embodiments, Compound 1 crystalline form Type E can be identified by an XRPD pattern having one or more characteristic diffractions at angles (2 theta±0.2) of 4.6, 9.8, 12.4, 13.1, 15.1, 15.8, 16.8, 17.5, 18.1, 19.0, 20.1, 21.9, 23.2, 23.7, 26.7, and 27.8. In some embodiments, Compound 1 crystalline form Type E can be identified by XRPD, having one or more characteristic diffractions at angles (2 theta±0.2) of 4.59, 9.76, 12.36, 13.12, 15.12, 15.75, 16.84, 17.48, 18.06, 19.02, 20.05, 21.93, 23.18, 23.70, 26.72, and 27.81, corresponding to d-spacing (angstroms±0.2) of 19.27, 9.06, 7.16, 6.75, 5.86, 5.63, 5.27, 5.07, 4.91, 4.67, 4.43, 4.05, 3.84, 3.75, 3.34, and 3.21, respectively. In some embodiments, Compound 1 crystalline form Type E can be identified by XRPD, having one or more characteristic diffractions at angles (2 theta±0.2) of 4.6, 9.8, 12.4, 13.1, 15.1, 15.8, 16.8, 17.5, 18.1, 19.0, 20.1, 21.9, 23.2, 23.7, 26.7, and 27.8, corresponding to d-spacing (angstroms±0.2) of 19.3, 9.1, 7.2, 6.7, 5.9, 5.6, 5.3, 5.1, 4.9, 4.7, 4.4, 4.1, 3.8, 3.8, 3.3, and 3.2, respectively.

In some embodiments, Compound 1 crystalline form Type E is characterized by an X-ray Power Diffraction having one or more characteristic diffractions at angles (2 theta±0.2) of:
4.59
8.76
9.76
12.36
13.12
13.83
15.12
15.75
16.84
17.48
18.06
19.02
20.05
21.93
23.18
23.70
24.82
26.72
27.81
29.51
30.76
31.74
33.03
34.52
35.39
36.72
37.77
38.66

In some embodiments, Compound 1 crystalline form Type E is characterized by an X-ray Power Diffraction having one or more characteristic diffractions at angles (2 theta±0.2) of:
4.6
8.8
9.8
12.4
13.1
13.8
15.1
15.8
16.8
17.5
18.1
19.0
20.1
21.9
23.2
23.7
24.8
26.7
27.8
29.5
30.8
31.7
33.0
34.5
35.4
36.7
37.8
38.7

In some embodiments, Compound 1 crystalline form Type E is characterized by an X-ray Power Diffraction pattern having one or more characteristic diffractions at angles (2 theta 0.2) and corresponding d-spacing (angstroms±0.2) of:

| 2 theta | d-spacing |
|---|---|
| 4.59 | 19.27 |
| 8.76 | 10.09 |
| 9.76 | 9.06 |
| 12.36 | 7.16 |
| 13.12 | 6.75 |
| 13.83 | 6.40 |
| 15.12 | 5.86 |
| 15.75 | 5.63 |
| 16.84 | 5.27 |
| 17.48 | 5.07 |
| 18.06 | 4.91 |
| 19.02 | 4.67 |
| 20.05 | 4.43 |
| 21.93 | 4.05 |
| 23.18 | 3.84 |
| 23.70 | 3.75 |
| 24.82 | 3.59 |
| 26.72 | 3.34 |
| 27.81 | 3.21 |
| 29.51 | 3.03 |
| 30.76 | 2.91 |
| 31.74 | 2.82 |
| 33.03 | 2.71 |
| 34.52 | 2.60 |
| 35.39 | 2.54 |
| 36.72 | 2.45 |
| 37.77 | 2.38 |
| 38.66 | 2.33 |

In some embodiments, Compound 1 crystalline form Type E is characterized by an X-ray Power Diffraction pattern having one or more characteristic diffractions at angles (2 theta 0.2) and corresponding d-spacing (angstroms±0.2) of:

| 2 theta | d-spacing |
|---|---|
| 4.6 | 19.3 |
| 8.8 | 10.1 |
| 9.8 | 9.1 |
| 12.4 | 7.2 |
| 13.1 | 6.7 |
| 13.8 | 6.4 |
| 15.1 | 5.9 |
| 15.8 | 5.6 |
| 16.8 | 5.3 |
| 17.5 | 5.1 |
| 18.1 | 4.9 |
| 19.0 | 4.7 |
| 20.1 | 4.4 |
| 21.9 | 4.1 |
| 23.2 | 3.8 |
| 23.7 | 3.8 |
| 24.8 | 3.6 |
| 26.7 | 3.3 |
| 27.8 | 3.2 |
| 29.5 | 3.0 |
| 30.8 | 2.9 |
| 31.7 | 2.8 |
| 33.0 | 2.7 |
| 34.5 | 2.6 |
| 35.4 | 2.5 |
| 36.7 | 2.4 |
| 37.8 | 2.4 |
| 38.7 | 2.3 |

### Compound 1 Crystalline Form Type F

A novel Compound 1 crystalline form Type F can be identified by an X-ray Powder Diffraction (XRPD) pattern having one or more characteristic diffractions at angles (2 theta±0.2) of 5.45, 14.66, 16.00, 16.79, 20.01, 21.36, and 22.45. A novel Compound 1 crystalline form Type F can be identified by an X-ray Powder Diffraction (XRPD) pattern having one or more characteristic diffractions at angles (2 theta±0.2) of 5.4, 14.7, 16.0, 16.8, 20.0, 21.4, and 22.5. In some embodiments, Compound 1 crystalline form Type F can be identified by X-ray Powder Diffraction (XRPD), having one or more characteristic diffractions at angles (2 theta±0.2) of 5.45, 14.66, 16.00, 16.79, 20.01, 21.36, and 22.45, corresponding to d-spacing (angstroms±0.2) of 16.23, 6.04, 5.54, 5.28, 4.44, 4.16, and 3.96, respectively. In some embodiments, Compound 1 crystalline form Type F can be identified by X-ray Powder Diffraction (XRPD), having one or more characteristic diffractions at angles (2 theta±0.2) of 5.4, 14.7, 16.0, 16.8, 20.0, 21.4, and 22.5, corresponding to d-spacing (angstroms±0.2) of 16.2, 6.0, 5.5, 5.3, 4.4, 4.2, and 4.0, respectively.

In some embodiments, Compound 1 crystalline form Type F can be identified by an XRPD pattern having one or more characteristic diffractions at angles (2 theta±0.2) of 5.4, 14.7, 16.0, 16.8, and 21.4. In some embodiments, Compound 1 crystalline form Type F can be identified by XRPD, having one or more characteristic diffractions at angles (2 theta±0.2) of 5.4, 14.7, 16.0, 16.8, and 21.4, corresponding to d-spacing (angstroms±0.2) of 16.2, 6.0, 5.5, 5.3, and 4.2, respectively.

In some embodiments, Compound 1 crystalline form Type F can be identified by an XRPD pattern having one or more characteristic diffractions at angles (2 theta±0.2) of 5.45, 14.66, 16.00, 16.79, 18.99, 20.01, 21.36, 22.45, 23.25, and 25.32. In some embodiments, Compound 1 crystalline form Type F can be identified by an XRPD pattern having one or more characteristic diffractions at angles (2 theta±0.2) of 5.4, 14.7, 16.0, 16.8, 19.0, 20.0, 21.4, 22.5, 23.2, and 25.3. In some embodiments, Compound 1 crystalline form Type F can be identified by XRPD, having one or more characteristic diffractions at angles (2 theta±0.2) of 5.45, 14.66, 16.00, 16.79, 18.99, 20.01, 21.36, 22.45, 23.25, and 25.32, corresponding to d-spacing (angstroms±0.2) of 16.23, 6.04, 5.54, 5.28, 4.67, 4.44, 4.16, 3.96, 3.83, and 3.52, respectively. In some embodiments, Compound 1 crystalline form Type F can be identified by XRPD, having one or more characteristic diffractions at angles (2 theta±0.2) of 5.4, 14.7, 16.0, 16.8, 19.0, 20.0, 21.4, 22.5, 23.2, and 25.3, corresponding to d-spacing (angstroms±0.2) of 16.2, 6.0, 5.5, 5.3, 4.7, 4.4, 4.2, 4.0, 3.8, and 3.5, respectively.

In some embodiments, Compound 1 crystalline form Type F can be identified by an XRPD pattern having one or more characteristic diffractions at angles (2 theta±0.2) of 5.45, 12.87, 14.66, 16.00, 16.79, 17.36, 18.99, 20.01, 20.57, 21.36, 22.45, 23.25, 25.32, 26.57, 27.25, 27.97, and 30.02. In some embodiments, Compound 1 crystalline form Type F can be identified by an XRPD pattern having one or more characteristic diffractions at angles (2 theta±0.2) of 5.4, 12.9, 14.7, 16.0, 16.8, 17.4, 19.0, 20.0, 20.6, 21.4, 22.5, 23.2, 25.3, 26.6, 27.2, 28.0, and 30.0. In some embodiments, Compound 1 crystalline form Type F can be identified by XRPD, having one or more characteristic diffractions at angles (2 theta±0.2) of 5.45, 12.87, 14.66, 16.00, 16.79, 17.36, 18.99, 20.01, 20.57, 21.36, 22.45, 23.25, 25.32, 26.57, 27.25, 27.97, and 30.02, corresponding to d-spacing (angstroms±0.2) of 16.23, 6.88, 6.04, 5.54, 5.28, 5.11, 4.67, 4.44, 4.32, 4.16, 3.96, 3.83, 3.52, 3.35, 3.27, 3.19, and 2.98, respectively. In some embodiments, Compound 1 crystalline form Type F can be identified by XRPD, having one or more characteristic diffractions at angles (2 theta±0.2) of 5.4, 12.9, 14.7, 16.0, 16.8, 17.4, 19.0, 20.0, 20.6, 21.4, 22.5, 23.2, 25.3, 26.6, 27.2, 28.0, and 30.0, corresponding to d-spacing (angstroms±0.2) of 16.2, 6.9, 6.0, 5.5, 5.3, 5.1, 4.7, 4.4, 4.3, 4.2, 4.0, 3.8, 3.5, 3.4, 3.3, 3.2, and 3.0, respectively.

In some embodiments, Compound 1 crystalline form Type F is characterized by an X-ray Power Diffraction having one or more characteristic diffractions at angles (2 theta±0.2) of:
5.45
10.92
12.87
14.66
16.00
16.79
17.36
18.99
20.01
20.57
21.36
22.45
23.25
25.32
26.57
27.25
27.97
30.02
31.98
32.89
38.29
39.09

In some embodiments, Compound 1 crystalline form Type F is characterized by an X-ray Power Diffraction having one or more characteristic diffractions at angles (2 theta±0.2) of:
5.4
10.9
12.9
14.7
16.0
16.8
17.4
19.0
20.0
20.6
21.4
22.5
23.2
25.3
26.6
27.2
28.0
30.0
32.0
32.9
38.3
39.1

In some embodiments, Compound 1 crystalline form Type F is characterized by an X-ray Power Diffraction pattern having one or more characteristic diffractions at angles (2 theta±0.2) and corresponding d-spacing (angstroms±0.2) of:

| 2 theta | d-spacing |
|---|---|
| 5.45 | 16.23 |
| 10.92 | 8.10 |
| 12.87 | 6.88 |
| 14.66 | 6.04 |
| 16.00 | 5.54 |
| 16.79 | 5.28 |
| 17.36 | 5.11 |
| 18.99 | 4.67 |
| 20.01 | 4.44 |
| 20.57 | 4.32 |
| 21.36 | 4.16 |
| 22.45 | 3.96 |
| 23.25 | 3.83 |
| 25.32 | 3.52 |
| 26.57 | 3.35 |
| 27.25 | 3.27 |
| 27.97 | 3.19 |
| 30.02 | 2.98 |
| 31.98 | 2.80 |
| 32.89 | 2.72 |
| 38.29 | 2.35 |
| 39.09 | 2.30 |

In some embodiments, Compound 1 crystalline form Type F is characterized by an X-ray Power Diffraction pattern having one or more characteristic diffractions at angles (2 theta±0.2) and corresponding d-spacing (angstroms±0.2) of:

| 2 theta | d-spacing |
|---|---|
| 5.4 | 16.2 |
| 10.9 | 8.1 |
| 12.9 | 6.9 |
| 14.7 | 6.0 |
| 16.0 | 5.5 |
| 16.8 | 5.3 |
| 17.4 | 5.1 |
| 19.0 | 4.7 |
| 20.0 | 4.4 |
| 20.6 | 4.3 |
| 21.4 | 4.2 |
| 22.5 | 4.0 |
| 23.2 | 3.8 |
| 25.3 | 3.5 |
| 26.6 | 3.4 |
| 27.2 | 3.3 |
| 28.0 | 3.2 |
| 30.0 | 3.0 |
| 32.0 | 2.8 |
| 32.9 | 2.7 |
| 38.3 | 2.4 |
| 39.1 | 2.3 |

In some embodiments, Compound 1 crystalline form Type F is characterized by a thermogravimetric analysis (TGA) thermogram with a weight loss of about 6.2% up to 120° C. In some embodiments, Compound 1 crystalline form Type F is characterized by a differential scanning calorimetry (DSC) endotherm having a peak temperature of about 100.4° C. and an onset temperature of 125.9° C.

### Compound 1 Crystalline Form Type G

A novel Compound 1 crystalline form Type G can be identified by an X-ray Powder Diffraction (XRPD) pattern having one or more characteristic diffractions at angles (2 theta±0.2) of 5.36, 14.34, 16.58, and 21.35. A novel Compound 1 crystalline form Type G can be identified by an X-ray Powder Diffraction (XRPD) pattern having one or more characteristic diffractions at angles (2 theta±0.2) of 5.4, 14.3, 16.6, and 21.4. In some embodiments, Compound 1 crystalline form Type G can be identified by X-ray Powder Diffraction (XRPD), having one or more characteristic diffractions at angles (2 theta±0.2) of 5.36, 14.34, 16.58, and 21.35, corresponding to d-spacing (angstroms±0.2) of 16.48, 6.18, 5.35, and 4.16, respectively. In some embodiments, Compound 1 crystalline form Type G can be identified by X-ray Powder Diffraction (XRPD), having one or more characteristic diffractions at angles (2 theta±0.2) of 5.4, 14.3, 16.6, and 21.4, corresponding to d-spacing (angstroms±0.2) of 16.5, 6.2, 5.3, and 4.2, respectively.

In some embodiments, Compound 1 crystalline form Type G can be identified by an XRPD pattern having one or more characteristic diffractions at angles (2 theta±0.2) of 5.4, 14.3, 16.6, 21.3, and 22.3. In some embodiments, Compound 1 crystalline form Type G can be identified by XRPD, having one or more characteristic diffractions at angles (2 theta±0.2) of 5.4, 14.3, 16.6, 21.3, and 22.3, corresponding to d-spacing (angstroms±0.2) of 16.5, 6.2, 5.3, 4.2, and 4.0, respectively.

In some embodiments, Compound 1 crystalline form Type G can be identified by an XRPD pattern having one or more characteristic diffractions at angles (2 theta±0.2) of 5.36, 12.83, 14.34, 15.00, 16.58, 19.78, 21.35, 22.35, 25.33, and 26.43. In some embodiments, Compound 1 crystalline form Type G can be identified by an XRPD pattern having one or more characteristic diffractions at angles (2 theta±0.2) of 5.4, 12.8, 14.3, 15.0, 16.6, 19.8, 21.3, 22.3, 25.3, and 26.4. In some embodiments, Compound 1 crystalline form Type G can be identified by XRPD, having one or more characteristic diffractions at angles (2 theta±0.2) of 5.36, 12.83, 14.34, 15.00, 16.58, 19.78, 21.35, 22.35, 25.33, and 26.43, corresponding to d-spacing (angstroms±0.2) of 16.48, 6.90, 6.18, 5.91, 5.35, 4.49, 4.16, 3.98, 3.52, and 3.37, respectively. In some embodiments, Compound 1 crystalline form Type G can be identified by XRPD, having one or more characteristic diffractions at angles (2 theta±0.2) of 5.4, 12.8, 14.3, 15.0, 16.6, 19.8, 21.3, 22.3, 25.3, and 26.4, corresponding to d-spacing (angstroms±0.2) of 16.5, 6.9, 6.2, 5.9, 5.3, 4.5, 4.2, 4.0, 3.5, and 3.4, respectively.

In some embodiments, Compound 1 crystalline form Type G can be identified by an XRPD pattern having one or more characteristic diffractions at angles (2 theta±0.2) of 5.36, 12.83, 14.34, 15.00, 15.79, 16.58, 19.78, 21.35, 22.35, 25.33, 26.43, 27.35, and 30.21. In some embodiments, Compound 1 crystalline form Type G can be identified by an XRPD pattern having one or more characteristic diffractions at angles (2 theta±0.2) of 5.34 12.8, 14.3, 15.0, 15.8, 16.6, 19.8, 21.3, 22.3, 25.3, 26.4, 27.4, and 30.2. In some embodiments, Compound 1 crystalline form Type G can be identified by XRPD, having one or more characteristic diffractions at angles (2 theta±0.2) of 5.36, 12.83, 14.34, 15.00, 15.79, 16.58, 19.78, 21.35, 22.35, 25.33, 26.43, 27.35, and 30.21, corresponding to d-spacing (angstroms±0.2) of 16.48, 6.90, 6.18, 5.91, 5.61, 5.35, 4.49, 4.16, 3.98, 3.52, 3.37, 3.26, and 2.96, respectively. In some embodiments, Compound 1 crystalline form Type G can be identified by XRPD, having one or more characteristic diffractions at angles (2 theta±0.2) of 5.4, 12.8, 14.3, 15.0, 15.8, 16.6, 19.8, 21.3, 22.3, 25.3, 26.4, 27.4, and 30.2, corresponding to d-spacing (angstroms±0.2) of 16.5, 6.9, 6.2, 5.9, 5.6, 5.3, 4.5, 4.2, 4.0, 3.5, 3.4, 3.3, and 3.0, respectively.

In some embodiments, Compound 1 crystalline form Type G is characterized by an X-ray Power Diffraction having one or more characteristic diffractions at angles (2 theta±0.2) of:
5.36
8.73
12.83
14.34
15.00
15.79
16.58
18.54
19.78
21.35
22.35
23.38
25.33
26.43
27.35
30.21
32.32
38.04

In some embodiments, Compound 1 crystalline form Type G is characterized by an X-ray Power Diffraction having one or more characteristic diffractions at angles (2 theta±0.2) of:
5.4
8.7
12.8
14.3
15.0
15.8
16.6
18.5
19.8
21.3
22.3
23.4
25.3
26.4
27.4
30.2
32.3
38.0

In some embodiments, Compound 1 crystalline form Type G is characterized by an X-ray Power Diffraction pattern having one or more characteristic diffractions at angles (2 theta±0.2) and corresponding d-spacing (angstroms±0.2) of:

| 2 theta | d-spacing |
|---|---|
| 5.36 | 16.48 |
| 8.73 | 10.13 |
| 12.83 | 6.90 |
| 14.34 | 6.18 |
| 15.00 | 5.91 |
| 15.79 | 5.61 |
| 16.58 | 5.35 |
| 18.54 | 4.79 |
| 19.78 | 4.49 |
| 21.35 | 4.16 |
| 22.35 | 3.98 |
| 23.38 | 3.80 |
| 25.33 | 3.52 |
| 26.43 | 3.37 |
| 27.35 | 3.26 |
| 30.21 | 2.96 |
| 32.32 | 2.77 |
| 38.04 | 2.37 |

In some embodiments, Compound 1 crystalline form Type G is characterized by an X-ray Power Diffraction pattern having one or more characteristic diffractions at angles (2 theta±0.2) and corresponding d-spacing (angstroms±0.2) of:

| 2 theta | d-spacing |
|---|---|
| 5.4 | 16.5 |
| 8.7 | 10.1 |
| 12.8 | 6.9 |
| 14.3 | 6.2 |
| 15.0 | 5.9 |
| 15.8 | 5.6 |
| 16.6 | 5.3 |
| 18.6 | 4.8 |
| 19.8 | 4.5 |
| 21.3 | 4.2 |
| 22.3 | 4.0 |
| 23.4 | 3.8 |
| 25.3 | 3.5 |
| 26.4 | 3.4 |
| 27.4 | 3.3 |
| 30.2 | 3.0 |
| 32.3 | 2.8 |
| 38.0 | 2.4 |

### Compound 1 Crystalline Form Type H

A novel Compound 1 crystalline form Type H can be identified by an X-ray Powder Diffraction (XRPD) pattern having one or more characteristic diffractions at angles (2 theta±0.2) of 5.8, 14.7, 16.6, 20.0, 21.3, and 25.4. In some embodiments, Compound 1 crystalline form Type H can be identified by X-ray Powder Diffraction (XRPD), having one or more characteristic diffractions at angles (2 theta±0.2) of 5.8, 14.7, 16.6, 20.0, 21.3, and 25.4, corresponding to d-spacing (angstroms±0.2) of 15.3, 6.0, 5.4, 4.4, 4.2, and 3.5, respectively.

In some embodiments, Compound 1 crystalline form Type H is characterized by an X-ray Power Diffraction having one or more characteristic diffractions at angles (2 theta±0.2) of:
5.8
8.4
11.5
12.4
13.1
13.7
14.7
14.9
16.0
16.2
16.6
16.9
17.3
17.7
18.3
19.5
20.0
21.3
21.9
23.1
23.6
23.9
24.4
24.9
25.1
25.4
26.2
27.4
28.1
28.4
29.3
29.7
30.4
31.0
32.7
33.4
34.1
34.8
35.5
35.8
36.4
37.1
38.5

In some embodiments, Compound 1 crystalline form Type H is characterized by an X-ray Power Diffraction pattern having one or more characteristic diffractions at angles (2 theta±0.2) and corresponding d-spacing (angstroms±0.2) of:

| Pos. [°2Th.] | d-spacing [Å] |
|---|---|
| 5.8 | 15.3 |
| 8.4 | 10.5 |
| 11.5 | 7.7 |
| 12.4 | 7.2 |
| 13.1 | 6.8 |
| 13.7 | 6.5 |
| 14.7 | 6.0 |
| 14.9 | 5.9 |
| 16.0 | 5.6 |
| 16.2 | 5.5 |
| 16.6 | 5.4 |
| 16.9 | 5.3 |
| 17.3 | 5.1 |
| 17.7 | 5.0 |
| 18.3 | 4.8 |
| 19.5 | 4.6 |
| 20.0 | 4.4 |
| 21.3 | 4.2 |
| 21.9 | 4.1 |
| 23.1 | 3.9 |
| 23.6 | 3.8 |
| 23.9 | 3.7 |
| 24.4 | 3.7 |
| 24.9 | 3.6 |
| 25.1 | 3.5 |
| 25.4 | 3.5 |
| 26.2 | 3.4 |
| 27.4 | 3.3 |
| 28.1 | 3.2 |
| 28.4 | 3.1 |
| 29.3 | 3.0 |
| 29.7 | 3.0 |
| 30.4 | 2.9 |
| 31.0 | 2.9 |
| 32.7 | 2.7 |
| 33.4 | 2.7 |
| 34.1 | 2.6 |
| 34.8 | 2.6 |
| 35.5 | 2.5 |
| 35.8 | 2.5 |
| 36.4 | 2.5 |
| 37.1 | 2.4 |
| 38.5 | 2.3 |

### Compound 1 Crystalline Form Type I

A novel Compound 1 crystalline form Type I can be identified by an X-ray Powder Diffraction (XRPD) pattern having one or more characteristic diffractions at angles (2 theta±0.2) of 5.2, 14.6, 15.5, 20.2, and 21.1. In some embodiments, Compound 1 crystalline form Type I can be identified by X-ray Powder Diffraction (XRPD), having one or more characteristic diffractions at angles (2 theta±0.2) of 5.2, 14.6, 15.5, 20.2, and 21.1, corresponding to d-spacing (angstroms±0.2) of 17.1, 6.1, 5.7, 4.4, and 4.2, respectively.

In some embodiments, Compound 1 crystalline form Type I is characterized by an X-ray Power Diffraction having one or more characteristic diffractions at angles (2 theta±0.2) of:
5.2
8.8
10.3
12.6
14.6
15.5
16.1
16.3
16.6
17.1
17.6
18.7
18.9
20.2
20.5
20.7
21.1
21.5
22.0
22.3
23.7
24.8
25.2
26.0
26.3
26.5
26.8
27.0
27.5
27.7
28.1
29.6
30.0
30.4
31.3
32.0
32.5
33.2
34.0
34.6
36.9
38.2
38.9
39.5

In some embodiments, Compound 1 crystalline form Type I is characterized by an X-ray Power Diffraction pattern having one or more characteristic diffractions at angles (2 theta±0.2) and corresponding d-spacing (angstroms±0.2) of:

| Pos. [°2Th.] | d-spacing [Å] |
|---|---|
| 5.2 | 17.1 |
| 8.8 | 10.1 |
| 10.3 | 8.6 |
| 12.6 | 7.0 |
| 14.6 | 6.1 |
| 15.5 | 5.7 |
| 16.1 | 5.5 |
| 16.3 | 5.4 |
| 16.6 | 5.3 |
| 17.1 | 5.2 |
| 17.6 | 5.0 |
| 18.7 | 4.7 |
| 18.9 | 4.7 |
| 20.2 | 4.4 |
| 20.5 | 4.3 |
| 20.7 | 4.3 |
| 21.1 | 4.2 |
| 21.5 | 4.1 |
| 22.0 | 4.0 |
| 22.3 | 4.0 |
| 23.7 | 3.8 |
| 24.8 | 3.6 |
| 25.2 | 3.5 |
| 26.0 | 3.4 |
| 26.3 | 3.4 |
| 26.5 | 3.4 |
| 26.8 | 3.3 |
| 27.0 | 3.3 |
| 27.5 | 3.2 |
| 27.7 | 3.2 |
| 28.1 | 3.2 |
| 29.6 | 3.0 |
| 30.0 | 3.0 |
| 30.4 | 2.9 |
| 31.3 | 2.9 |
| 32.0 | 2.8 |
| 32.5 | 2.8 |
| 33.2 | 2.7 |
| 34.0 | 2.6 |
| 34.6 | 2.6 |
| 36.9 | 2.4 |
| 38.2 | 2.4 |
| 38.9 | 2.3 |
| 39.5 | 2.3 |

### Compound 1 Crystalline Form Type J

A novel Compound 1 crystalline form Type J can be identified by an X-ray Powder Diffraction (XRPD) pattern having one or more characteristic diffractions at angles (2 theta±0.2) of 4.5, 5.7, 22.8, 23.1, and 24.5. In some embodiments, Compound 1 crystalline form Type J can be identified by X-ray Powder Diffraction (XRPD), having one or more characteristic diffractions at angles (2 theta±0.2) of 4.5, 5.7, 22.8, 23.1, and 24.5, corresponding to d-spacing (angstroms 0.2) of 19.5, 15.4, 3.9, 3.8, and 3.6, respectively.

In some embodiments, Compound 1 crystalline form Type J is characterized by an X-ray Power Diffraction having one or more characteristic diffractions at angles (2 theta±0.2) of:
4.5
5.7
7.1
7.7
9.1
10.5
11.2
11.7
12.3
12.9
14.3
14.5
15.4
15.7
16.3
17.3
18.3
18.7
19.3
19.6
20.5
21.2
21.5
22.8
23.1
23.6
24.1
24.5
25.2
25.9
26.4
27.8
29.3
36.2
37.0

In some embodiments, Compound 1 crystalline form Type J is characterized by an X-ray Power Diffraction pattern having one or more characteristic diffractions at angles (2 theta±0.2) and corresponding d-spacing (angstroms±0.2) of:

| Pos. [°2Th.] | d-spacing [Å] |
|---|---|
| 4.5 | 19.5 |
| 5.7 | 15.4 |
| 7.1 | 12.7 |
| 7.7 | 11.5 |
| 9.1 | 9.7 |
| 10.5 | 8.4 |
| 11.2 | 7.9 |
| 11.7 | 7.5 |
| 12.3 | 7.2 |
| 12.9 | 6.8 |
| 14.3 | 6.2 |
| 14.5 | 6.1 |
| 15.4 | 5.8 |
| 15.7 | 5.7 |
| 16.3 | 5.4 |
| 17.3 | 5.1 |
| 18.3 | 4.9 |
| 18.7 | 4.7 |
| 19.3 | 4.6 |
| 19.6 | 4.5 |
| 20.5 | 4.3 |
| 21.2 | 4.2 |
| 21.5 | 4.1 |
| 22.8 | 3.9 |
| 23.1 | 3.8 |
| 23.6 | 3.8 |
| 24.1 | 3.7 |
| 24.5 | 3.6 |
| 25.2 | 3.5 |
| 25.9 | 3.4 |
| 26.4 | 3.4 |
| 27.8 | 3.2 |
| 29.3 | 3.0 |
| 36.2 | 2.5 |
| 37.0 | 2.4 |

### Compound 1 Crystalline Form Type K

A novel Compound 1 crystalline form Type K can be identified by an X-ray Powder Diffraction (XRPD) pattern having one or more characteristic diffractions at angles (2 theta±0.2) of 4.6, 15.4, 15.6, 16.1, 23.2, and 27.4. In some embodiments, Compound 1 crystalline form Type K can be identified by X-ray Powder Diffraction (XRPD), having one or more characteristic diffractions at angles (2 theta±0.2) of 4.6, 15.4, 15.6, 16.1, 23.2, and 27.4, corresponding to d-spacing (angstroms±0.2) of 19.2, 5.7, 5.7, 5.5, 3.8, and 3.3, respectively.

In some embodiments, Compound 1 crystalline form Type K is characterized by an X-ray Power Diffraction having one or more characteristic diffractions at angles (2 theta±0.2) of:
4.6
9.3
10.1
12.9
13.9
14.7
15.4
15.6
16.1
17.8
18.3
18.6
19.3
20.0
20.7
21.6
21.9
22.9
23.2
24.4
25.0
25.5
26.0
27.4
28.8
29.2
30.7
31.1
32.7
36.3

In some embodiments, Compound 1 crystalline form Type K is characterized by an X-ray Power Diffraction pattern having one or more characteristic diffractions at angles (2 theta±0.2) and corresponding d-spacing (angstroms±0.2) of:

| Pos. [°2Th.] | d-spacing [Å] |
|---|---|
| 4.6 | 19.2 |
| 9.3 | 9.5 |
| 10.1 | 8.7 |
| 12.9 | 6.8 |
| 13.9 | 6.4 |
| 14.7 | 6.0 |
| 15.4 | 5.7 |
| 15.6 | 5.7 |
| 16.1 | 5.5 |
| 17.8 | 5.0 |
| 18.3 | 4.9 |
| 18.6 | 4.8 |
| 19.3 | 4.6 |
| 20.0 | 4.4 |
| 20.7 | 4.3 |
| 21.6 | 4.1 |
| 21.9 | 4.1 |
| 22.9 | 3.9 |
| 23.2 | 3.8 |
| 24.4 | 3.6 |
| 25.0 | 3.6 |
| 25.5 | 3.5 |
| 26.0 | 3.4 |
| 27.4 | 3.3 |
| 28.8 | 3.1 |
| 29.2 | 3.1 |
| 30.7 | 2.9 |
| 31.1 | 2.9 |
| 32.7 | 2.7 |
| 36.3 | 2.5 |

### Compound 1 Crystalline Form Type L

A novel Compound 1 crystalline form Type L can be identified by an X-ray Powder Diffraction (XRPD) pattern having one or more characteristic diffractions at angles (2 theta±0.2) of 5.9, 11.9, 17.8, 21.6, 23.9, and 36.1. In some embodiments, Compound 1 crystalline form Type L can be identified by X-ray Powder Diffraction (XRPD), having one or more characteristic diffractions at angles (2 theta±0.2) of 5.9, 11.9, 17.8, 21.6, 23.9, and 36.1, corresponding to d-spacing (angstroms±0.2) of 14.9, 7.5, 5.0, 4.1, 3.7, and 2.5, respectively.

In some embodiments, Compound 1 crystalline form Type L is characterized by an X-ray Power Diffraction having one or more characteristic diffractions at angles (2 theta±0.2) of:
5.9
8.4
11.9
13.3
14.7
15.0
16.2
16.7
16.9
17.8
18.9
20.4
21.2
21.6
22.2
23.9
24.6
25.5
25.7
26.1
26.8
28.1
28.8
29.9
30.6
31.9
32.4
33.6
34.2
35.6
36.1
38.2

In some embodiments, Compound 1 crystalline form Type L is characterized by an X-ray Power Diffraction pattern having one or more characteristic diffractions at angles (2 theta±0.2) and corresponding d-spacing (angstroms±0.2) of:

| Pos. [°2Th.] | d-spacing [Å] |
|---|---|
| 5.9 | 14.9 |
| 8.4 | 10.5 |
| 11.9 | 7.5 |
| 13.3 | 6.6 |
| 14.7 | 6.0 |
| 15.0 | 5.9 |
| 16.2 | 5.5 |
| 16.7 | 5.3 |
| 16.9 | 5.2 |
| 17.8 | 5.0 |
| 18.9 | 4.7 |
| 20.4 | 4.4 |
| 21.2 | 4.2 |
| 21.6 | 4.1 |
| 22.2 | 4.0 |
| 23.9 | 3.7 |
| 24.6 | 3.6 |
| 25.5 | 3.5 |
| 25.7 | 3.5 |
| 26.1 | 3.4 |
| 26.8 | 3.3 |
| 28.1 | 3.2 |
| 28.8 | 3.1 |
| 29.9 | 3.0 |
| 30.6 | 2.9 |
| 31.9 | 2.8 |
| 32.4 | 2.8 |
| 33.6 | 2.7 |
| 34.2 | 2.6 |
| 35.6 | 2.5 |
| 36.1 | 2.5 |
| 38.2 | 2.4 |

### Compound 1 Crystalline Form Type M

A novel Compound 1 crystalline form Type M can be identified by an X-ray Powder Diffraction (XRPD) pattern having one or more characteristic diffractions at angles (2 theta±0.2) of 4.5, 5.8, 9.7, 15.6, 21.9, and 26.7. In some embodiments, Compound 1 crystalline form Type M can be identified by X-ray Powder Diffraction (XRPD), having one or more characteristic diffractions at angles (2 theta±0.2) of 4.5, 5.8, 9.7, 15.6, 21.9, and 26.7, corresponding to d-spacing (angstroms±0.2) of 19.5, 15.3, 9.1, 5.7, 4.1, and 3.3, respectively.

In some embodiments, Compound 1 crystalline form Type M is characterized by an X-ray Power Diffraction having one or more characteristic diffractions at angles (2 theta±0.2) of:
4.5
5.8
6.1
8.7
9.0
9.7
12.3
13.1
13.7
14.5
15.1
15.6
16.8
17.4
18.0
18.5
19.5
20.0
21.4
21.9
22.3
22.9
23.3
23.5
24.1
25.0
25.8
26.3
26.7
27.8
28.1
29.4
30.8
31.7
33.0
35.3
37.8
38.6

In some embodiments, Compound 1 crystalline form Type M is characterized by an X-ray Power Diffraction pattern having one or more characteristic diffractions at angles (2 theta±0.2) and corresponding d-spacing (angstroms±0.2) of:

| Pos. [°2Th.] | d-spacing [Å] |
|---|---|
| 4.5 | 19.5 |
| 5.8 | 15.3 |
| 6.1 | 14.4 |
| 8.7 | 10.2 |
| 9.0 | 9.9 |
| 9.7 | 9.1 |
| 12.3 | 7.2 |
| 13.1 | 6.8 |
| 13.7 | 6.4 |
| 14.5 | 6.1 |
| 15.1 | 5.9 |
| 15.6 | 5.7 |
| 16.8 | 5.3 |
| 17.4 | 5.1 |
| 18.0 | 4.9 |
| 18.5 | 4.8 |
| 19.5 | 4.5 |
| 20.0 | 4.4 |
| 21.4 | 4.1 |
| 21.9 | 4.1 |
| 22.3 | 4.0 |
| 22.9 | 3.9 |
| 23.3 | 3.8 |
| 23.5 | 3.8 |
| 24.1 | 3.7 |
| 25.0 | 3.6 |
| 25.8 | 3.5 |
| 26.3 | 3.4 |
| 26.7 | 3.3 |
| 27.8 | 3.2 |
| 28.1 | 3.2 |
| 29.4 | 3.0 |
| 30.8 | 2.9 |
| 31.7 | 2.8 |
| 33.0 | 2.7 |
| 35.3 | 2.5 |
| 37.8 | 2.4 |
| 38.6 | 2.3 |

### Pharmaceutical Compositions Comprising Compound 1 Crystalline Form

In some embodiments, the present disclosure provides a pharmaceutical composition comprising a therapeutically effective amount of any crystalline solid form (Type A, Type B, Type C, Type D, Type E, Type F, or Type G) of Compound 1 as discussed above, and one or more pharmaceutically acceptable excipients. In some embodiments, the present disclosure provides a pharmaceutical composition comprising a therapeutically effective amount of any crystalline solid form (Type A, Type B, Type C, Type D, Type E, Type F, Type G, Type H, Type I, Type J, Type K, Type L, or Type M) of Compound 1 as discussed above, and one or more pharmaceutically acceptable excipients. In some embodiments, the present disclosure provides a pharmaceutical composition comprising any crystalline solid form (Type A, Type B, Type C, Type D, Type E, Type F, or Type G) of Compound 1 as discussed above, and one or more pharmaceutically acceptable excipients. In some embodiments, the present disclosure provides a pharmaceutical composition comprising any crystalline solid form (Type A, Type B, Type C, Type D, Type E, Type F, Type G, Type H, Type I, Type J, Type K, Type L, or Type M) of Compound 1 as discussed above, and one or more pharmaceutically acceptable excipients. In some embodiments, the pharmaceutical composition is for oral administration.

In some embodiments, the present disclosure provides a pharmaceutical composition comprising any crystalline solid form (Type A, Type B, Type C, Type D, Type E, Type F, or Type G) of Compound 1 as discussed above, and having a water content of about 0.5-5.0 weight %, preferably about 1.0-4.5 weight %, more preferably about 1.5-4.0 weight %, even more preferably about 2.0-3.5 weight %, still more preferably about 2.5-3.0 weight % relative to the weight of the pharmaceutical composition. In some embodiments, the present disclosure provides a pharmaceutical composition comprising any crystalline solid form (Type A, Type B, Type C, Type D, Type E, Type F, Type G, Type H, Type I, Type J, Type K, Type L, or Type M) of Compound 1 as discussed above, and having a water content of about 0.5-5.0 weight %, preferably about 1.0-4.5 weight %, more preferably about 1.5-4.0 weight %, even more preferably about 2.0-3.5 weight %, still more preferably about 2.5-3.0 weight % relative to the weight of the pharmaceutical composition. In some embodiments, the present disclosure provides a pharmaceutical composition comprising any crystalline solid form (Type A, Type B, Type C, Type D, Type E, Type F, or Type G) of Compound 1 as discussed above, and having a water content in an amount selected from the following ranges: about 0.5-1.0 weight %, about 1.0-1.5 weight %, about 1.5-2.0 weight %, about 2.5-3.0 weight %, about 3.0-3.5 weight %, about 3.5-4.0 weight %, about 4.0-4.5 weight %, and about 4.5-5.0 weight % relative to the weight of the pharmaceutical composition. In some embodiments, the present disclosure provides a pharmaceutical composition comprising any crystalline solid form (Type A, Type B, Type C, Type D, Type E, Type F, Type G, Type H, Type I, Type J, Type K, Type L, or Type M) of Compound 1 as discussed above, and having a water content in an amount selected from the following ranges: about 0.5-1.0 weight %, about 1.0-1.5 weight %, about 1.5-2.0 weight %, about 2.5-3.0 weight %, about 3.0-3.5 weight %, about 3.5-4.0 weight %, about 4.0-4.5 weight %, and about 4.5-5.0 weight % relative to the weight of the pharmaceutical composition. In some embodiments, the present disclosure provides a pharmaceutical composition comprising any crystalline solid form (Type A, Type B, Type C, Type D, Type E, Type F, or Type G) of Compound 1 as discussed above, and having a water content in an amount selected from the weight percentage: about 0.5 weight %, about 1.0 weight %, about 1.5 weight %, about 2.0 weight %, about 2.5 weight %, about 3.0 weight %, about 3.5 weight %, about 4.0 weight %, about 4.5 weight %, and about 5.0 weight % relative to the weight of the pharmaceutical composition. In some embodiments, the present disclosure provides a pharmaceutical composition comprising any crystalline solid form (Type A, Type B, Type C, Type D, Type E, Type F, Type G, Type H, Type I, Type J, Type K, Type L, or Type M) of Compound 1 as discussed above, and having a water content in an amount selected from the weight percentage: about 0.5 weight %, about 1.0 weight %, about 1.5 weight %, about 2.0 weight %, about 2.5 weight %, about 3.0 weight %, about 3.5 weight %, about 4.0 weight %, about 4.5 weight %, and about 5.0 weight % relative to the weight of the pharmaceutical composition.

In some embodiments, the present disclosure provides a pharmaceutical composition comprising a therapeutically effective amount of Compound 1 crystalline form Type A, and one or more pharmaceutically acceptable excipients. In some embodiments, the present disclosure provides a pharmaceutical composition comprising Compound 1 crystalline form Type A, and one or more pharmaceutically acceptable excipients. In some embodiments, the pharmaceutical composition is for oral administration. In some embodiments, the pharmaceutical composition is substantially free of other crystalline forms of Compound 1. In some embodiments, the pharmaceutical composition is substantially free of amorphous Compound 1.

In some embodiments, the present disclosure provides a pharmaceutical composition comprising a therapeutically effective amount of Compound 1 crystalline form Type B, and one or more pharmaceutically acceptable excipients. In some embodiments, the present disclosure provides a pharmaceutical composition comprising Compound 1 crystalline form Type B, and one or more pharmaceutically acceptable excipients. In some embodiments, the pharmaceutical composition is for oral administration. In some embodiments, the pharmaceutical composition is substantially free of other crystalline forms of Compound 1. In some embodiments, the pharmaceutical composition is substantially free of amorphous Compound 1.

In some embodiments, the present disclosure provides a pharmaceutical composition comprising a therapeutically effective amount of Compound 1 crystalline form Type C, and one or more pharmaceutically acceptable excipients. In some embodiments, the present disclosure provides a pharmaceutical composition comprising Compound 1 crystalline form Type C, and one or more pharmaceutically acceptable excipients. In some embodiments, the pharmaceutical composition is for oral administration. In some embodiments, the pharmaceutical composition is substantially free of other crystalline forms of Compound 1. In some embodiments, the pharmaceutical composition is substantially free of amorphous Compound 1.

In some embodiments, the present disclosure provides a pharmaceutical composition comprising a therapeutically effective amount of Compound 1 crystalline form Type D, and one or more pharmaceutically acceptable excipients. In some embodiments, the present disclosure provides a pharmaceutical composition comprising Compound 1 crystalline form Type D, and one or more pharmaceutically acceptable excipients. In some embodiments, the pharmaceutical composition is for oral administration. In some embodiments, the pharmaceutical composition is substantially free of other crystalline forms of Compound 1. In some embodiments, the pharmaceutical composition is substantially free of amorphous Compound 1.

In some embodiments, the present disclosure provides a pharmaceutical composition comprising a therapeutically effective amount of Compound 1 crystalline form Type E, and one or more pharmaceutically acceptable excipients. In some embodiments, the present disclosure provides a pharmaceutical composition comprising Compound 1 crystalline form Type E, and one or more pharmaceutically acceptable excipients. In some embodiments, the pharmaceutical composition is for oral administration. In some embodiments, the pharmaceutical composition is substantially free of other crystalline forms of Compound 1. In some embodiments, the pharmaceutical composition is substantially free of amorphous Compound 1.

In some embodiments, the present disclosure provides a pharmaceutical composition comprising a therapeutically effective amount of Compound 1 crystalline form Type F, and one or more pharmaceutically acceptable excipients. In some embodiments, the present disclosure provides a pharmaceutical composition comprising Compound 1 crystalline form Type F, and one or more pharmaceutically acceptable excipients. In some embodiments, the pharmaceutical composition is for oral administration. In some embodiments, the pharmaceutical composition is substantially free of other crystalline forms of Compound 1. In some embodiments, the pharmaceutical composition is substantially free of amorphous Compound 1.

In some embodiments, the present disclosure provides a pharmaceutical composition comprising a therapeutically effective amount of Compound 1 crystalline form Type G, and one or more pharmaceutically acceptable excipients. In some embodiments, the present disclosure provides a pharmaceutical composition comprising Compound 1 crystalline form Type G, and one or more pharmaceutically acceptable excipients. In some embodiments, the pharmaceutical composition is for oral administration. In some embodiments, the pharmaceutical composition is substantially free of other crystalline forms of Compound 1. In some embodiments, the pharmaceutical composition is substantially free of amorphous Compound 1.

In some embodiments, the present disclosure provides a pharmaceutical composition comprising a crystalline form of Compound 1. In some embodiments, a pharmaceutical composition comprises a crystalline form of Compound 1 and an amorphous form of Compound 1, wherein the amorphous form of Compound 1 is present in an amount selected from the following ranges: about 90 to about 99%, about 80 to about 89%, about 70 to about 79%, about 60 to about 69%, about 50 to about 59%, about 40 to about 49%, about 30 to about 39%, about 20 to about 29%, about 10 to about 19%, about 1 to about 9% and about 0 to about 0.99%. In some embodiments, a pharmaceutical composition comprising a crystalline form of Compound 1 is substantially free of amorphous Compound 1.

In some embodiments, the present disclosure provides a pharmaceutical composition comprising Compound 1 and its enantiomer (“Compound 2”). In some embodiments, a pharmaceutical composition comprises Compound 1 and its enantiomer Compound 2, wherein Compound 1 has an enantiomeric excess selected from the following ranges: at least about 99%, at least about 95%, at least about 90%, at least about 80%, about 90 to about 99%, about 80 to about 89%, about 70 to about 79%, about 60 to about 69%, about 50 to about 59%, about 40 to about 49%, about 30 to about 39%, about 20 to about 29%, about 10 to about 19%, about 1 to about 9% and about 0 to about 0.99%. In some embodiments, a pharmaceutical composition comprises Compound 1 and its enantiomer Compound 2, wherein the weight percentage of Compound 1 relative to the total weight of Compound 1 and Compound 2 is in a percentage selected from the following ranges: about 90 to about 99%, about 80 to about 89%, about 70 to about 79%, about 60 to about 69%, about 50 to about 59%, about 40 to about 49%, about 30 to about 39%, about 20 to about 29%, about 10 to about 19%, about 1 to about 9% and about 0 to about 0.99%.

Pharmaceutical compositions described herein can comprise a pharmaceutically acceptable carrier or one or more excipients. In some embodiments, pharmaceutical compositions described herein can be provided in a unit dosage form container (e.g., in a vial or bag, or the like). In some embodiments, pharmaceutical compositions described herein can be provided in an oral dosage form. In some embodiments, an oral dosage form is a tablet.

### Amorphous Solid Dispersion Comprising Compound 1

The present disclosure also provides an amorphous solid dispersion comprising Compound 1:

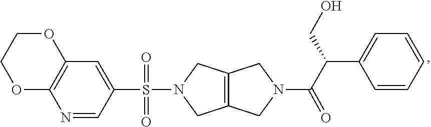 [SMILES: O=C([C@H](CO)c1ccccc1)N1CC2=C(C1)CN(S(=O)(=O)c1cnc3c(c1)OCCO3)C2]

and a polymer. In some embodiments, the polymer is selected from a group consisting of hydroxypropylmethyl cellulose (HPMC), hydroxypropylmethyl cellulose acetate succinate (HPMC AS), hydroxypropyl methyl cellulose phthalate (HPMCP), hydroxypropyl cellulose (HPC), ethylcellulose, cellulose acetate phthalate, polyvinylpyrrolidone (PVP), and a combination thereof, or is selected from a group consisting of polyvinylpyrrolidone (PVP), hydroxypropylmethyl cellulose (HPMC), hydroxypropylcellulose (HPC), hydroxypropylmethyl cellulose acetate succinate (HPMC AS), hydroxyethylcellulose (HEC), poly(methacrylic acid-co-methyl methacrylates) (e.g., Eudragit® L100-55), macrogol 15 hydroxystearate (e.g., Solutol® HS15), polyvinyl caprolactam-polyvinyl acetate-polyethylene glycol graft copolymer (e.g., Soluplus®), polyethylene glycol (PEG), and a combination thereof. In some embodiments, the polymer is hydroxypropylmethyl cellulose (HPMC) or hydroxypropylmethyl cellulose acetate succinate (HPMC AS). In some embodiments, the polymer is hydroxypropylmethyl cellulose acetate succinate (HPMC AS), including any grade thereof (e.g., HPMC AS MG).

Various amounts of Compound 1 and the polymer can be used in the amorphous solid dispersion. In some embodiments, the weight ratio of Compound 1 to the polymer in the amorphous solid dispersion can be selected from the following ranges: about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, and about 1:10. In some embodiments, the weight ratio of Compound 1 to the polymer in the amorphous solid dispersion is in a range of about 3:1 to about 1:3. In some embodiments, the weight ratio of Compound 1 to the polymer in the amorphous solid dispersion is in a range of about 2:1 to about 1:3. In some embodiments, the weight ratio of Compound 1 to the polymer in the amorphous solid dispersion is about 1:3. In some embodiments, the weight ratio of Compound 1 to the polymer in the amorphous solid dispersion is about 1:1. In some embodiments, the weight ratio of Compound 1 to the polymer in the amorphous solid dispersion is about 1:3, about 2:3, about 1:1, about 1.5:1, about 2:1, or about 3:1. In some embodiments, the weight ratio of Compound 1 to the polymer in the amorphous solid dispersion is about 1:3, about 2:3, about 1:1, about 1.5:1, or about 2:1.

In some embodiments, the amorphous solid dispersion is free or substantially free of crystalline Compound 1. In some embodiments, crystalline diffraction peaks are not observable by XRPD analysis (Method D) of the amorphous solid dispersion. In some embodiments, crystalline diffraction peaks are not observable by XRPD analysis (Method D) of the amorphous solid dispersion. In some embodiments, a single glass transition temperature (T_{G}) and no melt endotherm is observable by DSC analysis (Method B) of the amorphous solid dispersion.

In some embodiments, the amorphous solid dispersion is physically stable in that it remains free or substantially free of crystalline Compound 1 over time in accelerated stability studies. In some embodiments, crystalline diffraction peaks are not observable by XRPD analysis (Method D) of the amorphous solid dispersion after storage in a container as described in Example 20 for 5 months at 2-8° C. and ambient relative humidity, 5 months at 25° C. and 60% relative humidity, 1 month at 2-8° C. and ambient relative humidity, 1 month at 25° C. and 60% relative humidity, or 1 month at 40° C. and 75% relative humidity. In some embodiments, a single glass transition temperature (T_{G}) and no melt endotherm is observable by DSC analysis (Method B) of the amorphous solid dispersion after storage in a container as described in Example 20 for 5 months at 2-8° C. and ambient relative humidity, 5 months at 25° C. and 60% relative humidity, 1 month at 2-8° C. and ambient relative humidity, 1 month at 25° C. and 60% relative humidity, or 1 month at 40° C. and 75% relative humidity. In some embodiments, crystalline diffraction peaks are not observable by XRPD analysis (Method D) of the amorphous solid dispersion after storage in a sealed vial for 1 week at 60° C., storage in a sealed vial for 2 weeks at 60° C., storage in an unsealed vial for 1 week at 25° C. and 60% relative humidity, storage in an unsealed vial for 2 weeks at 25° C. and 60% relative humidity, storage in an unsealed vial for 1 week at 40° C. and 75% relative humidity, storage in an unsealed vial for 2 weeks at 40° C. and 75% relative humidity, storage in an unsealed vial for 1 week at 60° C. and 75% relative humidity, or storage in an unsealed vial for 2 weeks at 60° C. and 75% relative humidity. In some embodiments, a single glass transition temperature (T_{G}) and no melt endotherm is observable by DSC analysis (Method B) of the amorphous solid dispersion after storage in a sealed vial for 1 week at 60° C., storage in a sealed vial for 2 weeks at 60° C., storage in an unsealed vial for 1 week at 25° C. and 60% relative humidity, storage in an unsealed vial for 2 weeks at 25° C. and 60% relative humidity, storage in an unsealed vial for 1 week at 40° C. and 75% relative humidity, storage in an unsealed vial for 2 weeks at 40° C. and 75% relative humidity, storage in an unsealed vial for 1 week at 60° C. and 75% relative humidity, or storage in an unsealed vial for 2 weeks at 60° C. and 75% relative humidity.

In some embodiments, the amorphous solid dispersion is highly soluble, e.g., Compound 1 dissolves quickly and readily in biorelevant media. In some embodiments, Compound 1 has a concentration of at least 150 μg/mL, at least 200 μg/mL, at least 250 μg/mL, at least 300 μg/mL, or at least 350 μg/mL after 30 minutes in the kinetic solubility experiment described in Example 23. In some embodiments, Compound 1 has a Cmax of at least 300 μg/mL, at least 350 μg/mL, at least 400 μg/mL, at least 450 μg/mL, at least 500 μg/mL, at least 550 μg/mL, at least 600 μg/mL, at least 650 μg/mL, or at least 700 μg/mL in the kinetic solubility experiment described in Example 23. In some embodiments, Compound 1 has a concentration of at least 200 μg/mL, at least 250 μg/mL, at least 300 μg/mL, at least 350 μg/mL, at least 400 μg/mL, at least 450 μg/mL, at least 500 μg/mL, at least 550 μg/mL, or at least 600 μg/mL after 4 hours in the kinetic solubility experiment described in Example 23. In some embodiments, Compound 1 has a concentration of at least 150 μg/mL, at least 200 μg/mL, at least 250 μg/mL, or at least 300 μg/mL after 16 hours in the kinetic solubility experiment described in Example 23.

### Pharmaceutical Compositions Comprising Compound 1 Amorphous Solid Dispersion

The present disclosure further provides a pharmaceutical composition comprising a therapeutically effective amount of an amorphous solid dispersion comprising Compound 1, and one or more pharmaceutically acceptable excipients. In some embodiments, the pharmaceutical composition is for oral administration.

In some embodiments, the present disclosure provides a pharmaceutical composition comprising an amorphous solid dispersion comprising Compound 1, and having a water content of about 0.5-5.0 weight %, preferably about 1.0-4.5 weight %, more preferably about 1.5-4.0 weight %, even more preferably about 2.0-3.5 weight %, still more preferably about 2.5-3.0 weight % relative to the weight of the pharmaceutical composition. In some embodiments, the present disclosure provides a pharmaceutical composition comprising an amorphous solid dispersion comprising Compound 1, and having a water content in an amount selected from the following ranges: about 0.5-1.0 weight %, about 1.0-1.5 weight %, about 1.5-2.0 weight %, about 2.5-3.0 weight %, about 3.0-3.5 weight %, about 3.5-4.0 weight %, about 4.0-4.5 weight %, and about 4.5-5.0 weight % relative to the weight of the pharmaceutical composition. In some embodiments, the present disclosure provides a pharmaceutical composition comprising an amorphous solid dispersion comprising Compound 1, and having a water content in an amount selected from the weight percentage: about 0.5 weight %, about 1.0 weight %, about 1.5 weight %, about 2.0 weight %, about 2.5 weight %, about 3.0 weight %, about 3.5 weight %, about 4.0 weight %, about 4.5 weight %, and about 5.0 weight % relative to the weight of the pharmaceutical composition.

In some embodiments, the pharmaceutical composition comprises about 1-4 mg, about 10 mg, about 25 mg, about 50 mg, about 100 mg, about 200 mg, or about 300 mg of Compound 1. In some embodiments, the pharmaceutical composition comprises about 1-4 mg, about 1-3 mg, about 2-4 mg, about 2 mg, or about 3 mg of Compound 1. In some embodiments, the pharmaceutical composition comprises about 25 mg of Compound 1. In some embodiments, the pharmaceutical composition comprises about 100 mg of Compound 1. In some embodiments, the pharmaceutical composition comprises about 200 mg of Compound 1.

Pharmaceutical compositions described herein can comprise a pharmaceutically acceptable carrier or one or more excipients. In some embodiments, pharmaceutical compositions described herein can be provided in a unit dosage form container (e.g., in a vial or bag, or the like). In some embodiments, pharmaceutical compositions described herein can be provided in an oral dosage form. In some embodiments, an oral dosage form is a tablet. In some embodiments, the tablet is a minitablet.

In some embodiments, the pharmaceutical composition comprises one or more pharmaceutically acceptable excipients which comprise one or more of a filler, a dry binder, a glidant, a lubricant, a disintegrant, and a film coating agent. In some embodiments, the one or more pharmaceutically acceptable excipients comprise a filler, and the filler comprises microcrystalline cellulose. In some embodiments, the one or more pharmaceutically acceptable excipients comprise a filler, and the filler comprises lactose monohydrate. In some embodiments, the one or more pharmaceutically acceptable excipients comprise a dry binder, and the dry binder comprises crospovidone. In some embodiments, the one or more pharmaceutically acceptable excipients comprise a glidant, and the glidant comprises colloidal silicon dioxide. In some embodiments, the one or more pharmaceutically acceptable excipients comprise a lubricant, and the lubricant comprises magnesium stearate. In some embodiments, the one or more pharmaceutically acceptable excipients comprise a disintegrant, and the disintegrant comprises croscarmellose sodium. In some embodiments, the one or more pharmaceutically acceptable excipients comprise a lubricant, and the lubricant comprises magnesium stearate.

In some embodiments, a pharmaceutical composition comprises a tablet core. In some embodiments, the tablet core comprises an intra granular portion comprising the amorphous solid dispersion, and an extra granular portion blended with the intra granular portion. In some embodiments, a pharmaceutical composition further comprises a coating disposed on the tablet core.

Various amounts of Compound 1 relative to the tablet core can be used in a pharmaceutical composition comprising an amorphous solid dispersion comprising Compound 1. In some embodiments, the amorphous solid dispersion comprising Compound 1 can be about 10 weight %, about 20 weight %, about 30 weight %, about 40 weight %, about 50 weight %, about 60 weight %, about 70 weight %, about 80 weight %, or about 90 weight % of the tablet core. In some embodiments, the amorphous solid dispersion comprising Compound 1 is at least about 30 weight % of the tablet core. In some embodiments, the amorphous solid dispersion comprising Compound 1 is at least about 50 weight % of the tablet core. In some embodiments, the amorphous solid dispersion comprising Compound 1 is at least about 60 weight % of the tablet core. In some embodiments, the amorphous solid dispersion comprising Compound 1 is about 50 weight % of(the tablet core. In some embodiments, the amorphous solid dispersion comprising Compound 1 is about 50 to about 70 weight % of the tablet core. In some embodiments, the amorphous solid dispersion comprising Compound 1 is about 60 to about 65 weight % of the tablet core.

In some embodiments, the intra granular portion further comprises one or more of a filler, a dry binder, a glidant, and a lubricant. In some embodiments, the extra granular portion further comprises one or more of a filler, a disintegrant, and a lubricant.

In some embodiments, the tablet core has the following components:

| | % | |
|---|---|---|
| | Formulation | |
| Function | (weight) | Exemplary Component |
| API | 30-70% | Amorphous Solid Dispersion of Compound 1 |
| Filler | 15-40% | Microcrystalline Cellulose |
| Dry binder | 2-10% | Crospovidone |
| Glidant | 0.25-1.25% | Colloidal Silicon Dioxide |
| Lubricant | 0.25-1.00% | Magnesium Stearate |

In some embodiments, the tablet core has the following components:

| | % | |
|---|---|---|
| | Formulation | |
| Function | (weight) | Exemplary Component |
| API | 30-70% | Amorphous Solid Dispersion of Compound 1 |
| Filler | 15-50% | Microcrystalline Cellulose, Lactose Monohydrate |
| Dry binder | 2-10% | Crospovidone |
| Glidant | 0.25-1.25% | Colloidal Silicon Dioxide |
| Disintegrant | 2-3% | Croscarmellose Sodium |
| Lubricant | 0.25-1.00% | Magnesium Stearate |

In some embodiments, the tablet core has the following components:

| Component | Function | Range |
|---|---|---|
| SDD (1:1 | Active | 50-75% |
| drug:polymer w/w) | | |
| Microcrystalline | Filler | 15-30% |
| Cellulose | | |
| Lactose Monohydrate | Filler | 0-20% |
| Crosslinked | Dry Binder | 2-10% |
| polyvinylpyrrolidone | | |
| Colloidal Silicon | Glidant | <2% |
| Dioxide | | |
| Croscarmellose | Disintegrant | 2-10% |
| Sodium | | |
| Magnesium Stearate | Lubricant | <2% |

In some embodiments, the tablet core has the following components:

| Component | Function | Range |
|---|---|---|
| SDD (1.5:1 | Active | 50-75% |
| drug:polymer w/w) | | |
| Microcrystalline | Filler | 15-30% |
| Cellulose | | |
| Lactose Monohydrate | Filler | 0-20% |
| Crosslinked | Dry Binder | 2-10% |
| polyvinylpyrrolidone | | |
| Colloidal Silicon | Glidant | <2% |
| Dioxide | | |
| Croscarmellose | Disintegrant | 2-10% |
| Sodium | | |
| Magnesium Stearate | Lubricant | <2% |

In some embodiments, the tablet core has the following components:

| Component | Function | Range |
|---|---|---|
| SDD (1:1) | Active | 60-80% |
| Microcrystalline | Filler | 10-20% |
| Cellulose | | |
| Lactose Monohydrate | Filler | 5-10% |
| Crosslinked | Dry Binder | 2-10% |
| polyvinylpyrrolidone | | |
| Colloidal Silicon | Glidant | <2% |
| Dioxide | | |
| Magnesium Stearate | Lubricant | <2% |

In some embodiments, the tablet core has the following components:

| Component | Function | Range |
|---|---|---|
| SDD (1:1) | Active | 50-75% |
| Microcrystalline | Filler | 15-30% |
| Cellulose | | |
| Lactose Monohydrate | Filler | 0-20% |
| Crosslinked | Dry Binder | 2-10% |
| polyvinylpyrrolidone | | |
| Colloidal Silicon | Glidant | <2.5% |
| Dioxide | | |
| Croscarmellose | Disintegrant | 2-10% |
| Sodium | | |
| Magnesium Stearate | Lubricant | <2% |

In some embodiments, the Compound 1 oral unit dosage form can be a tablet comprising a total of about 10-35% by weight of Compound 1, with a total dose of about 100 mg or 200 mg, and a total weight of less than about 800 mg. In one embodiment, a tablet having a composition described in a table above comprises about 500% of an API formed as an amorphous solid dispersion of Compound 1 obtained from a 1:3 SDD process described in the examples below (e.g., about 12.5% of Compound 1 in the tablet). In one embodiment, a tablet having a composition described in a table above comprises about 3000 of an API formed as an amorphous solid dispersion of Compound 1 obtained from a 1:1 SDD process described in the examples below (e.g., about 15% of Compound 1 in the tablet, with a total of about 100 mg Compound 1 in the tablet). In one embodiment, a tablet having a composition described in a table above comprises about 62% of an API formed as an amorphous solid dispersion of Compound 1 obtained from a 1:1 SDD process described in the examples below (e.g., about 31% of Compound 1 in the tablet, with a total of about 200 mg of Compound 1 in the tablet).

In some embodiments, the oral unit dosage form is a minitablet comprising about 25-40% by weight and about 1-4 mg, about 1-3 mg, about 2-4 mg, about 2 mg, or about 3 mg of Compound 1. In some embodiments, the oral unit dosage form is a minitablet comprising about 25-35% by weight and about 1-4 mg, about 1-3 mg, about 2-4 mg, about 2 mg, or about 3 mg of Compound 1. In some embodiments, the oral unit dosage form is a minitablet comprising about 30-40% by weight and about 1-4 mg, about 1-3 mg, about 2-4 mg, about 2 mg, or about 3 mg of Compound 1.

### Methods for Preparing Amorphous Solid Dispersions of Compound 1

The present disclosure also provides a method for preparing an amorphous solid dispersion comprising Compound 1:

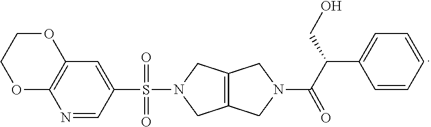 [SMILES: O=C([C@H](CO)c1ccccc1)N1CC2=C(C1)CN(S(=O)(=O)c1cnc3c(c1)OCCO3)C2]

In some embodiments, the method comprises mixing Compound 1, a polymer, and a solvent to afford a mixture, and spray-drying the mixture to afford an amorphous solid dispersion comprising Compound 1.

In some embodiments, the polymer used in the method is selected from a group consisting of hydroxypropylmethyl cellulose (HPMC), hydroxypropylmethyl cellulose acetate succinate (HPMC AS), hydroxypropyl methyl cellulose phthalate (HPMCP), hydroxypropyl cellulose (HPC), ethylcellulose, cellulose acetate phthalate, polyvinylpyrrolidone (PVP), and a combination thereof, or is selected from a group consisting of polyvinylpyrrolidone (PVP), hydroxypropylmethyl cellulose (HPMC), hydroxypropylcellulose (HPC), hydroxypropylmethyl cellulose acetate succinate (HPMC AS), hydroxyethylcellulose (HEC), poly(methacrylic acid-co-methyl methacrylates) (e.g., Eudragit® L100-55), macrogol 15 hydroxystearate (e.g., Solutol® HS15), polyvinyl caprolactam-polyvinyl acetate-polyethylene glycol graft copolymer (e.g., Soluplus®), polyethylene glycol (PEG), and a combination thereof. In some embodiments, the polymer is hydroxypropylmethyl cellulose (HPMC) or hydroxypropylmethyl cellulose acetate succinate (HPMC AS). In some embodiments, the polymer is hydroxypropylmethyl cellulose acetate succinate (HPMC AS), including any grade thereof (e.g., HPMC AS MG).

Various amounts of Compound 1 and the polymer can be used in the method to prepare the amorphous solid dispersion. In some embodiments, the weight ratio of Compound 1 to the polymer used in the method to prepare the amorphous solid dispersion can be selected from the following ranges: about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, and about 1:10. In some embodiments, the weight ratio of Compound 1 to the polymer used in the method to prepare the amorphous solid dispersion is in a range of about 3:1 to about 1:3. In some embodiments, the weight ratio of Compound 1 to the polymer used in the method to prepare the amorphous solid dispersion is in a range of about 2:1 to about 1:3. In some embodiments, the weight ratio of Compound 1 to the polymer used in the method to prepare the amorphous solid dispersion is about 1:3. In some embodiments, the weight ratio of Compound 1 to the polymer used in the method to prepare the amorphous solid dispersion is about 1:1. In some embodiments, the weight ratio of Compound 1 to the polymer used in the method to prepare the amorphous solid dispersion is about 1:3, about 2:3, about 1:1, about 1.5:1, about 2:1, or about 3:1. In some embodiments, the weight ratio of Compound 1 to the polymer used in the method to prepare the amorphous solid dispersion is about 1:3, about 2:3, about 1:1, about 1.5:1, or about 2:1.

Various solvents can be used in the method to prepare the amorphous solid dispersion. In some embodiments, the solvent is dichloromethane and methanol.

The present disclosure further provides a product prepared by a process comprising mixing Compound 1, a polymer, and a solvent to afford a mixture, and spray-drying the mixture to afford an amorphous solid dispersion comprising Compound 1

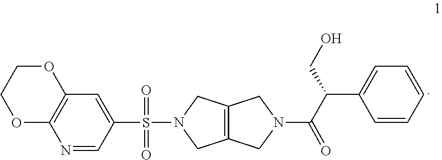 [SMILES: O=C([C@H](CO)c1ccccc1)N1CC2=C(C1)CN(S(=O)(=O)c1cnc3c(c1)OCCO3)C2]

In some embodiments, the polymer used in the process is selected from a group consisting of hydroxypropylmethyl cellulose (HPMC), hydroxypropylmethyl cellulose acetate succinate (HPMC AS), hydroxypropyl methyl cellulose phthalate (HPMCP), hydroxypropyl cellulose (HPC), ethylcellulose, cellulose acetate phthalate, polyvinylpyrrolidone (PVP), and a combination thereof, or is selected from a group consisting of polyvinylpyrrolidone (PVP), hydroxypropylmethyl cellulose (HPMC), hydroxypropylcellulose (HPC), hydroxypropylmethyl cellulose acetate succinate (HPMC AS), hydroxyethylcellulose (HEC), poly(methacrylic acid-co-methyl methacrylates) (e.g., Eudragit® L100-55), macrogol 15 hydroxystearate (e.g., Solutol® HS15), polyvinyl caprolactam-polyvinyl acetate-polyethylene glycol graft copolymer (e.g., Soluplus®), polyethylene glycol (PEG), and a combination thereof. In some embodiments, the polymer is hydroxypropylmethyl cellulose (HPMC) or hydroxypropylmethyl cellulose acetate succinate (HPMC AS). In some embodiments, the polymer is hydroxypropylmethyl cellulose acetate succinate (HPMC AS), including any grade thereof (e.g., HPMC AS MG).

Various amounts of Compound 1 and the polymer can be used in the process to prepare the amorphous solid dispersion. In some embodiments, the weight ratio of Compound 1 to the polymer used in the process to prepare the amorphous solid dispersion can be selected from the following ranges: about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, and about 1:10. In some embodiments, the weight ratio of Compound 1 to the polymer used in the process to prepare the amorphous solid dispersion is in a range of about 3:1 to about 1:3. In some embodiments, the weight ratio of Compound 1 to the polymer used in the process to prepare the amorphous solid dispersion is in a range of about 2:1 to about 1:3. In some embodiments, the weight ratio of Compound 1 to the polymer used in the process to prepare the amorphous solid dispersion is about 1:3. In some embodiments, the weight ratio of Compound 1 to the polymer used in the method to prepare the amorphous solid dispersion is about 1:1. In some embodiments, the weight ratio of Compound 1 to the polymer used in the method to prepare the amorphous solid dispersion is about 1:3, about 2:3, about 1:1, about 1.5:1, about 2:1, or about 3:1. In some embodiments, the weight ratio of Compound 1 to the polymer used in the method to prepare the amorphous solid dispersion is about 1:3, about 2:3, about 1:1, about 1.5:1, or about 2:1.

Various solvents can be used in the process to prepare the amorphous solid dispersion. In some embodiments, the solvent is dichloromethane and methanol.

### Pharmaceutical Compositions Comprising Compound 1

The present disclosure provides a pharmaceutical composition comprising Compound 1:

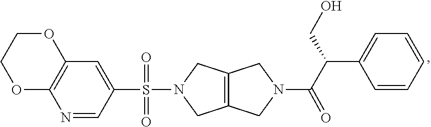 [SMILES: O=C([C@H](CO)c1ccccc1)N1CC2=C(C1)CN(S(=O)(=O)c1cnc3c(c1)OCCO3)C2]

obtained by a process comprising mixing Compound 1 in a solid form, a polymer, and a solvent to afford a mixture, and spray-drying the mixture to afford an amorphous solid dispersion comprising Compound 1.

In some embodiments, the solid form is Type A of Compound 1. In some embodiments, the solid form is Type B of Compound 1. In some embodiments, the solid form is Type C of Compound 1. In some embodiments, the solid form is Type D of Compound 1. In some embodiments, the solid form is Type E of Compound 1. In some embodiments, the solid form is Type F of Compound 1. In some embodiments, the solid form is Type G of Compound 1. In some embodiments, the solid form is Type H of Compound 1. In some embodiments, the solid form is Type I of Compound 1. In some embodiments, the solid form is Type J of Compound 1. In some embodiments, the solid form is Type K of Compound 1. In some embodiments, the solid form is Type L of Compound 1. In some embodiments, the solid form is Type M of Compound 1. In some embodiments, the solid form is selected from the group consisting of Type A, Type B, Type C, Type D, Type E, Type F, Type G, Type H, Type I, Type J, Type K, Type L, and Type M of Compound 1. In some embodiments, the solid form is amorphous form of Compound 1.

In some embodiments, the pharmaceutical composition obtained by the process has a water content of about 0.5-5.0 weight %, preferably about 1.0-4.5 weight %, more preferably about 1.5-4.0 weight %, even more preferably about 2.0-3.5 weight %, still more preferably about 2.5-3.0 weight % relative to the weight of the pharmaceutical composition. In some embodiments, the pharmaceutical composition obtained by the process has a water content in an amount selected from the following ranges: about 0.5-1.0 weight %, about 1.0-1.5 weight %, about 1.5-2.0 weight %, about 2.5-3.0 weight %, about 3.0-3.5 weight %, about 3.5-4.0 weight %, about 4.0-4.5 weight %, and about 4.5-5.0 weight % relative to the weight of the pharmaceutical composition. In some embodiments, the pharmaceutical composition obtained by the process has a water content in an amount selected from the weight percentage: about 0.5 weight %, about 1.0 weight %, about 1.5 weight %, about 2.0 weight %, about 2.5 weight %, about 3.0 weight %, about 3.5 weight %, about 4.0 weight %, about 4.5 weight %, and about 5.0 weight % relative to the weight of the pharmaceutical composition.

In some embodiments, the polymer used in the process is selected from a group consisting of hydroxypropylmethyl cellulose (HPMC), hydroxypropylmethyl cellulose acetate succinate (HPMC AS), hydroxypropyl methyl cellulose phthalate (HPMCP), hydroxypropyl cellulose (HPC), ethylcellulose, cellulose acetate phthalate, polyvinylpyrrolidone (PVP), and a combination thereof, or is selected from a group consisting of polyvinylpyrrolidone (PVP), hydroxypropylmethyl cellulose (HPMC), hydroxypropylcellulose (HPC), hydroxypropylmethyl cellulose acetate succinate (HPMC AS), hydroxyethylcellulose (HEC), poly(methacrylic acid-co-methyl methacrylates) (e.g., Eudragit® L100-55), macrogol 15 hydroxystearate (e.g., Solutol® HS15), polyvinyl caprolactam-polyvinyl acetate-polyethylene glycol graft copolymer (e.g., Soluplus®), polyethylene glycol (PEG), and a combination thereof. In some embodiments, the polymer is hydroxypropylmethyl cellulose (HPMC) or hydroxypropylmethyl cellulose acetate succinate (HPMC AS). In some embodiments, the polymer is hydroxypropylmethyl cellulose acetate succinate (HPMC AS), including any grade thereof (e.g., HPMC AS MG).

Various amounts of Compound 1 and the polymer can be used in the process to prepare the amorphous solid dispersion. In some embodiments, the weight ratio of Compound 1 to the polymer used in the process to prepare the amorphous solid dispersion can be selected from the following ranges: about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, and about 1:10. In some embodiments, the weight ratio of Compound 1 to the polymer used in the process to prepare the amorphous solid dispersion is in a range of about 3:1 to about 1:3. In some embodiments, the weight ratio of Compound 1 to the polymer used in the process to prepare the amorphous solid dispersion is in a range of about 2:1 to about 1:3. In some embodiments, the weight ratio of Compound 1 to the polymer used in the process to prepare the amorphous solid dispersion is about 1:3. In some embodiments, the weight ratio of Compound 1 to the polymer used in the method to prepare the amorphous solid dispersion is about 1:1. In some embodiments, the weight ratio of Compound 1 to the polymer used in the method to prepare the amorphous solid dispersion is about 1:3, about 2:3, about 1:1, about 1.5:1, about 2:1, or about 3:1. In some embodiments, the weight ratio of Compound 1 to the polymer used in the method to prepare the amorphous solid dispersion is about 1:3, about 2:3, about 1:1, about 1.5:1, or about 2:1.

Various solvents can be used in the process to prepare the amorphous solid dispersion. In some embodiments, the solvent is dichloromethane and methanol.

### Solid Oral Dosage Forms of Compound 1

The disclosure also provides solid oral dosage forms of Compound 1, such as tablets and capsules. In some embodiments, the solid oral dosage form comprises a stabilized amorphous compound (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one, wherein the stabilized amorphous compound does not show crystallinity by PXRD (Method D) after 2 weeks of storage at 60° C./75% RH (exposed). In some embodiments, the stabilized amorphous compound shows a single glass transition temperature (T_{G}) and no melt endotherm by DSC (Method B) after 2 weeks of storage at 60° C./75% RH (exposed).

In some embodiments, the solid oral dosage form contains a total of about 100 mg or about 200 mg of (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one. In some embodiments, the solid dosage form has a total weight of not more than 700 mg, 800 mg, 900 mg, 1000 mg or 1200 mg. In some embodiments, the solid oral dosage form is a tablet or capsule.

In some embodiments, the stabilized amorphous compound in the solid oral dosage form is in a spray dried dispersion with a polymer. In some embodiments, the polymer is selected from the group consisting of hydroxypropylmethyl cellulose (HPMC), hydroxypropylmethyl cellulose acetate succinate (HPMC AS), hydroxypropyl methyl cellulose phthalate (HPMCP), hydroxypropyl cellulose (HPC), ethylcellulose, cellulose acetate phthalate, polyvinylpyrrolidone (PVP), and a combination thereof, or is selected from a group consisting of polyvinylpyrrolidone (PVP), hydroxypropylmethyl cellulose (HPMC), hydroxypropylcellulose (HPC), hydroxypropylmethyl cellulose acetate succinate (HPMC AS), hydroxyethylcellulose (HEC), poly(methacrylic acid-co-methyl methacrylates) (e.g., Eudragit® L100-55), macrogol 15 hydroxystearate (e.g., Solutol® HS15), polyvinyl caprolactam-polyvinyl acetate-polyethylene glycol graft copolymer (e.g., Soluplus®), polyethylene glycol (PEG), and a combination thereof. In some embodiments, the polymer is HPMC AS. In some embodiments, the (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one is spray dried with HPMC AS in a weight ratio of 1:3 to 2:1. In some embodiments, the (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one is spray dried with HPMC AS in a weight ratio of 1:1.

The disclosure also relates to a (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one active pharmaceutical ingredient (API) composition comprising 0.05-5.0% by HPLC of (R)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one.

The disclosure also relates to a tablet comprising about 100 mg or about 200 mg of stabilized amorphous compound (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one as the active pharmaceutical ingredient (API), wherein the stabilized amorphous compound does not show crystallinity by PXRD (Method D) after 2 weeks of storage of the tablet at 60° C./75% RH (exposed). In some embodiments, the API comprises less than 5.0% by HPLC of (R)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one. In some embodiments, the API comprises less than 0.05% by HPLC of (R)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one. In some embodiments, the tablet has a total weight of less than 700 mg, 800 mg, 900 mg, 1000 mg or 1200 mg.

### Tablet Dosage Forms of Compound 1

The disclosure also provides tablet dosage forms of Compound 1. In some embodiments, the tablet dosage form comprises a tablet core, the tablet core comprising at least 10 weight % of Compound 1 in amorphous form:

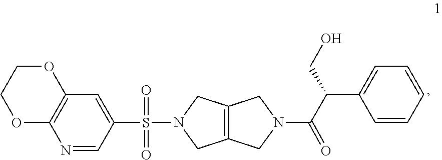 [SMILES: O=C([C@H](CO)c1ccccc1)N1CC2=C(C1)CN(S(=O)(=O)c1cnc3c(c1)OCCO3)C2]

wherein crystalline Compound 1 (Type A) is not observable by XRPD analysis (Method D) of the tablet core. In some embodiments, wherein the tablet core comprises at least 15 weight %, at least 20 weight %, at least 25 weight % or at least 30 weight % of Compound 1 in amorphous form. In some embodiments, the tablet core comprises about 200 mg of Compound 1 per tablet and has a total weight of no more than about 1200 mg, about 1100 mg, about 1000 mg, about 900 mg, about 800 mg, or about 700 mg per tablet per tablet.

In some embodiments, the tablet dosage form comprises a tablet core, the tablet core having a total weight of no more than about 1000 mg and comprising about 200 mg of Compound 1 in amorphous form per tablet, wherein crystalline Compound 1 (Type A) is not observable by XRPD analysis (Method D) of the tablet core. In some embodiments, the tablet core has a total weight of no more than about 800 mg per tablet.

In some embodiments, the tablet core comprises Compound 1 in highly enantiopure form. In some embodiments, the tablet core comprises 0.05-5.0% of Compound 2:

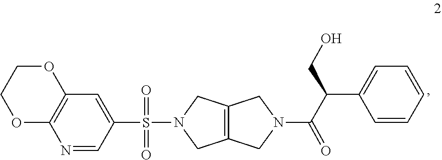 [SMILES: O=C([C@@H](CO)c1ccccc1)N1CC2=C(C1)CN(S(=O)(=O)c1cnc3c(c1)OCCO3)C2]

based on the total amount of Compound 1 and Compound 2. In some embodiments, the tablet core comprises 0.05-3.0% of Compound 2, based on the total amount of Compound 1 and Compound 2. In some embodiments, the tablet core comprises 0.05-2.0% of Compound 2, based on the total amount of Compound 1 and Compound 2. In some embodiments, the tablet core comprises 0.05-1.0% of Compound 2, based on the total amount of Compound 1 and Compound 2.

In some embodiments, the tablet dosage form is physically stable in that it remains free or substantially free of crystalline Compound 1 over time in accelerated stability studies. In some embodiments, crystalline Compound 1 (Type A) is not observable by XRPD analysis (Method D) of the tablet core after storage in a sealed container as described in Example 29 for 1 month at 25° C. and 60% relative humidity, storage in a sealed container as described in Example 29 for 2 months at 25° C. and 60% relative humidity, storage in a sealed container as described in Example 29 for 3 months at 25° C. and 60% relative humidity, storage in a sealed container as described in Example 29 for 1 month at 40° C. and 75% relative humidity, storage in a sealed container as described in Example 29 for 2 months at 40° C. and 75% relative humidity, storage in a sealed container as described in Example 29 for 3 months at 40° C. and 75% relative humidity.

In some embodiments, the tablet dosage form is a microtablet comprising a tablet core, the tablet core comprising at least 10 weight %, 15 weight %, 20 weight %, 25 weight %, 30 weight %, 25-35 weight %, 30-40 weight %, or 25-40 weight % and about 1-4 mg, about 1-3 mg, about 2-4 mg, about 2 mg, or about 3 mg of Compound 1 in amorphous form. In some embodiments, the microtablet is physically stable in that it remains free or substantially free of crystalline Compound 1 over time in accelerated stability studies. In some embodiments, crystalline Compound 1 (Type A) is not observable by XRPD analysis of the tablet core after storage under closed conditions as described in Example 33 for 4 weeks at 2-8° C., 25° C./50% RH, 40° C./75% RH, or 60° C. (closed) or under open conditions as described in Example 33 for 4 weeks at 40° C./75% RH.

In some embodiments, Compound 1 is present in an amorphous solid dispersion comprising Compound 1 and a polymer. In some embodiments, the polymer is selected from a group consisting of hydroxypropylmethyl cellulose (HPMC), hydroxypropylmethyl cellulose acetate succinate (HPMC AS), hydroxypropyl methyl cellulose phthalate (HPMCP), hydroxypropyl cellulose (HPC), ethylcellulose, cellulose acetate phthalate, polyvinylpyrrolidone (PVP), and a combination thereof, or is selected from a group consisting of polyvinylpyrrolidone (PVP), hydroxypropylmethyl cellulose (HPMC), hydroxypropylcellulose (HPC), hydroxypropylmethyl cellulose acetate succinate (HPMC AS), hydroxyethylcellulose (HEC), poly(methacrylic acid-co-methyl methacrylates) (e.g., Eudragit® L100-55), macrogol 15 hydroxystearate (e.g., Solutol® HS15), polyvinyl caprolactam-polyvinyl acetate-polyethylene glycol graft copolymer (e.g., Soluplus®), polyethylene glycol (PEG), and a combination thereof. In some embodiments, the polymer is hydroxypropylmethyl cellulose (HPMC) or hydroxypropylmethyl cellulose acetate succinate (HPMC AS). In some embodiments, the polymer is hydroxypropylmethyl cellulose acetate succinate (HPMC AS), including any grade thereof (e.g., HPMC AS MG).

In some embodiments, the weight ratio of Compound 1 to the polymer is in a range of about 3:1 to about 1:3. In some embodiments, the weight ratio of Compound 1 to the polymer is in a range of about 2:1 to about 1:3. In some embodiments, the weight ratio of Compound 1 to the polymer is about 1:3. In some embodiments, the weight ratio of Compound 1 to the polymer is about 1:1. In some embodiments, the weight ratio of Compound 1 to the polymer is about 1:3, about 2:3, about 1:1, about 1.5:1, about 2:1, or about 3:1. In some embodiments, the weight ratio of Compound 1 to the polymer is about 1:3, about 2:3, about 1:1, about 1.5:1, or about 2:1.

In some embodiments, the tablet core of the tablet dosage form further comprises one or more pharmaceutically acceptable excipients. In some embodiments, the one or more pharmaceutically acceptable excipients comprise one or more of a filler, a dry binder, a glidant, a lubricant, a disintegrant, and a film coating agent.

In some embodiments, the tablet core comprises an intra granular portion comprising Compound 1; and an extra granular portion blended with the intra granular portion. In some embodiments, the intragranular portion comprises an amorphous solid dispersion comprising Compound 1 and a polymer and one or more of a filler, a dry binder, a glidant, and a lubricant, and the extragranular portion comprises one or more of a filler, a disintegrant, and a lubricant. In some embodiments, the intragranular portion comprises:
an amorphous solid dispersion of Compound 1 in an amount of 30-70 weight % of the tablet core;
one or more fillers in an amount of 15-50 weight % of the tablet core;
one or more dry binders in an amount of 2.50-10 weight % of the tablet core;
one or more glidants in an amount of 0.50-1.50 weight % of the tablet core; and
one or more lubricants in an amount of 0.25-1 weight % of the tablet core; and the extragranular portion comprises:
one or more fillers in an amount of 5-15 weight % of the tablet core;
one or more disintegrants in an amount of 1.25-5 weight % of the tablet core; and
one or more lubricants in an amount of 0.25-1 weight % of the tablet core.

In some embodiments, the tablet dosage form comprises:
an amorphous solid dispersion of Compound 1 in an amount of 50-75 weight % of the tablet core;
one or more fillers in an amount of 15-50 weight % of the tablet core;
one or more dry binders in an amount of 2-10 weight % of the tablet core;
one or more glidants in an amount of <2 weight % of the tablet core;
one or more disintegrants in an amount of 2-10 weight % of the tablet core; and
one or more lubricants in an amount of <2 weight % of the tablet core.

In some embodiments, the amorphous solid dispersion comprises Compound 1 and a polymer (as described in any of the embodiments set forth herein). In some embodiments, the one or more fillers comprise microcrystalline cellulose or lactose monohydrate. In some embodiments, the one or more dry binders comprise crospovidone or crosslinked polyvinylpyrrolidone. In some embodiments, the one or more glidants comprise colloidal silicon dioxide or fumed silica. In some embodiments, the one or more lubricants comprise magnesium stearate. In some embodiments, the one or more disintegrants comprise crocarmellose sodium.

### Medical Uses of the Solid Forms and Pharmaceutical Compositions

In some embodiments, the disclosure relates to a method of treating a disease associated with decreased activity of PKR in a subject in need thereof which comprises administering to the subject an effective amount of a compound of Formula I in any of the forms described herein, including any embodiment thereof.

### Embodiments

In some embodiments, the disclosure relates to one or more of the following enumerated embodiments:

1. A crystalline solid form of Compound 1:

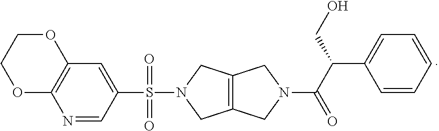 [SMILES: O=C([C@H](CO)c1ccccc1)N1CC2=C(C1)CN(S(=O)(=O)c1cnc3c(c1)OCCO3)C2]

2. The crystalline solid form of embodiment 1, wherein the crystalline solid form is Type A of (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one (“Compound 1”).
3. The crystalline solid form of embodiment 1 or 2, wherein Type A of Compound 1 is characterized by an X-ray powder diffraction (XRPD) pattern having diffractions at angles (2 theta±0.2) of 4.61, 15.66, 23.19, and 24.76.
4. The crystalline solid form of any one of embodiments 1-3, wherein Type A of Compound 1 is characterized by an XRPD pattern having diffractions at angles (2 theta±0.2) of 4.61, 15.66, 23.19, and 24.76, corresponding to d-spacing (angstroms±0.2) of 19.19, 5.66, 3.84, and 3.60, respectively.
5. The crystalline solid form of any one of embodiments 1-4, wherein Type A of Compound 1 is characterized by an XRPD pattern having diffractions at angles (2 theta±0.2) of 4.61, 7.22, 15.66, 20.48, 21.35, 21.66, 22.47, 23.19, 24.76, and 26.73.
6. The crystalline solid form of any one of embodiments 1-5, wherein Type A of Compound 1 is characterized by an XRPD pattern having diffractions at angles (2 theta±0.2) of 4.61, 7.22, 15.66, 20.48, 21.35, 21.66, 22.47, 23.19, 24.76, and 26.73, corresponding to d-spacing (angstroms±0.2) of 19.19, 12.25, 5.66, 4.34, 4.16, 4.10, 3.96, 3.84, 3.60, and 3.34, respectively.
7. The crystalline solid form of any one of embodiments 1-6, wherein Type A of Compound 1 is characterized by an XRPD pattern having diffractions at angles (2 theta±0.2) of:
4.61
5.80
7.22
7.68
11.21
12.31
14.44
15.66
16.95
18.02
19.20
20.48
21.35
21.66
22.47
23.19
24.76
26.73
28.01
28.49
29.35
30.25
32.14
34.12
36.46
   8. The crystalline solid form of any one of embodiments 1-7, wherein Type A of Compound 1 is characterized by an XRPD pattern having diffractions at angles (2 theta±0.2) corresponding to d-spacing (angstroms±0.2) of:

| 2 theta | d-spacing |
|---|---|
| 4.61 | 19.19 |
| 5.80 | 15.24 |
| 7.22 | 12.25 |
| 7.68 | 11.50 |
| 11.21 | 7.89 |
| 12.31 | 7.19 |
| 14.44 | 6.13 |
| 15.66 | 5.66 |
| 16.95 | 5.23 |
| 18.02 | 4.92 |
| 19.20 | 4.62 |
| 20.48 | 4.34 |
| 21.35 | 4.16 |
| 21.66 | 4.10 |
| 22.47 | 3.96 |
| 23.19 | 3.84 |
| 24.76 | 3.60 |
| 26.73 | 3.34 |
| 28.01 | 3.19 |
| 28.49 | 3.13 |
| 29.35 | 3.04 |
| 30.25 | 2.95 |
| 32.14 | 2.79 |
| 34.12 | 2.63 |
| 36.46 | 2.46 |

9. The crystalline solid form of any one of embodiments 1-8, wherein Type A of Compound 1 is characterized by a thermogravimetric analysis (TGA) thermogram with a weight loss of about 1.9% up to 100° C.
10. The crystalline solid form of any one of embodiments 1-9, wherein Type A of Compound 1 is characterized by a differential scanning calorimetry (DSC) endotherm having a peak temperature of about 85.9° C. and an onset temperature of about 146.0° C.
11. The crystalline solid form of any one of embodiments 1-10, wherein Type A of Compound 1 is characterized by a dynamic vapor sorption (DVS) of about 3.4% water uptake by weight up to 40% relative humidity.
12. The crystalline solid form of any one of embodiments 1-11, wherein Type A of Compound 1 is characterized by a dynamic vapor sorption (DVS) of about 1.0% water uptake by weight from 40% to 80% relative humidity.
13. The crystalline solid form of embodiment 1, wherein the crystalline solid form is Type B of Compound 1.
14. The crystalline solid form of embodiment 1 or 13, wherein Type B of Compound 1 is characterized by an X-ray powder diffraction (XRPD) pattern having diffractions at angles (2 theta±0.2) of 4.52, 15.57, 22.89, 23.34, and 25.13.
15. The crystalline solid form of any one of embodiments 1 and 13-14, wherein Type B of Compound 1 is characterized by an XRPD pattern having diffractions at angles (2 theta±0.2) of 4.52, 15.57, 22.89, 23.34, and 25.13, corresponding to d-spacing (angstroms±0.2) of 19.53, 5.69, 3.89, 3.81, and 3.54, respectively.
16. The crystalline solid form of any one of embodiments 1 and 13-15, wherein Type B of Compound 1 is characterized by an X-ray powder diffraction (XRPD) pattern having diffractions at angles (2 theta±0.2) of 4.52, 9.86, 15.57, 19.93, 22.19, 22.89, 23.34, 25.13, and 28.30.
17. The crystalline solid form of any one of embodiments 1 and 13-16, wherein Type B of Compound 1 is characterized by an XRPD pattern having diffractions at angles (2 theta±0.2) of 4.52, 9.86, 15.57, 19.93, 22.19, 22.89, 23.34, 25.13, and 28.30, corresponding to d-spacing (angstroms±0.2) of 19.53, 8.97, 5.69, 4.45, 4.00, 3.89, 3.81, 3.54, and 3.15, respectively.
18. The crystalline solid form of any one of embodiments 1 and 13-17, wherein Type B of Compound 1 is characterized by an XRPD pattern having diffractions at angles (2 theta±0.2) of:
4.52
8.98
9.86
12.37
13.18
15.57
16.86
18.21
19.11
19.93
20.92
22.19
22.89
23.34
25.13
25.80
26.71
28.30
29.39
   19. The crystalline solid form of any one of embodiments 1 and 13-18, wherein Type B of Compound 1 is characterized by an XRPD pattern having diffractions at angles (2 theta±0.2) corresponding to d-spacing (angstroms±0.2) of:

| 2 theta | d-spacing |
|---|---|
| 4.52 | 19.53 |
| 8.98 | 9.85 |
| 9.86 | 8.97 |
| 12.37 | 7.15 |
| 13.18 | 6.72 |
| 15.57 | 5.69 |
| 16.86 | 5.26 |
| 18.21 | 4.87 |
| 19.11 | 4.64 |
| 19.93 | 4.45 |
| 20.92 | 4.25 |
| 22.19 | 4.00 |
| 22.89 | 3.89 |
| 23.34 | 3.81 |
| 25.13 | 3.54 |
| 25.80 | 3.45 |
| 26.71 | 3.34 |
| 28.30 | 3.15 |
| 29.39 | 3.04 |

20. The crystalline solid form of any one of embodiments 1 and 13-19, wherein Type B of Compound 1 is characterized by a thermogravimetric analysis (TGA) thermogram with a weight loss of about 1.8% up to 100° C.
21. The crystalline solid form of any one of embodiments 1 and 13-20, wherein Type B of Compound 1 is characterized by a thermogravimetric analysis (TGA) thermogram with a weight loss of about 2.3% up to 120° C.
22. The crystalline solid form of any one of embodiments 1 and 13-21, wherein Type B of Compound 1 is characterized by a differential scanning calorimetry (DSC) endotherm having an onset temperature of about 138.2-139.2° C.
23. The crystalline solid form of any one of embodiments 1 and 13-22, wherein Type B of Compound 1 is characterized by a dynamic vapor sorption (DVS) of about 2.9% water uptake by weight up to 60% relative humidity.
24. The crystalline solid form of any one of embodiments 1 and 13-23, wherein Type B of Compound 1 is characterized by a dynamic vapor sorption (DVS) of about 0.4% water uptake by weight from 60% to 80% relative humidity.
25. The crystalline solid form of embodiment 1, wherein the crystalline solid form is Type C of Compound 1.
26. The crystalline solid form of embodiment 1 or 25, wherein Type C of Compound 1 is characterized by an X-ray powder diffraction (XRPD) pattern having diffractions at angles (2 theta±0.2) of 4.55, 18.85, 23.02, and 24.65.
27. The crystalline solid form of any one of embodiments 1 and 25-26, wherein Type C of Compound 1 is characterized by an XRPD pattern having diffractions at angles (2 theta±0.2) of 4.55, 18.85, 23.02, and 24.65, corresponding to d-spacing (angstroms±0.2) of 19.43, 4.71, 3.86, and 3.61, respectively.
28. The crystalline solid form of any one of embodiments 1 and 25-27, wherein Type C of Compound 1 is characterized by an X-ray powder diffraction (XRPD) pattern having diffractions at angles (2 theta±0.2) of 4.55, 7.34, 9.07, 11.17, 18.34, 18.85, 19.57, 21.66, 23.02, and 24.65.
29. The crystalline solid form of any one of embodiments 1 and 25-28, wherein Type C of Compound 1 is characterized by an XRPD pattern having diffractions at angles (2 theta±0.2) of 4.55, 7.34, 9.07, 11.17, 18.34, 18.85, 19.57, 21.66, 23.02, and 24.65, corresponding to d-spacing (angstroms±0.2) of 19.43, 12.05, 9.75, 7.92, 4.84, 4.71, 4.54, 4.10, 3.86, and 3.61, respectively.
30. The crystalline solid form of any one of embodiments 1 and 25-29, wherein Type C of Compound 1 is characterized by an XRPD pattern having diffractions at angles (2 theta±0.2) of:
4.55
7.34
9.07
11.17
12.29
14.51
15.66
18.34
18.85
19.57
20.38
21.66
23.02
24.65
26.39
28.28
30.09
32.31
33.91
37.19
   31. The crystalline solid form of any one of embodiments 1 and 25-30, wherein Type C of Compound 1 is characterized by an XRPD pattern having diffractions at angles (2 theta±0.2) corresponding to d-spacing (angstroms±0.2) of:

| 2 theta | d-spacing |
|---|---|
| 4.55 | 19.43 |
| 7.34 | 12.05 |
| 9.07 | 9.75 |
| 11.17 | 7.92 |
| 12.29 | 7.20 |
| 14.51 | 6.11 |
| 15.66 | 5.66 |
| 18.34 | 4.84 |
| 18.85 | 4.71 |
| 19.57 | 4.54 |
| 20.38 | 4.36 |
| 21.66 | 4.10 |
| 23.02 | 3.86 |
| 24.65 | 3.61 |
| 26.39 | 3.38 |
| 28.28 | 3.16 |
| 30.09 | 2.97 |
| 32.31 | 2.77 |
| 33.91 | 2.64 |
| 37.19 | 2.42 |

32. The crystalline solid form of any one of embodiments 1 and 25-31, wherein Type C of Compound 1 is characterized by a thermogravimetric analysis (TGA) thermogram with a weight loss of about 1.0% up to 100° C.
33. The crystalline solid form of any one of embodiments 1 and 25-32, wherein Type C of Compound 1 is characterized by a thermogravimetric analysis (TGA) thermogram with a weight loss of about 2.3% up to 130° C.
34. The crystalline solid form of any one of embodiments 1 and 25-33, wherein Type C of Compound 1 is characterized by a differential scanning calorimetry (DSC) endotherm having an onset temperature of about 152.2-154.2° C.
35. The crystalline solid form of any one of embodiments 1 and 25-34, wherein Type C of Compound 1 is characterized by a dynamic vapor sorption (DVS) of about 1.8% water uptake by weight up to 60% relative humidity.
36. The crystalline solid form of any one of embodiments 1 and 25-35, wherein Type C of Compound 1 is characterized by a dynamic vapor sorption (DVS) of about 0.5% water uptake by weight from 60% to 80% relative humidity.
37. The crystalline solid form of embodiment 1, wherein the crystalline solid form is Type D of Compound 1.
38. The crystalline solid form of embodiment 1 or 37, wherein Type D of Compound 1 is characterized by an X-ray powder diffraction (XRPD) pattern having diffractions at angles (2 theta±0.2) of 9.72, 13.08, 15.74, 21.90, and 23.59.
39. The crystalline solid form of any one of embodiments 1 and 37-38, wherein Type D of Compound 1 is characterized by an XRPD pattern having diffractions at angles (2 theta±0.2) of 9.72, 13.08, 15.74, 21.90, and 23.59, corresponding to d-spacing (angstroms±0.2) of 9.10, 6.77, 5.63, 4.06 and 3.77, respectively.
40. The crystalline solid form of any one of embodiments 1 and 37-39, wherein Type D of Compound 1 is characterized by an X-ray powder diffraction (XRPD) pattern having diffractions at angles (2 theta±0.2) of 4.27, 6.15, 8.71, 9.72, 12.31, 13.08, 13.76, 15.74, 18.02, 21.90, 23.59, and 26.71.
41. The crystalline solid form of any one of embodiments 1 and 37-40, wherein Type D of Compound 1 is characterized by an XRPD pattern having diffractions at angles (2 theta±0.2) of 4.27, 6.15, 8.71, 9.72, 12.31, 13.08, 13.76, 15.74, 18.02, 21.90, 23.59, and 26.71, corresponding to d-spacing (angstroms±0.2) of 20.68, 14.36, 10.16, 9.10, 7.19, 6.77, 6.44, 5.63, 4.92, 4.06, 3.77, and 3.34, respectively.
42. The crystalline solid form of any one of embodiments 1 and 37-41, wherein Type D of Compound 1 is characterized by an XRPD pattern having diffractions at angles (2 theta±0.2) of:
4.27
6.15
8.71
9.72
12.31
13.08
13.76
15.74
18.02
19.55
21.90
23.59
24.79
26.71
29.50
30.82
31.74
35.40
37.84
38.61
   43. The crystalline solid form of any one of embodiments 1 and 37-42, wherein Type D of Compound 1 is characterized by an XRPD pattern having diffractions at angles (2 theta±0.2) corresponding to d-spacing (angstroms±0.2) of:

| 2 theta | d-spacing |
|---|---|
| 4.27 | 20.68 |
| 6.15 | 14.36 |
| 8.71 | 10.16 |
| 9.72 | 9.10 |
| 12.31 | 7.19 |
| 13.08 | 6.77 |
| 13.76 | 6.44 |
| 15.74 | 5.63 |
| 18.02 | 4.92 |
| 19.55 | 4.54 |
| 21.90 | 4.06 |
| 23.59 | 3.77 |
| 24.79 | 3.59 |
| 26.71 | 3.34 |
| 29.50 | 3.03 |
| 30.82 | 2.90 |
| 31.74 | 2.82 |
| 35.40 | 2.54 |
| 37.84 | 2.38 |
| 38.61 | 2.33 |

44. The crystalline solid form of any one of embodiments 1 and 37-43, wherein Type D of Compound 1 is characterized by a thermogravimetric analysis (TGA) thermogram with a weight loss of about 9.6% up to 130° C.
45. The crystalline solid form of any one of embodiments 1 and 37-44, wherein Type D of Compound 1 is characterized by a differential scanning calorimetry (DSC) endotherm having an onset temperature of about 91.9° C.
46. The crystalline solid form of embodiment 1, wherein the crystalline solid form is Type E of Compound 1.
47. The crystalline solid form of embodiment 1 or 46, wherein Type E of Compound 1 is characterized by an X-ray powder diffraction (XRPD) pattern having diffractions at angles (2 theta±0.2) of 15.12, 15.75, 17.48, 20.05, 21.93, and 26.72.
48. The crystalline solid form of any one of embodiments 1 and 46-47, wherein Type E of Compound 1 is characterized by an XRPD pattern having diffractions at angles (2 theta±0.2) of 15.12, 15.75, 17.48, 20.05, 21.93, and 26.72, corresponding to d-spacing (angstroms±0.2) of 5.86, 5.63, 5.07, 4.43, 4.05, and 3.34, respectively.
49. The crystalline solid form of any one of embodiments 1 and 46-48, wherein Type E of Compound 1 is characterized by an X-ray powder diffraction (XRPD) pattern having diffractions at angles (2 theta±0.2) of 4.59, 15.12, 15.75, 17.48, 20.05, 21.93, 23.18, 23.70, and 26.72.
50. The crystalline solid form of any one of embodiments 1 and 46-49, wherein Type E of Compound 1 is characterized by an XRPD pattern having diffractions at angles (2 theta±0.2) of 4.59, 15.12, 15.75, 17.48, 20.05, 21.93, 23.18, 23.70, and 26.72, corresponding to d-spacing (angstroms±0.2) of 19.27, 5.86, 5.63, 5.07, 4.43, 4.05, 3.84, 3.75, and 3.34, respectively.
51. The crystalline solid form of any one of embodiments 1 and 46-50, wherein Type E of Compound 1 is characterized by an X-ray powder diffraction (XRPD) pattern having diffractions at angles (2 theta±0.2) of 4.59, 9.76, 12.36, 13.12, 15.12, 15.75, 16.84, 17.48, 18.06, 19.02, 20.05, 21.93, 23.18, 23.70, 26.72, and 27.81.
52. The crystalline solid form of any one of embodiments 1 and 46-51, wherein Type E of Compound 1 is characterized by an XRPD pattern having diffractions at angles (2 theta±0.2) of 4.59, 9.76, 12.36, 13.12, 15.12, 15.75, 16.84, 17.48, 18.06, 19.02, 20.05, 21.93, 23.18, 23.70, 26.72, and 27.81, corresponding to d-spacing (angstroms±0.2) of 19.27, 9.06, 7.16, 6.75, 5.86, 5.63, 5.27, 5.07, 4.91, 4.67, 4.43, 4.05, 3.84, 3.75, 3.34, and 3.21, respectively.
53. The crystalline solid form of any one of embodiments 1 and 46-52, wherein Type E of Compound 1 is characterized by an XRPD pattern having diffractions at angles (2 theta±0.2) of:
4.59
8.76
9.76
12.36
13.12
13.83
15.12
15.75
16.84
17.48
18.06
19.02
20.05
21.93
23.18
23.70
24.82
26.72
27.81
29.51
30.76
31.74
33.03
34.52
35.39
36.72
37.77
38.66
   54. The crystalline solid form of any one of embodiments 1 and 46-53, wherein Type E of Compound 1 is characterized by an XRPD pattern having diffractions at angles (2 theta±0.2) corresponding to d-spacing (angstroms±0.2) of:

| 2 theta | d-spacing |
|---|---|
| 4.59 | 19.27 |
| 8.76 | 10.09 |
| 9.76 | 9.06 |
| 12.36 | 7.16 |
| 13.12 | 6.75 |
| 13.83 | 6.40 |
| 15.12 | 5.86 |
| 15.75 | 5.63 |
| 16.84 | 5.27 |
| 17.48 | 5.07 |
| 18.06 | 4.91 |
| 19.02 | 4.67 |
| 20.05 | 4.43 |
| 21.93 | 4.05 |
| 23.18 | 3.84 |
| 23.70 | 3.75 |
| 24.82 | 3.59 |
| 26.72 | 3.34 |
| 27.81 | 3.21 |
| 29.51 | 3.03 |
| 30.76 | 2.91 |
| 31.74 | 2.82 |
| 33.03 | 2.71 |
| 34.52 | 2.60 |
| 35.39 | 2.54 |
| 36.72 | 2.45 |
| 37.77 | 2.38 |
| 38.66 | 2.33 |

55. The crystalline solid form of embodiment 1, wherein the crystalline solid form is Type F of Compound 1.
56. The crystalline solid form of embodiment 1 or 55, wherein Type F of Compound 1 is characterized by an X-ray powder diffraction (XRPD) pattern having diffractions at angles (2 theta±0.2) of 5.45, 14.66, 16.00, 16.79, 20.01, 21.36, and 22.45.
57. The crystalline solid form of any one of embodiments 1 and 55-56, wherein Type F of Compound 1 is characterized by an XRPD pattern having diffractions at angles (2 theta±0.2) of 5.45, 14.66, 16.00, 16.79, 20.01, 21.36, and 22.45, corresponding to d-spacing (angstroms±0.2) of 16.23, 6.04, 5.54, 5.28, 4.44, 4.16, and 3.96, respectively.
58. The crystalline solid form of any one of embodiments 1 and 55-57, wherein Type F of Compound 1 is characterized by an X-ray powder diffraction (XRPD) pattern having diffractions at angles (2 theta±0.2) of 5.45, 14.66, 16.00, 16.79, 18.99, 20.01, 21.36, 22.45, 23.25, and 25.32.
59. The crystalline solid form of any one of embodiments 1 and 55-58, wherein Type F of Compound 1 is characterized by an XRPD pattern having diffractions at angles (2 theta±0.2) of 5.45, 14.66, 16.00, 16.79, 18.99, 20.01, 21.36, 22.45, 23.25, and 25.32, corresponding to d-spacing (angstroms±0.2) of 16.23, 6.04, 5.54, 5.28, 4.67, 4.44, 4.16, 3.96, 3.83, and 3.52, respectively.
60. The crystalline solid form of any one of embodiments 1 and 55-59, wherein Type F of Compound 1 is characterized by an X-ray powder diffraction (XRPD) pattern having diffractions at angles (2 theta±0.2) of 5.45, 12.87, 14.66, 16.00, 16.79, 17.36, 18.99, 20.01, 20.57, 21.36, 22.45, 23.25, 25.32, 26.57, 27.25, 27.97, and 30.02.
61. The crystalline solid form of any one of embodiments 1 and 55-60, wherein Type F of Compound 1 is characterized by an XRPD pattern having diffractions at angles (2 theta±0.2) of 5.45, 12.87, 14.66, 16.00, 16.79, 17.36, 18.99, 20.01, 20.57, 21.36, 22.45, 23.25, 25.32, 26.57, 27.25, 27.97, and 30.02, corresponding to d-spacing (angstroms±0.2) of 16.23, 6.88, 6.04, 5.54, 5.28, 5.11, 4.67, 4.44, 4.32, 4.16, 3.96, 3.83, 3.52, 3.35, 3.27, 3.19, and 2.98, respectively.
62. The crystalline solid form of any one of embodiments 1 and 55-61, wherein Type F of Compound 1 is characterized by an XRPD pattern having diffractions at angles (2 theta±0.2) of:
5.45
10.92
12.87
14.66
16.00
16.79
17.36
18.99
20.01
20.57
21.36
22.45
23.25
25.32
26.57
27.25
27.97
30.02
31.98
32.89
38.29
39.09
   63. The crystalline solid form of any one of embodiments 1 and 55-62, wherein Type F of Compound 1 is characterized by an XRPD pattern having diffractions at angles (2 theta±0.2) corresponding to d-spacing (angstroms±0.2) of:

| 2 theta | d-spacing |
|---|---|
| 5.45 | 16.23 |
| 10.92 | 8.10 |
| 12.87 | 6.88 |
| 14.66 | 6.04 |
| 16.00 | 5.54 |
| 16.79 | 5.28 |
| 17.36 | 5.11 |
| 18.99 | 4.67 |
| 20.01 | 4.44 |
| 20.57 | 4.32 |
| 21.36 | 4.16 |
| 22.45 | 3.96 |
| 23.25 | 3.83 |
| 25.32 | 3.52 |
| 26.57 | 3.35 |
| 27.25 | 3.27 |
| 27.97 | 3.19 |
| 30.02 | 2.98 |
| 31.98 | 2.80 |
| 32.89 | 2.72 |
| 38.29 | 2.35 |
| 39.09 | 2.30 |

64. The crystalline solid form of any one of embodiments 1 and 55-63, wherein Type F of Compound 1 is characterized by a thermogravimetric analysis (TGA) thermogram with a weight loss of about 6.2% up to 120° C.
65. The crystalline solid form of any one of embodiments 1 and 55-64, wherein Type F of Compound 1 is characterized by a differential scanning calorimetry (DSC) endotherm having a peak temperature of about 100.4° C. and an onset temperature of 125.9° C.
66. The crystalline solid form of embodiment 1, wherein the crystalline solid form is Type G of Compound 1.
67. The crystalline solid form of embodiment 1 or 66, wherein Type G of Compound 1 is characterized by an X-ray powder diffraction (XRPD) pattern having diffractions at angles (2 theta±0.2) of 5.36, 14.34, 16.58, and 21.35.
68. The crystalline solid form of any one of embodiments 1 and 66-67, wherein Type G of Compound 1 is characterized by an XRPD pattern having diffractions at angles (2 theta±0.2) of 5.36, 14.34, 16.58, and 21.35, corresponding to d-spacing (angstroms±0.2) of 16.48, 6.18, 5.35, and 4.16, respectively.
69. The crystalline solid form of any one of embodiments 1 and 66-68, wherein Type G of Compound 1 is characterized by an X-ray powder diffraction (XRPD) pattern having diffractions at angles (2 theta±0.2) of 5.36, 12.83, 14.34, 15.00, 16.58, 19.78, 21.35, 22.35, 25.33, and 26.43.
70. The crystalline solid form of any one of embodiments 1 and 66-69, wherein Type G of Compound 1 is characterized by an XRPD pattern having diffractions at angles (2 theta±0.2) of 5.36, 12.83, 14.34, 15.00, 16.58, 19.78, 21.35, 22.35, 25.33, and 26.43, corresponding to d-spacing (angstroms±0.2) of 16.48, 6.90, 6.18, 5.91, 5.35, 4.49, 4.16, 3.98, 3.52, and 3.37, respectively.
71. The crystalline solid form of any one of embodiments 1 and 66-70, wherein Type G of Compound 1 is characterized by an X-ray powder diffraction (XRPD) pattern having diffractions at angles (2 theta±0.2) of 5.36, 12.83, 14.34, 15.00, 15.79, 16.58, 19.78, 21.35, 22.35, 25.33, 26.43, 27.35, and 30.21.
72. The crystalline solid form of any one of embodiments 1 and 66-71, wherein Type G of Compound 1 is characterized by an XRPD pattern having diffractions at angles (2 theta±0.2) of 5.36, 12.83, 14.34, 15.00, 15.79, 16.58, 19.78, 21.35, 22.35, 25.33, 26.43, 27.35, and 30.21, corresponding to d-spacing (angstroms±0.2) of 16.48, 6.90, 6.18, 5.91, 5.61, 5.35, 4.49, 4.16, 3.98, 3.52, 3.37, 3.26, and 2.96, respectively.
73. The crystalline solid form of any one of embodiments 1 and 66-72, wherein Type G of Compound 1 is characterized by an XRPD pattern having diffractions at angles (2 theta±0.2) of:
5.36
8.73
12.83
14.34
15.00
15.79
16.58
18.54
19.78
21.35
22.35
23.38
25.33
26.43
27.35
30.21
32.32
38.04
   74. The crystalline solid form of any one of embodiments 1 and 66-73, wherein Type G of Compound 1 is characterized by an XRPD pattern having diffractions at angles (2 theta±0.2) corresponding to d-spacing (angstroms±0.2) of:

| 2 theta | d-spacing |
|---|---|
| 5.36 | 16.48 |
| 8.73 | 10.13 |
| 12.83 | 6.90 |
| 14.34 | 6.18 |
| 15.00 | 5.91 |
| 15.79 | 5.61 |
| 16.58 | 5.35 |
| 18.54 | 4.79 |
| 19.78 | 4.49 |
| 21.35 | 4.16 |
| 22.35 | 3.98 |
| 23.38 | 3.80 |
| 25.33 | 3.52 |
| 26.43 | 3.37 |
| 27.35 | 3.26 |
| 30.21 | 2.96 |
| 32.32 | 2.77 |
| 38.04 | 2.37 |

75. A pharmaceutical composition comprising a therapeutically effective amount of the crystalline solid form of any one of embodiments 1-74, and one or more pharmaceutically acceptable excipients.
76. The pharmaceutical composition of embodiment 75, wherein the pharmaceutical composition is for oral administration.
77. The pharmaceutical composition of embodiment 75 or 76, wherein the pharmaceutical composition has a water content of about 0.5-5.0 weight %.
78. The pharmaceutical composition of any one of embodiments 75-77, wherein the pharmaceutical composition has a water content of about 1.5-4.0 weight %.
79. The pharmaceutical composition of any one of embodiments 75-78, wherein the pharmaceutical composition has a water content of about 2.5-3.0 weight %.
80. An amorphous solid dispersion comprising Compound 1:

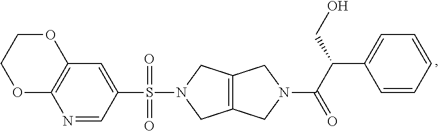 [SMILES: O=C([C@H](CO)c1ccccc1)N1CC2=C(C1)CN(S(=O)(=O)c1cnc3c(c1)OCCO3)C2]

and a polymer.
   81. The amorphous solid dispersion of embodiment 80, wherein the polymer is selected from a group consisting of hydroxypropylmethyl cellulose (HPMC), hydroxypropylmethyl cellulose acetate succinate (HPMC AS), hydroxypropyl methyl cellulose phthalate (HPMCP), hydroxypropyl cellulose (HPC), ethylcellulose, cellulose acetate phthalate, polyvinylpyrrolidone (PVP), and a combination thereof.
   82. The amorphous solid dispersion of embodiment 80 or 81, wherein the polymer is hydroxypropylmethyl cellulose (HPMC) or hydroxypropylmethyl cellulose acetate succinate (HPMC AS).
   83. The amorphous solid dispersion of any one of embodiments 80-82, wherein the weight ratio of Compound 1 to the polymer is in a range of about 3:1 to about 1:3.
   84. The amorphous solid dispersion of any one of embodiments 80-83, wherein the weight ratio of Compound 1 to the polymer is about 1:3.
   85. A pharmaceutical composition comprising a therapeutically effective amount of the amorphous solid dispersion of any one of embodiments 80-84, and one or more pharmaceutically acceptable excipients.
   86. The pharmaceutical composition of embodiment 85, wherein the pharmaceutical composition is for oral administration.
   87. The pharmaceutical composition of embodiment 85 or 86, wherein the pharmaceutical composition is in a tablet dosage form.
   88. The pharmaceutical composition of any one of embodiments 85-87, wherein the pharmaceutical composition has a water content of about 0.5-5.0 weight %.
   89. The pharmaceutical composition of any one of embodiments 85-88, wherein the pharmaceutical composition has a water content of about 1.5-4.0 weight %.
   90. The pharmaceutical composition of any one of embodiments 85-89, wherein the pharmaceutical composition has a water content of about 2.5-3.0 weight %.
   91. The pharmaceutical composition of any one of embodiments 85-90, wherein the pharmaceutical composition comprises about 10 mg, about 25 mg, about 50 mg, about 100 mg, about 200 mg, or about 300 mg of Compound 1.
   92. The pharmaceutical composition of any one of embodiments 85-91, wherein the pharmaceutical composition comprises about 25 mg of Compound 1.
   93. The pharmaceutical composition of any one of embodiments 85-91, wherein the pharmaceutical composition comprises about 100 mg of Compound 1.
   94. The pharmaceutical composition of any one of embodiments 85-93, wherein the one or more pharmaceutically acceptable excipients comprise one or more of a filler, a dry binder, a glidant, a lubricant, a disintegrant, and a film coating agent.
   95. The pharmaceutical composition of any one of embodiments 85-94, wherein the one or more pharmaceutically acceptable excipients comprise a filler, and the filler comprises microcrystalline cellulose.
   96. The pharmaceutical composition of any one of embodiments 85-95, wherein the one or more pharmaceutically acceptable excipients comprise a dry binder, and the dry binder comprises crospovidone.
   97. The pharmaceutical composition of any one of embodiments 85-96, wherein the one or more pharmaceutically acceptable excipients comprise a glidant, and the glidant comprises colloidal silicon dioxide.
   98. The pharmaceutical composition of any one of embodiments 85-97, wherein the one or more pharmaceutically acceptable excipients comprise a lubricant, and the lubricant comprises magnesium stearate.
   99. The pharmaceutical composition of any one of embodiments 85-98, wherein the one or more pharmaceutically acceptable excipients comprise a disintegrant, and the disintegrant comprises croscarmellose sodium.
   100. The pharmaceutical composition of any one of embodiments 85-99, wherein the one or more pharmaceutically acceptable excipients comprise a lubricant, and the lubricant comprises magnesium stearate.
   101. The pharmaceutical composition of any one of embodiments 85-100, comprising a tablet core, the tablet core comprising:
an intra granular portion comprising the amorphous solid dispersion; and
an extra granular portion blended with the intra granular portion.
   102. The pharmaceutical composition of embodiment 101, further comprising a coating disposed on the tablet core.
   103. The pharmaceutical composition of embodiment 101 or 102, wherein the amorphous solid dispersion is about 50 weight % of the tablet core.
   104. The pharmaceutical composition of any one of embodiments 101-103, wherein the intra granular portion further comprises one or more of a filler, a dry binder, a glidant, and a lubricant.
   105. The pharmaceutical composition of any one of embodiments 101-104, wherein the extra granular portion further comprises one or more of a filler, a disintegrant, and a lubricant.
   106. A method for preparing an amorphous solid dispersion comprising Compound 1:

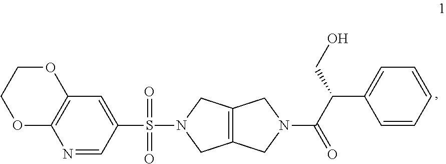 [SMILES: O=C([C@H](CO)c1ccccc1)N1CC2=C(C1)CN(S(=O)(=O)c1cnc3c(c1)OCCO3)C2]

comprising:
   mixing Compound 1, a polymer, and a solvent to afford a mixture; and
   spray-drying the mixture to afford an amorphous solid dispersion comprising Compound 1.
      107. The method of embodiment 106, wherein the polymer is selected from a group consisting of hydroxypropylmethyl cellulose (HPMC), hydroxypropylmethyl cellulose acetate succinate (HPMC AS), hydroxypropyl methyl cellulose phthalate (HPMCP), hydroxypropyl cellulose (HPC), ethylcellulose, cellulose acetate phthalate, polyvinylpyrrolidone (PVP), and a combination thereof.
      108. The method of embodiment 106 or 107, wherein the polymer is hydroxypropylmethyl cellulose (HPMC) or hydroxypropylmethyl cellulose acetate succinate (HPMC AS).
      109. The method of any one of embodiments 106-108, wherein the weight ratio of Compound 1 to the polymer is in a range of about 3:1 to about 1:3.
      110. The method of any one of embodiments 106-109, wherein the weight ratio of Compound 1 to the polymer is about 1:3.
      111. The method of any one of embodiments 106-110, wherein the solvent is dichloromethane and methanol.
      112. A product prepared by a process comprising:
mixing Compound 1, a polymer, and a solvent to afford a mixture; and
spray-drying the mixture to afford an amorphous solid dispersion comprising Compound 1:

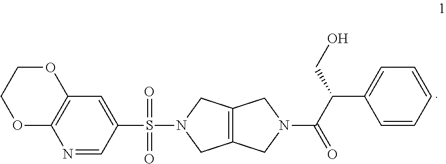 [SMILES: O=C([C@H](CO)c1ccccc1)N1CC2=C(C1)CN(S(=O)(=O)c1cnc3c(c1)OCCO3)C2]

113. The product of embodiment 112, wherein the polymer is selected from a group consisting of hydroxypropylmethyl cellulose (HPMC), hydroxypropylmethyl cellulose acetate succinate (HPMC AS), hydroxypropyl methyl cellulose phthalate (HPMCP), hydroxypropyl cellulose (HPC), ethylcellulose, cellulose acetate phthalate, polyvinylpyrrolidone (PVP), and a combination thereof.
114. The product of embodiment 112 or 113, wherein the polymer is hydroxypropylmethyl cellulose (HPMC) or hydroxypropylmethyl cellulose acetate succinate (HPMC AS).
115. The product of any one of embodiments 112-114, wherein the weight ratio of Compound 1 to the polymer is in a range of about 3:1 to about 1:3.
116. The product of any one of embodiments 112-115, wherein the weight ratio of Compound 1 to the polymer is about 1:3.
117. The product of any one of embodiments 112-116, wherein the solvent is dichloromethane and methanol.
118. A pharmaceutical composition comprising Compound 1:

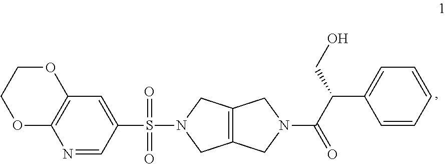 [SMILES: O=C([C@H](CO)c1ccccc1)N1CC2=C(C1)CN(S(=O)(=O)c1cnc3c(c1)OCCO3)C2]

obtained by a process comprising:
   mixing Compound 1 in a solid form, a polymer, and a solvent to afford a mixture; and
   spray-drying the mixture to afford an amorphous solid dispersion comprising Compound 1.
      119. The pharmaceutical composition of embodiment 118, wherein the solid form is Type A of Compound 1.
      120. The pharmaceutical composition of embodiment 118, wherein the solid form is Type B of Compound 1.
      121. The pharmaceutical composition of embodiment 118, wherein the solid form is Type C of Compound 1.
      122. The pharmaceutical composition of embodiment 118, wherein the solid form is Type D of Compound 1.
      123. The pharmaceutical composition of embodiment 118, wherein the solid form is Type E of Compound 1.
      124. The pharmaceutical composition of embodiment 118, wherein the solid form is Type F of Compound 1.
      125. The pharmaceutical composition of embodiment 118, wherein the solid form is Type G of Compound 1.
      126. The pharmaceutical composition of embodiment 118, wherein the solid form is amorphous form of Compound 1.
      127. The pharmaceutical composition of any one of embodiments 118-126, wherein the pharmaceutical composition has a water content of about 0.5-5.0 weight %.
      128. The pharmaceutical composition of any one of embodiments 118-127, wherein the pharmaceutical composition has a water content of about 1.5-4.0 weight %.
      129. The pharmaceutical composition of any one of embodiments 118-128, wherein the pharmaceutical composition has a water content of about 2.5-3.0 weight %.
      130. The pharmaceutical composition of any one of embodiments 118-129, wherein the polymer is selected from a group consisting of hydroxypropylmethyl cellulose (HPMC), hydroxypropylmethyl cellulose acetate succinate (HPMC AS), hydroxypropyl methyl cellulose phthalate (HPMCP), hydroxypropyl cellulose (HPC), ethylcellulose, cellulose acetate phthalate, polyvinylpyrrolidone (PVP), and a combination thereof.
      131. The pharmaceutical composition of any one of embodiments 118-130, wherein the polymer is hydroxypropylmethyl cellulose (HPMC) or hydroxypropylmethyl cellulose acetate succinate (HPMC AS).
      132. The pharmaceutical composition of any one of embodiments 118-131, wherein the weight ratio of Compound 1 to the polymer is in a range of about 3:1 to about 1:3.
      133. The pharmaceutical composition of any one of embodiments 118-132, wherein the weight ratio of Compound 1 to the polymer is about 1:3.
      134. The pharmaceutical composition of any one of embodiments 118-133, wherein the solvent is dichloromethane and methanol.

In some embodiments, the disclosure relates to one or more of the following enumerated embodiments:

1. A crystalline solid form of Compound 1:

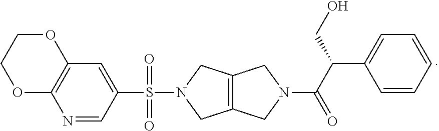 [SMILES: O=C([C@H](CO)c1ccccc1)N1CC2=C(C1)CN(S(=O)(=O)c1cnc3c(c1)OCCO3)C2]

2. The crystalline solid form of embodiment 1, wherein the crystalline solid form is Type A of (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one (“Compound 1”).
3. The crystalline solid form of embodiment 2, wherein Type A of Compound 1 is characterized by an X-ray powder diffraction (XRPD) pattern having diffractions at angles (2 theta±0.2) of 4.6, 15.7, 23.2, and 24.8.
4. The crystalline solid form of embodiment 2 or 3, wherein Type A of Compound 1 is characterized by an XRPD pattern having diffractions at angles (2 theta±0.2) of 4.6, 15.7, 23.2, and 24.8, corresponding to d-spacing (angstroms±0.2) of 19.2, 5.7, 3.8, and 3.6, respectively.
5. The crystalline solid form of any one of embodiments 2-4, wherein Type A of Compound 1 is characterized by an X-ray powder diffraction (XRPD) pattern having diffractions at angles (2 theta±0.2) of 4.6, 7.2, 15.7, 21.3, 23.2, and 24.8.
6. The crystalline solid form of any one of embodiments 2-5, wherein Type A of Compound 1 is characterized by an XRPD pattern having diffractions at angles (2 theta±0.2) of 4.6, 7.2, 15.7, 21.3, 23.2, and 24.8, corresponding to d-spacing (angstroms±0.2) of 19.2, 12.3, 5.7, 4.2, 3.8, and 3.6, respectively.
7. The crystalline solid form of any one of embodiment 2-6, wherein Type A of Compound 1 is characterized by an XRPD pattern having diffractions at angles (2 theta±0.2) of 4.6, 7.2, 15.7, 20.5, 21.3, 21.7, 22.5, 23.2, 24.8, and 26.7.
8. The crystalline solid form of any one of embodiment 2-7, wherein Type A of Compound 1 is characterized by an XRPD pattern having diffractions at angles (2 theta±0.2) of 4.6, 7.2, 15.7, 20.5, 21.3, 21.7, 22.5, 23.2, 24.8, and 26.7, corresponding to d-spacing (angstroms±0.2) of 19.2, 12.2, 5.7, 4.3, 4.2, 4.1, 4.0, 3.8, 3.6, and 3.3, respectively.
9. The crystalline solid form of any one of embodiments 2-8, wherein Type A of Compound 1 is characterized by an XRPD pattern having diffractions at angles (2 theta±0.2) of:
4.6
5.8
7.2
7.7
11.2
12.3
14.4
15.7
16.9
18.0
19.2
20.5
21.3
21.7
22.5
23.2
24.8
26.7
28.0
28.5
29.4
30.3
32.1
34.1
36.5.
   10. The crystalline solid form of any one of embodiments 2-9, wherein Type A of Compound 1 is characterized by an XRPD pattern having diffractions at angles (2 theta±0.2) corresponding to d-spacing (angstroms±0.2) of:

| 2 theta | d-spacing |
|---|---|
| 4.6 | 19.2 |
| 5.8 | 15.2 |
| 7.2 | 12.2 |
| 7.7 | 11.5 |
| 11.2 | 7.9 |
| 12.3 | 7.2 |
| 14.4 | 6.1 |
| 15.7 | 5.7 |
| 16.9 | 5.2 |
| 18.0 | 4.9 |
| 19.2 | 4.6 |
| 20.5 | 4.3 |
| 21.3 | 4.2 |
| 21.7 | 4.1 |
| 22.5 | 4.0 |
| 23.2 | 3.8 |
| 24.8 | 3.6 |
| 26.7 | 3.3 |
| 28.0 | 3.2 |
| 28.5 | 3.1 |
| 29.4 | 3.0 |
| 30.3 | 3.0 |
| 32.1 | 2.8 |
| 34.1 | 2.6 |
| 36.5 | 2.5. |

11. The crystalline solid form of any one of embodiments 2-10, wherein Type A of Compound 1 is characterized by a thermogravimetric analysis (TGA) thermogram with a weight loss of about 1.9% up to 100° C.
12. The crystalline solid form of any one of embodiments 2-11, wherein Type A of Compound 1 is characterized by a differential scanning calorimetry (DSC) endotherm having a peak temperature of about 85.9° C. and an onset temperature of about 146.0° C.
13. The crystalline solid form of any one of embodiments 2-12, wherein Type A of Compound 1 is characterized by a dynamic vapor sorption (DVS) of about 3.4% water uptake by weight up to 40% relative humidity.
14. The crystalline solid form of any one of embodiments 2-13, wherein Type A of Compound 1 is characterized by a dynamic vapor sorption (DVS) of about 1.0% water uptake by weight from 40% to 80% relative humidity.
15. The crystalline solid form of embodiment 1, wherein the crystalline solid form is Type B of Compound 1.
16. The crystalline solid form of embodiment 15, wherein Type B of Compound 1 is characterized by an X-ray powder diffraction (XRPD) pattern having diffractions at angles (2 theta±0.2) of 4.5, 15.6, 22.9, 23.3, and 25.1.
17. The crystalline solid form of embodiment 15 or 16, wherein Type B of Compound 1 is characterized by an XRPD pattern having diffractions at angles (2 theta±0.2) of 4.5, 15.6, 22.9, 23.3, and 25.1, corresponding to d-spacing (angstroms±0.2) of 19.5, 5.7, 3.9, 3.8, and 3.5, respectively.
18. The crystalline solid form of any one of embodiments 15-17, wherein Type B of Compound 1 is characterized by an X-ray powder diffraction (XRPD) pattern having diffractions at angles (2 theta±0.2) of 4.5, 15.6, 22.2, 22.9, 23.3, and 25.1.
19. The crystalline solid form of any one of embodiments 15-18, wherein Type B of Compound 1 is characterized by an XRPD pattern having diffractions at angles (2 theta±0.2) of 4.5, 15.6, 22.2, 22.9, 23.3, and 25.1, corresponding to d-spacing (angstroms±0.2) of 19.5, 5.7, 4.0, 3.9, 3.8, and 3.5, respectively.
20. The crystalline solid form of any one of embodiments 15-19, wherein Type B of Compound 1 is characterized by an X-ray powder diffraction (XRPD) pattern having diffractions at angles (2 theta±0.2) of 4.5, 9.9, 15.6, 19.9, 22.2, 22.9, 23.3, 25.1, and 28.3.
21. The crystalline solid form of any one of embodiments 15-20, wherein Type B of Compound 1 is characterized by an XRPD pattern having diffractions at angles (2 theta±0.2) of 4.5, 9.9, 15.6, 19.9, 22.2, 22.9, 23.3, 25.1, and 28.3, corresponding to d-spacing (angstroms 0.2) of 19.5, 9.0, 5.7, 4.5, 4.0, 3.9, 3.8, 3.5, and 3.2, respectively.
22. The crystalline solid form of any one of embodiments 15-21, wherein Type B of Compound 1 is characterized by an XRPD pattern having diffractions at angles (2 theta±0.2) of:
4.5
9.0
9.9
12.4
13.2
15.6
16.9
18.2
19.1
19.9
20.9
22.2
22.9
23.3
25.1
25.8
26.7
28.3
29.4.
   23. The crystalline solid form of any one of embodiments 15-22, wherein Type B of Compound 1 is characterized by an XRPD pattern having diffractions at angles (2 theta±0.2) corresponding to d-spacing (angstroms±0.2) of:

| 2 theta | d-spacing |
|---|---|
| 4.5 | 19.5 |
| 9.0 | 9.9 |
| 9.9 | 9.0 |
| 12.4 | 7.2 |
| 13.2 | 6.7 |
| 15.6 | 5.7 |
| 16.9 | 5.3 |
| 18.2 | 4.9 |
| 19.1 | 4.6 |
| 19.9 | 4.5 |
| 20.9 | 4.2 |
| 22.2 | 4.0 |
| 22.9 | 3.9 |
| 23.3 | 3.8 |
| 25.1 | 3.5 |
| 25.8 | 3.5 |
| 26.7 | 3.3 |
| 28.3 | 3.2 |
| 29.4 | 3.0. |

24. The crystalline solid form of any one of embodiments 15-23, wherein Type B of Compound 1 is characterized by a thermogravimetric analysis (TGA) thermogram with a weight loss of about 1.8% up to 100° C.
25. The crystalline solid form of any one of embodiments 15-24, wherein Type B of Compound 1 is characterized by a thermogravimetric analysis (TGA) thermogram with a weight loss of about 2.3% up to 120° C.
26. The crystalline solid form of any one of embodiments 15-25, wherein Type B of Compound 1 is characterized by a differential scanning calorimetry (DSC) endotherm having an onset temperature of about 138.2-139.2° C.
27. The crystalline solid form of any one of embodiments 15-26, wherein Type B of Compound 1 is characterized by a dynamic vapor sorption (DVS) of about 2.9% water uptake by weight up to 60% relative humidity.
28. The crystalline solid form of any one of embodiments 15-27, wherein Type B of Compound 1 is characterized by a dynamic vapor sorption (DVS) of about 0.4% water uptake by weight from 60% to 80% relative humidity.
29. The crystalline solid form of embodiment 1, wherein the crystalline solid form is Type C of Compound 1.
30. The crystalline solid form of embodiment 29, wherein Type C of Compound 1 is characterized by an X-ray powder diffraction (XRPD) pattern having diffractions at angles (2 theta±0.2) of 4.5, 18.9, 23.0, and 24.7.
31. The crystalline solid form of embodiment 29 or 30, wherein Type C of Compound 1 is characterized by an XRPD pattern having diffractions at angles (2 theta±0.2) of 4.5, 18.9, 23.0, and 24.7, corresponding to d-spacing (angstroms±0.2) of 19.4, 4.7, 3.9, and 3.6, respectively.
32. The crystalline solid form of any one of embodiments 29-31, wherein Type C of Compound 1 is characterized by an X-ray powder diffraction (XRPD) pattern having diffractions at angles (2 theta±0.2) of 4.5, 7.3, 11.2, 18.9, 23.0, and 24.7.
33. The crystalline solid form of any one of embodiments 29-32, wherein Type C of Compound 1 is characterized by an XRPD pattern having diffractions at angles (2 theta±0.2) of 4.5, 7.3, 11.2, 18.9, 23.0, and 24.7, corresponding to d-spacing (angstroms±0.2) of 19.4, 12.0, 7.9, 4.7, 3.9, and 3.6, respectively.
34. The crystalline solid form of any one of embodiments 29-33, wherein Type C of Compound 1 is characterized by an X-ray powder diffraction (XRPD) pattern having diffractions at angles (2 theta±0.2) of 4.5, 7.3, 9.1, 11.2, 18.3, 18.9, 19.6, 21.7, 23.0, and 24.7.
35. The crystalline solid form of any one of embodiments 9-34, wherein Type C of Compound 1 is characterized by an XRPD pattern having diffractions at angles (2 theta±0.2) of 4.5, 7.3, 9.1, 11.2, 18.3, 18.9, 19.6, 21.7, 23.0, and 24.7, corresponding to d-spacing (angstroms±0.2) of 19.4, 12.0, 9.8, 7.9, 4.8, 4.7, 4.5, 4.1, 3.9, and 3.6, respectively.
36. The crystalline solid form of any one of embodiments 29-35, wherein Type C of Compound 1 is characterized by an XRPD pattern having diffractions at angles (2 theta±0.2) of:
4.5
7.3
9.1
11.2
12.3
14.5
15.7
18.3
18.9
19.6
20.4
21.7
23.0
24.7
26.4
28.3
30.1
32.3
33.9
37.2.
   37. The crystalline solid form of any one of embodiments 29-36, wherein Type C of Compound 1 is characterized by an XRPD pattern having diffractions at angles (2 theta±0.2) corresponding to d-spacing (angstroms±0.2) of:

| 2 theta | d-spacing |
|---|---|
| 4.5 | 19.4 |
| 7.3 | 12.0 |
| 9.1 | 9.8 |
| 11.2 | 7.9 |
| 12.3 | 7.2 |
| 14.5 | 6.1 |
| 15.7 | 5.7 |
| 18.3 | 4.8 |
| 18.9 | 4.7 |
| 19.6 | 4.5 |
| 20.4 | 4.4 |
| 21.7 | 4.1 |
| 23.0 | 3.9 |
| 24.7 | 3.6 |
| 26.4 | 3.4 |
| 28.3 | 3.2 |
| 30.1 | 3.0 |
| 32.3 | 2.8 |
| 33.9 | 2.6 |
| 37.2 | 2.4. |

38. The crystalline solid form of any one of embodiments 29-37, wherein Type C of Compound 1 is characterized by a thermogravimetric analysis (TGA) thermogram with a weight loss of about 1.0% up to 100° C.
39. The crystalline solid form of any one of embodiments 29-38, wherein Type C of Compound 1 is characterized by a thermogravimetric analysis (TGA) thermogram with a weight loss of about 2.3% up to 130° C.
40. The crystalline solid form of any one of embodiments 29-39, wherein Type C of Compound 1 is characterized by a differential scanning calorimetry (DSC) endotherm having an onset temperature of about 152.2-154.2° C.
41. The crystalline solid form of any one of embodiments 29-40, wherein Type C of Compound 1 is characterized by a dynamic vapor sorption (DVS) of about 1.8% water uptake by weight up to 60% relative humidity.
42. The crystalline solid form of any one of embodiments 29-41, wherein Type C of Compound 1 is characterized by a dynamic vapor sorption (DVS) of about 0.5% water uptake by weight from 60% to 80% relative humidity.
43. The crystalline solid form of embodiment 1, wherein the crystalline solid form is Type D of Compound 1.
44. The crystalline solid form of embodiment 43, wherein Type D of Compound 1 is characterized by an X-ray powder diffraction (XRPD) pattern having diffractions at angles (2 theta±0.2) of 9.7, 13.1, 15.7, 21.9, and 23.6.
45. The crystalline solid form of embodiment 43 or 44, wherein Type D of Compound 1 is characterized by an XRPD pattern having diffractions at angles (2 theta±0.2) of 9.7, 13.1, 15.7, 21.9, and 23.6, corresponding to d-spacing (angstroms±0.2) of 9.1, 6.8, 5.6, 4.1 and 3.8, respectively.
46. The crystalline solid form of any one of embodiments 43-45, wherein Type D of Compound 1 is characterized by an X-ray powder diffraction (XRPD) pattern having diffractions at angles (2 theta±0.2) of 6.2, 9.7, 13.1, 15.7, 21.9, and 23.6 and not having a diffraction at an angle (2 theta±0.2) of 23.3.
47. The crystalline solid form of any one of embodiments 43-46, wherein Type D of Compound 1 is characterized by an XRPD pattern having diffractions at angles (2 theta±0.2) of 6.2, 9.7, 13.1, 15.7, 21.9, and 23.6, corresponding to d-spacing (angstroms±0.2) of 14.4, 9.1, 6.8, 5.6, 4.1 and 3.8, respectively, and not having a diffraction at an angle (2 theta±0.2) of 23.3.
48. The crystalline solid form of any one of embodiments 43-47, wherein Type D of Compound 1 is characterized by an X-ray powder diffraction (XRPD) pattern having diffractions at angles (2 theta±0.2) of 4.3, 6.2, 8.7, 9.7, 12.3, 13.1, 13.8, 15.7, 18.0, 21.9, 23.6, and 26.7.
49. The crystalline solid form of any one of embodiments 43-48, wherein Type D of Compound 1 is characterized by an XRPD pattern having diffractions at angles (2 theta±0.2) of 4.3, 6.2, 8.7, 9.7, 12.3, 13.1, 13.8, 15.7, 18.0, 21.9, 23.6, and 26.7, corresponding to d-spacing (angstroms±0.2) of 20.7, 14.4, 10.2, 9.1, 7.2, 6.8, 6.4, 5.6, 4.9, 4.1, 3.8, and 3.3, respectively.
50. The crystalline solid form of any one of embodiments 43-49, wherein Type D of Compound 1 is characterized by an XRPD pattern having diffractions at angles (2 theta±0.2) of:
4.3
6.2
8.7
9.7
12.3
13.1
13.8
15.7
18.0
19.5
21.9
23.6
24.8
26.7
29.5
30.8
31.7
35.4
37.8
38.6.
   51. The crystalline solid form of any one of embodiments 43-50, wherein Type D of Compound 1 is characterized by an XRPD pattern having diffractions at angles (2 theta±0.2) corresponding to d-spacing (angstroms±0.2) of:

| 2 theta | d-spacing |
|---|---|
| 4.3 | 20.7 |
| 6.2 | 14.4 |
| 8.7 | 10.2 |
| 9.7 | 9.1 |
| 12.3 | 7.2 |
| 13.1 | 6.8 |
| 13.8 | 6.4 |
| 15.7 | 5.6 |
| 18.0 | 4.9 |
| 19.5 | 4.5 |
| 21.9 | 4.1 |
| 23.6 | 3.8 |
| 24.8 | 3.6 |
| 26.7 | 3.3 |
| 29.5 | 3.0 |
| 30.8 | 2.9 |
| 31.7 | 2.8 |
| 35.4 | 2.5 |
| 37.8 | 2.4 |
| 38.6 | 2.3. |

52. The crystalline solid form of any one of embodiments 43-51, wherein Type D of Compound 1 is characterized by a thermogravimetric analysis (TGA) thermogram with a weight loss of about 9.6% up to 130° C.
53. The crystalline solid form of any one of embodiments 43-52, wherein Type D of Compound 1 is characterized by a differential scanning calorimetry (DSC) endotherm having an onset temperature of about 91.9° C.
54. The crystalline solid form of embodiment 1, wherein the crystalline solid form is Type E of Compound 1.
55. The crystalline solid form of embodiment 54, wherein Type E of Compound 1 is characterized by an X-ray powder diffraction (XRPD) pattern having diffractions at angles (2 theta±0.2) of 15.1, 15.8, 17.5, 20.1, 21.9, and 26.7.
56. The crystalline solid form of embodiment 54 or 55, wherein Type E of Compound 1 is characterized by an XRPD pattern having diffractions at angles (2 theta±0.2) of 15.1, 15.8, 17.5, 20.1, 21.9, and 26.7, corresponding to d-spacing (angstroms±0.2) of 5.9, 5.6, 5.1, 4.4, 4.1, and 3.3, respectively.
57. The crystalline solid form of any one of embodiments 54-56, wherein Type E of Compound 1 is characterized by an X-ray powder diffraction (XRPD) pattern having diffractions at angles (2 theta±0.2) of 15.1, 15.8, 17.5, 20.1, 21.9, and 26.7.
58. The crystalline solid form of any one of embodiments 54-57, wherein Type E of Compound 1 is characterized by an XRPD pattern having diffractions at angles (2 theta±0.2) of 15.1, 15.8, 17.5, 19.0, 20.1, 21.9, and 26.7, corresponding to d-spacing (angstroms±0.2) of 5.9, 5.6, 5.1, 4.7, 4.4, 4.1, and 3.3, respectively.
59. The crystalline solid form of any one of embodiments 54-56, wherein Type E of Compound 1 is characterized by an X-ray powder diffraction (XRPD) pattern having diffractions at angles (2 theta±0.2) of 4.6, 15.1, 15.8, 17.5, 20.1, 21.9, 23.2, 23.7, and 26.7.
60. The crystalline solid form of any one of embodiments 54-56, wherein Type E of Compound 1 is characterized by an XRPD pattern having diffractions at angles (2 theta±0.2) of 4.6, 15.1, 15.8, 17.5, 20.1, 21.9, 23.2, 23.7, and 26.7, corresponding to d-spacing (angstroms 0.2) of 19.3, 5.9, 5.6, 5.1, 4.4, 4.1, 3.8, 3.8, and 3.3, respectively.
61. The crystalline solid form of any one of embodiments 54-60, wherein Type E of Compound 1 is characterized by an X-ray powder diffraction (XRPD) pattern having diffractions at angles (2 theta±0.2) of 4.6, 9.8, 12.4, 13.1, 15.1, 15.8, 16.8, 17.5, 18.1, 19.0, 20.1, 21.9, 23.2, 23.7, 26.7, and 27.8.
62. The crystalline solid form of any one of embodiments 54-61, wherein Type E of Compound 1 is characterized by an XRPD pattern having diffractions at angles (2 theta±0.2) of 4.6, 9.8, 12.4, 13.1, 15.1, 15.8, 16.8, 17.5, 18.1, 19.0, 20.1, 21.9, 23.2, 23.7, 26.7, and 27.8, corresponding to d-spacing (angstroms±0.2) of 19.3, 9.1, 7.2, 6.7, 5.9, 5.6, 5.3, 5.1, 4.9, 4.7, 4.4, 4.1, 3.8, 3.8, 3.3, and 3.2, respectively.
63. The crystalline solid form of any one of embodiments 54-62, wherein Type E of Compound 1 is characterized by an XRPD pattern having diffractions at angles (2 theta±0.2) of:
4.6
8.8
9.8
12.4
13.1
13.8
15.1
15.8
16.8
17.5
18.1
19.0
20.1
21.9
23.2
23.7
24.8
26.7
27.8
29.5
30.8
31.7
33.0
34.5
35.4
36.7
37.8
38.7.
   64. The crystalline solid form of any one of embodiments 54-63, wherein Type E of Compound 1 is characterized by an XRPD pattern having diffractions at angles (2 theta±0.2) corresponding to d-spacing (angstroms±0.2) of:

| 2 theta | d-spacing |
|---|---|
| 4.6 | 19.3 |
| 8.8 | 10.1 |
| 9.8 | 9.1 |
| 12.4 | 7.2 |
| 13.1 | 6.7 |
| 13.8 | 6.4 |
| 15.1 | 5.9 |
| 15.8 | 5.6 |
| 16.8 | 5.3 |
| 17.5 | 5.1 |
| 18.1 | 4.9 |
| 19.0 | 4.7 |
| 20.1 | 4.4 |
| 21.9 | 4.1 |
| 23.2 | 3.8 |
| 23.7 | 3.8 |
| 24.8 | 3.6 |
| 26.7 | 3.3 |
| 27.8 | 3.2 |
| 29.5 | 3.0 |
| 30.8 | 2.9 |
| 31.7 | 2.8 |
| 33.0 | 2.7 |
| 34.5 | 2.6 |
| 35.4 | 2.5 |
| 36.7 | 2.4 |
| 37.8 | 2.4 |
| 38.7 | 2.3. |

65. The crystalline solid form of embodiment 1, wherein the crystalline solid form is Type F of Compound 1.
66. The crystalline solid form of embodiment 65, wherein Type F of Compound 1 is characterized by an X-ray powder diffraction (XRPD) pattern having diffractions at angles (2 theta±0.2) of 5.4, 14.7, 16.0, 16.8, and 21.4.
67. The crystalline solid form of embodiment 65 or 66, wherein Type F of Compound 1 is characterized by an XRPD pattern having diffractions at angles (2 theta±0.2) of 5.4, 14.7, 16.0, 16.8, and 21.4, corresponding to d-spacing (angstroms±0.2) of 16.2, 6.0, 5.5, 5.3, and 4.2, respectively.
68. The crystalline solid form of any one of embodiments 65-67, wherein Type F of Compound 1 is characterized by an X-ray powder diffraction (XRPD) pattern having diffractions at angles (2 theta±0.2) of 5.4, 14.7, 16.0, 16.8, 20.0, 21.4, and 22.5.
69. The crystalline solid form of any one of embodiments 65-68, wherein Type F of Compound 1 is characterized by an XRPD pattern having diffractions at angles (2 theta±0.2) of 5.4, 14.7, 16.0, 16.8, 20.0, 21.4, and 22.5, corresponding to d-spacing (angstroms±0.2) of 16.2, 6.0, 5.5, 5.3, 4.4, 4.2, and 4.0, respectively.
70. The crystalline solid form of any one of embodiments 65-69, wherein Type F of Compound 1 is characterized by an X-ray powder diffraction (XRPD) pattern having diffractions at angles (2 theta±0.2) of 5.4, 14.7, 16.0, 16.8, 19.0, 20.0, 21.4, 22.5, 23.2, and 25.3.
71. The crystalline solid form of any one of embodiments 65-70, wherein Type F of Compound 1 is characterized by an XRPD pattern having diffractions at angles (2 theta±0.2) of 5.4, 14.7, 16.0, 16.8, 19.0, 20.0, 21.4, 22.5, 23.2, and 25.3, corresponding to d-spacing (angstroms±0.2) of 16.2, 6.0, 5.5, 5.3, 4.7, 4.4, 4.2, 4.0, 3.8, and 3.5, respectively.
72. The crystalline solid form of any one of embodiments 65-71, wherein Type F of Compound 1 is characterized by an X-ray powder diffraction (XRPD) pattern having diffractions at angles (2 theta±0.2) of 5.4, 12.9, 14.7, 16.0, 16.8, 17.4, 19.0, 20.0, 20.6, 21.4, 22.5, 23.2, 25.3, 26.6, 27.2, 28.0, and 30.0.
73. The crystalline solid form of any one of embodiments 65-72, wherein Type F of Compound 1 is characterized by an XRPD pattern having diffractions at angles (2 theta±0.2) of 5.4, 12.9, 14.7, 16.0, 16.8, 17.4, 19.0, 20.0, 20.6, 21.4, 22.5, 23.2, 25.3, 26.6, 27.2, 28.0, and 30.0, corresponding to d-spacing (angstroms±0.2) of 16.2, 6.9, 6.0, 5.5, 5.3, 5.1, 4.7, 4.4, 4.3, 4.2, 4.0, 3.8, 3.5, 3.4, 3.3, 3.2, and 3.0, respectively.
74. The crystalline solid form of any one of embodiments 65-73, wherein Type F of Compound 1 is characterized by an XRPD pattern having diffractions at angles (2 theta±0.2) of:
5.4
10.9
12.9
14.7
16.0
16.8
17.4
19.0
20.0
20.6
21.4
22.5
23.2
25.3
26.6
27.2
28.0
30.0
32.0
32.9
38.3
39.1
   75. The crystalline solid form of any one of embodiments 65-74, wherein Type F of Compound 1 is characterized by an XRPD pattern having diffractions at angles (2 theta±0.2) corresponding to d-spacing (angstroms±0.2) of:

| 2 theta | d-spacing |
|---|---|
| 5.4 | 16.23 |
| 10.9 | 8.1 |
| 12.9 | 6.9 |
| 14.7 | 6.0 |
| 16.0 | 5.5 |
| 16.8 | 5.3 |
| 17.4 | 5.1 |
| 19.0 | 4.7 |
| 20.0 | 4.4 |
| 20.6 | 4.3 |
| 21.4 | 4.2 |
| 22.5 | 4.0 |
| 23.2 | 3.8 |
| 25.3 | 3.5 |
| 26.6 | 3.4 |
| 27.2 | 3.3 |
| 28.0 | 3.2 |
| 30.0 | 3.0 |
| 32.0 | 2.8 |
| 32.9 | 2.7 |
| 38.3 | 2.4 |
| 39.1 | 2.3 |

76. The crystalline solid form of any one of embodiments 65-75, wherein Type F of Compound 1 is characterized by a thermogravimetric analysis (TGA) thermogram with a weight loss of about 6.2% up to 120° C.
77. The crystalline solid form of any one of embodiments 65-76, wherein Type F of Compound 1 is characterized by a differential scanning calorimetry (DSC) endotherm having a peak temperature of about 100.4° C. and an onset temperature of 125.9° C.
78. The crystalline solid form of embodiment 1, wherein the crystalline solid form is Type G of Compound 1.
79. The crystalline solid form of embodiment 78, wherein Type G of Compound 1 is characterized by an X-ray powder diffraction (XRPD) pattern having diffractions at angles (2 theta±0.2) of 5.4, 14.3, 16.6, and 21.3.
80. The crystalline solid form of embodiment 78 or 79, wherein Type G of Compound 1 is characterized by an XRPD pattern having diffractions at angles (2 theta±0.2) of 5.4, 14.3, 16.6, and 21.3, corresponding to d-spacing (angstroms±0.2) of 16.5, 6.2, 5.3, and 4.2, respectively.
81. The crystalline solid form of any one of embodiments 78-80, wherein Type G of Compound 1 is characterized by an X-ray powder diffraction (XRPD) pattern having diffractions at angles (2 theta±0.2) of 5.4, 14.3, 16.6, 21.3, and 22.3.
82. The crystalline solid form of any one of embodiments 78-81, wherein Type G of Compound 1 is characterized by an XRPD pattern having diffractions at angles (2 theta±0.2) of 5.4, 14.3, 16.6, 21.3, and 22.3, corresponding to d-spacing (angstroms±0.2) of 16.5, 6.2, 5.3, 4.2, and 4.0, respectively.
83. The crystalline solid form of any one of embodiments 78-82, wherein Type G of Compound 1 is characterized by an X-ray powder diffraction (XRPD) pattern having diffractions at angles (2 theta±0.2) of 5.4, 12.8, 14.3, 15.0, 16.6, 19.8, 21.3, 22.3, 25.3, and 26.4.
84. The crystalline solid form of any one of embodiments 78-83, wherein Type G of Compound 1 is characterized by an XRPD pattern having diffractions at angles (2 theta±0.2) of 5.4, 12.8, 14.3, 15.0, 16.6, 19.8, 21.3, 22.3, 25.3, and 26.4, corresponding to d-spacing (angstroms±0.2) of 16.5, 6.9, 6.2, 5.9, 5.3, 4.5, 4.2, 4.0, 3.5, and 3.4, respectively.
85. The crystalline solid form of any one of embodiments 78-84, wherein Type G of Compound 1 is characterized by an X-ray powder diffraction (XRPD) pattern having diffractions at angles (2 theta±0.2) of 5.4, 12.8, 14.3, 15.0, 15.8, 16.6, 19.8, 21.3, 22.3, 25.3, 26.4, 27.4, and 30.2.
86. The crystalline solid form of any one of embodiments 78-85, wherein Type G of Compound 1 is characterized by an XRPD pattern having diffractions at angles (2 theta±0.2) of 5.4, 12.8, 14.3, 15.0, 15.8, 16.6, 19.8, 21.3, 22.3, 25.3, 26.4, 27.4, and 30.2, corresponding to d-spacing (angstroms±0.2) of 16.5, 6.9, 6.2, 5.9, 5.6, 5.3, 4.5, 4.2, 4.0, 3.5, 3.4, 3.3, and 3.0, respectively.
87. The crystalline solid form of any one of embodiments 78-86, wherein Type G of Compound 1 is characterized by an XRPD pattern having diffractions at angles (2 theta±0.2) of:
5.4
8.7
12.8
14.3
15.0
15.8
16.6
18.5
19.8
21.3
22.3
23.4
25.3
26.4
27.4
30.2
32.3
38.0
   88. The crystalline solid form of any one of embodiments 78-87, wherein Type G of Compound 1 is characterized by an XRPD pattern having diffractions at angles (2 theta±0.2) corresponding to d-spacing (angstroms±0.2) of:

| 2 theta | d-spacing |
|---|---|
| 5.4 | 16.5 |
| 8.7 | 10.1 |
| 12.8 | 6.9 |
| 14.3 | 6.2 |
| 15.0 | 5.9 |
| 15.8 | 5.6 |
| 16.6 | 5.3 |
| 18.5 | 4.8 |
| 19.8 | 4.5 |
| 21.3 | 4.2 |
| 22.3 | 4.0 |
| 23.4 | 3.8 |
| 25.3 | 3.5 |
| 26.4 | 3.4 |
| 27.4 | 3.3 |
| 30.2 | 3.0 |
| 32.3 | 2.8 |
| 38.0 | 2.4 |

89. The crystalline solid form of embodiment 1, wherein the crystalline solid form is Type H of Compound 1.
90. The crystalline solid form of embodiment 89, wherein Type H of Compound 1 is characterized by an X-ray powder diffraction (XRPD) pattern having diffractions at angles (2 theta±0.2) of 5.8, 14.7, 16.6, 20.0, 21.3, and 25.4.
91. The crystalline solid form of embodiment 89 or 90, wherein Type H of Compound 1 is characterized by an XRPD pattern having diffractions at angles (2 theta±0.2) of 5.8, 14.7, 16.6, 20.0, 21.3, and 25.4, corresponding to d-spacing (angstroms±0.2) of 15.3, 6.0, 5.4, 4.4, 4.2, and 3.5, respectively.
92. The crystalline solid form of any one of embodiments 89-91, wherein Type H of Compound 1 is characterized by an XRPD pattern having diffractions at angles (2 theta±0.2) of:
5.8
8.4
11.5
12.4
13.1
13.7
14.7
14.9
16.0
16.2
16.6
16.9
17.3
17.7
18.3
19.5
20.0
21.3
21.9
23.1
23.6
23.9
24.4
24.9
25.1
25.4
26.2
27.4
28.1
28.4
29.3
29.7
30.4
31.0
32.7
33.4
34.1
34.8
35.5
35.8
36.4
37.1
38.5
   93. The crystalline solid form of any one of embodiments 89-92, wherein Type H of Compound 1 is characterized by an XRPD pattern having diffractions at angles (2 theta±0.2) corresponding to d-spacing (angstroms±0.2) of:

| Pos. [°2Th.] | d-spacing [Å] |
|---|---|
| 5.8 | 15.3 |
| 8.4 | 10.5 |
| 11.5 | 7.7 |
| 12.4 | 7.2 |
| 13.1 | 6.8 |
| 13.7 | 6.5 |
| 14.7 | 6.0 |
| 14.9 | 5.9 |
| 16.0 | 5.6 |
| 16.2 | 5.5 |
| 16.6 | 5.4 |
| 16.9 | 5.3 |
| 17.3 | 5.1 |
| 17.7 | 5.0 |
| 18.3 | 4.8 |
| 19.5 | 4.6 |
| 20.0 | 4.4 |
| 21.3 | 4.2 |
| 21.9 | 4.1 |
| 23.1 | 3.9 |
| 23.6 | 3.8 |
| 23.9 | 3.7 |
| 24.4 | 3.7 |
| 24.9 | 3.6 |
| 25.1 | 3.5 |
| 25.4 | 3.5 |
| 26.2 | 3.4 |
| 27.4 | 3.3 |
| 28.1 | 3.2 |
| 28.4 | 3.1 |
| 29.3 | 3.0 |
| 29.7 | 3.0 |
| 30.4 | 2.9 |
| 31.0 | 2.9 |
| 32.7 | 2.7 |
| 33.4 | 2.7 |
| 34.1 | 2.6 |
| 34.8 | 2.6 |
| 35.5 | 2.5 |
| 35.8 | 2.5 |
| 36.4 | 2.5 |
| 37.1 | 2.4 |
| 38.5 | 2.3 |

94. The crystalline solid form of embodiment 1, wherein the crystalline solid form is Type I of Compound 1.
95. The crystalline solid form of embodiment 94, wherein Type I of Compound 1 is characterized by an X-ray powder diffraction (XRPD) pattern having diffractions at angles (2 theta±0.2) of 5.2, 14.6, 15.5, 20.2, and 21.1.
96. The crystalline solid form of embodiment 94 or 95, wherein Type I of Compound 1 is characterized by an XRPD pattern having diffractions at angles (2 theta±0.2) of 5.2, 14.6, 15.5, 20.2, and 21.1, corresponding to d-spacing (angstroms±0.2) of 17.1, 6.1, 5.7, 4.4, and 4.2, respectively.
97. The crystalline solid form of any one of embodiments 94-96, wherein Type I of Compound 1 is characterized by an XRPD pattern having diffractions at angles (2 theta±0.2) of:
5.2
8.8
10.3
12.6
14.6
15.5
16.1
16.3
16.6
17.1
17.6
18.7
18.9
20.2
20.5
20.7
21.1
21.5
22.0
22.3
23.7
24.8
25.2
26.0
26.3
26.5
26.8
27.0
27.5
27.7
28.1
29.6
30.0
30.4
31.3
32.0
32.5
33.2
34.0
34.6
36.9
38.2
38.9
39.5
   98. The crystalline solid form of any one of embodiments 94-97, wherein Type I of Compound 1 is characterized by an XRPD pattern having diffractions at angles (2 theta±0.2) corresponding to d-spacing (angstroms±0.2) of:

| Pos. [°2Th.] | d-spacing [Å] |
|---|---|
| 5.2 | 17.1 |
| 8.8 | 10.1 |
| 10.3 | 8.6 |
| 12.6 | 7.0 |
| 14.6 | 6.1 |
| 15.5 | 5.7 |
| 16.1 | 5.5 |
| 16.3 | 5.4 |
| 16.6 | 5.3 |
| 17.1 | 5.2 |
| 17.6 | 5.0 |
| 18.7 | 4.7 |
| 18.9 | 4.7 |
| 20.2 | 4.4 |
| 20.5 | 4.3 |
| 20.7 | 4.3 |
| 21.1 | 4.2 |
| 21.5 | 4.1 |
| 22.0 | 4.0 |
| 22.3 | 4.0 |
| 23.7 | 3.8 |
| 24.8 | 3.6 |
| 25.2 | 3.5 |
| 26.0 | 3.4 |
| 26.3 | 3.4 |
| 26.5 | 3.4 |
| 26.8 | 3.3 |
| 27.0 | 3.3 |
| 27.5 | 3.2 |
| 27.7 | 3.2 |
| 28.1 | 3.2 |
| 29.6 | 3.0 |
| 30.0 | 3.0 |
| 30.4 | 2.9 |
| 31.3 | 2.9 |
| 32.0 | 2.8 |
| 32.5 | 2.8 |
| 33.2 | 2.7 |
| 34.0 | 2.6 |
| 34.6 | 2.6 |
| 36.9 | 2.4 |
| 38.2 | 2.4 |
| 38.9 | 2.3 |
| 39.5 | 2.3 |

99. The crystalline solid form of embodiment 1, wherein the crystalline solid form is Type J of Compound 1.
100. The crystalline solid form of embodiment 99, wherein Type J of Compound 1 is characterized by an X-ray powder diffraction (XRPD) pattern having diffractions at angles (2 theta±0.2) of 4.5, 5.7, 22.8, 23.1, and 24.5.
101. The crystalline solid form of embodiment 99 or 100, wherein Type J of Compound 1 is characterized by an XRPD pattern having diffractions at angles (2 theta±0.2) of 4.5, 5.7, 22.8, 23.1, and 24.5, corresponding to d-spacing (angstroms±0.2) of 19.5, 15.4, 3.9, 3.8, and 3.6, respectively.
102. The crystalline solid form of any one of embodiments 99-101, wherein Type J of Compound 1 is characterized by an XRPD pattern having diffractions at angles (2 theta±0.2) of:
4.5
5.7
7.1
7.7
9.1
10.5
11.2
11.7
12.3
12.9
14.3
14.5
15.4
15.7
16.3
17.3
18.3
18.7
19.3
19.6
20.5
21.2
21.5
22.8
23.1
23.6
24.1
24.5
25.2
25.9
26.4
27.8
29.3
36.2
37.0
   103. The crystalline solid form of any one of embodiments 99-102, wherein Type J of Compound 1 is characterized by an XRPD pattern having diffractions at angles (2 theta±0.2) corresponding to d-spacing (angstroms±0.2) of:

| Pos. [°2Th.] | d-spacing [Å] |
|---|---|
| 4.5 | 19.5 |
| 5.7 | 15.4 |
| 7.1 | 12.7 |
| 7.7 | 11.5 |
| 9.1 | 9.7 |
| 10.5 | 8.4 |
| 11.2 | 7.9 |
| 11.7 | 7.5 |
| 12.3 | 7.2 |
| 12.9 | 6.8 |
| 14.3 | 6.2 |
| 14.5 | 6.1 |
| 15.4 | 5.8 |
| 15.7 | 5.7 |
| 16.3 | 5.4 |
| 17.3 | 5.1 |
| 18.3 | 4.9 |
| 18.7 | 4.7 |
| 19.3 | 4.6 |
| 19.6 | 4.5 |
| 20.5 | 4.3 |
| 21.2 | 4.2 |
| 21.5 | 4.1 |
| 22.8 | 3.9 |
| 23.1 | 3.8 |
| 23.6 | 3.8 |
| 24.1 | 3.7 |
| 24.5 | 3.6 |
| 25.2 | 3.5 |
| 25.9 | 3.4 |
| 26.4 | 3.4 |
| 27.8 | 3.2 |
| 29.3 | 3.0 |
| 36.2 | 2.5 |
| 37.0 | 2.4 |

104. The crystalline solid form of embodiment 1, wherein the crystalline solid form is Type K of Compound 1.
105. The crystalline solid form of embodiment 104, wherein Type K of Compound 1 is characterized by an X-ray powder diffraction (XRPD) pattern having diffractions at angles (2 theta±0.2) of 4.6, 15.4, 15.6, 16.1, 23.2, and 27.4.
106. The crystalline solid form of embodiment 104 or 105, wherein Type K of Compound 1 is characterized by an XRPD pattern having diffractions at angles (2 theta±0.2) of 4.6, 15.4, 15.6, 16.1, 23.2, and 27.4, corresponding to d-spacing (angstroms±0.2) of 19.2, 5.7, 5.7, 5.5, 3.8, and 3.3, respectively.
107. The crystalline solid form of any one of embodiments 104-106, wherein Type K of Compound 1 is characterized by an XRPD pattern having diffractions at angles (2 theta±0.2) of:
4.6
9.3
10.1
12.9
13.9
14.7
15.4
15.6
16.1
17.8
18.3
18.6
19.3
20.0
20.7
21.6
21.9
22.9
23.2
24.4
25.0
25.5
26.0
27.4
28.8
29.2
30.7
31.1
32.7
36.3
   108. The crystalline solid form of any one of embodiments 104-107, wherein Type K of Compound 1 is characterized by an XRPD pattern having diffractions at angles (2 theta±0.2) corresponding to d-spacing (angstroms±0.2) of:

| Pos. [°2Th.] | d-spacing [Å] |
|---|---|
| 4.6 | 19.2 |
| 9.3 | 9.5 |
| 10.1 | 8.7 |
| 12.9 | 6.8 |
| 13.9 | 6.4 |
| 14.7 | 6.0 |
| 15.4 | 5.7 |
| 15.6 | 5.7 |
| 16.1 | 5.5 |
| 17.8 | 5.0 |
| 18.3 | 4.9 |
| 18.6 | 4.8 |
| 19.3 | 4.6 |
| 20.0 | 4.4 |
| 20.7 | 4.3 |
| 21.6 | 4.1 |
| 21.9 | 4.1 |
| 22.9 | 3.9 |
| 23.2 | 3.8 |
| 24.4 | 3.6 |
| 25.0 | 3.6 |
| 25.5 | 3.5 |
| 26.0 | 3.4 |
| 27.4 | 3.3 |
| 28.8 | 3.1 |
| 29.2 | 3.1 |
| 30.7 | 2.9 |
| 31.1 | 2.9 |
| 32.7 | 2.7 |
| 36.3 | 2.5 |

109. The crystalline solid form of embodiment 1, wherein the crystalline solid form is Type L of Compound 1.
110. The crystalline solid form of embodiment 109, wherein Type L of Compound 1 is characterized by an X-ray powder diffraction (XRPD) pattern having diffractions at angles (2 theta±0.2) of 5.9, 11.9, 17.8, 21.6, 23.9, and 36.1.
111. The crystalline solid form of embodiment 109 or 110, wherein Type L of Compound 1 is characterized by an XRPD pattern having diffractions at angles (2 theta±0.2) of 5.9, 11.9, 17.8, 21.6, 23.9, and 36.1, corresponding to d-spacing (angstroms±0.2) of 14.9, 7.5, 5.0, 4.1, 3.7, and 2.5, respectively.
112. The crystalline solid form of any one of embodiments 109-111, wherein Type L of Compound 1 is characterized by an XRPD pattern having diffractions at angles (2 theta±0.2) of:
5.9
8.4
11.9
13.3
14.7
15.0
16.2
16.7
16.9
17.8
18.9
20.4
21.2
21.6
22.2
23.9
24.6
25.5
25.7
26.1
26.8
28.1
28.8
29.9
30.6
31.9
32.4
33.6
34.2
35.6
36.1
38.2
   113. The crystalline solid form of any one of embodiments 109-112, wherein Type L of Compound 1 is characterized by an XRPD pattern having diffractions at angles (2 theta±0.2) corresponding to d-spacing (angstroms±0.2) of:

| Pos. [°2Th.] | d-spacing [Å] |
|---|---|
| 5.9 | 14.9 |
| 8.4 | 10.5 |
| 11.9 | 7.5 |
| 13.3 | 6.6 |
| 14.7 | 6.0 |
| 15.0 | 5.9 |
| 16.2 | 5.5 |
| 16.7 | 5.3 |
| 16.9 | 5.2 |
| 17.8 | 5.0 |
| 18.9 | 4.7 |
| 20.4 | 4.4 |
| 21.2 | 4.2 |
| 21.6 | 4.1 |
| 22.2 | 4.0 |
| 23.9 | 3.7 |
| 24.6 | 3.6 |
| 25.5 | 3.5 |
| 25.7 | 3.5 |
| 26.1 | 3.4 |
| 26.8 | 3.3 |
| 28.1 | 3.2 |
| 28.8 | 3.1 |
| 29.9 | 3.0 |
| 30.6 | 2.9 |
| 31.9 | 2.8 |
| 32.4 | 2.8 |
| 33.6 | 2.7 |
| 34.2 | 2.6 |
| 35.6 | 2.5 |
| 36.1 | 2.5 |
| 38.2 | 2.4 |

114. The crystalline solid form of embodiment 1, wherein the crystalline solid form is Type M of Compound 1.
115. The crystalline solid form of embodiment 114, wherein Type M of Compound 1 is characterized by an X-ray powder diffraction (XRPD) pattern having diffractions at angles (2 theta±0.2) of 4.5, 5.8, 9.7, 15.6, 21.9, and 26.7.
116. The crystalline solid form of embodiment 114 or 115, wherein Type M of Compound 1 is characterized by an XRPD pattern having diffractions at angles (2 theta±0.2) of 4.5, 5.8, 9.7, 15.6, 21.9, and 26.7, corresponding to d-spacing (angstroms±0.2) of 19.5, 15.3, 9.1, 5.7, 4.1, and 3.3, respectively.
117. The crystalline solid form of any one of embodiments 114-116, wherein Type M of Compound 1 is characterized by an XRPD pattern having diffractions at angles (2 theta±0.2) of:
4.5
5.8
6.1
8.7
9.0
9.7
12.3
13.1
13.7
14.5
15.1
15.6
16.8
17.4
18.0
18.5
19.5
20.0
21.4
21.9
22.3
22.9
23.3
23.5
24.1
25.0
25.8
26.3
26.7
27.8
28.1
29.4
30.8
31.7
33.0
35.3
37.8
38.6
   118. The crystalline solid form of any one of embodiments 114-117, wherein Type M of Compound 1 is characterized by an XRPD pattern having diffractions at angles (2 theta±0.2) corresponding to d-spacing (angstroms±0.2) of:

| Pos. [°2Th.] | d-spacing [Å] |
|---|---|
| 4.5 | 19.5 |
| 5.8 | 15.3 |
| 6.1 | 14.4 |
| 8.7 | 10.2 |
| 9.0 | 9.9 |
| 9.7 | 9.1 |
| 12.3 | 7.2 |
| 13.1 | 6.8 |
| 13.7 | 6.4 |
| 14.5 | 6.1 |
| 15.1 | 5.9 |
| 15.6 | 5.7 |
| 16.8 | 5.3 |
| 17.4 | 5.1 |
| 18.0 | 4.9 |
| 18.5 | 4.8 |
| 19.5 | 4.5 |
| 20.0 | 4.4 |
| 21.4 | 4.1 |
| 21.9 | 4.1 |
| 22.3 | 4.0 |
| 22.9 | 3.9 |
| 23.3 | 3.8 |
| 23.5 | 3.8 |
| 24.1 | 3.7 |
| 25.0 | 3.6 |
| 25.8 | 3.5 |
| 26.3 | 3.4 |
| 26.7 | 3.3 |
| 27.8 | 3.2 |
| 28.1 | 3.2 |
| 29.4 | 3.0 |
| 30.8 | 2.9 |
| 31.7 | 2.8 |
| 33.0 | 2.7 |
| 35.3 | 2.5 |
| 37.8 | 2.4 |
| 38.6 | 2.3 |

119. The crystalline solid form of embodiment 1, wherein the crystalline solid form is selected from the group consisting of:
1) Type A of Compound 1, wherein Type A of Compound 1 is characterized by an X-ray powder diffraction (XRPD) pattern having diffractions at angles (2 theta±0.2) of 4.6, 7.2, 15.7, 21.4, 23.2, and 24.8;
2) Type B of Compound 1, wherein Type B of Compound 1 is characterized by an X-ray powder diffraction (XRPD) pattern having diffractions at angles (2 theta±0.2) of 4.5, 15.6, 22.2, 22.9, 23.3, and 25.1;
3) Type C of Compound 1, wherein Type C of Compound 1 is characterized by an X-ray powder diffraction (XRPD) pattern having diffractions at angles (2 theta±0.2) of 4.5, 7.3, 11.2, 18.9, 23.0, and 24.7;
4) Type D of Compound 1, wherein Type D of Compound 1 is characterized by an X-ray powder diffraction (XRPD) pattern having diffractions at angles (2 theta±0.2) of 6.2, 9.7, 13.1, 15.7, 21.9, and 23.6 and not having a diffraction at an angle (2 theta±0.2) of 23.3;
5) Type E of Compound 1, wherein Type E of Compound 1 is characterized by an X-ray powder diffraction (XRPD) pattern having diffractions at angles (2 theta±0.2) of 15.1, 15.8, 17.5, 20.1, 21.9, and 26.7;
6) Type F of Compound 1, wherein Type F of Compound 1 is characterized by an X-ray powder diffraction (XRPD) pattern having diffractions at angles (2 theta±0.2) of 5.5, 14.7, 16.0, 16.8, and 21.4;
7) Type G of Compound 1, wherein Type G of Compound 1 is characterized by an X-ray powder diffraction (XRPD) pattern having diffractions at angles (2 theta±0.2) of 5.4, 14.3, 16.6, 21.3, and 22.3;
8) Type H of Compound 1, wherein Type H of Compound 1 is characterized by an X-ray powder diffraction (XRPD) pattern having diffractions at angles (2 theta±0.2) of 5.8, 14.7, 16.6, 20.0, 21.3, and 25.4;
9) Type I of Compound 1, wherein Type I of Compound 1 is characterized by an X-ray powder diffraction (XRPD) pattern having diffractions at angles (2 theta±0.2) of 5.2, 14.6, 15.5, 20.2, and 21.1;
10) Type J of Compound 1, wherein Type J of Compound 1 is characterized by an X-ray powder diffraction (XRPD) pattern having diffractions at angles (2 theta±0.2) of 4.5, 5.7, 22.8, 23.1, and 24.5;
11) Type K of Compound 1, wherein Type K of Compound 1 is characterized by an X-ray powder diffraction (XRPD) pattern having diffractions at angles (2 theta±0.2) of 4.6, 15.4, 15.6, 16.1, 23.2, and 27.4;
12) Type L of Compound 1, wherein Type L of Compound 1 is characterized by an X-ray powder diffraction (XRPD) pattern having diffractions at angles (2 theta±0.2) of 5.9, 11.9, 17.8, 21.6, 23.9, and 36.1; and
13) Type M of Compound 1, wherein Type M of Compound 1 is characterized by an X-ray powder diffraction (XRPD) pattern having diffractions at angles (2 theta±0.2) of 4.5, 5.8, 9.7, 15.6, 21.9, and 26.7.
   120. A pharmaceutical composition comprising a therapeutically effective amount of the crystalline solid form of any one of embodiments 1-119, and one or more pharmaceutically acceptable excipients.
   121. The pharmaceutical composition of embodiment 120, wherein the pharmaceutical composition is for oral administration.
   122. The pharmaceutical composition of embodiment 120 or 121, wherein the pharmaceutical composition has a water content of about 0.5-5.0 weight %.
   123. The pharmaceutical composition of any one of embodiments 120-122, wherein the pharmaceutical composition has a water content of about 1.5-4.0 weight %.
   124. The pharmaceutical composition of any one of embodiments 120-123, wherein the pharmaceutical composition has a water content of about 2.5-3.0 weight %.
   125. An amorphous solid dispersion comprising Compound 1:

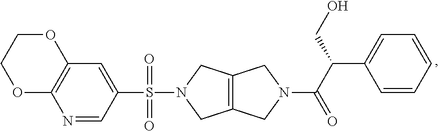 [SMILES: O=C([C@H](CO)c1ccccc1)N1CC2=C(C1)CN(S(=O)(=O)c1cnc3c(c1)OCCO3)C2]

and a polymer.
   126. The amorphous solid dispersion of embodiment 125, wherein the polymer is selected from a group consisting of hydroxypropylmethyl cellulose (HPMC), hydroxypropylmethyl cellulose acetate succinate (HPMC AS), hydroxypropyl methyl cellulose phthalate (HPMCP), hydroxypropyl cellulose (HPC), ethylcellulose, cellulose acetate phthalate, polyvinylpyrrolidone (PVP), and a combination thereof, or is selected from a group consisting of polyvinylpyrrolidone (PVP), hydroxypropylmethyl cellulose (HPMC), hydroxypropylcellulose (HPC), hydroxypropylmethyl cellulose acetate succinate (HPMC AS), hydroxyethylcellulose (HEC), poly(methacrylic acid-co-methyl methacrylates) (e.g., Eudragit® L100-55), macrogol 15 hydroxystearate (e.g., Solutol® HS15), polyvinyl caprolactam-polyvinyl acetate-polyethylene glycol graft copolymer (e.g., Soluplus®), polyethylene glycol (PEG), and a combination thereof.
   127. The amorphous solid dispersion of embodiment 125 or 126, wherein the polymer is hydroxypropylmethyl cellulose (HPMC) or hydroxypropylmethyl cellulose acetate succinate (HPMC AS).
   128. The amorphous solid dispersion of any one of embodiments 125-127, wherein the polymer is hydroxypropylmethyl cellulose acetate succinate (HPMC AS).
   129. The amorphous solid dispersion of any one of embodiments 125-128, wherein the weight ratio of Compound 1 to the polymer is in a range of about 3:1 to about 1:3 or about 2:1 to about 1:3.
   130. The amorphous solid dispersion of any one of embodiments 125-129, wherein the weight ratio of Compound 1 to the polymer is about 1:3.
   131. The amorphous solid dispersion of any one of embodiments 125-129, wherein the weight ratio of Compound 1 to the polymer is about 1:1.
   132. The amorphous solid dispersion of any one of embodiments 125-129, wherein the weight ratio of Compound 1 to the polymer is about 1:3, about 2:3, about 1:1, about 1.5:1, about 2:1, or about 3:1.
   133. The amorphous solid dispersion of any one of embodiments 125-132, wherein crystalline diffraction peaks are not observable by XRPD analysis (Method D) of the amorphous solid dispersion.
   134. The amorphous solid dispersion of any one of embodiments 125-133, wherein crystalline diffraction peaks are not observable by XRPD analysis (Method D) of the amorphous solid dispersion after storage in a container as described in Example 20 for 5 months at 2-8° C. and ambient relative humidity.
   135. The amorphous solid dispersion of any one of embodiments 125-134, wherein crystalline diffraction peaks are not observable by XRPD analysis (Method D) of the amorphous solid dispersion after storage in a container as described in Example 20 for 5 months at 25° C. and 60% relative humidity.
   136. The amorphous solid dispersion of any one of embodiments 125-135, wherein crystalline diffraction peaks are not observable by XRPD analysis (Method D) of the amorphous solid dispersion after storage in a container as described in Example 20 for 1 month at 2-8° C. and ambient relative humidity.
   137. The amorphous solid dispersion of any one of embodiments 125-136, wherein crystalline diffraction peaks are not observable by XRPD analysis (Method D) of the amorphous solid dispersion after storage in a container as described in Example 20 for 1 month at 25° C. and 60% relative humidity.
   138. The amorphous solid dispersion of any one of embodiments 125-137, wherein crystalline diffraction peaks are not observable by XRPD analysis (Method D) of the amorphous solid dispersion after storage in a container as described in Example 20 for 1 month at 40° C. and 75% relative humidity.
   139. The amorphous solid dispersion of any one of embodiments 125-138, wherein a single glass transition temperature (T_{G}) and no melt endotherm is observable by DSC analysis (Method B) of the amorphous solid dispersion.
   140. The amorphous solid dispersion of any one of embodiments 125-139, wherein a single glass transition temperature (T_{G}) and no melt endotherm is observable by DSC analysis (Method B) of the amorphous solid dispersion after storage in a container as described in Example 20 for 5 months at 2-8° C. and ambient relative humidity.
   141. The amorphous solid dispersion of any one of embodiments 125-140, wherein a single glass transition temperature (T_{G}) and no melt endotherm is observable by DSC analysis (Method B) of the amorphous solid dispersion after storage in a container as described in Example 20 for 5 months at 25° C. and 60% relative humidity.
   142. The amorphous solid dispersion of any one of embodiments 125-141, wherein a single glass transition temperature (T_{G}) and no melt endotherm is observable by DSC analysis (Method B) of the amorphous solid dispersion after storage in a container as described in Example 20 for 1 month at 2-8° C. and ambient relative humidity.
   143. The amorphous solid dispersion of any one of embodiments 125-142, wherein a single glass transition temperature (T_{G}) and no melt endotherm is observable by DSC analysis (Method B) of the amorphous solid dispersion after storage in a container as described in Example 20 for 1 month at 25° C. and 60% relative humidity.
   144. The amorphous solid dispersion of any one of embodiments 125-143, wherein a single glass transition temperature (T_{G}) and no melt endotherm is observable by DSC analysis (Method B) of the amorphous solid dispersion after storage in a container as described in Example 20 for 1 month at 40° C. and 75% relative humidity.
   145. The amorphous solid dispersion of any one of embodiments 125-144, wherein crystalline diffraction peaks are not observable by XRPD analysis (Method D) of the amorphous solid dispersion.
   146. The amorphous solid dispersion of any one of embodiments 125-145, wherein crystalline diffraction peaks are not observable by XRPD analysis (Method D) of the amorphous solid dispersion after storage in a sealed vial for 1 week at 60° C.
   147. The amorphous solid dispersion of any one of embodiments 125-146, wherein crystalline diffraction peaks are not observable by XRPD analysis (Method D) of the amorphous solid dispersion after storage in a sealed vial for 2 weeks at 60° C.
   148. The amorphous solid dispersion of any one of embodiments 125-147, wherein crystalline diffraction peaks are not observable by XRPD analysis (Method D) of the amorphous solid dispersion after storage in an unsealed vial for 1 week at 25° C. and 60% relative humidity.
   149. The amorphous solid dispersion of any one of embodiments 125-148, wherein crystalline diffraction peaks are not observable by XRPD analysis (Method D) of the amorphous solid dispersion after storage in an unsealed vial for 2 weeks at 25° C. and 60% relative humidity.
   150. The amorphous solid dispersion of any one of embodiments 125-149, wherein crystalline diffraction peaks are not observable by XRPD analysis (Method D) of the amorphous solid dispersion after storage in an unsealed vial for 1 week at 40° C. and 75% relative humidity.
   151. The amorphous solid dispersion of any one of embodiments 125-150, wherein crystalline diffraction peaks are not observable by XRPD analysis (Method D) of the amorphous solid dispersion after storage in an unsealed vial for 2 weeks at 40° C. and 75% relative humidity.
   152. The amorphous solid dispersion of any one of embodiments 125-151, wherein crystalline diffraction peaks are not observable by XRPD analysis (Method D) of the amorphous solid dispersion after storage in an unsealed vial for 1 week at 60° C. and 75% relative humidity.
   153. The amorphous solid dispersion of any one of embodiments 125-152, wherein crystalline diffraction peaks are not observable by XRPD analysis (Method D) of the amorphous solid dispersion after storage in an unsealed vial for 2 weeks at 60° C. and 75% relative humidity.
   154. The amorphous solid dispersion of any one of embodiments 125-136, wherein a single glass transition temperature (T_{G}) and no melt endotherm is observable by DSC analysis (Method B) of the amorphous solid dispersion.
   155. The amorphous solid dispersion of any one of embodiments 125-154, wherein a single glass transition temperature (T_{G}) and no melt endotherm is observable by DSC analysis (Method B) of the amorphous solid dispersion after storage in a sealed vial for 1 week or 2 weeks at 60° C.
   156. The amorphous solid dispersion of any one of embodiments 125-155, wherein a single glass transition temperature (T_{G}) and no melt endotherm is observable by DSC analysis (Method B) of the amorphous solid dispersion after storage in an unsealed vial for 1 week or 2 weeks at 25° C. and 60% relative humidity.
   157. The amorphous solid dispersion of any one of embodiments 125-156, wherein a single glass transition temperature (T_{G}) and no melt endotherm is observable by DSC analysis (Method B) of the amorphous solid dispersion after storage in an unsealed vial for 1 week or 2 weeks at 40° C. and 75% relative humidity.
   158. The amorphous solid dispersion of any one of embodiments 125-157, wherein a single glass transition temperature (T_{G}) and no melt endotherm is observable by DSC analysis (Method B) of the amorphous solid dispersion after storage in an unsealed vial for 1 week or 2 weeks at 60° C. and 75% relative humidity.
   159. The amorphous solid dispersion of any one of embodiments 125-158, wherein Compound 1 has a concentration of at least 300 μg/mL after 30 minutes in the kinetic solubility experiment described in Example 23.
   160. The amorphous solid dispersion of any one of embodiments 125-159, wherein Compound 1 has a Cmax of at least 600 μg/mL in the kinetic solubility experiment described in Example 23.
   161. The amorphous solid dispersion of any one of embodiments 125-160, wherein Compound 1 has a concentration of at least 450 μg/mL after 4 hours in the kinetic solubility experiment described in Example 23.
   162. The amorphous solid dispersion of any one of embodiments 125-158, wherein Compound 1 has a concentration of at least 200 μg/mL after 16 hours in the kinetic solubility experiment described in Example 23.
   163. A pharmaceutical composition comprising a therapeutically effective amount of the amorphous solid dispersion of any one of embodiments 125-162, and one or more pharmaceutically acceptable excipients.
   164. The pharmaceutical composition of embodiment 163, wherein the pharmaceutical composition is for oral administration.
   165. The pharmaceutical composition of embodiment 163 or 164, wherein the pharmaceutical composition is in a tablet dosage form.
   166. The pharmaceutical composition of any one of embodiments 163-165, wherein the pharmaceutical composition has a water content of about 0.5-5.0 weight %.
   167. The pharmaceutical composition of any one of embodiments 163-166, wherein the pharmaceutical composition has a water content of about 1.5-4.0 weight %.
   168. The pharmaceutical composition of any one of embodiments 163-167, wherein the pharmaceutical composition has a water content of about 2.5-3.0 weight %.
   169. The pharmaceutical composition of any one of embodiments 163-168, wherein the pharmaceutical composition comprises about 10 mg, about 25 mg, about 50 mg, about 100 mg, about 200 mg, or about 300 mg of Compound 1.
   170. The pharmaceutical composition of any one of embodiments 163-169, wherein the pharmaceutical composition comprises about 25 mg of Compound 1.
   171. The pharmaceutical composition of any one of embodiments 163-169, wherein the pharmaceutical composition comprises about 100 mg of Compound 1.
   172. The pharmaceutical composition of any one of embodiments 163-169, wherein the pharmaceutical composition comprises about 200 mg of Compound 1.
   173. The pharmaceutical composition of any one of embodiments 163-172, wherein the one or more pharmaceutically acceptable excipients comprise one or more of a filler, a dry binder, a glidant, a lubricant, a disintegrant, and a film coating agent.
   174. The pharmaceutical composition of any one of embodiments 163-173, wherein the one or more pharmaceutically acceptable excipients comprise a filler, and the filler comprises microcrystalline cellulose.
   175. The pharmaceutical composition of any one of embodiments 163-174, wherein the one or more pharmaceutically acceptable excipients comprise a filler, and the filler comprises lactose monohydrate.
   176. The pharmaceutical composition of any one of embodiments 163-175, wherein the one or more pharmaceutically acceptable excipients comprise a dry binder, and the dry binder comprises crospovidone.
   177. The pharmaceutical composition of any one of embodiments 163-176, wherein the one or more pharmaceutically acceptable excipients comprise a glidant, and the glidant comprises colloidal silicon dioxide.
   178. The pharmaceutical composition of any one of embodiments 163-177, wherein the one or more pharmaceutically acceptable excipients comprise a lubricant, and the lubricant comprises magnesium stearate.
   179. The pharmaceutical composition of any one of embodiments 163-178, wherein the one or more pharmaceutically acceptable excipients comprise a disintegrant, and the disintegrant comprises croscarmellose sodium.
   180. The pharmaceutical composition of any one of embodiments 163-179, comprising a tablet core, the tablet core comprising:
an intra granular portion comprising the amorphous solid dispersion; and
an extra granular portion blended with the intra granular portion.
   181. The pharmaceutical composition of embodiment 180, further comprising a coating disposed on the tablet core.
   182. The pharmaceutical composition of embodiment 180 or 181, wherein the amorphous solid dispersion is at least about 30 weight % of the tablet core.
   183. The pharmaceutical composition of any one of embodiments 180-182, wherein the amorphous solid dispersion is at least about 50 weight % of the tablet core.
   184. The pharmaceutical composition of any one of embodiments 180-183, wherein the amorphous solid dispersion is at least about 60 weight % of the tablet core.
   185. The pharmaceutical composition of any one of embodiments 180-184, wherein the amorphous solid dispersion is about 50 weight % of the tablet core.
   186. The pharmaceutical composition of any one of embodiments 180-185, wherein the amorphous solid dispersion is about 50 to about 70 weight % of the tablet core.
   187. The pharmaceutical composition of any one of embodiments 180-186, wherein the amorphous solid dispersion is about 60 to about 65 weight % of the tablet core.
   188. The pharmaceutical composition of any one of embodiments 180-187, wherein the intra granular portion further comprises one or more of a filler, a dry binder, a glidant, and a lubricant.
   189. The pharmaceutical composition of any one of embodiments 180-188, wherein the extra granular portion further comprises one or more of a filler, a disintegrant, and a lubricant.
   190. A method for preparing an amorphous solid dispersion comprising Compound 1:

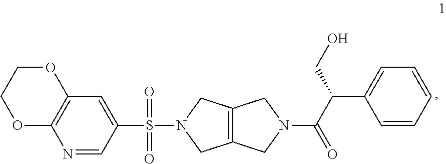 [SMILES: O=C([C@H](CO)c1ccccc1)N1CC2=C(C1)CN(S(=O)(=O)c1cnc3c(c1)OCCO3)C2]

comprising:
   mixing Compound 1, a polymer, and a solvent to afford a mixture; and
   spray-drying the mixture to afford an amorphous solid dispersion comprising Compound 1.
      191. The method of embodiment 190, wherein the polymer is selected from a group consisting of hydroxypropylmethyl cellulose (HPMC), hydroxypropylmethyl cellulose acetate succinate (HPMC AS), hydroxypropyl methyl cellulose phthalate (HPMCP), hydroxypropyl cellulose (HPC), ethylcellulose, cellulose acetate phthalate, polyvinylpyrrolidone (PVP), and a combination thereof, or is selected from a group consisting of polyvinylpyrrolidone (PVP), hydroxypropylmethyl cellulose (HPMC), hydroxypropylcellulose (HPC), hydroxypropylmethyl cellulose acetate succinate (HPMC AS), hydroxyethylcellulose (HEC), poly(methacrylic acid-co-methyl methacrylates) (e.g., Eudragit® L100-55), macrogol 15 hydroxystearate (e.g., Solutol® HS15), polyvinyl caprolactam-polyvinyl acetate-polyethylene glycol graft copolymer (e.g., Soluplus®), polyethylene glycol (PEG), and a combination thereof.
      192. The method of embodiment 190 or 191, wherein the polymer is hydroxypropylmethyl cellulose (HPMC) or hydroxypropylmethyl cellulose acetate succinate (HPMC AS).
      193. The method of any one of embodiments 190-192, wherein the polymer is hydroxypropylmethyl cellulose acetate succinate (HPMC AS).
      194. The method of any one of embodiments 190-193, wherein the weight ratio of Compound 1 to the polymer is in a range of about 3:1 to about 1:3 or about 2:1 to about 1:3.
      195. The method of any one of embodiments 190-194, wherein the weight ratio of Compound 1 to the polymer is about 1:3.
      196. The method of any one of embodiments 190-194, wherein the weight ratio of Compound 1 to the polymer is about 1:1.
      197. The method of any one of embodiments 190-194, wherein the weight ratio of Compound 1 to the polymer is about 1:3, about 2:3, about 1:1, about 1.5:1, about 2:1, or about 3:1.
      198. The method of any one of embodiments 190-197, wherein the solvent is dichloromethane and methanol.
      199. A product prepared by a process comprising:
mixing Compound 1, a polymer, and a solvent to afford a mixture; and
spray-drying the mixture to afford an amorphous solid dispersion comprising Compound 1:

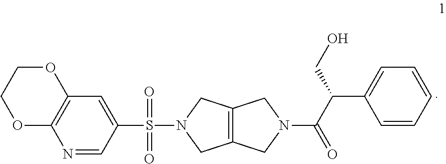 [SMILES: O=C([C@H](CO)c1ccccc1)N1CC2=C(C1)CN(S(=O)(=O)c1cnc3c(c1)OCCO3)C2]

200. The product of embodiment 199, wherein the polymer is selected from a group consisting of hydroxypropylmethyl cellulose (HPMC), hydroxypropylmethyl cellulose acetate succinate (HPMC AS), hydroxypropyl methyl cellulose phthalate (HPMCP), hydroxypropyl cellulose (HPC), ethylcellulose, cellulose acetate phthalate, polyvinylpyrrolidone (PVP), and a combination thereof, or is selected from a group consisting of polyvinylpyrrolidone (PVP), hydroxypropylmethyl cellulose (HPMC), hydroxypropylcellulose (HPC), hydroxypropylmethyl cellulose acetate succinate (HPMC AS), hydroxyethylcellulose (HEC), poly(methacrylic acid-co-methyl methacrylates) (e.g., Eudragit® L100-55), macrogol 15 hydroxystearate (e.g., Solutol® HS15), polyvinyl caprolactam-polyvinyl acetate-polyethylene glycol graft copolymer (e.g., Soluplus®), polyethylene glycol (PEG), and a combination thereof.
201. The product of embodiment 199 or 200, wherein the polymer is hydroxypropylmethyl cellulose (HPMC) or hydroxypropylmethyl cellulose acetate succinate (HPMC AS).
202. The product of any one of embodiments 199-201, wherein the polymer is hydroxypropylmethyl cellulose acetate succinate (HPMC AS).
203. The product of any one of embodiments 199-202, wherein the weight ratio of Compound 1 to the polymer is in a range of about 3:1 to about 1:3 or about 2:1 to about 1:3.
204. The product of any one of embodiments 199-203, wherein the weight ratio of Compound 1 to the polymer is about 1:3.
205. The product of any one of embodiments 199-203, wherein the weight ratio of Compound 1 to the polymer is about 1:1.
206. The product of any one of embodiments 199-203, wherein the weight ratio of Compound 1 to the polymer is about 1:3, about 2:3, about 1:1, about 1.5:1, about 2:1, or about 3:1.
207. The product of any one of embodiments 199-206, wherein the solvent is dichloromethane and methanol.
208. A pharmaceutical composition comprising Compound 1:

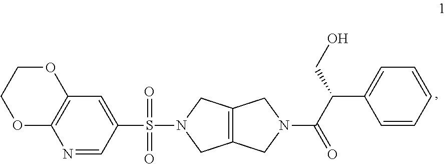 [SMILES: O=C([C@H](CO)c1ccccc1)N1CC2=C(C1)CN(S(=O)(=O)c1cnc3c(c1)OCCO3)C2]

obtained by a process comprising:
   mixing Compound 1 in a solid form, a polymer, and a solvent to afford a mixture; and
   spray-drying the mixture to afford an amorphous solid dispersion comprising Compound 1.
      209. The pharmaceutical composition of embodiment 208, wherein the solid form is Type A of Compound 1.
      210. The pharmaceutical composition of embodiment 208, wherein the solid form is Type B of Compound 1.
      211. The pharmaceutical composition of embodiment 208, wherein the solid form is Type C of Compound 1.
      212. The pharmaceutical composition of embodiment 208, wherein the solid form is Type D of Compound 1.
      213. The pharmaceutical composition of embodiment 208, wherein the solid form is Type E of Compound 1.
      214. The pharmaceutical composition of embodiment 208, wherein the solid form is Type F of Compound 1.
      215. The pharmaceutical composition of embodiment 208, wherein the solid form is Type G of Compound 1.
      216. The pharmaceutical composition of embodiment 208, wherein the solid form is Type H of Compound 1.
      217. The pharmaceutical composition of embodiment 208, wherein the solid form is Type I of Compound 1.
      218. The pharmaceutical composition of embodiment 208, wherein the solid form is Type J of Compound 1.
      219. The pharmaceutical composition of embodiment 208, wherein the solid form is Type K of Compound 1.
      220. The pharmaceutical composition of embodiment 208, wherein the solid form is Type L of Compound 1.
      221. The pharmaceutical composition of embodiment 208, wherein the solid form is Type M of Compound 1.
      222. The pharmaceutical composition of embodiment 208, wherein the solid form is selected from the group consisting of Type A, Type B, Type C, Type D, Type E, Type F, Type G, Type H, Type I, Type J, Type K, Type L, and Type M of Compound 1.
      223. The pharmaceutical composition of embodiment 208, wherein the solid form is amorphous form of Compound 1.
      224. The pharmaceutical composition of any one of embodiments 208-223, wherein the pharmaceutical composition has a water content of about 0.5-5.0 weight %.
      225. The pharmaceutical composition of any one of embodiments 208-224, wherein the pharmaceutical composition has a water content of about 1.5-4.0 weight %.
      226. The pharmaceutical composition of any one of embodiments 208-225, wherein the pharmaceutical composition has a water content of about 2.5-3.0 weight %.
      227. The pharmaceutical composition of any one of embodiments 208-226, wherein the polymer is selected from a group consisting of hydroxypropylmethyl cellulose (HPMC), hydroxypropylmethyl cellulose acetate succinate (HPMC AS), hydroxypropyl methyl cellulose phthalate (HPMCP), hydroxypropyl cellulose (HPC), ethylcellulose, cellulose acetate phthalate, polyvinylpyrrolidone (PVP), and a combination thereof, or is selected from a group consisting of polyvinylpyrrolidone (PVP), hydroxypropylmethyl cellulose (HPMC), hydroxypropylcellulose (HPC), hydroxypropylmethyl cellulose acetate succinate (HPMC AS), hydroxyethylcellulose (HEC), poly(methacrylic acid-co-methyl methacrylates) (e.g., Eudragit® L100-55), macrogol 15 hydroxystearate (e.g., Solutol® HS15), polyvinyl caprolactam-polyvinyl acetate-polyethylene glycol graft copolymer (e.g., Soluplus®), polyethylene glycol (PEG), and a combination thereof.
      228. The pharmaceutical composition of any one of embodiments 208-227, wherein the polymer is hydroxypropylmethyl cellulose (HPMC) or hydroxypropylmethyl cellulose acetate succinate (HPMC AS).
      229. The pharmaceutical composition of any one of embodiments 208-228, wherein the polymer is hydroxypropylmethyl cellulose acetate succinate (HPMC AS).
      230. The pharmaceutical composition of any one of embodiments 208-229, wherein the weight ratio of Compound 1 to the polymer is in a range of about 3:1 to about 1:3 or about 2:1 to about 1:3.
      231. The pharmaceutical composition of any one of embodiments 208-230, wherein the weight ratio of Compound 1 to the polymer is about 1:3.
      232. The pharmaceutical composition of any one of embodiments 208-230, wherein the weight ratio of Compound 1 to the polymer is about 1:1.
      233. The pharmaceutical composition of any one of embodiments 208-230, wherein the weight ratio of Compound 1 to the polymer is about 1:3, about 2:3, about 1:1, about 1.5:1, about 2:1, or about 3:1.
      234. The pharmaceutical composition of any one of embodiments 208-233, wherein the solvent is dichloromethane and methanol.
      235. A tablet dosage form comprising a tablet core, the tablet core comprising at least 10 weight % of Compound 1 in amorphous form:

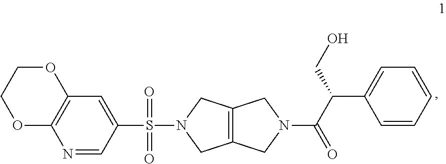 [SMILES: O=C([C@H](CO)c1ccccc1)N1CC2=C(C1)CN(S(=O)(=O)c1cnc3c(c1)OCCO3)C2]

wherein crystalline Compound 1 (Type A) is not observable by XRPD analysis (Method D) of the tablet core.
   236. The tablet dosage form of embodiment 235, wherein the tablet core comprises at least 15 weight % of Compound 1 in amorphous form.
   237. The tablet dosage form of embodiment 235 or 236, wherein the tablet core comprises at least 30 weight % of Compound 1 in amorphous form.
   238. The tablet dosage form of any one of embodiments 235-237, wherein the tablet core comprises about 200 mg of Compound 1 per tablet and has a total weight of no more than about 1200 mg per tablet.
   239. The tablet dosage form of embodiment 238, wherein the tablet core has a total weight of no more than about 1100 mg, about 1000 mg, about 900 mg, about 800 mg, or about 700 mg per tablet.
   240. A tablet dosage form comprising a tablet core, the tablet core having a total weight of no more than about 1000 mg and comprising about 200 mg of Compound 1 in amorphous form per tablet:

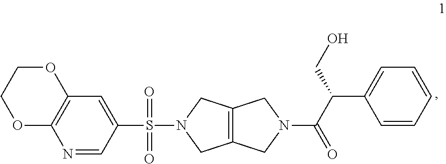 [SMILES: O=C([C@H](CO)c1ccccc1)N1CC2=C(C1)CN(S(=O)(=O)c1cnc3c(c1)OCCO3)C2]

wherein crystalline Compound 1 (Type A) is not observable by XRPD analysis (Method D) of the tablet core.
   241. The tablet dosage form of embodiment 240, wherein the tablet core has a total weight of no more than about 800 mg per tablet.
   242. The tablet dosage form of any one of embodiments 235-241, wherein the tablet core comprises 0.05-5.0% of Compound 2:

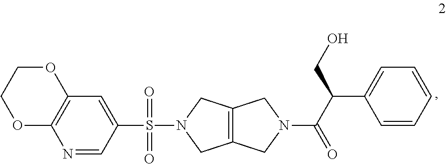 [SMILES: O=C([C@@H](CO)c1ccccc1)N1CC2=C(C1)CN(S(=O)(=O)c1cnc3c(c1)OCCO3)C2]

based on the total amount of Compound 1 and Compound 2.
   243. The tablet dosage form of embodiment 242, wherein the tablet core comprises 0.05-3.0% of Compound 2, based on the total amount of Compound 1 and Compound 2.
   244. The tablet dosage form of embodiment 242 or 243, wherein the tablet core comprises 0.05-2.0% of Compound 2, based on the total amount of Compound 1 and Compound 2.
   245. The tablet dosage form of any one of embodiments 242-244, wherein the tablet core comprises 0.05-1.0% of Compound 2, based on the total amount of Compound 1 and Compound 2.
   246. The tablet dosage form of any one of embodiments 235-245, wherein crystalline Compound 1 (Type A) is not observable by XRPD analysis (Method D) of the tablet core after storage in a sealed container as described in Example 29 for 1 month at 25° C. and 60% relative humidity.
   247. The tablet dosage form of any one of embodiments 235-246, wherein crystalline Compound 1 (Type A) is not observable by XRPD analysis (Method D) of the tablet core after storage in a sealed container as described in Example 29 for 2 months at 25° C. and 60% relative humidity.
   248. The tablet dosage form of any one of embodiments 235-247, wherein crystalline Compound 1 (Type A) is not observable by XRPD analysis (Method D) of the tablet core after storage in a sealed container as described in Example 29 for 3 months at 25° C. and 60% relative humidity.
   249. The tablet dosage form of any one of embodiments 235-248, wherein crystalline Compound 1 (Type A) is not observable by XRPD analysis (Method D) of the tablet core after storage in a sealed container as described in Example 29 for 1 month at 40° C. and 75% relative humidity.
   250. The tablet dosage form of any one of embodiments 235-249, wherein crystalline Compound 1 (Type A) is not observable by XRPD analysis (Method D) of the tablet core after storage in a sealed container as described in Example 29 for 2 months at 40° C. and 75% relative humidity.
   251. The tablet dosage form of any one of embodiments 235-250, wherein crystalline Compound 1 (Type A) is not observable by XRPD analysis (Method D) of the tablet core after storage in a sealed container as described in Example 29 for 3 months at 40° C. and 75% relative humidity.
   252. The tablet dosage form of any one of embodiments 235-251, wherein Compound 1 is present in an amorphous solid dispersion comprising Compound 1 and a polymer.
   253. The tablet dosage form of embodiment 252, wherein the polymer is selected from a group consisting of hydroxypropylmethyl cellulose (HPMC), hydroxypropylmethyl cellulose acetate succinate (HPMC AS), hydroxypropyl methyl cellulose phthalate (HPMCP), hydroxypropyl cellulose (HPC), ethylcellulose, cellulose acetate phthalate, polyvinylpyrrolidone (PVP), and a combination thereof, or is selected from a group consisting of polyvinylpyrrolidone (PVP), hydroxypropylmethyl cellulose (HPMC), hydroxypropylcellulose (HPC), hydroxypropylmethyl cellulose acetate succinate (HPMC AS), hydroxyethylcellulose (HEC), poly(methacrylic acid-co-methyl methacrylates) (e.g., Eudragit® L100-55), macrogol 15 hydroxystearate (e.g., Solutol® HS15), polyvinyl caprolactam-polyvinyl acetate-polyethylene glycol graft copolymer (e.g., Soluplus®), polyethylene glycol (PEG), and a combination thereof.
   254. The tablet dosage form of embodiment 252 or 253, wherein the polymer is hydroxypropylmethyl cellulose (HPMC) or hydroxypropylmethyl cellulose acetate succinate (HPMC AS).
   255. The tablet dosage form of any one of embodiments 252-254, wherein the polymer is hydroxypropylmethyl cellulose acetate succinate (HPMC AS).
   256. The tablet dosage form of any one of embodiments 252-255, wherein the weight ratio of Compound 1 to the polymer is in a range of about 3:1 to about 1:3 or about 2:1 to about 1:3.
   257. The tablet dosage form of any one of embodiments 252-256, wherein the weight ratio of Compound 1 to the polymer is about 1:3.
   258. The tablet dosage form of any one of embodiments 252-256, wherein the weight ratio of Compound 1 to the polymer is about 1:1.
   259. The tablet dosage form of any one of embodiments 252-256, wherein the weight ratio of Compound 1 to the polymer is about 1:3, about 2:3, about 1:1, about 1.5:1, about 2:1, or about 3:1.
   260. The tablet dosage form of any one of embodiments 235-259, further comprising one or more pharmaceutically acceptable excipients.
   261. The tablet dosage form of embodiment 260, wherein the one or more pharmaceutically acceptable excipients comprise one or more of a filler, a dry binder, a glidant, a lubricant, a disintegrant, and a film coating agent.
   262. The tablet dosage form of any one of embodiments 235-261, wherein the tablet core comprises:
an intra granular portion comprising Compound 1; and
an extra granular portion blended with the intra granular portion.
   263. The tablet dosage form of embodiment 262, wherein the intragranular portion comprises an amorphous solid dispersion comprising Compound 1 and a polymer and one or more of a filler, a dry binder, a glidant, and a lubricant, and the extragranular portion comprises one or more of a filler, a disintegrant, and a lubricant.
   264. The tablet dosage form of any one of embodiments 235-263, wherein the intragranular portion comprises:
an amorphous solid dispersion of Compound 1 in an amount of 30-70 weight % of the tablet core;
one or more fillers in an amount of 15-50 weight % of the tablet core;
one or more dry binders in an amount of 2.50-10 weight % of the tablet core;
one or more glidants in an amount of 0.50-1.50 weight % of the tablet core; and
one or more lubricants in an amount of 0.25-1 weight % of the tablet core; and the extragranular portion comprises:
one or more fillers in an amount of 5-15 weight % of the tablet core;
one or more disintegrants in an amount of 1.25-5 weight % of the tablet core; and
one or more lubricants in an amount of 0.25-1 weight % of the tablet core; or wherein the tablet dosage form comprises:
an amorphous solid dispersion of Compound 1 in an amount of 50-75 weight % of the tablet core;
one or more fillers in an amount of 15-50 weight % of the tablet core;
one or more dry binders in an amount of 2-10 weight % of the tablet core;
one or more glidants in an amount of <2 weight % of the tablet core;
one or more disintegrants in an amount of 2-10 weight % of the tablet core; and
one or more lubricants in an amount of <2 weight % of the tablet core.
   265. The tablet dosage form of embodiment 264, wherein the amorphous solid dispersion comprises Compound 1 and a polymer.
   266. The tablet dosage form of embodiment 265, wherein the polymer is selected from a group consisting of hydroxypropylmethyl cellulose (HPMC), hydroxypropylmethyl cellulose acetate succinate (HPMC AS), hydroxypropyl methyl cellulose phthalate (HPMCP), hydroxypropyl cellulose (HPC), ethylcellulose, cellulose acetate phthalate, polyvinylpyrrolidone (PVP), and a combination thereof, or is selected from a group consisting of polyvinylpyrrolidone (PVP), hydroxypropylmethyl cellulose (HPMC), hydroxypropylcellulose (HPC), hydroxypropylmethyl cellulose acetate succinate (HPMC AS), hydroxyethylcellulose (HEC), poly(methacrylic acid-co-methyl methacrylates) (e.g., Eudragit® L100-55), macrogol 15 hydroxystearate (e.g., Solutol® HS15), polyvinyl caprolactam-polyvinyl acetate-polyethylene glycol graft copolymer (e.g., Soluplus®), polyethylene glycol (PEG), and a combination thereof.
   267. The tablet dosage form of embodiment 265 or 266, wherein the polymer is hydroxypropylmethyl cellulose acetate succinate (HPMC AS).
   268. The tablet dosage form of any one of embodiments 265-267, wherein the weight ratio of Compound 1 to the polymer is in a range of about 3:1 to about 1:3 or about 2:1 to about 1:3.
   269. The tablet dosage form of any one of embodiments 265-268, wherein the weight ratio of Compound 1 to the polymer is about 1:3.
   270. The tablet dosage form of any one of embodiments 265-269, wherein the weight ratio of Compound 1 to the polymer is about 1:1.
   271. The tablet dosage form of any one of embodiments 265-270, wherein the weight ratio of Compound 1 to the polymer is about 1:3, about 2:3, about 1:1, about 1.5:1, about 2:1, or about 3:1.
   272. The tablet dosage form of any one of embodiments 264-271, wherein the one or more fillers comprise microcrystalline cellulose or lactose monohydrate.
   273. The tablet dosage form of any one of embodiments 264-272, wherein the one or more dry binders comprise crospovidone or crosslinked polyvinylpyrrolidone.
   274. The tablet dosage form of any one of embodiments 264-273, wherein the one or more glidants comprise colloidal silicon dioxide or fumed silica.
   275. The tablet dosage form of any one of embodiments 264-274, wherein the one or more lubricants comprise magnesium stearate.
   276. The tablet dosage form of any one of embodiments 264-275, wherein the one or more disintegrants comprise crocarmellose sodium.
   277. A solid oral dosage form comprising a stabilized amorphous compound (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one, wherein the stabilized amorphous compound does not show crystallinity by PXRD (Method D) after 2 weeks of storage at 60° C./75% RH (exposed).
   278. The solid oral dosage form of embodiment 277, wherein the stabilized amorphous compound shows a single glass transition temperature (T_{G}) and no melt endotherm by DSC (Method B) after 2 weeks of storage at 60° C./75% RH (exposed).
   279. The solid oral dosage form of embodiment 277 or 278, wherein the solid oral dosage form contains a total of 200 mg of (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one.
   280. The solid oral dosage form of any one of embodiments 277-279, wherein the solid oral dosage form has a total weight of not more than 800 mg.
   281. The solid oral dosage form of any one of embodiments 277-280, wherein the solid oral dosage form is a tablet or capsule.
   282. The solid oral dosage form of any one of embodiments 277-281, wherein the stabilized amorphous compound is in a spray dried dispersion with a polymer.
   283. The solid oral dosage form of embodiment 282, wherein the polymer is selected from the group consisting of hydroxypropylmethyl cellulose (HPMC), hydroxypropylmethyl cellulose acetate succinate (HPMC AS), hydroxypropyl methyl cellulose phthalate (HPMCP), hydroxypropyl cellulose (HPC), ethylcellulose, cellulose acetate phthalate, polyvinylpyrrolidone (PVP), and a combination thereof, or is selected from a group consisting of polyvinylpyrrolidone (PVP), hydroxypropylmethyl cellulose (HPMC), hydroxypropylcellulose (HPC), hydroxypropylmethyl cellulose acetate succinate (HPMC AS), hydroxyethylcellulose (HEC), poly(methacrylic acid-co-methyl methacrylates) (e.g., Eudragit® L100-55), macrogol 15 hydroxystearate (e.g., Solutol® HS15), polyvinyl caprolactam-polyvinyl acetate-polyethylene glycol graft copolymer (e.g., Soluplus®), polyethylene glycol (PEG), and a combination thereof.
   284. The solid oral dosage form of embodiment 283, wherein the polymer is HPMC AS.
   285. The solid oral dosage form of embodiment 284, wherein the (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one is spray dried with HPMC AS in a weight ratio of 1:3 to 2:1.
   286. The solid oral dosage form of embodiment 284, wherein the (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one is spray dried with HPMC AS in a weight ratio of 1:1.
   287. A (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one active pharmaceutical ingredient (API) composition comprising 0.05-5.0% by HPLC of (R)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one.
   288. A tablet comprising 200 mg of stabilized amorphous compound (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one as the active pharmaceutical ingredient (API), wherein the stabilized amorphous compound does not show crystallinity by PXRD (Method D) after 2 weeks of storage of the tablet at 60° C./75% RH (exposed).
   289. The tablet of embodiment 288, wherein the API comprises less than 5.0% by HPLC of (R)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one.
   290. The tablet of embodiment 288 or 289, wherein the API comprises less than 0.05% by HPLC of (R)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one.
   291. The tablet of any one of embodiments 288-290, having a total weight of less than 800 mg.

### EXAMPLES

The present teachings include descriptions provided in the Examples that are not intended to limit the scope of any claim. The following non-limiting examples are provided to further illustrate the present teachings. Those of skill in the art, in light of the present application, will appreciate that many changes can be made in the specific embodiments that are provided herein and still obtain a like or similar result without departing from the spirit and scope of the present teachings.

Abbreviations

| | |
|---|---|
| ACN | Acetonitrile |
| API | Active Pharmaceutical Ingredient |
| AUCₗₐₛₜ | Area under the curve from zero to |
| | the last measurable point |
| DCM | Dichloromethane |
| DIEA | Diisopropylethylamine |
| DMAc | Dimethylacetamide |
| DMF | Dimethylformamide |
| DMSO | Dimethyl sulfoxide |
| DSC | Differential scanning calorimetry |
| DVS | Dynamic vapor sorption |
| EtOAc | Ethyl acetate |
| EtOH | Ethanol |
| FaSSIF | Fasted state simulated intestinal |
| | fluid |
| FeSSIF | Fed state simulated intestinal fluid |
| h | Hour |
| HATU | 1-[bis(dimethylamino)methylene]- |
| | 1H-1,2,3-triazolo[4,5-b]pyridinium |
| | 3-oxide hexafluoro-phosphate |
| HPLC | High-performance liquid |
| | chromatography |
| HPMC | Hydroxypropyl Methylcellulose |
| AS-MG | Acetate Succinate MG |
| IPA | Isopropanol |
| LCMS | Liquid chromatography mass |
| | spectrometry |
| MeOH | Methanol |
| MIBK | Methyl isobutyl ketone |
| min | Minute |
| MTBE | Methyl tert-butyl ether |
| n-Bu | n-butyl |
| ND | Not determined |
| NMP | N-methyl pyrrolidone |
| NMR | Nuclear magnetic resonance |
| PTFE | Polytetrafluoroethylene |
| RH | Relative humidity |
| RRT | Relative retention time |
| RT | Room Temperature |
| Rt | Retention time |
| scfh | Standard cubic feet per hour |
| SDD | Spray-dried dispersion |
| SEM | Scanning electron microscopy |
| SGF | Simulated gastric fluid |
| SIF | Simulated intestinal fluid |
| TEA | Triethylamine |
| TFA | Trifluoroacetic acid |
| T_{G} | Glass transition temperature |
| TGA | Thermogravimetric analysis |
| THF | Tetrahydrofuran |
| TLC | Thin layer chromatography |
| TRS | Total related substances |
| UPLC | Ultra performance liquid |
| | chromatography |
| XRPD | X-ray powder diffraction |

### Instrumentation and Methods

Unless otherwise indicated, the following instrumentation and methods were used in the working examples described herein.

### X-Ray Powder Diffraction (XRPD or PXRD)

Method A. XRPD analysis was performed with a Panalytical λ'Pert3 Powder XRPD on a Si zero-background holder. The 2θ position was calibrated against Panalytical 640 Si powder standard. Details of the XRPD method used in the experiments are listed in Table 1.

TABLE 1

| | Parameters for Reflection Mode |
|---|---|
| X-Ray wavelength | Cu, kα, Kα1 (Å): 1.540598, Kα2 (Å): 1.544426 |
| | Kα2/Kα1 intensity ratio: 0.50 |
| X-Ray tube setting | 45 kV, 40 mA |
| Divergence slit | Automatic |
| Scan mode | Continuous |
| Scan range (°2TH) | 3°-40° |
| Step size (°2TH) | 0.0262606 |
| Scan speed (°/s) | 0.066482 |

Method B. XRPD analysis was performed with a Rigaku X-Ray Powder Diffractomer MiniFlex 600 with the parameters listed in Table 2.

TABLE 2

| Parameter | Setting | |
|---|---|---|
| Soller (inc.) | 5.0 | deg |
| IHS | 10.0 | mm |
| SS | 1.250 | deg |
| DS | 1.250 | deg |
| Soller (rec) | 5.0 | deg |
| RS | 0.3 | mm |

| | |
|---|---|
| Scan Axis | Theta/2-Theta |
| Mode | Continuous |
| Start (deg) | 2.0000 |
| Stop (deg) | 40.0000 |
| Step (deg) | 0.020 |
| Speed (deg/min) | 2.5 |
| Spin | Yes |
| Voltage (kV) | 40 |
| Current (mA) | 15 |

Method C. XRPD analysis was performed with a Panalytical X'Pert3 powder diffractometer in reflection mode. Details of the XRPD method used in the experiments are as follows:

| | Parameters |
|---|---|
| X-Ray wavelength | CuKα, Kα1 (Å): 1.540598, Kα2 (Å): 1.544426 |
| | Kα2/Kα1 intensity ratio: 0.50 |
| X-Ray tube setting | 45 kV, 40 mA |
| Divergence slit (°) | 1/8 |
| Scan mode | Continuous |
| Scan range (°2TH) | 3°-40° |
| Scan step time (s) | 46.665 |
| Step size (°2TH) | 0.0263 |
| Test time | ~5 min |

Method D. XRPD analysis was performed with the following parameters:

| | Parameters |
|---|---|
| Start position (°2TH) | 2.00 |
| Stop position (°2TH) | 40.00 |
| DS (°) | 1.250 |
| RS (mm) | 0.3 |
| SS (°) | 1.250 |
| Step size (°) | 0.02 |
| Rate (°/minute) | 0.50 |

### Thermal Analysis (TGA and DSC)

Method A. TGA was conducted using a TA Q500 TGA from TA Instruments. DSC was performed using a TA Q2000 DSC from TA Instruments. Detailed parameters used are listed in Table 3.

TABLE 3

| Parameters | TGA | DSC |
|---|---|---|
| Method | Ramp | Ramp |
| Sample pan | Platinum, open | Aluminum, crimped |
| Temperature | RT-desired temperature | 25° C.-desired temperature |
| Heating rate | 10° C./min | 10° C./min |
| Purge gas | N₂ | N₂ |

Method B. DSC analysis was conducted with the following procedure: Perform DSC modulated 1.00° C. for 60 seconds with a ramp rate of 2° C./min to 250° C. Use a standby temperature range of 20° to 25° C.

### Dynamic Vapor Sorption

Dynamic Vapor Sorption (DVS) was measured with a Surface Measurement System (SMS) DVS Intrinsic. Parameters for DVS analysis are listed in Table 4.

TABLE 4

| Parameters | Values |
|---|---|
| Temperature | 25° C. |
| Sample size | 10-20 mg |
| Gas and flow rate | N₂, 200 mL/min |
| dm/dt | 0.002%/min |
| Min. dm/dt stability duration | 10 min |
| Max. equilibrium time | 360 min |
| RH range | Room RH-95% RH-0% RH-95% RH |
| RH step size | 10% |

### High-Pressure Liquid Chromatography (HPLC)

Method A. The HPLC parameters and gradient set forth in Tables 5 and 6, respectively, were used for sample analysis.

TABLE 5

| Parameter | Condition | |
|---|---|---|
| HPLC System | Waters Alliance HPLC equipped with UV Detector | |
| Column | Aglient ZORBAX StableBond-Aq, 4.6 × 150 mm, 3.5 μm, | |
| | Part No. 863953-914 | |
| Column Temperature | 40.0 ± 3.0° C. | |
| Sample Temperature | Ambient | |
| Detection Wavelength | 210 | nm |
| Diluent | 25:75 Water:ACN (v/v) | |
| Mobile Phase A | [90:10] 20 mM NaH₂PO₄ · H₂O, pH 2.0:ACN (v/v) | |
| Mobile Phase B | [20:80] 20 mM NaH₂PO₄ · H₂O, pH 2.0:ACN (v/v) | |
| Needle Wash | 50:50 MeOH:Water (v/v) | |
| Seal Wash/Purge | 10:90 MeOH:Water (v/v) | |
| Injection Volume | 20 | μL |

TABLE 6

| Time | Flow Rate | % Mobile | % Mobile |
|---|---|---|---|
| (Minutes) | (mL/min) | Phase A | Phase B |
| 0.0 | 1.0 | 100.0 | 0.0 |
| 5.0 | 1.0 | 80.0 | 20.0 |
| 18.0 | 1.0 | 40.0 | 60.0 |
| 20.0 | 1.0 | 0.0 | 100.0 |
| 24.0 | 1.0 | 0.0 | 100.0 |
| 25.0 | 1.0 | 100.0 | 0.0 |
| 35.0 | 1.0 | 100.0 | 0.0 |

### Ultra Performance Liquid Chromatography

The UPLC parameters and linear method gradients disclosed in Table 7 and Table 8, respectively, were used for sample analysis.

TABLE 7

| Parameter | Condition | |
|---|---|---|
| System | Waters H-Class UPLC with TUV detector | |
| Column | Acquity UPLC BEH Shield RP18, 2.1 × 50 mm, 1.7 μm | |
| Column Temperature | 40.0 ± 3.0° C. | |
| Sample Temperature | Ambient | |
| Mobile Phase A | 0.1% Phosphoric Acid in [90:10] Water:ACN | |
| Mobile Phase B/Needle | 0.1% Phosphoric Acid in ACN | |
| Wash | | |
| Flow Rate | 0.500 | mL/min |
| Gradient | See Table 8 | |
| Injection Volume | 2.0 | μL |
| Run Time | 6.50 | minutes |
| Detection Wavelength | 293 | nm |
| Sampling Rate | 20 | points/sec |

TABLE 8

| Time | Flow Rate | % Mobile | % Mobile |
|---|---|---|---|
| (Minutes) | (mL/min) | Phase A | Phase B |
| 0.00 | 0.500 | 100.0 | 0.0 |
| 1.00 | 0.500 | 100.0 | 0.0 |
| 3.50 | 0.500 | 0.0 | 100.0 |
| 4.00 | 0.500 | 0.0 | 100.0 |
| 4.01 | 0.500 | 100.0 | 0.0 |
| 6.50 | 0.500 | 100.0 | 0.0 |

### Water Content

Water content was determined by USP <921>, Method 1c.

### Dissolution

Except where otherwise indicated, dissolution of the tablets is performed with USP Apparatus 2 (paddles) by USP<711>. The determination of assay is achieved by quantitation against an external reference standard using a reversed phase gradient UPLC method. The UPLC method utilizes an Acquity UPLC BEH Shield column with two mobile phases, both consisting of acetonitrile, water, and phosphate buffer.

### Example 1—Synthesis of (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one (1)

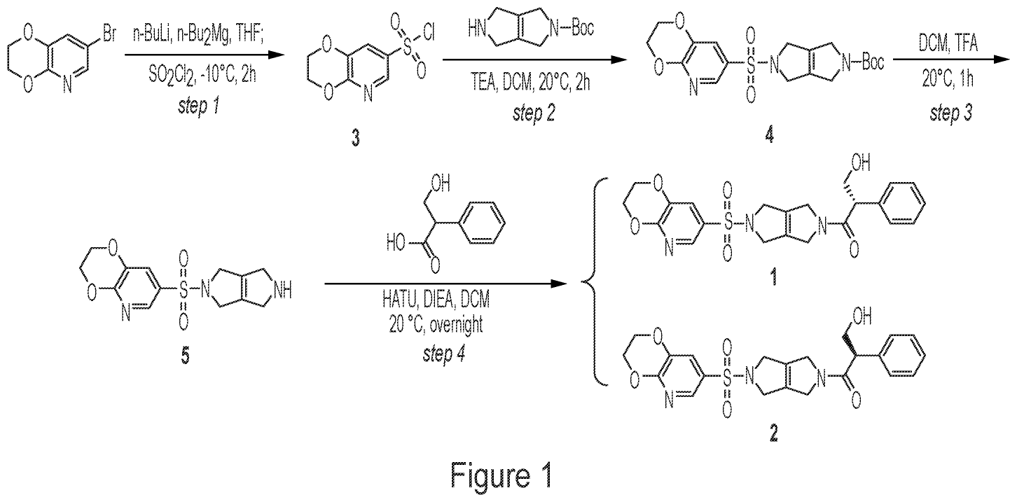

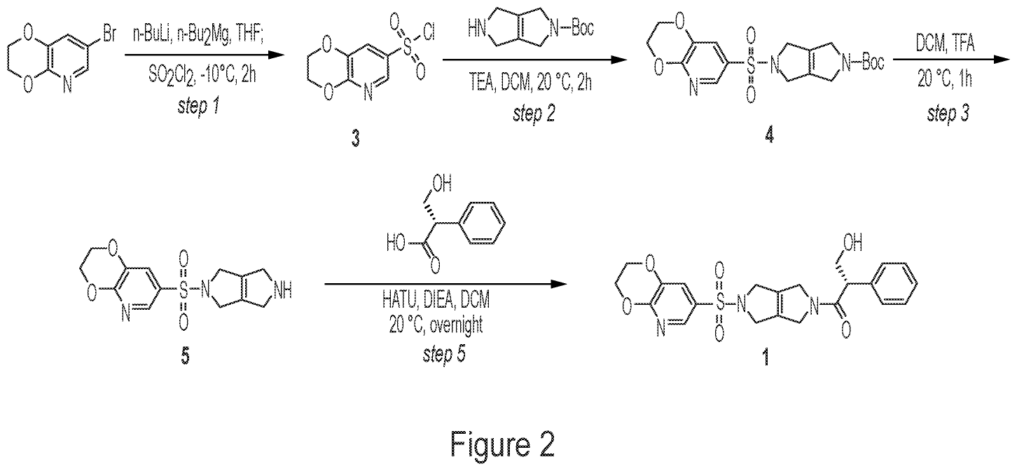

The PKR Activating Compound 1 can be obtained by the method described herein and the reaction schemes shown in FIGS. 1 and 2. Compound 1 has a molecular weight of 457.50 Da.

### Step 1. 2H,3H-[1,4]dioxino[2,3-b]pyridine-7-sulfonyl Chloride (3)

Into a 100 mL round-bottom flask purged and maintained with an inert atmosphere of nitrogen was placed a solution of n-BuLi in hexane (2.5 M, 2 mL, 5.0 mmol, 0.54 equiv) and a solution of n-Bu₂Mg in heptanes (1.0 M, 4.8 mL, 4.8 mmol, 0.53 equiv). The resulting solution was stirred for 10 min at RT (20° C.). This was followed by the dropwise addition of a solution of 7-bromo-2H,3H-[1,4]dioxino[2,3-b]pyridine (2 g, 9.26 mmol, 1.00 equiv) in tetrahydrofuran (16 mL) with stirring at −10° C. in 10 min. The resulting mixture was stirred for 1 h at −10° C. The reaction mixture was slowly added to a solution of sulfuryl chloride (16 mL) at −10° C. The resulting mixture was stirred for 0.5 h at −10° C. The reaction was then quenched by the careful addition of 30 mL of saturated ammonium chloride solution at 0° C. The resulting mixture was extracted with 3×50 mL of dichloromethane. The organic layers were combined, dried over anhydrous sodium sulfate, filtered and concentrated under vacuum. The residue was purified by silica gel column chromatography, eluting with ethyl acetate/petroleum ether (1:3). This provided 1.3 g (60%) of 2H,3H-[1,4]dioxino[2,3-b]pyridine-7-sulfonyl chloride as a white solid. LCMS m/z: calculated for C₇H₆ClNO₄S: 235.64; found: 236 [M+H]⁺.

### Step 2. Tert-Butyl 5-[2H,3H-[1,4]dioxino[2,3-b]pyridine-7-sulfonyl]-H,2H,3H,4H,5H,6H-pyrrolo[3,4-c]pyrrole-2-carboxylate (4)

Into a 100-mL round-bottom flask was placed 2H,3H-[1,4]dioxino[2,3-b]pyridine-7-sulfonyl chloride (1.3 g, 5.52 mmol, 1.00 equiv), tert-butyl 1H,2H,3H,4H,5H,6H-pyrrolo[3,4-c]pyrrole-2-carboxylate (1.16 g, 5.52 mmol), dichloromethane (40 mL), and triethylamine (1.39 g, 13.74 mmol, 2.49 equiv). The solution was stirred for 2 h at 20° C., then diluted with 40 mL of water. The resulting mixture was extracted with 3×30 mL of dichloromethane. The organic layers were combined, dried over anhydrous sodium sulfate, filtered and concentrated under vacuum. The residue was purified by silica gel column chromatography, eluting with dichloromethane/methanol (10:1). This provided 1.2 g (53%) of tert-butyl 5-[2H,3H-[1,4]dioxino[2,3-b]pyridine-7-sulfonyl]-1H,2H,3H,4H,5H,6H-pyrrolo[3,4-c]pyrrole-2-carboxylate as a yellow solid. LCMS m/z: calculated for C₁₈H₂₃N₃O₆S: 409.46; found: 410 [M+H]+.

### Step 3. 2-[2H,3H-[,4]dioxino[2,3-b]pyridine-7-sulfonyl]-1H,2H,3H,4H,5H,6H-pyrrolo[3,4-c]pyrrole (5)

Into a 100-mL round-bottom flask was placed tert-butyl 5-[2H,3H-[1,4]dioxino[2,3-b]pyridine-7-sulfonyl]-1H,2H,3H,4H,5H,6H-pyrrolo[3,4-c]pyrrole-2-carboxylate (1.2 g, 2.93 mmol, 1.00 equiv), dichloromethane (30 mL), and trifluoroacetic acid (6 mL). The solution was stirred for 1 h at 20° C. The resulting mixture was concentrated under vacuum. The residue was dissolved in 10 mL of methanol and the pH was adjusted to 8 with sodium bicarbonate (2 mol/L). The resulting solution was extracted with 3×10 mL of dichloromethane. The organic layers were combined, dried over anhydrous sodium sulfate, filtered and concentrated under vacuum. The crude product was purified by silica gel column chromatography, eluting with dichloromethane/methanol (10:1). This provided 650 mg (72%) of 2-[2H,3H-[1,4]dioxino[2,3-b]pyridine-7-sulfonyl]-1H,2H,3H,4H,5H,6H-pyrrolo[3,4-c]pyrrole as a yellow solid. LCMS m/z: calculated for C₁₃H₁₅N₃O₄S: 309.34; found: 310 [M+H]⁺.

### Step 4. (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one (1) and (R)-1-(5-[2H, 3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one (2)

Into a 100 mL round-bottom flask was placed 2-[2H,3H-[1,4]dioxino[2,3-b]pyridine-7-sulfonyl]-1H,2H,3H,4H,5H,6H-pyrrolo[3,4-c]pyrrole (150 mg, 0.48 mmol, 1.00 equiv), 3-hydroxy-2-phenylpropanoic acid (97 mg, 0.58 mmol, 1.20 equiv), dichloromethane (10 mL), HATU (369 mg, 0.97 mmol, 2.00 equiv) and DIEA (188 mg, 1.46 mmol, 3.00 equiv). The resulting solution was stirred overnight at 20° C. The reaction mixture was diluted with 20 mL of water and was then extracted with 3×20 mL of dichloromethane. The organic layers were combined, dried over anhydrous sodium sulfate, filtered and concentrated under vacuum. The residue was purified by prep-TLC eluted with dichloromethane/methanol (20:1) and further purified by prep-HPLC (Column: XBridge C18 OBD Prep Column, 100 Å, 5 μm, 19 mm×250 mm; Mobile Phase A: water (10 mmol/L NH₄HCO₃), Mobile Phase B: MeCN; Gradient: 15% B to 45% B over 8 min; Flow rate: 20 mL/min; UV Detector: 254 nm). The two enantiomers were separated by prep-Chiral HPLC (Column, Daicel CHIRALPAK® IF, 2.0 cm×25 cm, 5 m; mobile phase A: DCM, phase B: MeOH (hold 60% MeOH over 15 min); Flow rate: 16 mL/min; Detector, UV 254 & 220 nm). This resulted in peak 1 (2, Rt: 8.47 min) 9.0 mg (4%) of (R)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one as a yellow solid; and peak 2 (1, Rt: 11.83 min) 10.6 mg (5%) of (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one as a yellow solid.

(1): ¹H NMR (400 MHz, DMSO-d₆) δ 8.13 (d, J=2.0 Hz, 1H), 7.61 (d, J=2.0 Hz, 1H), 7.31-7.20 (m, 5H), 4.75 (t, J=5.2 Hz, 1H), 4.50-4.47 (m, 2H), 4.40-4.36 (m, 1H), 4.32-4.29 (m, 2H), 4.11-3.87 (m, 8H), 3.80-3.77 (m, 1H), 3.44-3.41 (m, 1H). LC-MS (ESI) m/z: calculated for C₂₂H₂₃N₃O₆S: 457.13; found: 458.0 [M+H]⁺.

(2): ¹H NMR (400 MHz, DMSO-d₆) δ 8.13 (d, J=2.0 Hz, 1H), 7.60 (d, J=2.0 Hz, 1H), 7.31-7.18 (m, 5H), 4.75 (t, J=5.2 Hz, 1H), 4.52-4.45 (m, 2H), 4.40-4.36 (m, 1H), 4.34-4.26 (m, 2H), 4.11-3.87 (m, 8H), 3.80-3.78 (m, 1H), 3.44-3.43 (m, 1H). LC-MS (ESI) m/z: calculated for C₂₂H₂₃N₃O₆S: 457.13; found: 458.0 [M+H]⁺.

### Step 5. (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one (1)

Alternatively, Compound 1 can be synthesized using the procedure described here as Step 5.

3-Hydroxy-2-phenylpropanoic acid (1 g) was separated by Prep-SFC with the following conditions: Instrument Name: SHIMADZU LC-20AD, LC parameters: Pump Mode: Binary gradient, Start Conc. of Pump B: 100.0%, Total Flow: 170 mL/min, Phase A, Phase B: MeOH (0.1% HAC), Column Name: CHIRALPAK AD-H, Length: 100 mm, Internal Diameter: 4.6 mm, Particle Size: 5 μm, Column Temp: 20° C., PDA Model: SPD-M20A, Wavelength: from 190 nm to 500 nm. This provided peak 1: (Rt=5.76 min) 380 mg of (S)-3-hydroxy-2-phenylpropanoic acid as a white solid, and peak 2: (Rt=6.87 min) 370 mg of (R)-3-hydroxy-2-phenylpropanoic acid as a white solid. ¹H NMR (300 MHz, DMSO-d₆): δ ppm 12.31 (br s, 1H), 7.40-7.20 (m, 5H), 4.94 (br s, 1H), 3.92 (t, J=9 Hz, 1H), 3.67-3.54 (m, 2H). S-enantiomer: α_{D}^{16.7}=−110 (C 0.02, water); [literature: −79 ] R-enantiomer: α_{D}^{16.7}=+125 (C 0.02, water).

A solution of 7-((3,4,5,6-tetrahydropyrrolo[3,4-c]pyrrol-2(1H)-yl)sulfonyl)-2,3-dihydro-[1,4]dioxino[2,3-b]pyridine (130.9 mg, 0.423 mmol) in DMF (2.5 ml) was cooled on an ice bath, then treated with (S)-3-hydroxy-2-phenylpropanoic acid (84.8 mg, 0.510 mmol), HATU (195.5 mg, 0.514 mmol), and DIEA (0.30 mL, 1.718 mmol) and stirred at ambient temperature overnight. The solution was diluted with EtOAc (20 mL), washed sequentially with water (20 mL) and brine (2×20 mL), dried (MgSO4), filtered, treated with silica gel, and evaporated under reduced pressure. The material was chromatographed by Biotage MPLC (10 g silica gel column, 0 to 5% MeOH in DCM) to provide a white, slightly sticky solid. The sample was readsorbed onto silica gel and chromatographed (10 g silica gel column, 0 to 100% EtOAc in hexanes) to provide (2S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one (106.5 mg, 0.233 mmol, 55% yield) as a white solid.

### Example 2—Preparation and Characterization of Type A of Compound 1

### Preparation of Type A of Compound 1

A 1 L round-bottom flask with overhead stirring, a temperature probe, and an N₂ inlet was charged with 7-((3,4,5,6-tetrahydropyrrolo[3,4-c]pyrrol-2(1H)-yl)sulfonyl)-2,3-dihydro-[1,4]dioxino[2,3-b]pyridine (33.24 g, 96 mmol), (S)-3-hydroxy-2-phenylpropanoic acid (19.08 g, 115 mmol), and DMF (361 ml). The mixture was cooled to 0° C., HATU (43.7 g, 115 mmol) was added, and a mild ˜5° C. exotherm was observed. DIEA (70.2 ml, 402 mmol) was added dropwise over 20 minutes, and the pot was held near 0° C. The reaction mixture was sampled after 2 h and then after 3 h. After 3 h, an additional 50 mL of DMF was added to thin the reaction mixture.

After 3.5 h at 0° C., 37 volumes of DCM was added to the reaction mixture, and the solution was transferred to a 4 L separatory funnel and washed with water (2060 mL). The organic layer was then washed 3×2060 mL of brine (26% W/W NaCl) and dried overnight with MgSO4. The solution was concentrated on a rotary evaporator to afford a waxy white solid (>80 g).

The solids were triturated with 500 mL of 5:4 EtOAc/hexanes, filtered, washed with 100 mL of 1:1 EtOAc/Hexanes, and dried in a vacuum oven at ambient temperature to afford 50.3 g of a white solid.

The resulting material was ground in a mortar, charged to a 3 L round-bottom flask with overhead stirring and slurried with 1900 mL of ethanol. The slurry was heated to 76° C., and water was added dropwise. After 40 mL of water had added, the mixture was filtered with a Buchner funnel. The filtrate was charged back to the round-bottom flask, stirred overnight, and cooled slowly to room temperature.

The resulting slurry was cooled to 10° C., stirred for 1 h, and filtered. The round-bottom flask and filter cake were washed with 100 mL of ethanol. The filter cake was dried on the funnel for 1 h, and overnight in a vacuum oven at ambient temperature to afford 38.43 g of Compound 1 a white solid, which was designated as Type A of Compound 1.

### Characterization of Type A of Compound 1

Type A was characterized by XRPD (Method A), TGA (Method A), DSC (Method A), and DVS analysis.

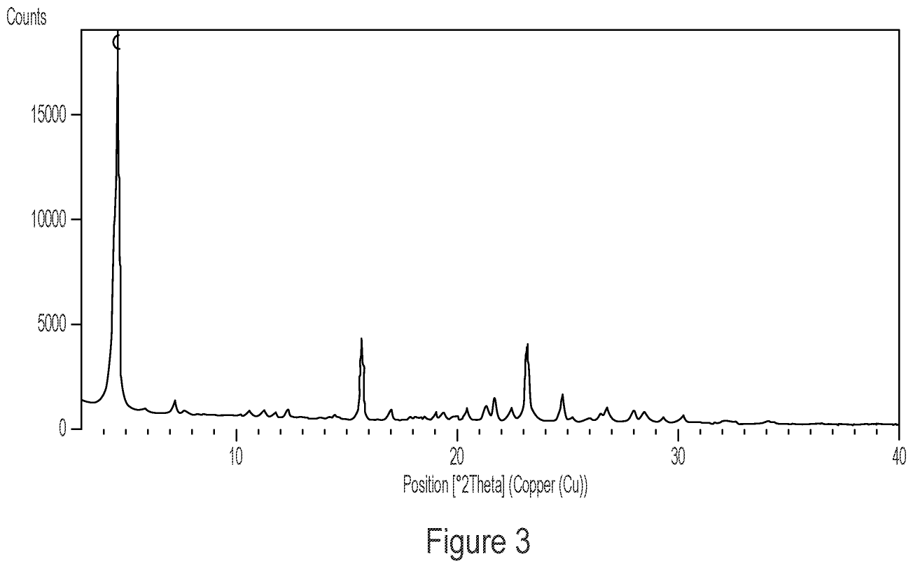

The XRPD pattern for Type A is depicted in FIG. 3, and the corresponding data are summarized in the following table:

| Pos. [°2Th.] | d-spacing [Å] |
|---|---|
| 4.61 | 19.19 |
| 5.80 | 15.24 |
| 7.22 | 12.25 |
| 7.68 | 11.50 |
| 11.21 | 7.89 |
| 12.31 | 7.19 |
| 14.44 | 6.13 |
| 15.66 | 5.66 |
| 16.95 | 5.23 |
| 18.02 | 4.92 |
| 19.20 | 4.62 |
| 20.48 | 4.34 |
| 21.35 | 4.16 |
| 21.66 | 4.10 |
| 22.47 | 3.96 |
| 23.19 | 3.84 |
| 24.76 | 3.60 |
| 26.73 | 3.34 |
| 28.01 | 3.19 |
| 28.49 | 3.13 |
| 29.35 | 3.04 |
| 30.25 | 2.95 |
| 32.14 | 2.79 |
| 34.12 | 2.63 |
| 36.46 | 2.46 |

The foregoing XRPD data for Type A can also be rounded to a single decimal place, as summarized in the following table:

| Pos. [°2Th.] | d-spacing [Å] |
|---|---|
| 4.6 | 19.2 |
| 5.8 | 15.2 |
| 7.2 | 12.2 |
| 7.7 | 11.5 |
| 11.2 | 7.9 |
| 12.3 | 7.2 |
| 14.4 | 6.1 |
| 15.7 | 5.7 |
| 16.9 | 5.2 |
| 18.0 | 4.9 |
| 19.2 | 4.6 |
| 20.5 | 4.3 |
| 21.3 | 4.2 |
| 21.7 | 4.1 |
| 22.5 | 4.0 |
| 23.2 | 3.8 |
| 24.8 | 3.6 |
| 26.7 | 3.3 |
| 28.0 | 3.2 |
| 28.5 | 3.1 |
| 29.4 | 3.0 |
| 30.3 | 3.0 |
| 32.1 | 2.8 |
| 34.1 | 2.6 |
| 36.5 | 2.5 |

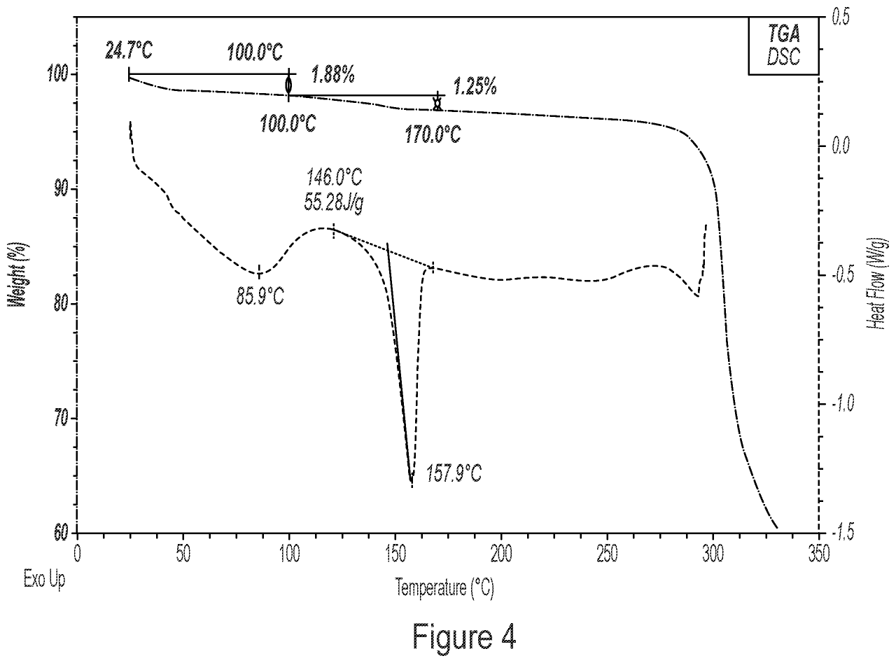

The TGA and DSC curves for Type A are shown in FIG. 4. As shown in FIG. 4, Type A showed 1.9% weight loss up to 100° C. by TGA and two endotherms at 85.9° C. (peak temperature) and 146.0° C. (onset temperature) by DSC.

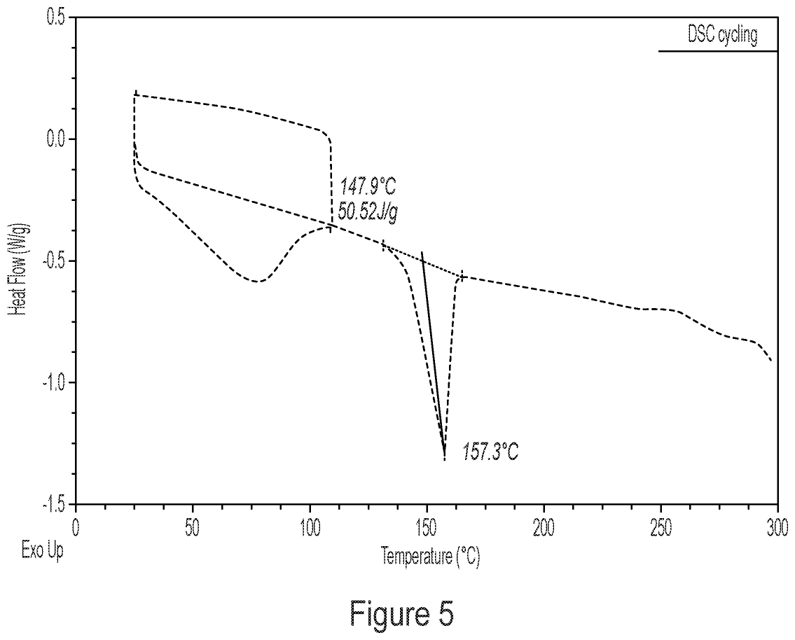

By DSC cycling as shown in FIG. 5, Type A was heated to 120° C. and cooled to 25° C., then heated up to 300° C. No endotherm below 100° C. was observed in the second heating cycle. XRPD analysis after DSC cycling showed no form change compared to Type A (FIG. 3).

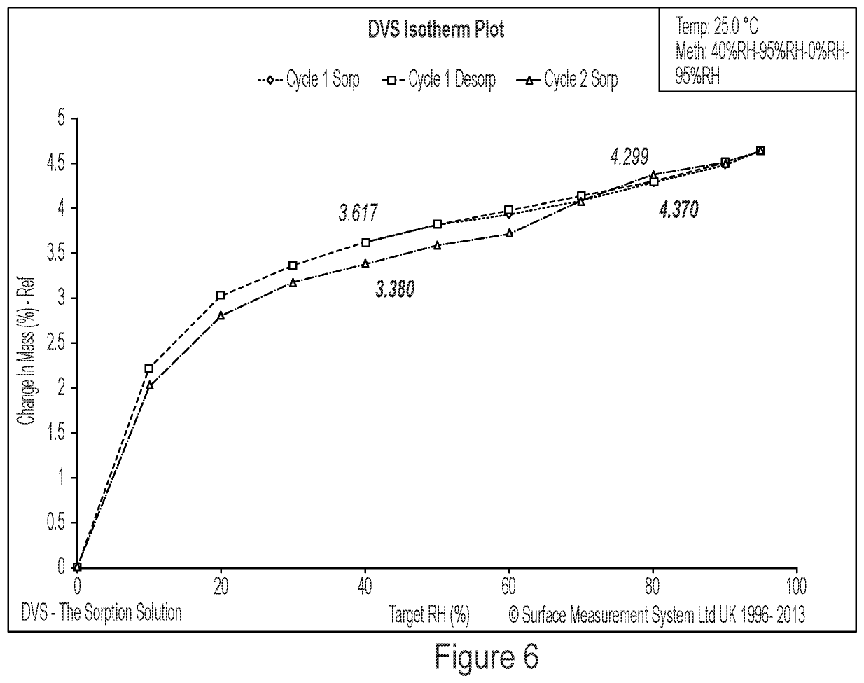

DVS results showed a 3.4% water uptake up to 40% RH (ambient condition), and 1.0% water uptake from 40% RH to 80% RH at RT, indicating that Type A is hygroscopic (FIG. 6). No form change was observed for Type A before and after DVS test at RT, as determined by XRPD.

Based on the foregoing analytical data, Type A is believed to be a channel hydrate.

### Example 3—Polymorph Screening of Compound 1

Polymorph screening experiments were performed using a series of crystallization and solid transition methods.

### Solid Vapor Diffusion

Solid vapor diffusion experiments were conducted using 13 different solvents. Approximately 15 mg of Compound 1 (Type A) was weighed into a 4-mL vial, which was placed into a 20-mL vial with 3 mL of volatile solvent. The 20-mL vial was sealed with a cap and kept at RT for 7 days, allowing solvent vapor to interact with the sample. The solids were characterized by XRPD analysis (Method A), and the results summarized in Table 9 showed that Type A or a mixture of Types A and D were obtained.

TABLE 9

| Solvent | Solid Form |
|---|---|
| H₂O | Type A |
| EtOH | Type A |
| Toluene | Type A |
| Acetone | Type A |
| ACN | Type A |
| DCM | Type A |
| THF | Type D + Type A |
| CHCl₃ | Type A |
| MeOH | Type A |
| IPA | Type A |
| 1,4-Dioxane | Type A |
| DMSO | Type A |
| EtOAc | Type A |

### Slurry Conversion at 4° C., RT or 50° C.

Slurry experiments were conducted at RT in different solvent systems. About 15 mg of Compound 1 (Type A) was suspended in 0.3 mL of solvent in a 2-mL glass vial. After the suspension was stirred magnetically for 7 days at 4° C., RT or 50° C., the remaining solids were isolated for XRPD analysis (Method A). Results summarized in Table 10 indicated that Type A, B, C and D, or mixtures thereof, were obtained.

TABLE 10

| Solvent (v:v) | Temperature (° C.) | Solid Form |
|---|---|---|
| H₂O | 50° C. | Type B |
| MeOH | | Type B |
| EtOAc | | Type A |
| MIBK | | Type A |
| Toluene | | Type A |
| Acetone | RT | Type A |
| 1,4-Dioxane | | Type C |
| DMSO/H₂O (1/9) | | Type B |
| THF | 4° C. | Type D |
| ACN | | Type A |
| CHCl₃/Toluene (1/3) | | Type B + |
| | | Type A |
| DCM/n-Heptane (1/3) | | Type A |
| EtOH | 50° C. | Type A |
| IPA | | Type A |
| IPAc | | Type A |
| MTBE | | Type A |
| n-Heptane | | Type A |
| 2-MeTHF | | Type A |
| 1,4-Dioxane | | Type A |
| THF | | Type A |
| CHCl₃/MeOH (v/v, 1:3) | | Type A |
| Acetone/H₂O (v/v, 1:3) | | Type B |
| CHCl₃/EtOAc (v/v, 1:3) | | Type A |
| ACN/IPA (v/v, 1:3) | | Type A |
| Acetone/IPAc (v/v, 1:3) | | Type A |
| DCM/EtOH (v/v, 1:3) | | Type A |
| 1,4-Dioxane/MeOH (v/v, 1:1) | | Type A |
| 1,4-Dioxane/Toluene (v/v, 1:1) | | Type A |

### Liquid Vapor Diffusion

Approximately 15 mg of Compound 1 (Type A) was dissolved in an appropriate solvent to obtain a clear solution in a 4-mL vial. This solution was then placed into a 20-mL vial with 3 mL of anti-solvent. The 20-mL vial was sealed with a cap and kept at RT, allowing sufficient time for organic vapor to interact with the solution. After 7 days, solids were isolated for XRPD analysis (Method A). The results summarized in Table 11 showed that Type A and B were generated.

TABLE 11

| Solvent | Anti-solvent | Solid Form |
|---|---|---|
| THF | MeOH | Type A |
| | EtOAc | Type A |
| 1,4-Dioxane | Toluene | Type A |
| | MTBE | Type A |
| 1,4-Dioxane | MeOH | Type B |
| | EtOAc | Type A |
| | n-Heptane | Type F |
| Acetone | H2O | Type B |
| | IPA | Type A |
| | MIBK | Type A |
| CHCl₃ | EtOH | Type A |
| | 2-MeTHF | Type A |
| | IPAc | Type A |

Another series of liquid vapor diffusion experiments was performed under the conditions set forth in Table 12. Compound 1 (Type A) was weighed into a 3 mL glass vial with the addition of the corresponding solvent or solvent mixture. After being vortexed and ultrasonically shaken, the suspension was filtered, and the filtrate was transferred to a clean 4 mL shell vial. A small amount of Compound 1 (Type A) was added as a seed crystal. Subsequently, the shell vial was sealed with a polyethylene plug with one pinhole and enclosed in a 20 mL glass vial containing 3 mL of the anti-solvent at room temperature for liquid vapor diffusion. The solid forms resulting from the experiments were characterized by XRPD analysis (Method C).

TABLE 12

| Amount of | | Solvent | | |
|---|---|---|---|---|
| Compound 1 | | Volume | | |
| (mg) | Solvent | (mL) | Anti-Solvent | Solid Form |
| 3.0 | MeOH | 0.5 | MTBE | Type B |
| 10.0 | ACN | 0.5 | MTBE | Type B |
| 10.1 | ACN | 0.5 | H₂O | Type B |
| 34.3 | CHCl₃ | 0.3 | n-pentane | Type A |
| 4.9 | Acetone | 0.5 | MTBE | Type A |
| 17.5 | 1,4-dioxane | 0.5 | H₂O | Clear |
| | | | | solution |
| 17.6 | 1,4-dioxane | 0.5 | n-heptane | Type A |
| 11.5 | THF | 0.5 | MeOH | Type D |
| 12.1 | THF | 0.5 | EtOAc | Type M |
| 10.4 | THF | 0.5 | n-pentane | Type D |
| 31.9 | DMSO | 0.2 | H₂O | Type A |
| 10.8 | DMSO | 0.1 | IPA | Type A |
| 32.1 | DMF | 0.2 | Toluene | Type H |
| 10.9 | DMF | 0.1 | IPAc | Type I |
| 30.8 | NMP | 0.2 | MTBE | Type J |
| 10.3 | NMP | 0.1 | H₂O | Clear |
| | | | | solution |

### Slow Evaporation

Slow evaporation experiments were performed under 4 conditions. Briefly, about 15 mg of Compound 1 (Type A) was mixed with 1.0-2.5 mL of solvent in a 4-mL glass vial. If the solids were not dissolved completely, suspensions were filtered using a PTFE membrane (pore size of 0.2 m) and the filtrates were used for the follow-up steps. The visually clear solutions were subjected to evaporation at RT with vials sealed by Parafilm© (3-5 pinholes). The solids were isolated for XRPD analysis (Method A), and the results summarized in Table 13 indicated that only Type A was obtained.

TABLE 13

| Solvent | Solid Form |
|---|---|
| Acetone | Type A |
| THF | Type A |
| CHCl₃ | Type A |
| DCM | Type A |

Another series of slow evaporation experiments was performed under the conditions set forth in Table 14. Compound 1 (Type A) was weighed into a 3 mL glass vial with the addition of the corresponding solvent or solvent mixture. After being vortexed and ultrasonically shaken, the suspension was filtered, and the filtrate was transferred to a clean 4 mL shell vial. A small amount of Compound 1 (Type A) was added as a seed crystal. Subsequently, the shell vial was sealed with a polyethylene plug with one pinhole and placed in a fume hood at room temperature for slow evaporation. The solid forms resulting from the experiments were characterized by XRPD analysis (Method C).

TABLE 14

| Amount of | | Solvent | |
|---|---|---|---|
| Compound 1 | | Volume | |
| (mg) | Solvent | (mL) | Solid Form |
| 3.0 | MeOH | 1.0 | Type B |
| 3.2 | EtOAc | 1.0 | Type B |
| 10.0 | ACN | 0.5 | Type B |
| 4.6 | Acetone | 0.5 | Type A |
| 10.1 | THF | 0.5 | Type D |
| 5.2 | MeOH/Acetone (1:1 v/v) | 0.5 | Type B |
| 3.2 | EtOAc/2-MeTHF (1:1 v/v) | 0.5 | Type B |
| 4.5 | Acetone/EtOH (1:1 v/v) | 0.5 | Type A |
| 4.7 | Acetone/EtOAc (1:1 v/v) | 0.5 | Type A |
| 13.0 | Acetone/H₂O (5:1 v/v) | 0.5 | Type B |
| 13.8 | ACN/EtOH (1:1 v/v) | 0.5 | Type A |
| 17.3 | ACN/IPA (4:1 v/v) | 0.5 | Type A |
| 11.1 | ACN/EtOAc (1:1 v/v) | 0.5 | Type A |
| 5.0 | ACN/MIBK (1:1 v/v) | 0.5 | Type A |
| 14.4 | ACN/2-MeTHF (1:1 v/v) | 0.5 | Type A |
| 27.7 | ACN/n-heptane (10:1 v/v) | 0.5 | Type B |
| 9.8 | ACN/H₂O (5:1 v/v) | 0.5 | Type B |
| 7.3 | DCM/IPAc (1:1 v/v) | 0.5 | Type A |
| 10.8 | DCM/n-heptane (1:1 v/v) | 0.5 | Type A |
| 22.7 | CHCl₃/IPA (1:1 v/v) | 0.5 | Type A |
| 22.9 | CHCl₃/toluene (1:1 v/v) | 0.5 | Type A |
| 10.7 | THF/EtOH (1:1 v/v) | 0.5 | Type A |
| 9.6 | THF/H₂O (5:1 v/v) | 0.5 | Type B |
| 32.9 | 1,4-dioxane/H₂O (4:1 v/v) | 0.5 | Gel |

### Anti-Solvent Addition

A total of 8 anti-solvent addition experiments were carried out. About 15 mg of Compound 1 (Type A) was dissolved in 0.2-4.0 mL of solvent to obtain a clear solution. The solution was magnetically stirred followed by addition of 0.2 mL anti-solvent per step until precipitate appeared or the total amount of anti-solvent reached 15.0 mL. The obtained precipitate was isolated for XRPD analysis (Method A). Results in Table 15 showed that Type A and amorphous material were generated.

TABLE 15

| Solvent/Anti-solvent | Solid Form |
|---|---|
| DMSO/H₂O | Type A + Amorphous |
| CHCl₃/Heptane | Amorphous |
| DMSO/MeOH | Amorphous |
| Acetone/H₂O | Amorphous |
| DMAc/IPA | Amorphous |
| ACN/EtOH | Type A + Amorphous |
| CHCl₃/Toluene | Amorphous |
| DCM/Heptane | Amorphous |

### Reverse Anti-Solvent Addition

A total of 2 reverse anti-solvent addition experiments were carried out. About 15 mg of Compound 1 (type A) was dissolved in 0.2 mL solvent to obtain a clear solution. The solution was added into 2 mL anti-solvent. The obtained precipitate was isolated for XRPD analysis (Method A). Results summarized in Table 16 indicated Type A or a mixture of Type A and amorphous material were generated.

TABLE 16

| Solvent/Anti-solvent | Solid Form |
|---|---|
| DMSO/H₂O | Type A + Amorphous |
| CHCl₃/Heptane | Type A |

### Slow Cooling

Slow cooling experiments were performed under the conditions set forth in Table 17. Compound 1 (Type A) was weighed into a 3 mL glass vial with the addition of the corresponding solvent or solvent mixture. After being vortexed and ultrasonically shaken to accelerate dissolution, the suspension was placed in a biochemical incubator and equilibrated at 50° C. for 30 minutes. The hot suspension was then filtered with a syringe filter (0.045 μm PTFE filter membrane, and the hot filtrate was transferred to a clean 3 mL vial (pre-heated at 50° C.). The vial was sealed and placed in an incubator for slow cooling from 50° C. to 5° C. at a rate of 0.01° C./minute. The solid forms resulting from the experiments were characterized by XRPD analysis (Method C).

TABLE 17

| Amount of | | Solvent | |
|---|---|---|---|
| Compound 1 | | Volume | Solid |
| (mg) | Solvent | (mL) | Form |
| 20.5 | MeOH | 1.5 | Type B |
| 15.7 | EtOH | 1.5 | Type A |
| 20.3 | EtOAc | 1.5 | Type B |
| 14.9 | MIBK | 1.5 | Clear |
| | | | solution |
| 15.4 | 2-MeTHF | 1.5 | Type K |
| 40.3 | ACN/H₂O (2:1 v/v) | 0.8 | Type B |
| 40.6 | Acetone/H₂O (2:1 v/v) | 1.0 | Type B |
| 40.3 | Acetone/n-heptane (3:1 v/v) | 1.0 | Type A |
| 40.4 | 1,4-dioxane/H₂O (1:1 v/v) | 1.0 | Type B |
| 39.7 | THF/H₂O (1:1 v/v)* | 1.0 | Type L |

*After slow cooling, only a few small crystals were observed. System was kept at 5° C. for 22 days, at which time plate crystals (Type L) were observed.

### Example 4—Preparation and Characterization of Type B of Compound 1

### Preparation of Type B of Compound 1

Type B was prepared on a 100 mg scale from a slurry of Type A in methanol at 50° C., via a method analogous to the method for slurry conversion described in Example 3.

### Characterization of Type B of Compound 1

Type B was characterized by XRPD (Method A), TGA (Method A), DSC (Method A), and DVS analysis.

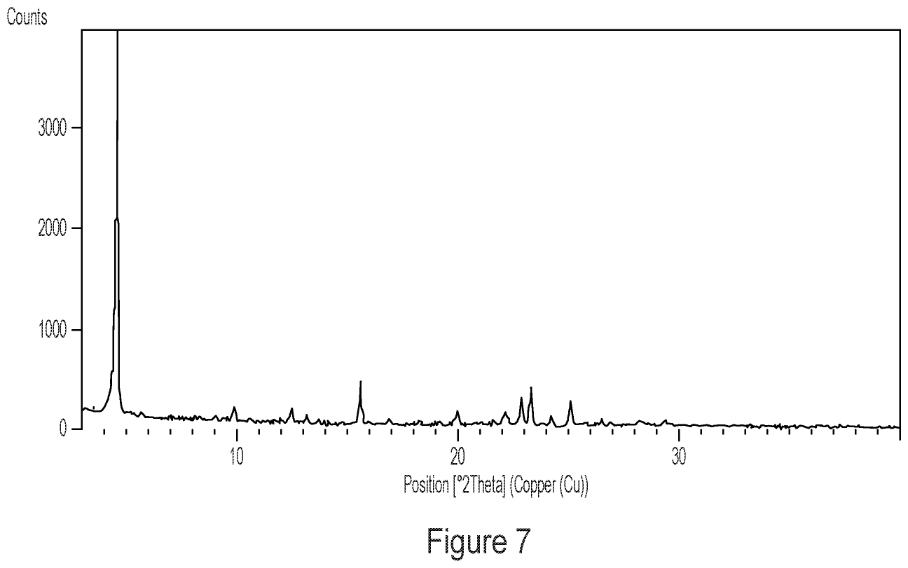

The XRPD pattern for Type B is depicted in FIG. 7, and the corresponding data are summarized in the following table:

| Pos. [°2Th.] | d-spacing [Å] |
|---|---|
| 4.52 | 19.53 |
| 8.98 | 9.85 |
| 9.86 | 8.97 |
| 12.37 | 7.15 |
| 13.18 | 6.72 |
| 15.57 | 5.69 |
| 16.86 | 5.26 |
| 18.21 | 4.87 |
| 19.11 | 4.64 |
| 19.93 | 4.45 |
| 20.92 | 4.25 |
| 22.19 | 4.00 |
| 22.89 | 3.89 |
| 23.34 | 3.81 |
| 25.13 | 3.54 |
| 25.80 | 3.45 |
| 26.71 | 3.34 |
| 28.30 | 3.15 |
| 29.39 | 3.04 |

The foregoing XRPD data for Type B can also be rounded to a single decimal place, as summarized in the following table

| Pos. [°2Th.] | d-spacing [Å] |
|---|---|
| 4.5 | 19.5 |
| 9.0 | 9.9 |
| 9.9 | 9.0 |
| 12.4 | 7.2 |
| 13.2 | 6.7 |
| 15.6 | 5.7 |
| 16.9 | 5.3 |
| 18.2 | 4.9 |
| 19.1 | 4.6 |
| 19.9 | 4.5 |
| 20.9 | 4.2 |
| 22.2 | 4.0 |
| 22.9 | 3.9 |
| 23.3 | 3.8 |
| 25.1 | 3.5 |
| 25.8 | 3.5 |
| 26.7 | 3.3 |
| 28.3 | 3.2 |
| 29.4 | 3.0 |

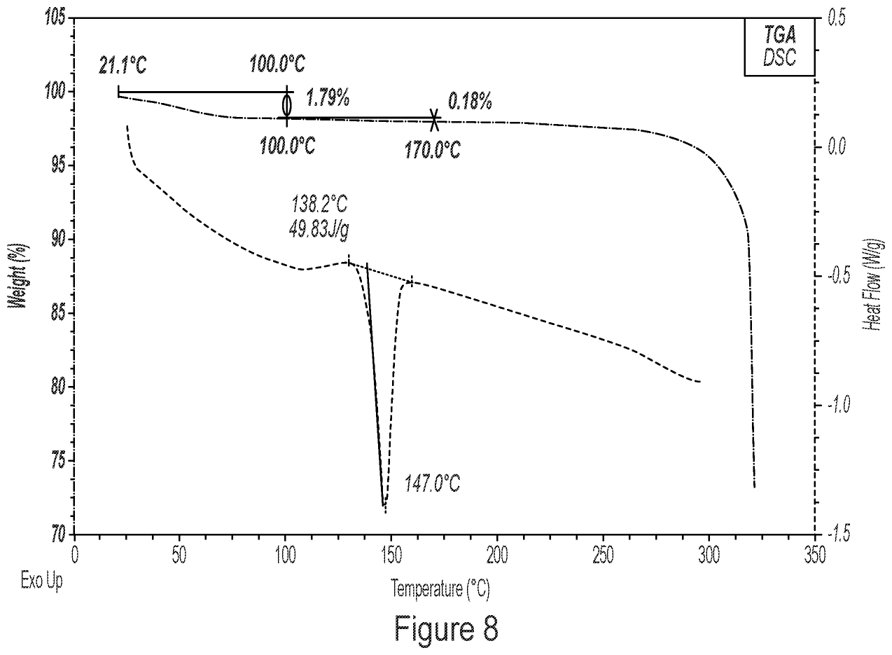

The TGA and DSC curves for Type B are shown in FIG. 8. As shown in FIG. 8, Type B showed 1.8% weight loss up to 100° C. and an endotherm at 138.2° C. (onset temperature) possibly due to melting.

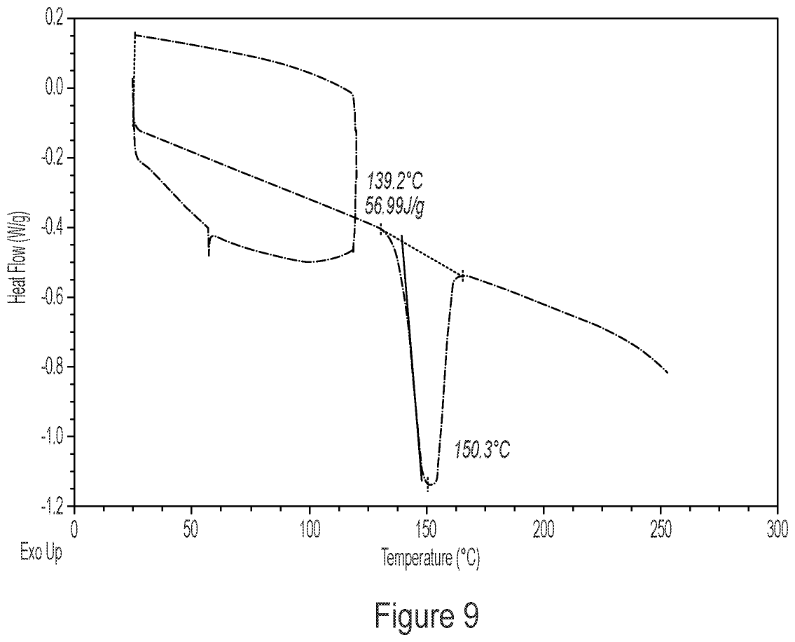

By DSC cycling (RT-120° C.-RT-250° C.) as shown in FIG. 9, only one melting endotherm at 139.2° C. (onset temperature) was observed, with no broad endotherm below 120° C. in the second heating cycle.

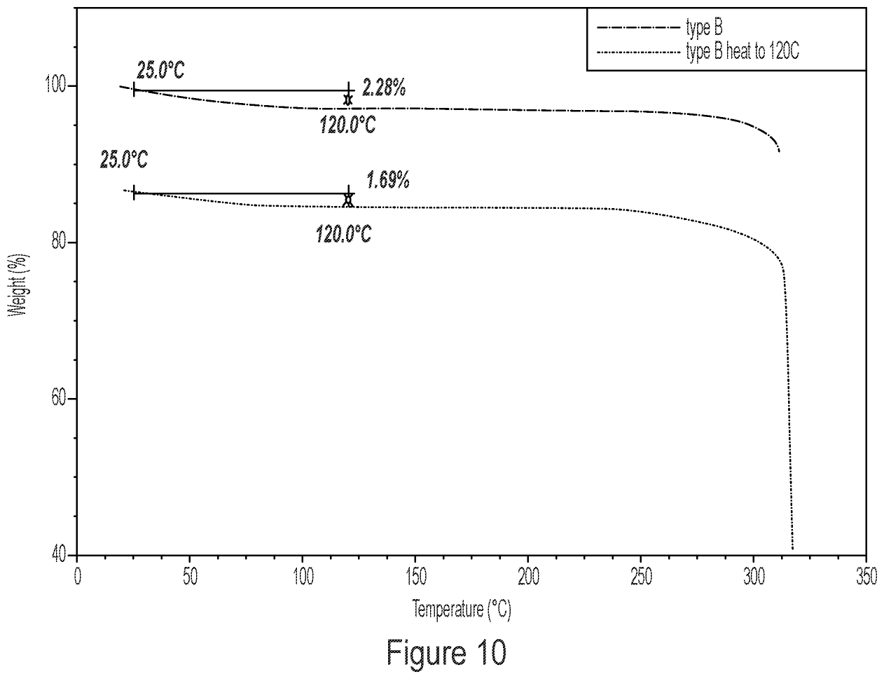

As shown in FIG. 10, Type B showed a 1.7% weight loss up to 120° C. by instant TGA test after heating to 120° C. and exposing to ambient condition for only one minute. The normal TGA curve of Type B showed 2.3% weight loss up 120° C. without pre-heating treatment.

After heating to 120° C. and cooling to RT by DSC cycling, no form change was observed by XRPD analysis.

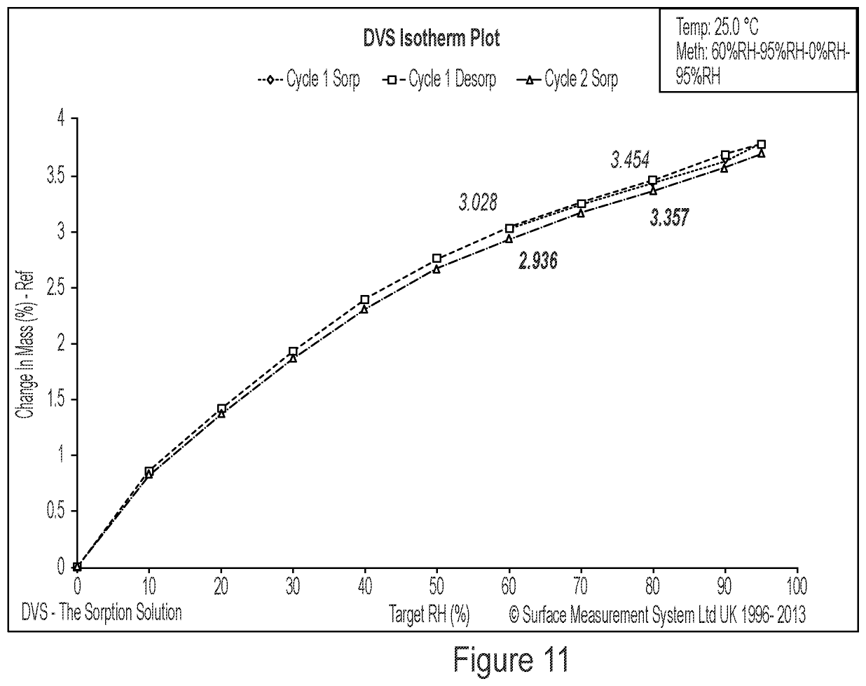

By DVS analysis (FIG. 11), Type B showed a 2.9% water uptake up to 60% RH (ambient condition), and 0.4% water uptake from 60% RH to 80% RH at RT, indicating that Type B is hygroscopic. No form change was observed for Type B before and after DVS test at RT, as determined by XRPD analysis.

Based on the foregoing analytical data, Type B is believed to be a channel hydrate.

### Example 5—Preparation and Characterization of Type C of Compound 1

### Preparation of Type C of Compound 1

Type C was prepared on a 100 mg scale from a slurry of Type A in 1,4-Dioxane at RT, via a method analogous to the method for slurry conversion described in Example 3.

### Characterization of Type C of Compound 1

Type C was characterized by XRPD (Method A), TGA (Method A), DSC (Method A), and DVS analysis.

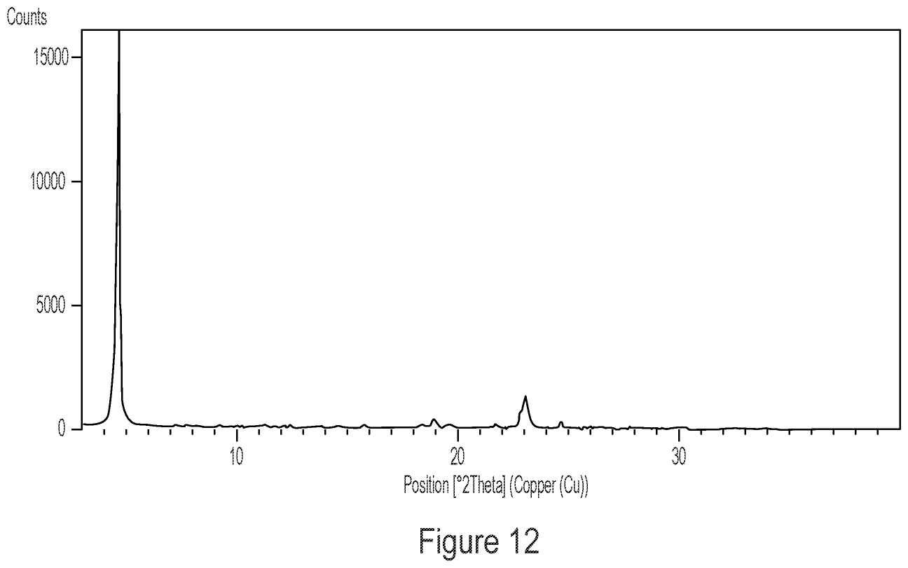

The XRPD pattern for Type C is depicted in FIG. 12, and the corresponding data are summarized in the following table:

| Pos. [°2Th.] | d-spacing [Å] |
|---|---|
| 4.55 | 19.43 |
| 7.34 | 12.05 |
| 9.07 | 9.75 |
| 11.17 | 7.92 |
| 12.29 | 7.20 |
| 14.51 | 6.11 |
| 15.66 | 5.66 |
| 18.34 | 4.84 |
| 18.85 | 4.71 |
| 19.57 | 4.54 |
| 20.38 | 4.36 |
| 21.66 | 4.10 |
| 23.02 | 3.86 |
| 24.65 | 3.61 |
| 26.39 | 3.38 |
| 28.28 | 3.16 |
| 30.09 | 2.97 |
| 32.31 | 2.77 |
| 33.91 | 2.64 |
| 37.19 | 2.42 |

The foregoing XRPD data for Type C can also be rounded to a single decimal place, as summarized in the following table:

| Pos. [°2Th.] | d-spacing [Å] |
|---|---|
| 4.5 | 19.4 |
| 7.3 | 12.0 |
| 9.1 | 9.7 |
| 11.2 | 7.9 |
| 12.3 | 7.2 |
| 14.5 | 6.1 |
| 15.7 | 5.7 |
| 18.3 | 4.8 |
| 18.9 | 4.7 |
| 19.6 | 4.5 |
| 20.4 | 4.4 |
| 21.7 | 4.1 |
| 23.0 | 3.9 |
| 24.7 | 3.6 |
| 26.4 | 3.4 |
| 28.3 | 3.2 |
| 30.1 | 3.0 |
| 32.3 | 2.8 |
| 33.9 | 2.6 |
| 37.2 | 2.4 |

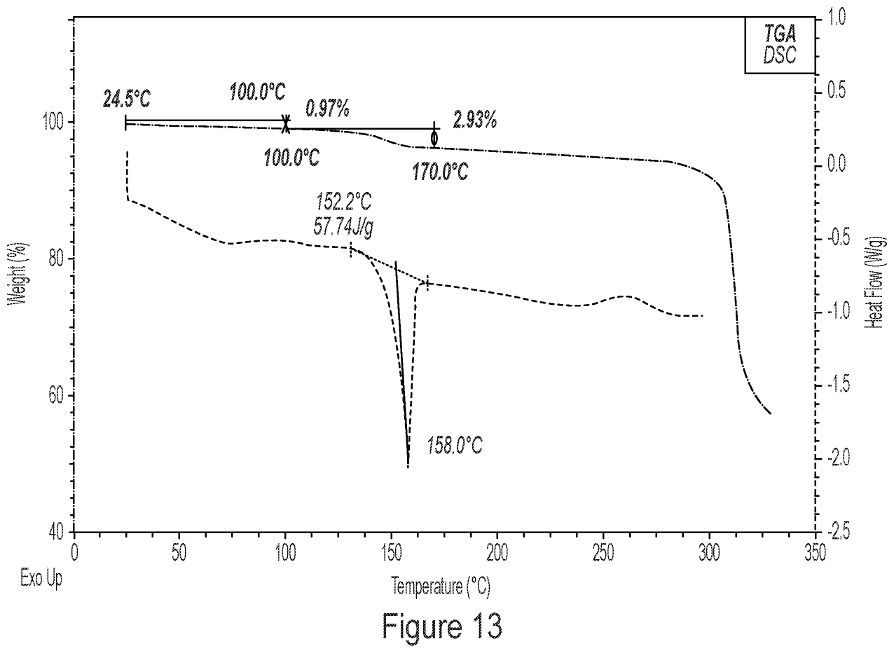

The TGA and DSC curves for Type C are shown in FIG. 13. As shown in FIG. 13, Type C showed 1.0% weight loss up to 100° C. and an endotherm at 152.2° C. (onset temperature) possibly due to melting.

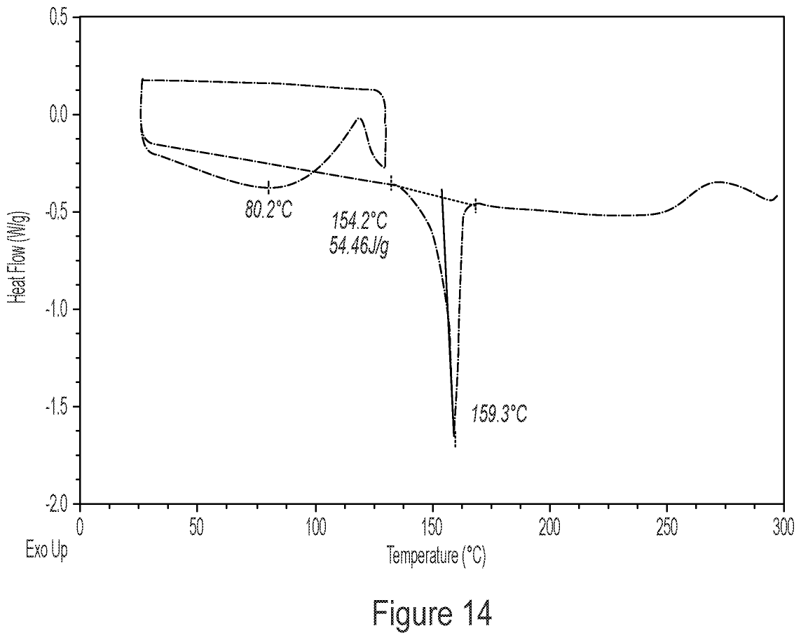

By using DSC cycling (RT-120° C.-RT-250° C.), only a melting endotherm at 154.2° C. (onset temperature) was observed, with no broad endotherm below 120° C. in the second heating cycle (FIG. 14).

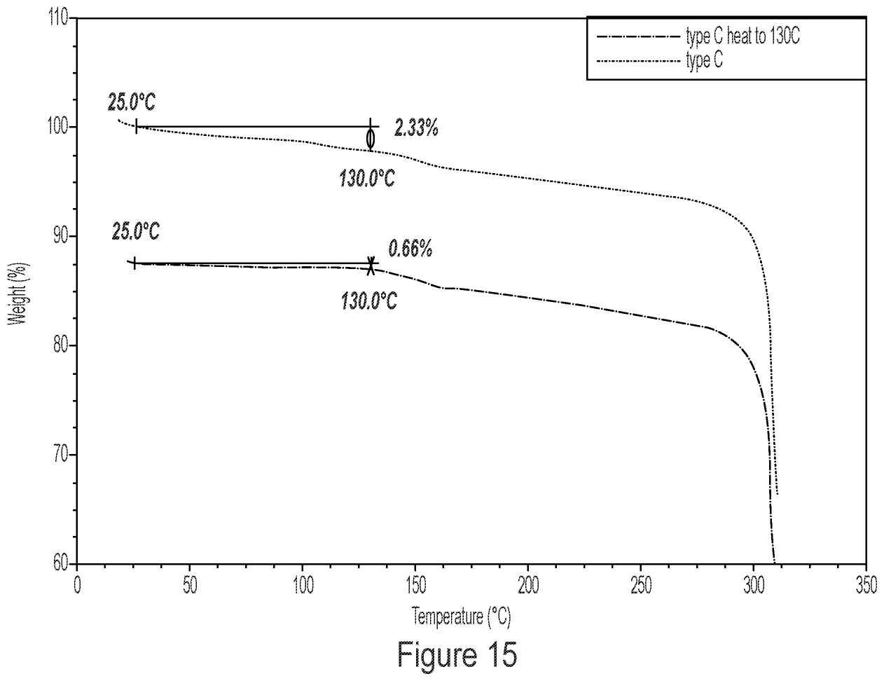

As shown in FIG. 15, Type C showed a 0.7% weight loss up to 130° C. by instant TGA test after heating to 120° C. and exposing to ambient conditions for one minute. The normal TGA curve of the Type C showed 2.3% weight loss up 130° C. without pre-heating treatment.

After heating to 120° C. and cooling to RT by DSC cycling, no form change was observed by XRPD analysis.

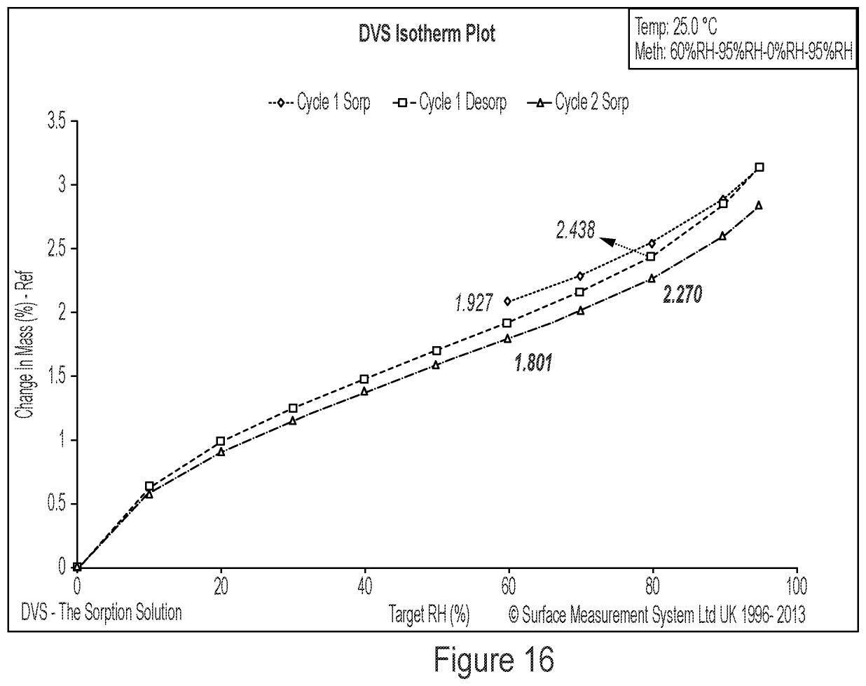

By DVS analysis (FIG. 16), Type C showed a 1.8% water uptake up to 60% RH (ambient condition), and 0.5% water uptake from 60% RH to 80% RH at RT, indicating that Type C is hygroscopic. No form change was observed for Type C before and after DVS test at RT, as determined by XRPD analysis.

Based on the foregoing analytical data, Type B is believed to be a channel hydrate.

### Example 6—Preparation and Characterization of Type D of Compound 1

### Preparation of Type D of Compound 1

Type D was prepared from a slurry of Type A in Tetrahydrofuran (THF) at 4° C., via a method analogous to the method for slurry conversion described in Example 3.

### Characterization of Type D of Compound 1

Type D was characterized by XRPD (Method A), TGA (Method A), DSC (Method A), and ¹H NMR analysis.

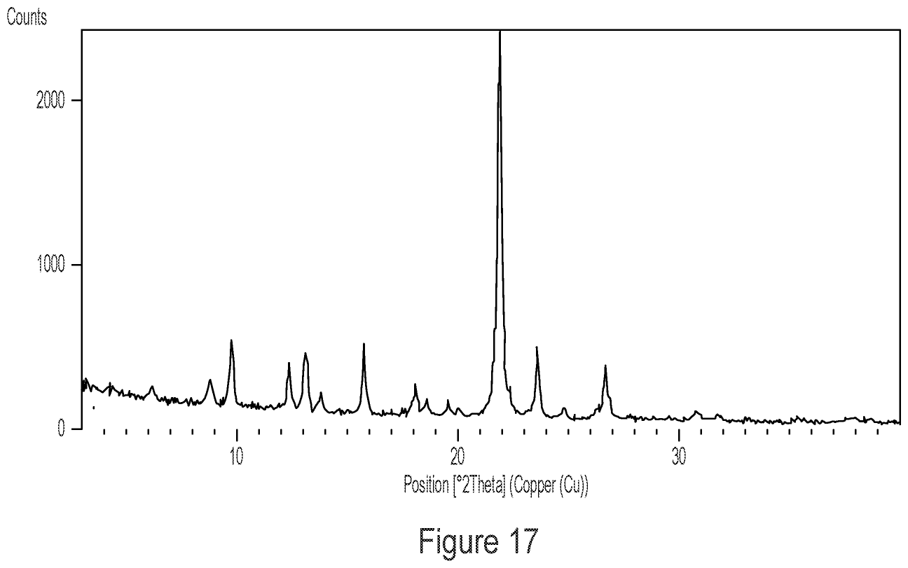

The XRPD pattern for Type D is depicted in FIG. 17, and the corresponding data are summarized in the following table:

| Pos. | d-spacing |
|---|---|
| [°2Th.] | [Å] |
| 4.27 | 20.68 |
| 6.15 | 14.36 |
| 8.71 | 10.16 |
| 9.72 | 9.10 |
| 12.31 | 7.19 |
| 13.08 | 6.77 |
| 13.76 | 6.44 |
| 15.74 | 5.63 |
| 18.02 | 4.92 |
| 19.55 | 4.54 |
| 21.90 | 4.06 |
| 23.59 | 3.77 |
| 24.79 | 3.59 |
| 26.71 | 3.34 |
| 29.50 | 3.03 |
| 30.82 | 2.90 |
| 31.74 | 2.82 |
| 35.40 | 2.54 |
| 37.84 | 2.38 |
| 38.61 | 2.33 |

The foregoing XRPD data for Type D can also be rounded to a single decimal place, as summarized in the following table:

| Pos. [°2Th.] | d-spacing [Å] |
|---|---|
| 4.3 | 20.7 |
| 6.2 | 14.4 |
| 8.7 | 10.2 |
| 9.7 | 9.1 |
| 12.3 | 7.2 |
| 13.1 | 6.8 |
| 13.8 | 6.4 |
| 15.7 | 5.6 |
| 18.0 | 4.9 |
| 19.5 | 4.5 |
| 21.9 | 4.1 |
| 23.6 | 3.8 |
| 24.8 | 3.6 |
| 26.7 | 3.3 |
| 29.5 | 3.0 |
| 30.8 | 2.9 |
| 31.7 | 2.8 |
| 35.4 | 2.5 |
| 37.8 | 2.4 |
| 38.6 | 2.3 |

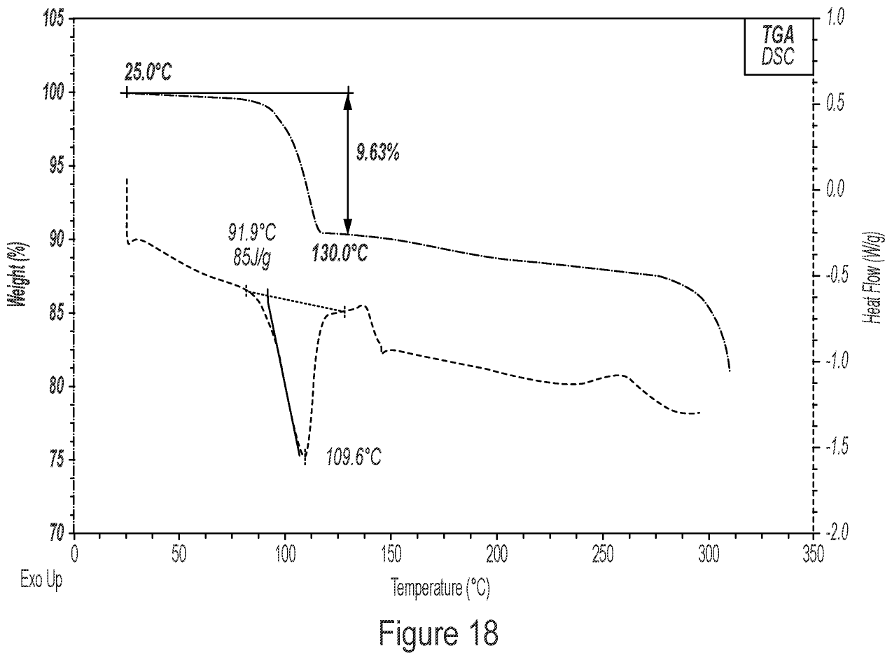

The TGA and DSC curves for Type D are shown in FIG. 18. As shown in FIG. 18, Type D showed 9.6% weight loss up to 130° C. by TGA and an endotherm at 91.9° C. (onset temperature) by DSC.

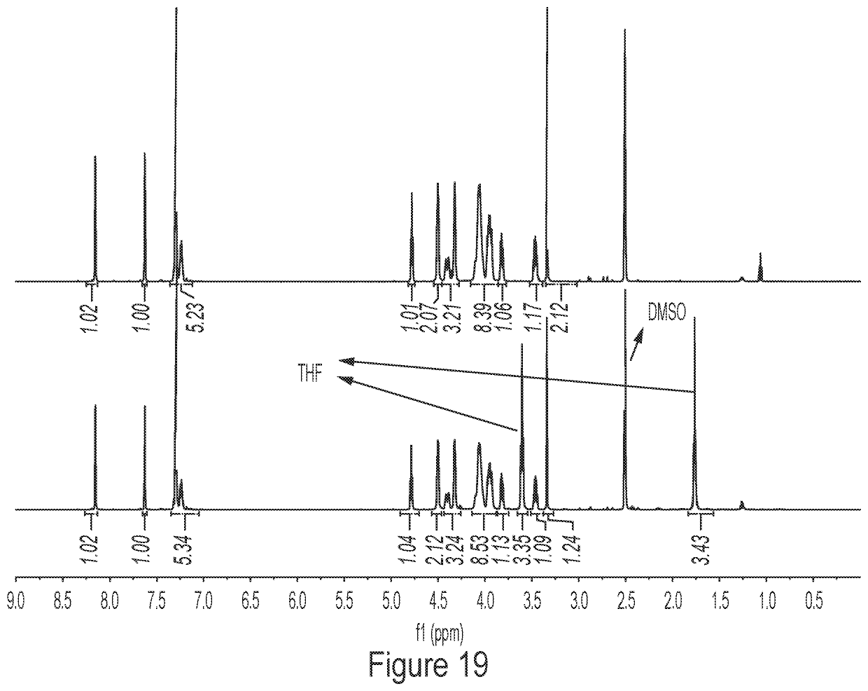

The ¹H NMR spectra of Type A and Type D are shown in FIG. 19. Type D appears to be a THE solvate, as indicated by the ¹H NMR spectrum (600 MHz, DMSO-d6), which detected the presence of THF protons at ˜1.76 and ˜3.60 ppm.

### Example 7—Preparation and Characterization of Type E of Compound 1

### Characterization of Type E of Compound 1

Type E was characterized by XRPD (Method A) analysis.

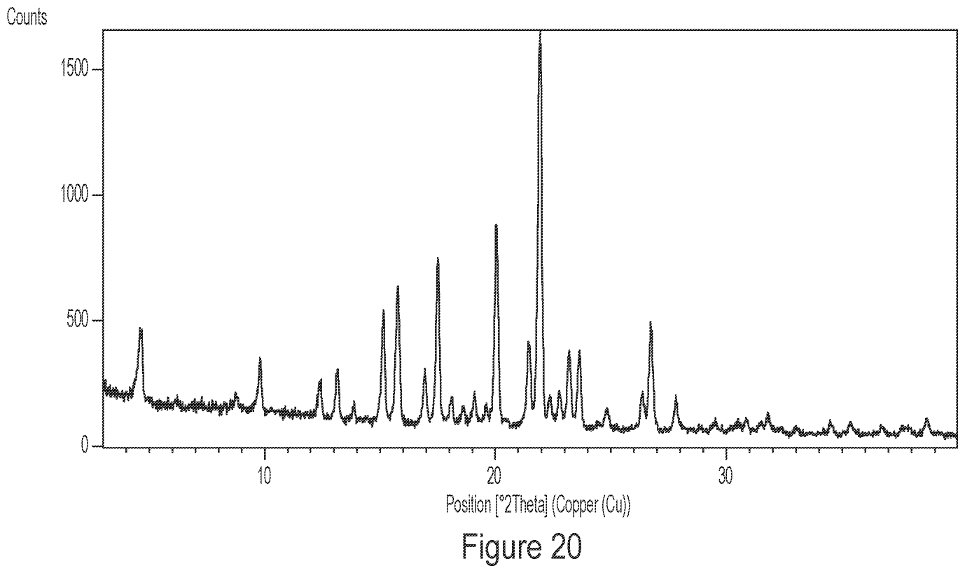

The XRPD pattern for Type E is depicted in FIG. 20, and the corresponding data are summarized in the following table:

| Pos. [°2Th.] | d-spacing [Å] |
|---|---|
| 4.59 | 19.27 |
| 8.76 | 10.09 |
| 9.76 | 9.06 |
| 12.36 | 7.16 |
| 13.12 | 6.75 |
| 13.83 | 6.40 |
| 15.12 | 5.86 |
| 15.75 | 5.63 |
| 16.84 | 5.27 |
| 17.48 | 5.07 |
| 18.06 | 4.91 |
| 19.02 | 4.67 |
| 20.05 | 4.43 |
| 21.93 | 4.05 |
| 23.18 | 3.84 |
| 23.70 | 3.75 |
| 24.82 | 3.59 |
| 26.72 | 3.34 |
| 27.81 | 3.21 |
| 29.51 | 3.03 |
| 30.76 | 2.91 |
| 31.74 | 2.82 |
| 33.03 | 2.71 |
| 34.52 | 2.60 |
| 35.39 | 2.54 |
| 36.72 | 2.45 |
| 37.77 | 2.38 |
| 38.66 | 2.33 |

The foregoing XRPD data for Type E can also be rounded to a single decimal place, as summarized in the following table:

| Pos. [°2Th.] | d-spacing [Å] |
|---|---|
| 4.6 | 19.3 |
| 8.8 | 10.1 |
| 9.8 | 9.1 |
| 12.4 | 7.2 |
| 13.1 | 6.7 |
| 13.8 | 6.4 |
| 15.1 | 5.9 |
| 15.8 | 5.6 |
| 16.8 | 5.3 |
| 17.5 | 5.1 |
| 18.1 | 4.9 |
| 19.0 | 4.7 |
| 20.0 | 4.4 |
| 21.9 | 4.1 |
| 23.2 | 3.8 |
| 23.7 | 3.8 |
| 24.8 | 3.6 |
| 26.7 | 3.3 |
| 27.8 | 3.2 |
| 29.5 | 3.0 |
| 30.8 | 2.9 |
| 31.7 | 2.8 |
| 33.0 | 2.7 |
| 34.5 | 2.6 |
| 35.4 | 2.5 |
| 36.7 | 2.4 |
| 37.8 | 2.4 |
| 38.7 | 2.3 |

### Example 8—Preparation and Characterization of Type F of Compound 1

### Preparation of Type F of Compound 1

Type F of Compound 1 was produced via liquid vapor diffusion in 1,4-Dioxane/heptane at RT.

### Characterization of Type F of Compound 1

Type F was characterized by XRPD (Method A), TGA, and DSC analysis (Method A).

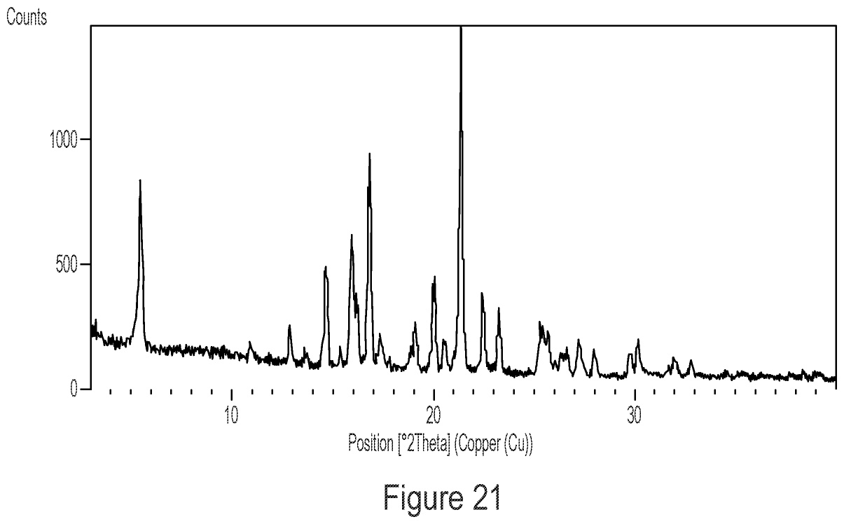

The XRPD pattern for Type F is shown in FIG. 21, and the corresponding data are summarized in the following table:

| Pos. [°2Th.] | d-spacing [Å] |
|---|---|
| 5.45 | 16.23 |
| 10.92 | 8.10 |
| 12.87 | 6.88 |
| 14.66 | 6.04 |
| 16.00 | 5.54 |
| 16.79 | 5.28 |
| 17.36 | 5.11 |
| 18.99 | 4.67 |
| 20.01 | 4.44 |
| 20.57 | 4.32 |
| 21.36 | 4.16 |
| 22.45 | 3.96 |
| 23.25 | 3.83 |
| 25.32 | 3.52 |
| 26.57 | 3.35 |
| 27.25 | 3.27 |
| 27.97 | 3.19 |
| 30.02 | 2.98 |
| 31.98 | 2.80 |
| 32.89 | 2.72 |
| 38.29 | 2.35 |
| 39.09 | 2.30 |

The foregoing XRPD data for Type F can also be rounded to a single decimal place, as summarized in the following table:

| Pos. [°2Th.] | d-spacing [Å] |
|---|---|
| 5.4 | 16.2 |
| 10.9 | 8.1 |
| 12.9 | 6.9 |
| 14.7 | 6.0 |
| 16.0 | 5.5 |
| 16.8 | 5.3 |
| 17.4 | 5.1 |
| 19.0 | 4.7 |
| 20.0 | 4.4 |
| 20.6 | 4.3 |
| 21.4 | 4.2 |
| 22.5 | 4.0 |
| 23.2 | 3.8 |
| 25.3 | 3.5 |
| 26.6 | 3.4 |
| 27.2 | 3.3 |
| 28.0 | 3.2 |
| 30.0 | 3.0 |
| 32.0 | 2.8 |
| 32.9 | 2.7 |
| 38.3 | 2.4 |
| 39.1 | 2.3 |

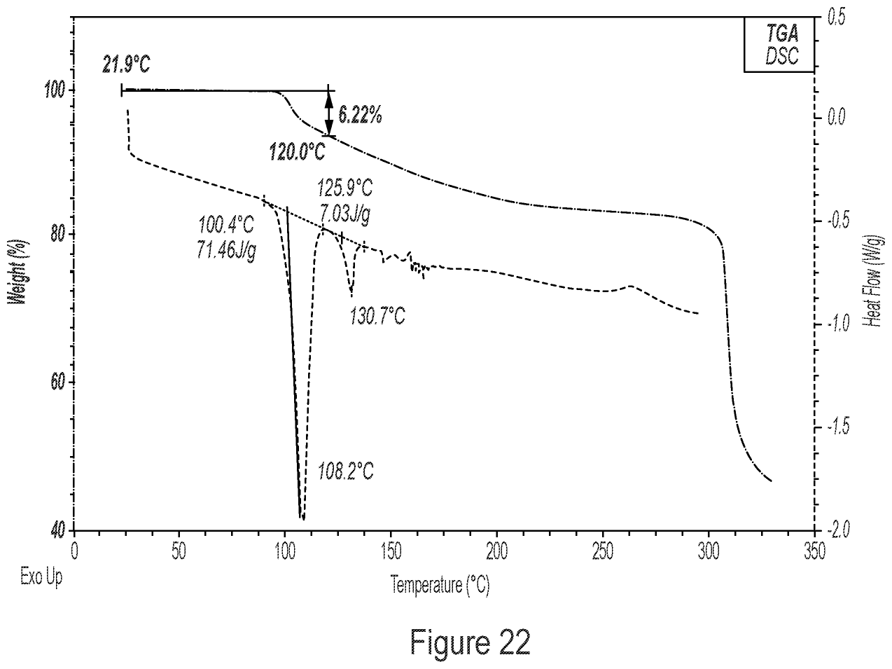

The TGA and DSC curves for Type F are shown in FIG. 22. As shown in FIG. 22, Type F showed 6.2% weight loss up to 120° C. by TGA and two endotherms at 100.4° C. and 125.9° C. (onset temperature) by DSC.

### Example 9—Preparation and Characterization of Type G of Compound 1

### Preparation of Type G of Compound 1

Type G was prepared from a slurry of Type A in methyl ethyl ketone at room temperature.

### Characterization of Type G of Compound 1

Type G was characterized by XRPD (Method A) analysis.

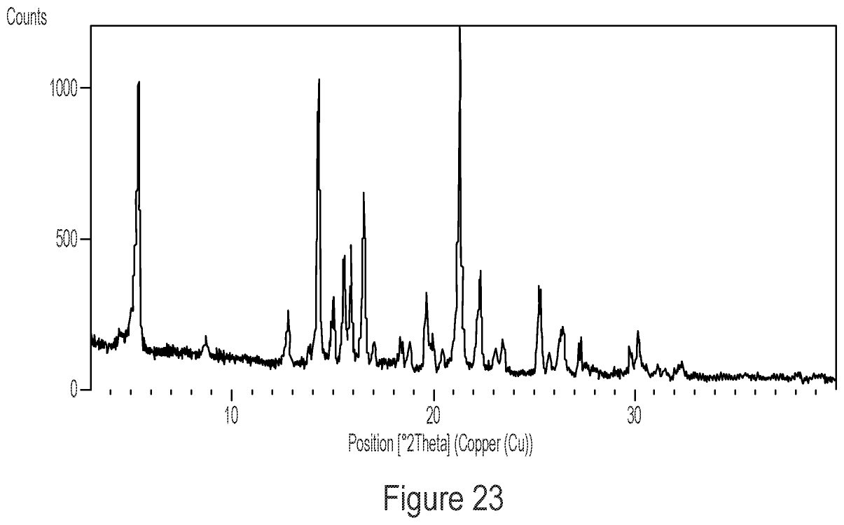

The XRPD pattern for Type G is depicted in FIG. 23, and the corresponding data are summarized in the following table:

| Pos. [°2Th.] | d-spacing [Å] |
|---|---|
| 5.36 | 16.48 |
| 8.73 | 10.13 |
| 12.83 | 6.90 |
| 14.34 | 6.18 |
| 15.00 | 5.91 |
| 15.79 | 5.61 |
| 16.58 | 5.35 |
| 18.54 | 4.79 |
| 19.78 | 4.49 |
| 21.35 | 4.16 |
| 22.35 | 3.98 |
| 23.38 | 3.80 |
| 25.33 | 3.52 |
| 26.43 | 3.37 |
| 27.35 | 3.26 |
| 30.21 | 2.96 |
| 32.32 | 2.77 |
| 38.04 | 2.37 |

The foregoing XRPD data for Type G can also be rounded to a single decimal place, as summarized in the following table:

| Pos. [°2Th.] | d-spacing [Å] |
|---|---|
| 5.4 | 16.5 |
| 8.7 | 10.1 |
| 12.8 | 6.9 |
| 14.3 | 6.2 |
| 15.0 | 5.9 |
| 15.8 | 5.6 |
| 16.6 | 5.3 |
| 18.5 | 4.8 |
| 19.8 | 4.5 |
| 21.3 | 4.2 |
| 22.3 | 4.0 |
| 23.4 | 3.8 |
| 25.3 | 3.5 |
| 26.4 | 3.4 |
| 27.4 | 3.3 |
| 30.2 | 3.0 |
| 32.3 | 2.8 |
| 38.0 | 2.4 |

### Example 10—Preparation and Characterization of Type H of Compound 1

### Preparation of Type H of Compound 1

Type H was prepared by liquid vapor diffusion, as described in Example 3.

### Characterization of Type H of Compound 1

Type H was characterized by XRPD (Method C) analysis.

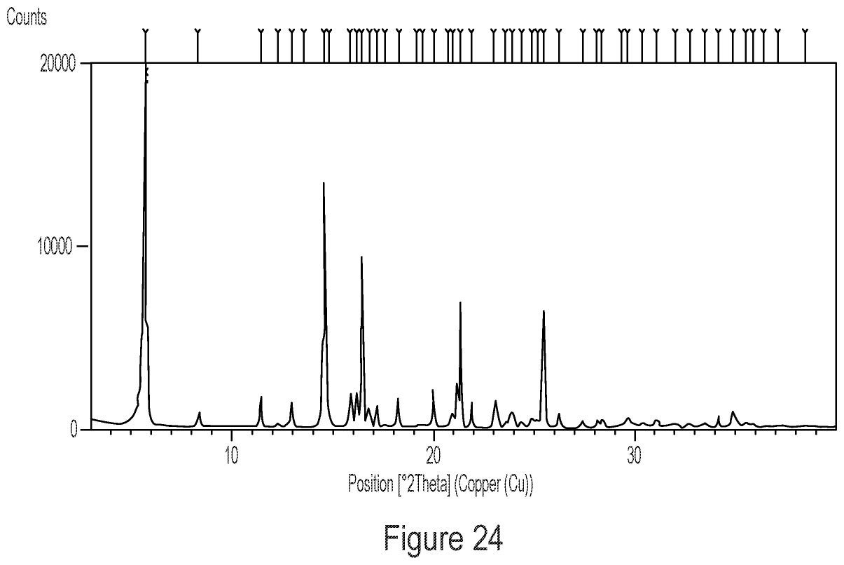

The XRPD pattern for Type H is depicted in FIG. 24 and the corresponding data are summarized in the following table:

| Pos. [°2Th.] | d-spacing [Å] |
|---|---|
| 5.8 | 15.3 |
| 8.4 | 10.5 |
| 11.5 | 7.7 |
| 12.4 | 7.2 |
| 13.1 | 6.8 |
| 13.7 | 6.5 |
| 14.7 | 6.0 |
| 14.9 | 5.9 |
| 16.0 | 5.6 |
| 16.2 | 5.5 |
| 16.6 | 5.4 |
| 16.9 | 5.3 |
| 17.3 | 5.1 |
| 17.7 | 5.0 |
| 18.3 | 4.8 |
| 19.5 | 4.6 |
| 20.0 | 4.4 |
| 21.3 | 4.2 |
| 21.9 | 4.1 |
| 23.1 | 3.9 |
| 23.6 | 3.8 |
| 23.9 | 3.7 |
| 24.4 | 3.7 |
| 24.9 | 3.6 |
| 25.1 | 3.5 |
| 25.4 | 3.5 |
| 26.2 | 3.4 |
| 27.4 | 3.3 |
| 28.1 | 3.2 |
| 28.4 | 3.1 |
| 29.3 | 3.0 |
| 29.7 | 3.0 |
| 30.4 | 2.9 |
| 31.0 | 2.9 |
| 32.7 | 2.7 |
| 33.4 | 2.7 |
| 34.1 | 2.6 |
| 34.8 | 2.6 |
| 35.5 | 2.5 |
| 35.8 | 2.5 |
| 36.4 | 2.5 |
| 37.1 | 2.4 |
| 38.5 | 2.3 |

### Example 11—Preparation and Characterization of Type I of Compound 1

### Preparation of Type I of Compound 1

Type I was prepared by liquid vapor diffusion, as described in Example 3.

### Characterization of Type I of Compound 1

Type I was characterized by XRPD (Method C) analysis.

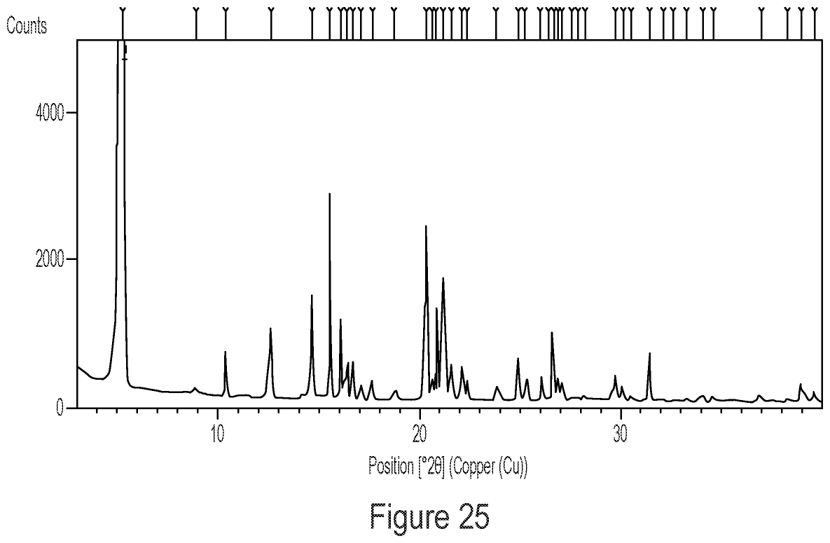

The XRPD pattern for Type I is depicted in FIG. 25, and the corresponding data are summarized in the following table:

| Pos. [°2Th.] | d-spacing [Å] |
|---|---|
| 5.2 | 17.1 |
| 8.8 | 10.1 |
| 10.3 | 8.6 |
| 12.6 | 7.0 |
| 14.6 | 6.1 |
| 15.5 | 5.7 |
| 16.1 | 5.5 |
| 16.3 | 5.4 |
| 16.6 | 5.3 |
| 17.1 | 5.2 |
| 17.6 | 5.0 |
| 18.7 | 4.7 |
| 18.9 | 4.7 |
| 20.2 | 4.4 |
| 20.5 | 4.3 |
| 20.7 | 4.3 |
| 21.1 | 4.2 |
| 21.5 | 4.1 |
| 22.0 | 4.0 |
| 22.3 | 4.0 |
| 23.7 | 3.8 |
| 24.8 | 3.6 |
| 25.2 | 3.5 |
| 26.0 | 3.4 |
| 26.3 | 3.4 |
| 26.5 | 3.4 |
| 26.8 | 3.3 |
| 27.0 | 3.3 |
| 27.5 | 3.2 |
| 27.7 | 3.2 |
| 28.1 | 3.2 |
| 29.6 | 3.0 |
| 30.0 | 3.0 |
| 30.4 | 2.9 |
| 31.3 | 2.9 |
| 32.0 | 2.8 |
| 32.5 | 2.8 |
| 33.2 | 2.7 |
| 34.0 | 2.6 |
| 34.6 | 2.6 |
| 36.9 | 2.4 |
| 38.2 | 2.4 |
| 38.9 | 2.3 |
| 39.5 | 2.3 |

### Example 12—Preparation and Characterization of Type J of Compound 1

### Preparation of Type J of Compound 1

Type J was prepared by liquid vapor diffusion, as described in Example 3.

### Characterization of Type J of Compound 1

Type J was characterized by XRPD (Method C) analysis.

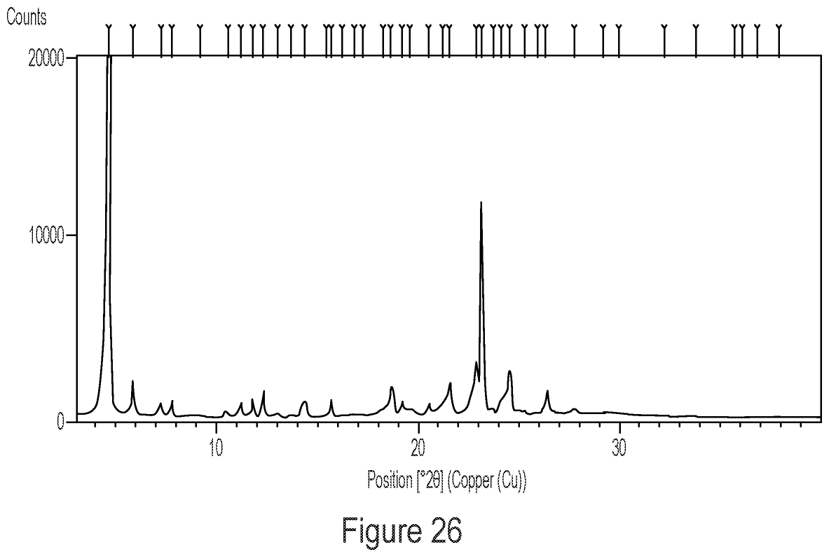

The XRPD pattern for Type J is depicted in FIG. 26, and the corresponding data are summarized in the following table:

| Pos. [°2Th.] | d-spacing [Å] |
|---|---|
| 4.5 | 19.5 |
| 5.7 | 15.4 |
| 7.1 | 12.7 |
| 7.7 | 11.5 |
| 9.1 | 9.7 |
| 10.5 | 8.4 |
| 11.2 | 7.9 |
| 11.7 | 7.5 |
| 12.3 | 7.2 |
| 12.9 | 6.8 |
| 14.3 | 6.2 |
| 14.5 | 6.1 |
| 15.4 | 5.8 |
| 15.7 | 5.7 |
| 16.3 | 5.4 |
| 17.3 | 5.1 |
| 18.3 | 4.9 |
| 18.7 | 4.7 |
| 19.3 | 4.6 |
| 19.6 | 4.5 |
| 20.5 | 4.3 |
| 21.2 | 4.2 |
| 21.5 | 4.1 |
| 22.8 | 3.9 |
| 23.1 | 3.8 |
| 23.6 | 3.8 |
| 24.1 | 3.7 |
| 24.5 | 3.6 |
| 25.2 | 3.5 |
| 25.9 | 3.4 |
| 26.4 | 3.4 |
| 27.8 | 3.2 |
| 29.3 | 3.0 |
| 36.2 | 2.5 |
| 37.0 | 2.4 |

### Example 13—Preparation and Characterization of Type K of Compound 1

### Preparation of Type K of Compound 1

Type K was prepared by slow cooling, as described in Example 3.

### Characterization of Type K of Compound 1

Type K was characterized by XRPD (Method C) analysis.

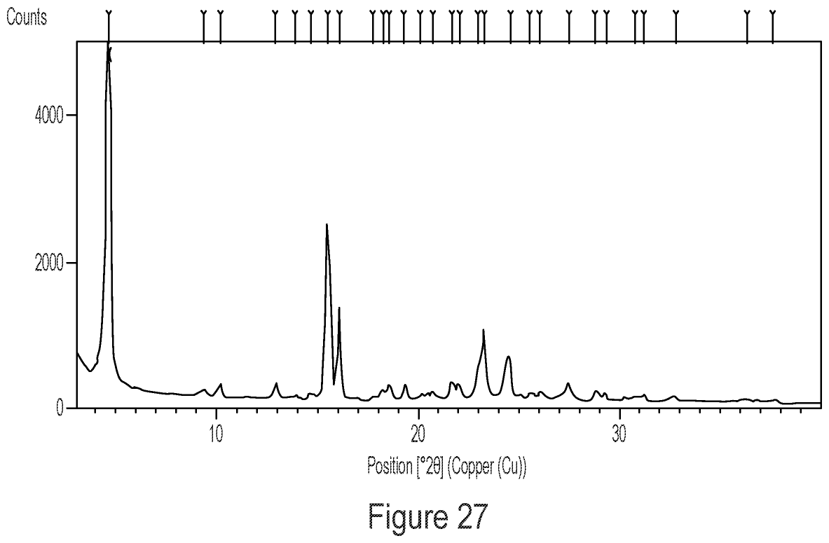

The XRPD pattern for Type K is depicted in FIG. 27, and the corresponding data are summarized in the following table:

| Pos. [°2Th.] | d-spacing [Å] |
|---|---|
| 4.6 | 19.2 |
| 9.3 | 9.5 |
| 10.1 | 8.7 |
| 12.9 | 6.8 |
| 13.9 | 6.4 |
| 14.7 | 6.0 |
| 15.4 | 5.7 |
| 15.6 | 5.7 |
| 16.1 | 5.5 |
| 17.8 | 5.0 |
| 18.3 | 4.9 |
| 18.6 | 4.8 |
| 19.3 | 4.6 |
| 20.0 | 4.4 |
| 20.7 | 4.3 |
| 21.6 | 4.1 |
| 21.9 | 4.1 |
| 22.9 | 3.9 |
| 23.2 | 3.8 |
| 24.4 | 3.6 |
| 25.0 | 3.6 |
| 25.5 | 3.5 |
| 26.0 | 3.4 |
| 27.4 | 3.3 |
| 28.8 | 3.1 |
| 29.2 | 3.1 |
| 30.7 | 2.9 |
| 31.1 | 2.9 |
| 32.7 | 2.7 |
| 36.3 | 2.5 |

### Example 14—Preparation and Characterization of Type L of Compound 1

### Preparation of Type L of Compound 1

Type L was prepared by slow cooling, as described in Example 3.

### Characterization of Type L of Compound 1

Type L was characterized by XRPD (Method C) analysis.

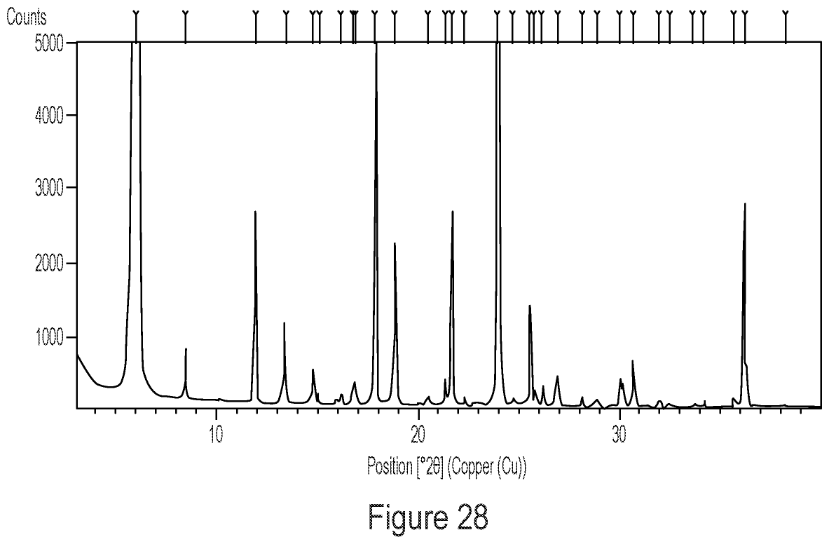

The XRPD pattern for Type L is depicted in FIG. 28, and the corresponding data are summarized in the following table:

| Pos. [°2Th.] | d-spacing [Å] |
|---|---|
| 5.9 | 14.9 |
| 8.4 | 10.5 |
| 11.9 | 7.5 |
| 13.3 | 6.6 |
| 14.7 | 6.0 |
| 15.0 | 5.9 |
| 16.2 | 5.5 |
| 16.7 | 5.3 |
| 16.9 | 5.2 |
| 17.8 | 5.0 |
| 18.9 | 4.7 |
| 20.4 | 4.4 |
| 21.2 | 4.2 |
| 21.6 | 4.1 |
| 22.2 | 4.0 |
| 23.9 | 3.7 |
| 24.6 | 3.6 |
| 25.5 | 3.5 |
| 25.7 | 3.5 |
| 26.1 | 3.4 |
| 26.8 | 3.3 |
| 28.1 | 3.2 |
| 28.8 | 3.1 |
| 29.9 | 3.0 |
| 30.6 | 2.9 |
| 31.9 | 2.8 |
| 32.4 | 2.8 |
| 33.6 | 2.7 |
| 34.2 | 2.6 |
| 35.6 | 2.5 |
| 36.1 | 2.5 |
| 38.2 | 2.4 |

Single crystal X-ray analysis revealed that Type L is a THF/water co-solvate of Compound 1, with Compound 1, THF, and water present in a 1:1:1 ratio.

### Example 15—Preparation and Characterization of Type M of Compound 1

### Preparation of Type M of Compound 1

Type M was prepared by liquid vapor diffusion, as described in Example 3.

### Characterization of Type M of Compound 1

Type M was characterized by XRPD (Method C) analysis.

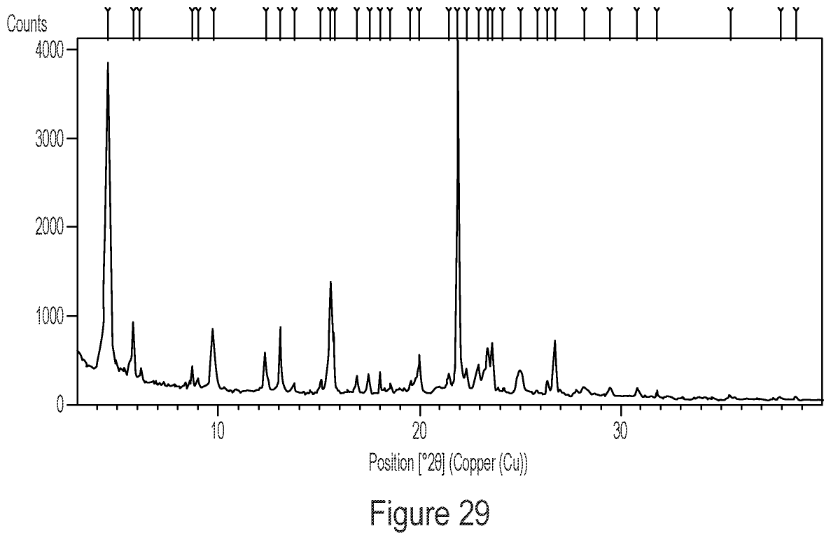

The XRPD pattern for Type M is depicted in FIG. 29, and the corresponding data are summarized in the following table:

| Pos. [°2Th.] | d-spacing [Å] |
|---|---|
| 4.5 | 19.5 |
| 5.8 | 15.3 |
| 6.1 | 14.4 |
| 8.7 | 10.2 |
| 9.0 | 9.9 |
| 9.7 | 9.1 |
| 12.3 | 7.2 |
| 13.1 | 6.8 |
| 13.7 | 5.4 |
| 14.5 | 6.1 |
| 15.1 | 5.9 |
| 15.6 | 5.7 |
| 16.8 | 5.3 |
| 17.4 | 5.1 |
| 18.0 | 4.9 |
| 18.5 | 4.8 |
| 19.5 | 4.5 |
| 20.0 | 4.4 |
| 21.4 | 4.1 |
| 21.9 | 4.1 |
| 22.3 | 4.0 |
| 22.9 | 3.9 |
| 23.3 | 3.8 |
| 23.5 | 3.8 |
| 24.1 | 3.7 |
| 25.0 | 3.6 |
| 25.8 | 3.5 |
| 26.3 | 3.4 |
| 26.7 | 3.3 |
| 27.8 | 3.2 |
| 28.1 | 3.2 |
| 29.4 | 3.0 |
| 30.8 | 2.9 |
| 31.7 | 2.8 |
| 33.0 | 2.7 |
| 35.3 | 2.5 |
| 37.8 | 2.4 |
| 38.6 | 2.3 |

### Example 16—Preparation and Characterization of the Spray-Dried Dispersion of Compound 1

A Spray Dried Dispersion (SDD) of Compound 1 was prepared. The SDD was made up of Compound 1 and a polymer (Hydroxypropylmethyl Cellulose AS-MG) at a 1:3 weight ratio. Compound 1 and the polymer were dissolved in organic solvents (Dichloromethane and Methanol) and spray dried to obtain amorphous an amorphous drug substance. The SDD comprising Compound 1 and HPMC AS (1:3) is referred to herein as SDD 0.

A spray solution was prepared at 7.8% solids content (1:3 Compound 1:HPMC AS-MG) in 80:20 DCM:Methanol per Table 18. An API correction factor of 0.966 was used to prepare the spray solution. The spray solution was prepped by adding DCM and Methanol to a 36 L stainless steel mixing vessel. HPMC AS-MG was added to the solvent system while mixing with a top down mixer at a medium vortex. Compound 1 was then added to the solution. The solution had a yellow/brown clear appearance, however white fiber particulates were seen in the solution.

TABLE 18

| Component | Formulation % | Weight, g |
|---|---|---|
| Compound 1 | 2.00% | 595.0 |
| HPMC AS-MG | 5.81% | 1724.3 |
| DCM | 73.75% | 21896.0 |
| Methanol | 18.44% | 5474.0 |
| Total | 100.0% | 29689.3 |

Correction Factor: 0.9660

A Mobile Minor spray-drying apparatus was setup per Table 19 and warmed up for approximately one hour prior to spraying. Wash solution (80:20 DCM:Methanol) was sprayed prior to the active solution to allow the nozzle to equilibrate. The Compound 1 active solution was sprayed per the settings in Table 19. The spray-dried dispersion was dried overnight (˜20 hours) in a Shel Vacuum Oven at 50° C. and −25 in Hg vacuum under a nitrogen purge at 15 scfh. The resulting spray-dried dispersion was confirmed to be dry by GC analysis. This run generated approximately 2.1 kg of spray-dried dispersion.

TABLE 19

| Parameter | Set Point | |
|---|---|---|
| Inline Filter | Swagelok 140 μm | |
| | Stainless Steel | |
| Nozzle | 0.3 mm, 60° Angle | |
| Inlet Air Flow | 80 | kg/hr |
| Inlet Air Temperature | 104° | C. |
| Pump Stroke Length | 5.70 | mm |
| Nozzle Pressure | 600 | psi |
| Feed Rate (g/min) | 184 | g/min |

| | |
|---|---|
| Outlet Temp (° C.) | 36 |
| Set Condenser Air Temp (° C.) | −10 |
| Actual Condenser Air Temp (° C.) | −3 |
| Chiller Temp (° C.) | −20 |
| Feed Temp | Ambient |

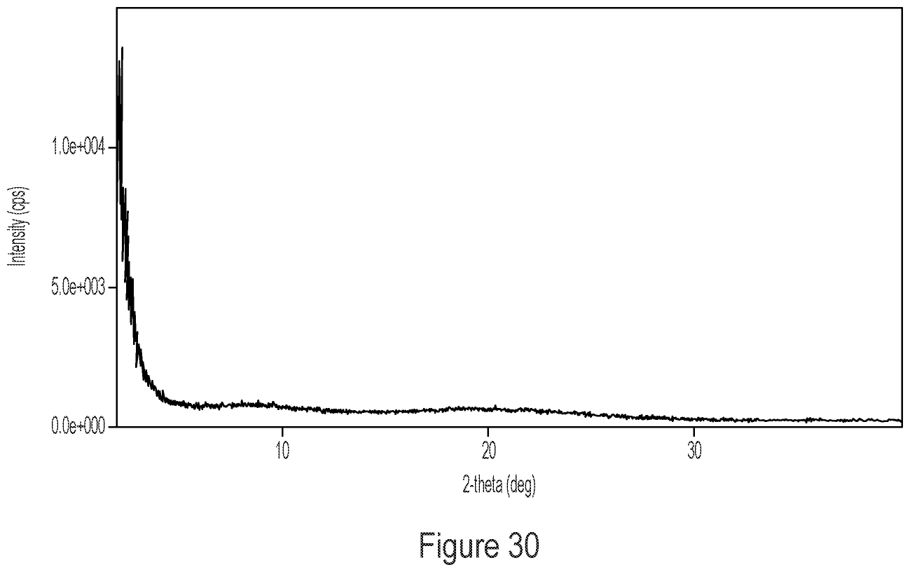

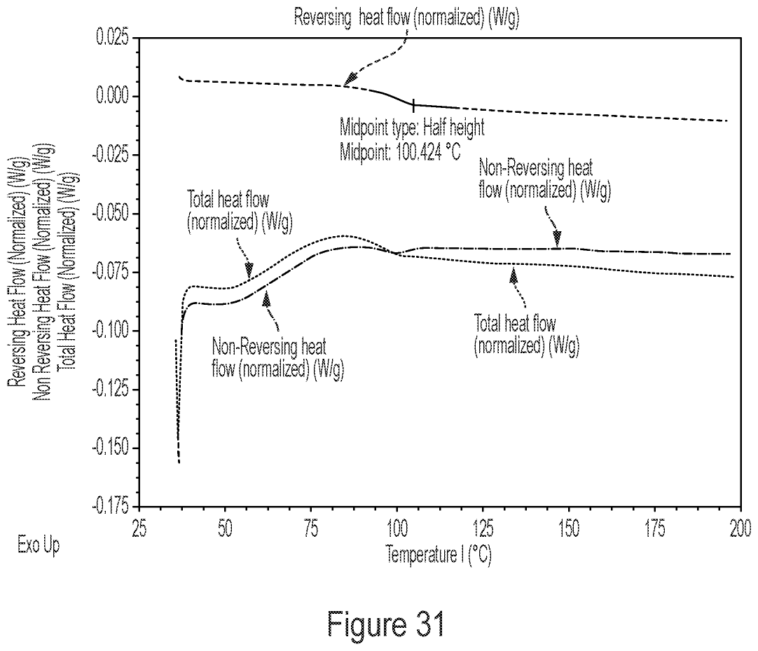

The SDD was characterized by XRPD (Method B) and DSC analysis (ambient to 200° C., 2° C./minute ramp), as shown in FIGS. 30 and 31, respectively. The SDD was determined to be homogenous and amorphous, as shown by the amorphous diffractogram, lack of a crystalline melt, and a single Tg at 100° C.

### Example 17—Bioavailability of the Spray-Dried Dispersion (SDD) of Compound 1 in Rats and Mice

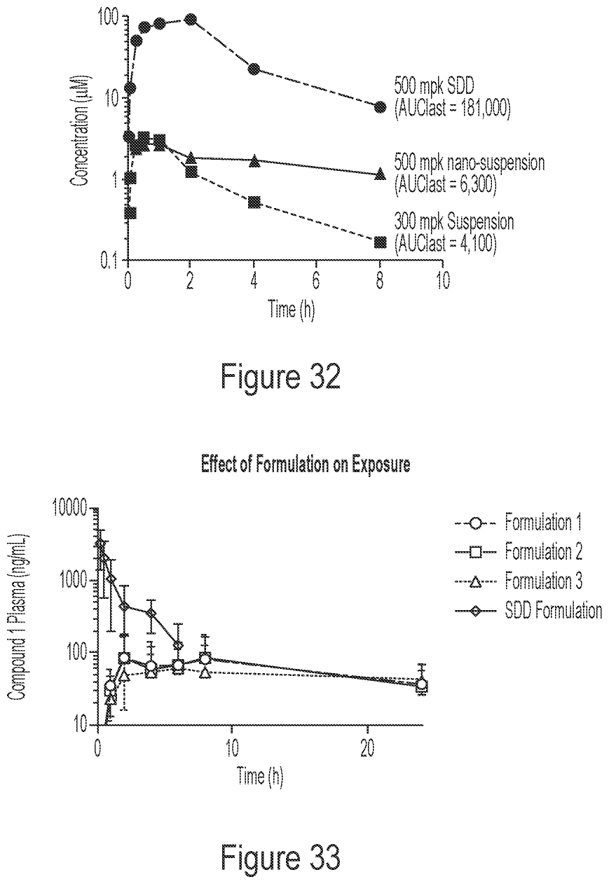

The systemic exposure of Compound 1 in rats and mice was evaluated by dosing a SDD made up of Compound 1 and HPMC AS-MG (1:3) (SDD 0, which can be prepared as described in Example 16) dispersed in an aqueous vehicle (0.5% Hydroxypropylmethyl Cellulose in water). The SDD formulation (“500 mpk SDD”) dosed at 500 mg/kg to rats showed an AUCₗₐₛₜ that was 40× greater than the maximum exposure obtained with the standard formulation (“300 mpk Suspension” made up of Compound 1 (Type A) in 10% Propylene Glycol, 10% Cremophore, 80% Water), as shown in Table 20 and FIG. 32. Additionally, the exposure of a 500 mpk Nano-Suspension made up of nanoparticles of Compound 1 (Type A) was evaluated, as shown in FIG. 32. Robust exposure was observed with SDD formulation in mouse as well.

TABLE 20

| | t_{1/2} | tₘₐₓ | Cₘₐₓ | AUCₗₐₛₜ |
|---|---|---|---|---|
| Animal | (h) | (h) | (ng/mL) | (h*ng/ml) |
| Rat | 3.22 | 1.67 | 44400 | 180603 |
| Mouse | 2.54 | 0.5 | 75200 | 113369 |

### Example 18—Bioavailability of the Spray-Dried Dispersion of Compound 1 in Monkeys

Several formulations of Compound 1, including an SDD made up of Compound 1 and TIPMC AS-MG (1:3) (SDD 0, which can be prepared as described in Example 16), were evaluated in monkeys. The compositions of the tested formulations are listed in Table 21.

TABLE 21

| Formulation | Dosage Form | Composition |
|---|---|---|
| Formulation | Capsule; Size 0 | Compound 1 (Type A), micronized |
| #1 | White Opaque | 49.9% |
| (with Bile | Gelatin | Avicel PH101 23.5% |
| Salt) | | AcDiSol 5.0% |
| | | SLS 10.1% |
| | | Na Taurocholate 10.0% |
| | | Mg Stearate 0.5% |
| | | Silicon Dioxide 1.0% |
| Formulation | Capsule; Size 0 | Compound 1 (Type A) micronized API |
| #2 | White Opaque | 49.9% |
| (Formulated | Gelatin | Avicel PH101 33.3% |
| Capsule) | | AcDiSol 5.0% |
| | | SLS 10.3% |
| | | Mg Stearate 0.5% |
| | | SiO2 1.0% |
| Formulation | Capsule; Size 0 | Compound 1 (Type A) |
| #3 | White Opaque | micronized API only |
| (Micronized | Gelatin | |
| fill) | | |
| Formulation | Suspension | Compound 1 Spray Dried Dispersion |
| #4 | | 0.5% Hydroxypropylmethyl Cellulose |
| (SDD) | | in Water |

The formulations were evaluated for pharmacokinetic parameters in monkeys and are shown in FIG. 33. The profiles show that the SDD formulation (Formulation 4) provided a significant enhancement in overall exposure compared to the encapsulated formulations (Formulations 1, 2, and 3). The bioavailability enhancement with the SDD formulation is approximately 50-62%, which is several fold higher compared to the other formulations, at a dose equivalent to 100 mg.

### Example 19—Time-Dependent Solubility of Type A of Compound 1 and the Spray-Dried Dispersion of Compound 1 in Biorelevant Media

The solubility of Type A of Compound 1 was evaluated in aqueous media. Aqueous solubility samples of Type A of Compound 1 were saturated with solid content. Samples were shaken at 37° C. for 24 hours. Each aqueous media was also sampled after 30 minutes and filtered/diluted using the same procedure to be used for the t=24-hour samples. Sample pH was only measured after 24-hour equilibration.

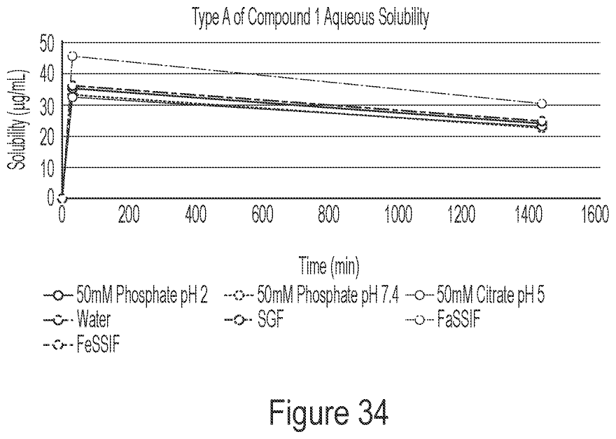

Following 24-hour equilibration, the pH of the saturated samples was measured, and the mixtures were centrifuged through 0.22 m nylon filters at 15,000 rpm for approximately 2 minutes. All centrifuged samples were diluted with method diluent and analyzed by HPLC (Method A). If no solids were present after overnight equilibration, the solubility was reported as “≥” to the determined value. The concentrations reported are based on a single point calibration at the method nominal and are reported as the free form. Results are shown in Table 22 and FIG. 34.

TABLE 22

| | t = 30 | t = 24 | Measured |
|---|---|---|---|
| | minutes | hours | pH |
| | Solubility | Solubility | (after |
| Sample | (μg/mL) | (μg/mL) | 24-hours) |
| Water | 36.29 | 24.98 | 8.69 |
| 50 mM Phosphate pH 2.0 | 35.31 | 24.16 | 1.98 |
| 50 mM Citrate pH 5.0 | 32.53 | 23.49 | 5.01 |
| 50 mM Phosphate pH 7.4 | 33.43 | 22.72 | 7.36 |
| Simulated Gastric Fluid (SGF) | 39.65 | 26.60 | 1.07 |
| Fasted-State Simulated Intestinal | 45.84 | 30.48 | 6.52 |
| Fluid (FaSSIF) | | | |
| Fed-State Simulated Intestinal | 67.04 | 48.18 | 5.01 |
| Fluid (FeSSIF) | | | |

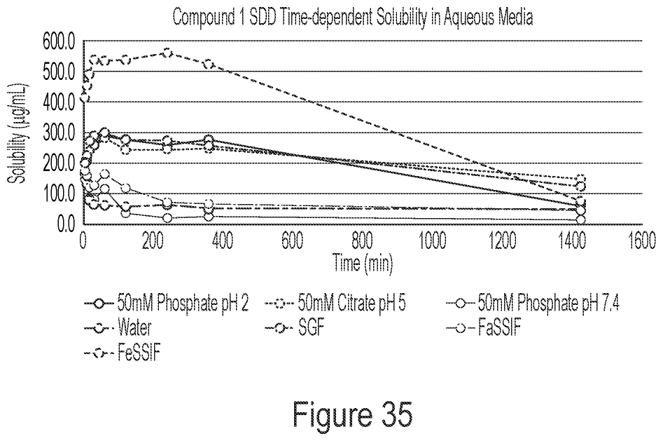

Solubility of the 1:3 Compound 1: HPMC-AS-MG spray-dried dispersion (SDD 0, which can be prepared as described in Example 16) was assessed in aqueous media at various timepoints over 24-hours. Individual saturated samples were prepared for each anticipated time point by adding ˜10 mg of SDD material to 1.5 mL of solvent. Samples were placed at 37° C. on a thermal shaker at 600 RPM and pulled at t=2 min, 5 min, 15 min, 30 min, 1 hr, 2 hr, 4 hr, 6 hr, and 24-hours. The mixtures were centrifuged through 0.22 m nylon filters at 15,000 rpm for approximately 5 minutes. All centrifuged samples were diluted with method diluent and analyzed by HPLC (Method A). Sample pH was measured only at 24-hours. Results are shown in Table 23 and FIG. 35.

TABLE 23

| | Solubility (μg/mL) at Actual Time (min) | | | | | | | | | |
|---|---|---|---|---|---|---|---|---|---|---|
| Media | 2 | 8 | 15 | 29 | 59 | 120 | 240 | 360 | 1435 | Final pH |
| 50 mM Phosphate pH 2 | 169.5 | 207.2 | 238.7 | 258.9 | 288.4 | 278.0 | 258.5 | 276.0 | 65.0 | 1.91 |
| 50 mM Citrate pH 5 | 163.5 | 213.2 | 239.1 | 260.5 | 281.7 | 244.9 | 245.7 | 248.1 | 149.1 | 4.96 |
| 50 mM Phosphate pH 7.4 | 148.4 | 138.6 | 86.1 | 83.1 | 117.3 | 38.1 | 22.8 | 25.6 | 15.5 | 7.19 |
| Water | 203.7 | 132.4 | 80.4 | 65.7 | 64.0 | 58.8 | 64.3 | 50.9 | 48.9 | 5.11 |
| SGF | 199.8 | 225.7 | 272.8 | 289.9 | 299.6 | 276.8 | 273.5 | 256.7 | 125.1 | 1.05 |
| FaSSIF | 175.5 | 160.0 | 120.5 | 128.5 | 164.1 | 5005.9¹ | 72.4 | 67.7 | 46.4 | 5.65 |
| FeSSIF | 413.1 | 453.5 | 490.1 | 536.0 | 532.9 | 536.0 | 558.5 | 522.2 | 76.2 | 5.02 |

¹Solids observed in centrifuge filter, resuspended in case of supersaturation, however high value suggests faulty filter.
Time point omitted from the solubility profile in FIG. 35.

The solubility of the SDD was significant, particularly at earlier time points. Solubility of the SDD after four hours was 72.4 μg/mL in FaSSIF and 558.5 μg/mL in FeSSIF. Four-hour solubility of the SDD is 273.5 μg/mL in SGF. The solubility decreased after 24-hour equilibration in all aqueous media tested.

### Example 20—Stability Assessment of the Spray-Dried Dispersion of Compound 1

Stability studies were conducted on two distinct lots of the 1:3 Compound 1: HPMC-AS-MG spray-dried dispersion (SDD 0, which can be prepared as described in Example 16) under the conditions outlined in Table 24. The results of the stability study for each lot and storage condition are reported in the Tables identified in Table 24. The results for Lot 1 at the 5 month time point and Lot 2 at the 1 month time point remained consistent with the T=0 time points.

TABLE 24

| Lot No. | Container | Storage Condition | Table |
|---|---|---|---|
| Lot 1 | Double-bagged | 2-8° C./Ambient RH | Table 25 |
| | LDPE bags, Zip-tied, | 25 ± 2° C./60 ± 5% RH | Table 26 |
| | inside a sealed Mylar | | |
| | pouch with a 1 g | | |
| | desiccant packet | | |
| Lot 2 | Double-bagged | 2-8° C./Ambient RH | Table 27 |
| | LDPE bags, Zip-tied, | 25 ± 2° C./60 ± 5% RH | Table 28 |
| | inside a sealed Mylar | 40 ± 2° C./75 ± 5% RH | Table 29 |
| | pouch with a 1 g | | |
| | desiccant packet | | |

TABLE 25
Storage Conditions: 2-8º C./Ambient RH Lot 1

| | Time Point (months) | |
|---|---|---|
| Test Method | 0 | 5 |
| Water Content | 2.12% | 1.07% |
| DSC | T_{G} at | T_{G} at |
| (Method B) | 97.707° C., | 97.555° C., |
| | absence of melt | absence of melt |
| XRPD | No measurable | No measurable |
| (Method D) | crystalline | crystalline |
| | material | material |
| | observed | |

TABLE 26
Storage Conditions: 25 ± 2º C./60 ± 5% RH Lot 1

| | Time Point (months) | |
|---|---|---|
| Test Method | 0 | 5 |
| Water Content | 2.12% | 1.03% |
| DSC | T_{G} at | T_{G} at |
| (Method B) | 97.707° C., | 98.630° C., |
| | absence of melt | absence of melt |
| XRPD | No measurable | No measurable |
| (Method D) | crystalline | crystalline |
| | material | material |
| | observed | observed |

TABLE 27
Storage Conditions: 2-8° C./Ambient RH Lot 2

| | Time Point (months) | |
|---|---|---|
| Test Method | 0 | 1 |
| Water Content | 1.24% | 0.95% |
| DSC | T_{G} at | T_{G} at |
| (Method B) | 100.345° C., | 99.456° C., |
| | absence of melt | absence of melt |
| XRPD | XRPD | XRPD |
| (Method D) | Diffractogram | Diffractogram |
| | showed amorphous | showed amorphous |
| | halo without any | halo without any |
| | distinct peaks | distinct peaks |

TABLE 28
Storage Conditions: 25 ± 2° C./60 ± 5% RH Lot 2

| | Time Point (months) | |
|---|---|---|
| Test Method | 0 | 1 |
| Water Content | 1.24% | 0.77% |
| DSC | T_{G} at | T_{G} at |
| (Method B) | 100.345° C., | 99.343° C., |
| | absence of melt | absence of melt |
| XRPD | XRPD | XRPD |
| (Method D) | Diffractogram | Diffractogram |
| | showed amorphous | showed amorphous |
| | halo without any | halo without any |
| | distinct peaks | distinct peaks |

TABLE 29
Storage Conditions: 40 ± 2° C./75 ± 5% RH Lot 2

| | Time Point (months) | |
|---|---|---|
| Test Method | 0 | 1 |
| Water Content | 1.24% | 0.74% |
| DSC | T_{G} at | T_{G} at |
| (Method B) | 100.345° C., | 98.367° C., |
| | absence of melt | absence of melt |
| XRPD | XRPD | XRPD |
| (Method D) | Diffractogram | Diffractogram |
| | showed amorphous | showed amorphous |
| | halo without any | halo without any |
| | distinct peaks | distinct peaks |

### Example 21—Preparation and Characterization of Spray-Dried Dispersions of Compound 1

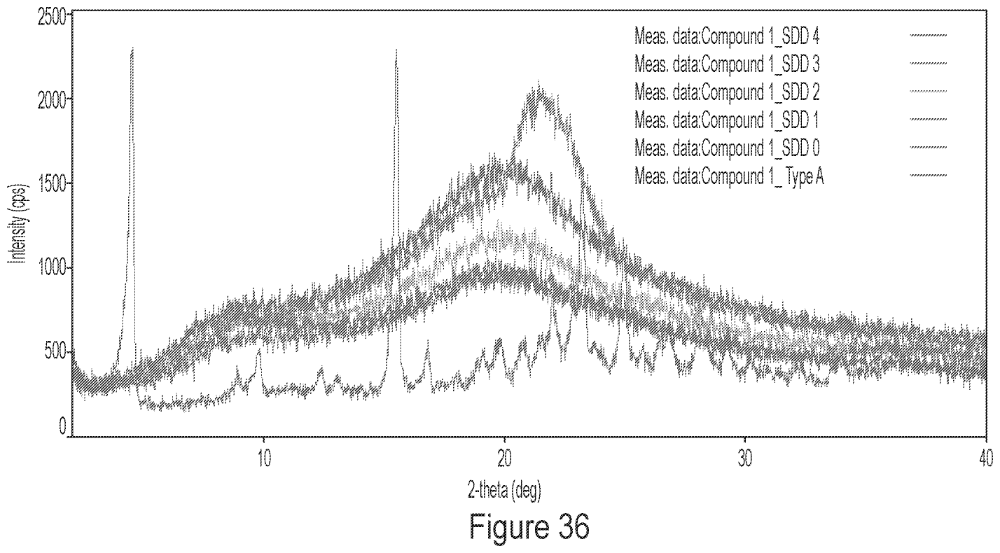

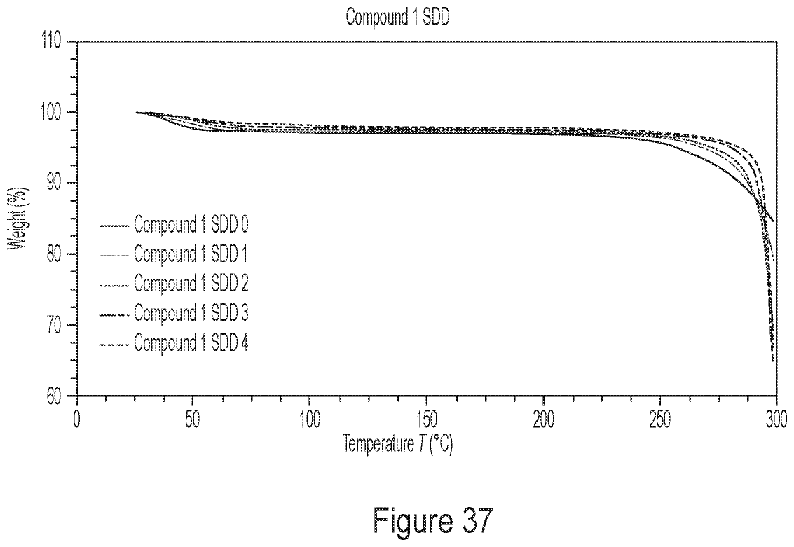

Spray solutions having varying ratios of Compound 1 to polymer (Hydroxypropylmethyl Cellulose AS-MG) were prepared at 8% solids content in 80:20 DCM:MeOH (Table 30). The spray solutions were spray dried using a Procept 4M8-Trix unit with the settings detailed in Table 31. The resulting spray dried dispersions (SDDs) were dried at 50° C. at ˜25 in. Hg in a nitrogen purged vacuum oven for 19 hours. The SDDs were evaluated by XRPD analysis (Method D; FIG. 36) and DSC analysis (Method B; FIG. 37). The SDDs appeared amorphous by PXRD analysis, with no crystalline diffraction peaks observed. A single well defined T_{G} was seen by DSC for all dispersions. No melt endotherm was observed, further verifying the amorphous nature of all spray dried dispersions. Residual solvent analysis of the spray dried dispersions dried for 19 hours showed varying levels for dichloromethane. An observed trend is that levels of dichloromethane increase with increased ratios of Compound 1 to Polymer.

TABLE 30

| | Weight Ratio | | |
|---|---|---|---|
| Sample | (Compound 1:Polymer) | % Compound 1 | % Polymer |
| SDD 1 | 2:3 | 40% | 60% |
| SDD 2 | 1:1 | 50% | 50% |
| SDD 3 | 2:1 | 66.7% | 33.3% |
| SDD 4 | 3:1 | 75% | 25% |

TABLE 31

| Parameter | Setting |
|---|---|
| Nozzle Orifice | 1.0 mm |
| Inlet Air Speed | 0.35-0.39 m³/min |
| Inlet Temp | 50° C.-60° C. |
| Flow Rate | ~10 g/min |
| Pump Speed | 70-80% |
| Atom. Gas Flow | 15 L/min |
| Outlet Temp | 36° C. |

### Example 22—Preparation and Characterization of Spray-Dried Dispersions of Compound 1

Spray solutions having varying ratios of Compound 1 to polymer (Hydroxypropylmethyl Cellulose AS-MG) were prepared at 12% solids content in 80:20 DCM:MeOH (Table 32). The spray solutions were sprayed on a GEA Mobile Minor spray dryer, and the SDDs were collected and dried at 50° C. and −25 in Hg under a N₂ purge.

TABLE 32

| | Weight Ratio |
|---|---|
| Sample | (Compound 1:Polymer) |
| SDD 5 | 1:1 |
| SDD 6 | 1.5:1 |

### Example 23—Kinetic Solubility of Compound 1 SDDs

A μDISS Profiler™ instrument from Pion, Inc was used to quantify concentrations during equilibrium solubility experiments involving SDD 0 (which can be prepared as described in Example 16) and SDDs 1-4 (which can be prepared as described in Example 21). The unit consists of six photodiode array (PDA) spectrophotometers, each with its own dedicated fiber optic dip probe, center-positioned in the glass vial holding 10 mL of media. The concentration measurements are performed directly in the assay media, with processed results plotted in “real time.”

Probes with 2-mm path length tips were selected for quantification of Compound 1 in SDD. The developed calibration curves were used for quantification of Compound 1 in the samples during kinetic solubility experiments at each time point. The 2-mm path length tips were selected for detecting concentrations of Compound 1 in both SGF and FaSSIF media.

Standard calibration curves were generated in the respective assay media using a serial addition protocol. A stock solution of Compound 1 was prepared in DMSO at ˜20 mg/mL. Calculated aliquots of the stock were added to the respective buffers in order to prepare several standard solutions spanning specific concentration ranges. Concentrations of the standard solutions ranged from ˜50 to ˜300 μg/mL for channels in both SGF and FaSSIF media respectively. The area under the 2^{nd} derivative curves was used to calculate the concentrations. The wavelength range was selected for the compounds in such a way that sensitivity issues were avoided. Linearity of the standard curves in the selected wavelength regions were characterized by r²≥0.999.

Area under the 2nd derivative curve in 285-300 nm (SGF) and 305-320 nm (FaSSIF) range were used to calculate the standard curves in respective media. The corresponding standard curves were used to determine concentrations of Compound 1 in solubility assays.

Required amount of SDD materials, equivalent to 20 mg of Compound 1 were weighed into 20 mL glass vials. The vials were then transferred to the instrument for analysis. A clean stir bar was added to the vial with sample. 16 mL of SGF buffer was transferred to the vials before beginning the experiment to achieve an upper limit of ˜1.25 mg/mL. The stirring was maintained at 220 RPM and the temperature of the medium at 37° C. Kinetic solubility data was collected for 30 minutes in SGF media. The data showed that all the SDD's at different loadings exhibited very similar release profile and achieved about same concentration of Compound 1 (-300 μg/mL).

At the 30-minute interval, the media was converted to FaSSIF 6.5. The final volume in the vials was increased to 20 mL from 16 mL (1.00 mg/mL). The resulting samples were then analyzed using the μDiss Profiler for ˜18H in FaSSIF.

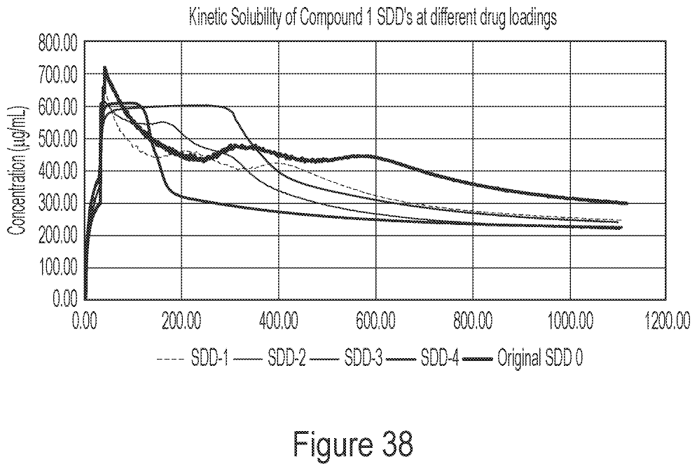

The SDD 0 formulation with 25% drug loading reached higher solubility of ˜700 μg/mL but did not remain in the supersaturated state for longer time. Although the solubility of SDD 0 at 4H was slightly lower than the 40% API loading SDD 1, the equilibrium solubility after ˜16 hours was higher than all other SDD systems. SDD 3 and SDD 4 did not reach as high of a concentration (spring effect) as SDD 0. However, SDD 3 and SDD 4 both stayed at supersaturated state for longer time when compared to all other SDD systems tested. However, the equilibrium solubility for SDD 3 and SDD 4 after 16 hours was lower than SDD 0. SDD 2 and SDD 3 show marked enhancement in the solubility and prolonged supersaturation. The Kinetic solubility profiles are shown in FIG. 38.

The results are summarized in Table 33. All the solubility Results reported in the table are an average of n=2 replicates.

TABLE 33

| | SDD 0 | SDD 1 | SDD 2 | SDD 3 | SDD 4 |
|---|---|---|---|---|---|
| Parameter | (1:3) | (2:3) | (1:1) | (2:1) | (3:1) |
| 30 min SGF | 293.34 | 338.27 | 371.81 | 340.56 | 374.24 |
| (μg/mL) | | | | | |
| Cmax | 708.65 | 658.09 | 618.76 | 603.12 | 609.61 |
| (μg/mL) | | | | | |
| 4 H | 438.55 | 450.60 | 476.06 | 603.03 | 307.06 |
| (μg/mL) | | | | | |
| Eq Sol (16 H) | 301.75 | 250.53 | 221.95 | 243.60 | 226.20 |
| (μg/mL) | | | | | |

### Example 24—Stability of Spray Dried Dispersions of Compound 1

Spray dried dispersions of Compound 1 (SDD 0 (which can be prepared as described in Example 16) and SDDs 1-4 (which can be prepared as described in Example 21)) were set up on a short-term stability study in two different storage configurations at various storage conditions. Samples were set up as “Sealed” and “Exposed”. Sealed samples were placed into crimp sealed vials and stored in a single storage condition; 60° C. The exposed samples were placed into a vial that was covered lightly with perforated foil to allow exposure to the humidity conditions. The exposed samples were stored at 40° C./75% RH and 60° C./75% RH. Samples were pulled after T=1 and 2 weeks for PXRD analysis (Method D).

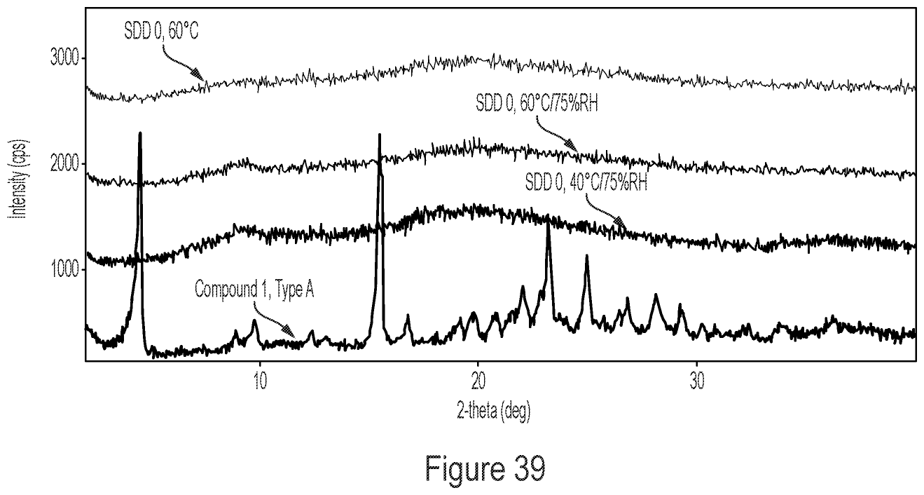

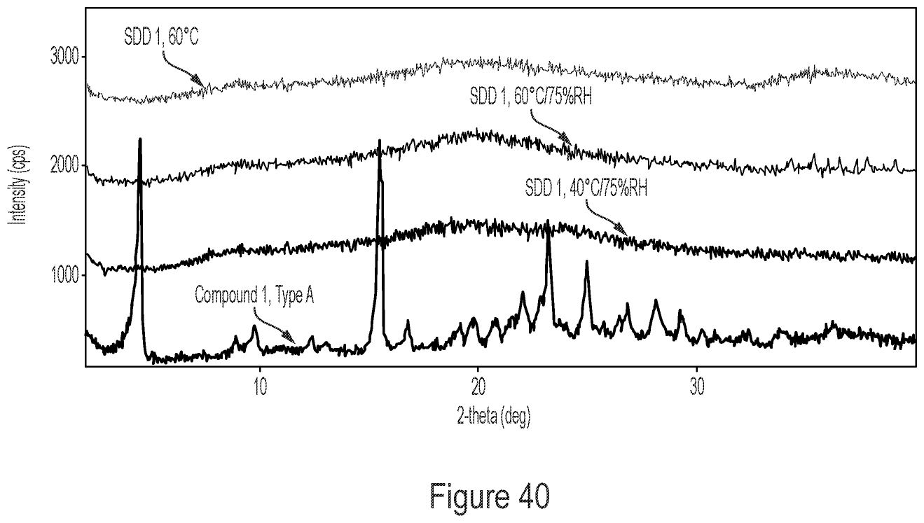

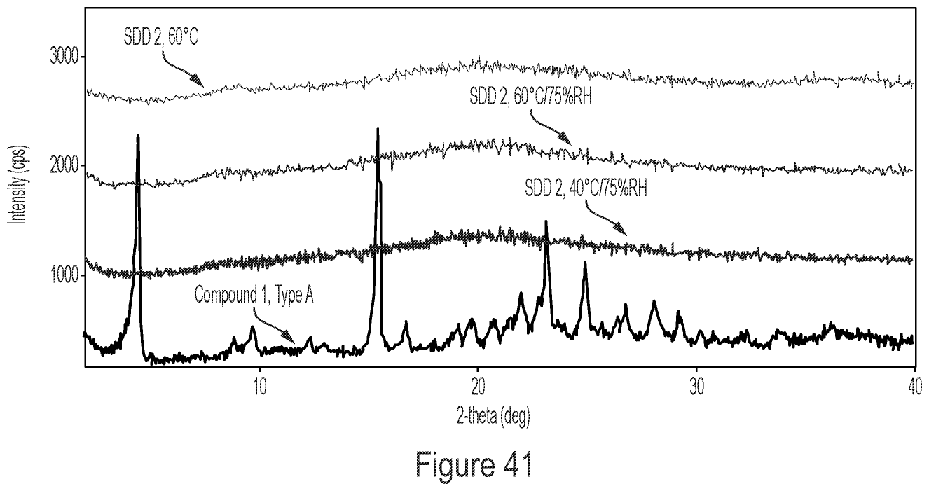

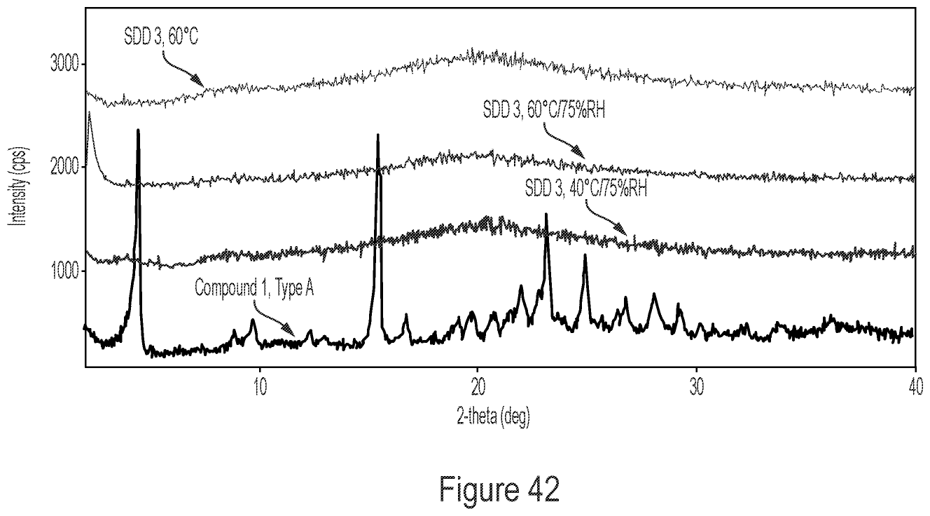

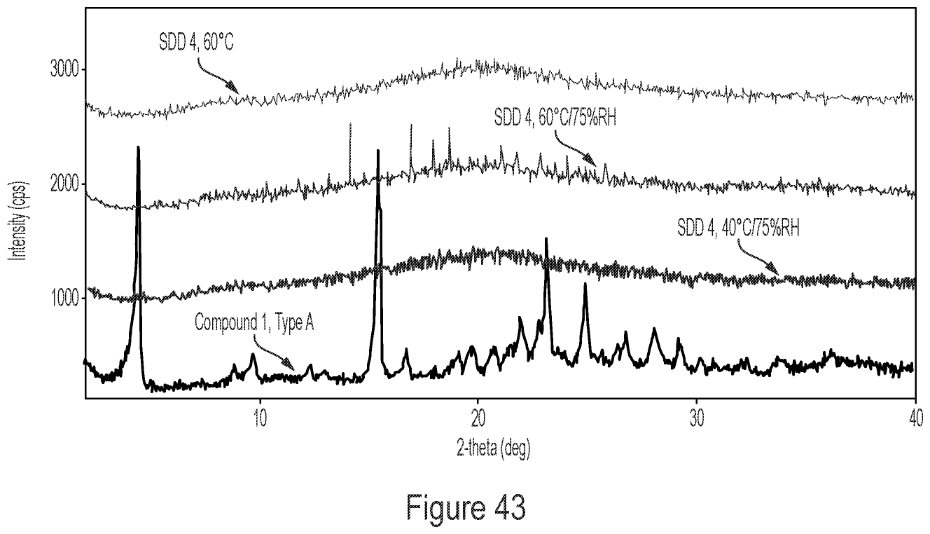

PXRD diffractograms taken after 2 weeks for SDDs 0-4 are provided in FIGS. 39-43, respectively, and the results are summarized in Table 34. SDDs 0, 1, 2 and 3 showed amorphous character by PXRD at all tested conditions through 2 weeks. SDD 4 showed crystallinity by PXRD after 2 weeks of storage at 60° C./75% RH (exposed). SDD 4 also showed two glass transition temperatures by DSC (Method B) after 2 weeks of storage at 60° C./75% RH (exposed), suggesting phase separation.

TABLE 34

| | | Time Point (weeks) | |
|---|---|---|---|
| SDD | Storage Condition | 1 | 2 |
| SDD 0 | 60° C. Sealed | No crystalline | No crystalline |
| (1:3) | | diffraction peaks | diffraction peaks |
| | | observed by PXRD | observed by PXRD |
| | 40° C./75% | No crystalline | No crystalline |
| | RH Exposed | diffraction peaks | diffraction peaks |
| | | observed by PXRD | observed by PXRD |
| | 60° C./75% | No crystalline | No crystalline |
| | RH Exposed | diffraction peaks | diffraction peaks |
| | | observed by PXRD | observed by PXRD |
| SDD 1 | 60° C. Sealed | No crystalline | No crystalline |
| (2:3) | | diffraction peaks | diffraction peaks |
| | | observed by PXRD | observed by PXRD |
| | 40° C./75% | No crystalline | No crystalline |
| | RH Exposed | diffraction peaks | diffraction peaks |
| | | observed by PXRD | observed by PXRD |
| | 60° C./75% | No crystalline | No crystalline |
| | RH Exposed | diffraction peaks | diffraction peaks |
| | | observed by PXRD | observed by PXRD |
| SDD 2 | 60° C. Sealed | No crystalline | No crystalline |
| (1:1) | | diffraction peaks | diffraction peaks |
| | | observed by PXRD | observed by PXRD |
| | 40° C./75% | No crystalline | No crystalline |
| | RH Exposed | diffraction peaks | diffraction peaks |
| | | observed by PXRD | observed by PXRD |
| | 60° C./75% | No crystalline | No crystalline |
| | RH Exposed | diffraction peaks | diffraction peaks |
| | | observed by PXRD | observed by PXRD |
| SDD 3 | 60° C. Sealed | No crystalline | No crystalline |
| (2:1) | | diffraction peaks | diffraction peaks |
| | | observed by PXRD | observed by PXRD |
| | 40° C./75% | No crystalline | No crystalline |
| | RH Exposed | diffraction peaks | diffraction peaks |
| | | observed by PXRD | observed by PXRD |
| | 60° C./75% | No crystalline | No crystalline |
| | RH Exposed | diffraction peaks | diffraction peaks |
| | | observed by PXRD | observed by PXRD |
| SDD 4 | 60° C. Sealed | No crystalline | No crystalline |
| (3:1) | | diffraction peaks | diffraction peaks |
| | | observed by PXRD | observed by PXRD |
| | 40° C./75% | No crystalline | No crystalline |
| | RH Exposed | diffraction peaks | diffraction peaks |
| | | observed by PXRD | observed by PXRD |
| | 60° C./75% | No crystalline | Crystalline |
| | RH Exposed | diffraction peaks | diffraction peaks |
| | | observed by PXRD | observed by PXRD |

### Example 25—Stability of Spray Dried Dispersions of Compound 1

Spray dried dispersions of Compound 1 (SDDs 5 and 6 (Example 22)) were stored in two different storage configurations at various storage conditions. Samples were set up as “Sealed” and “Exposed”. Sealed samples were stored in an amber crimp sealed vial at the following conditions: 2-8° C., 25° C./60%₀RH, 40° C./75%₀RH, and 60° C. Exposed samples were stored in an amber crimp vial covered with foil, which was perforated to allow exposure to humidity, at the following conditions: 25° C./75% RH, 40° C./75% RH, and 60° C./75% RH.

Samples were analyzed by PXRD (Method D) and/or DSC (Method B) analysis at T=0, 1, and 2 weeks. Results are summarized in Table 35. No crystalline diffraction peaks were observed by PXRD in any sample. Moreover, a single T_{G} and no melt endotherm was seen by DSC in all samples.

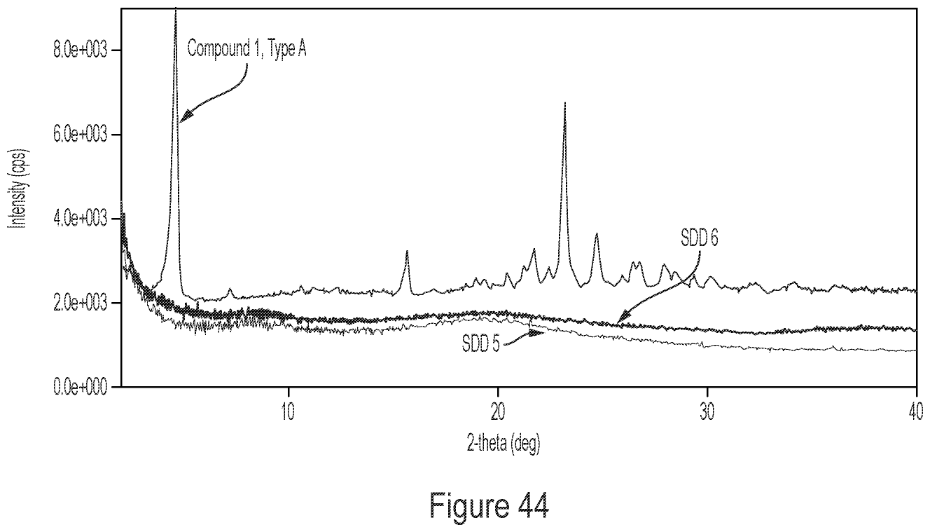

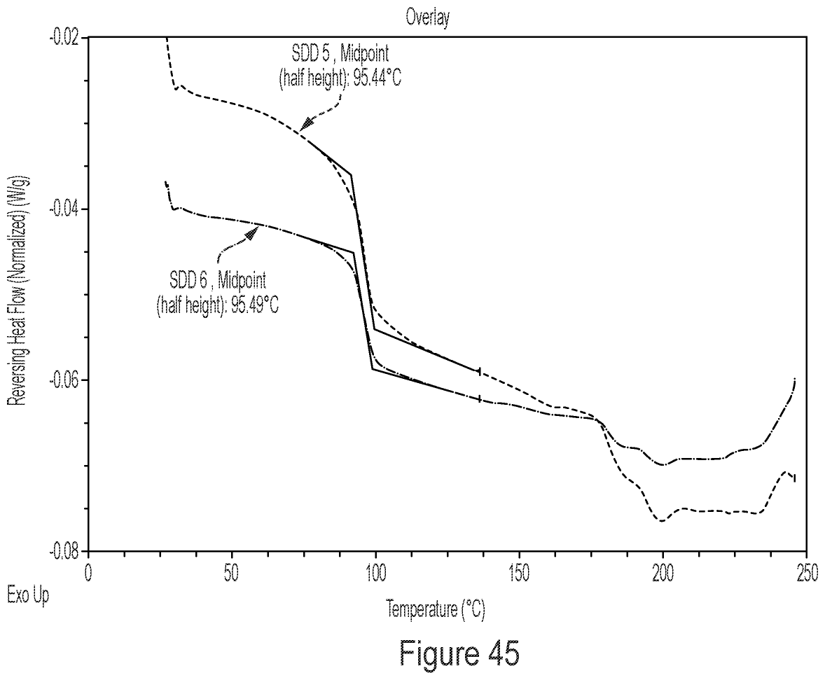

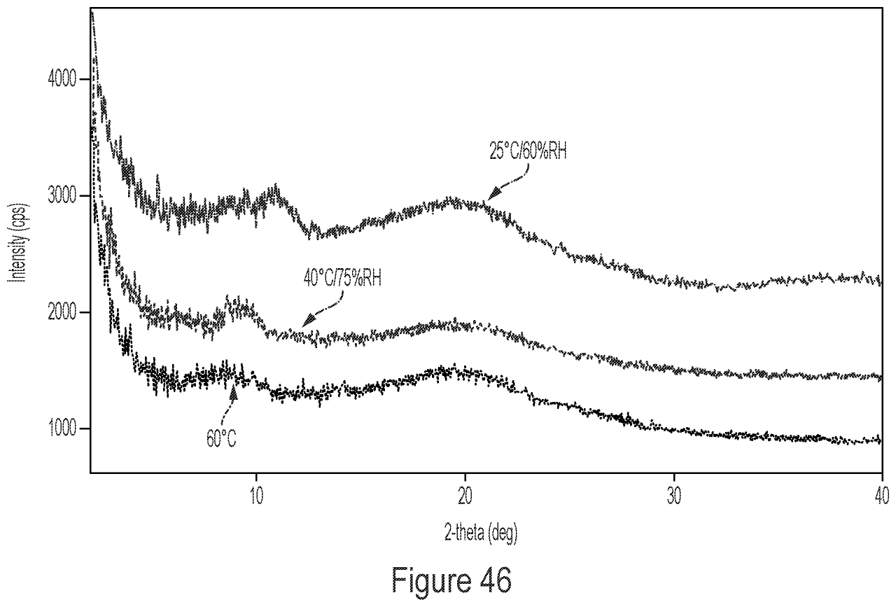

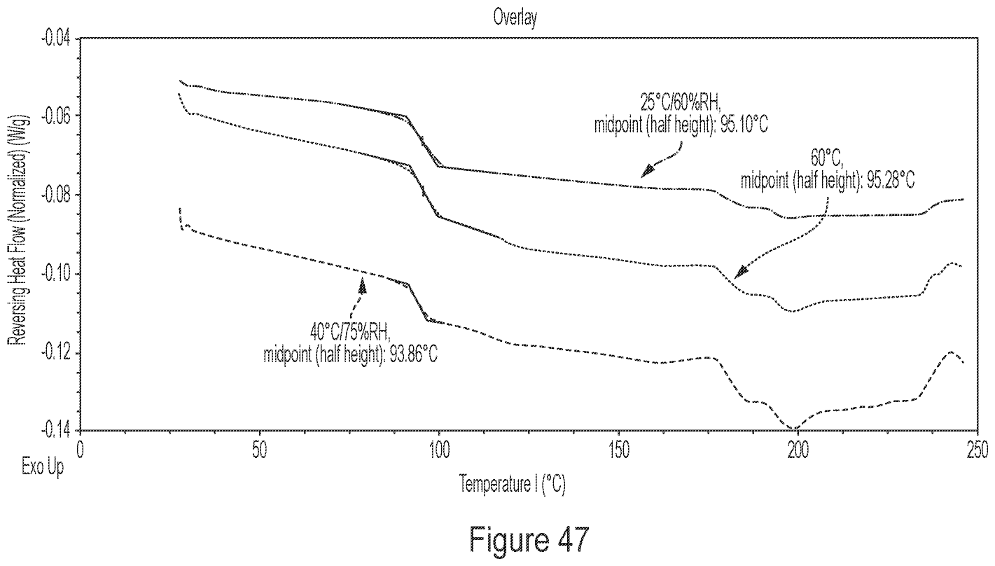

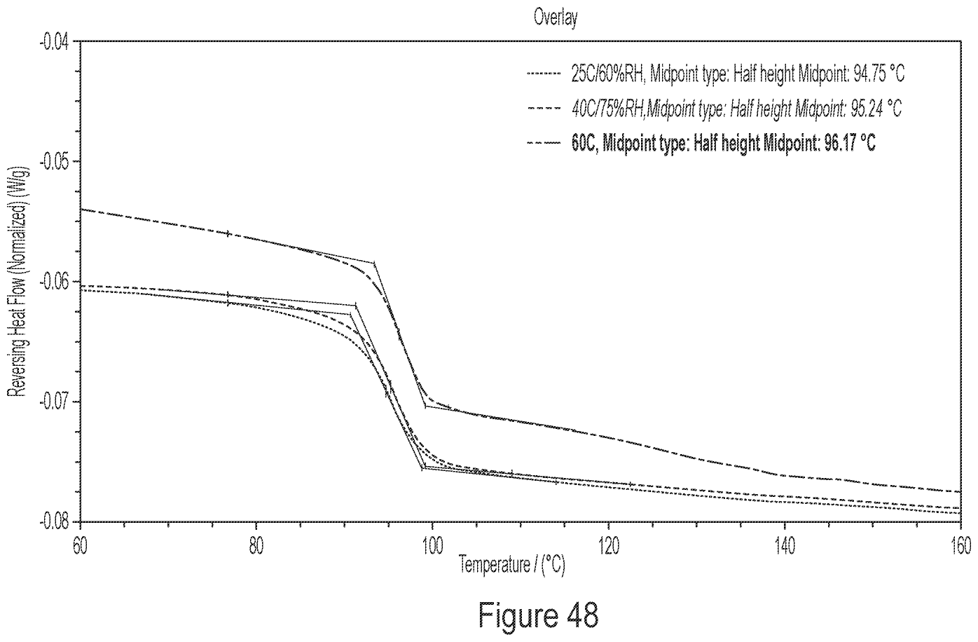

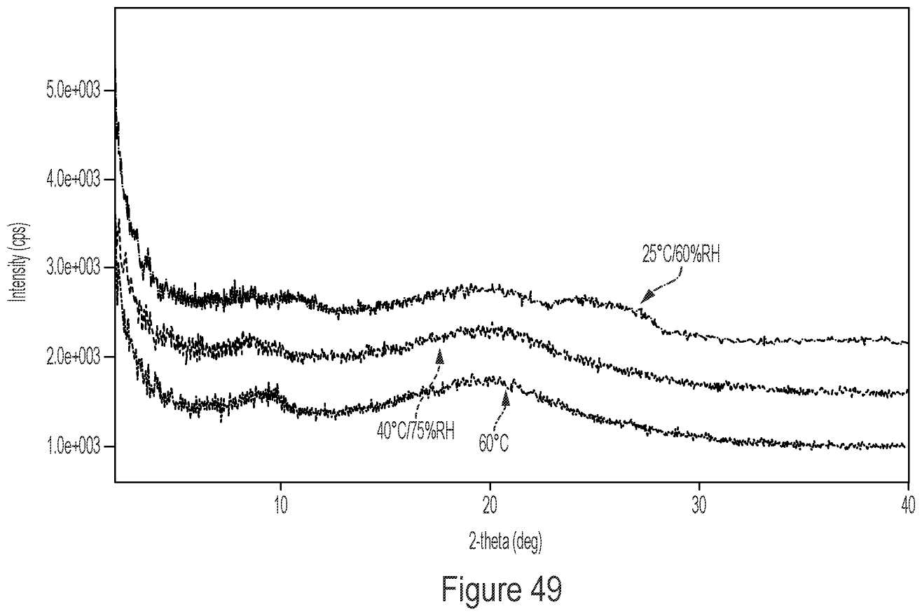

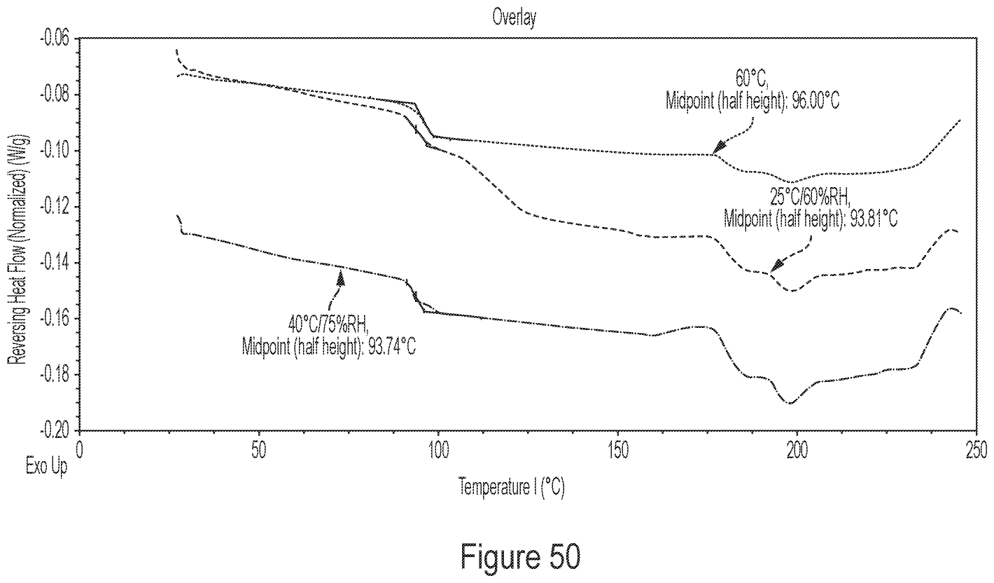

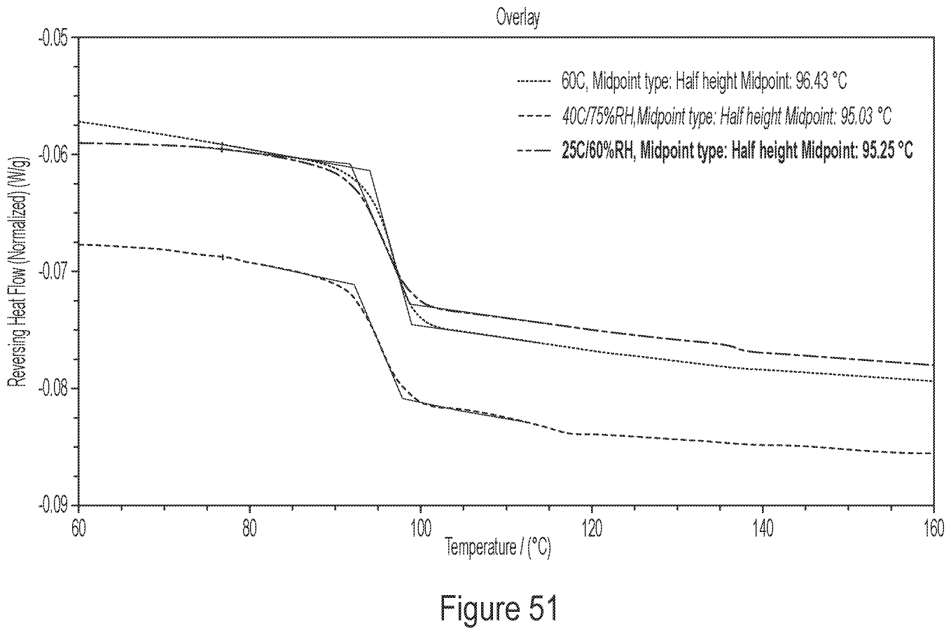

TABLE 35

| | Storage | Time Point (weeks) | | |
|---|---|---|---|---|
| SDD | Condition | 0 | 1 | 2 |
| SDD 5 | N/A (T = 0 time | No crystalline | N/A | N/A |
| (1:1) | point) | diffraction peaks | | |
| | | observed by PXRD | | |
| | | (FIG. 44); A | | |
| | | single T_{G} and no | | |
| | | melt endotherm | | |
| | | was seen by DSC | | |
| | | (FIG. 45) | | |
| | 60° C. Sealed | N/A | No crystalline | A single T_{G} and no |
| | | | diffraction peaks | melt endotherm |
| | | | observed by PXRD | was seen by DSC |
| | | | (FIG. 46); A | (FIG. 48) |
| | | | single T_{G} and no | |
| | | | melt endotherm | |
| | | | was seen by DSC | |
| | | | (FIG. 47) | |
| | 25° C./60% | N/A | No crystalline | A single T_{G} and no |
| | RH Exposed | | diffraction peaks | melt endotherm |
| | | | observed by PXRD | was seen by DSC |
| | | | (FIG. 46); A | (FIG. 48) |
| | | | single T_{G} and no | |
| | | | melt endotherm | |
| | | | was seen by DSC | |
| | | | (FIG. 47) | |
| | 40° C./75% | N/A | No crystalline | A single T_{G} and no |
| | RH Exposed | | diffraction peaks | melt endotherm |
| | | | observed by PXRD | was seen by DSC |
| | | | (FIG. 46); A | (FIG. 48) |
| | | | single T_{G} and no | |
| | | | melt endotherm | |
| | | | was seen by DSC | |
| | | | (FIG. 47) | |
| SDD 6 | N/A (T = 0 time | No crystalline | N/A | N/A |
| (1.5:1) | point) | diffraction peaks | | |
| | | observed by PXRD | | |
| | | (FIG. 44); A | | |
| | | single T_{G} and no | | |
| | | melt endotherm | | |
| | | was seen by DSC | | |
| | | (FIG. 45) | | |
| | 60° C. Sealed | N/A | No crystalline | A single T_{G} and no |
| | | | diffraction peaks | melt endotherm |
| | | | observed by PXRD | was seen by DSC |
| | | | (FIG. 49); A | (FIG. 51) |
| | | | single T_{G} and no | |
| | | | melt endotherm | |
| | | | was seen by DSC | |
| | | | (FIG. 50) | |
| | 25° C./60% | N/A | No crystalline | A single T_{G} and no |
| | RH Exposed | | diffraction peaks | melt endotherm |
| | | | observed by PXRD | was seen by DSC |
| | | | (FIG. 49); A | (FIG. 51) |
| | | | single T_{G} and no | |
| | | | melt endotherm | |
| | | | was seen by DSC | |
| | | | (FIG. 50) | |
| | 40° C./75% | N/A | No crystalline | A single T_{G} and no |
| | RH Exposed | | diffraction peaks | melt endotherm |
| | | | observed by PXRD | was seen by DSC |
| | | | (FIG. 49); A | (FIG. 51) |
| | | | single T_{G} and no | |
| | | | melt endotherm | |
| | | | was seen by DSC | |
| | | | (FIG. 50) | |

Kinetic dissolution of SDD 5 and SDD 6 samples was determined at T=0 and T=1 week (40° C./75% RH Exposed; and 40° C./75% RH Sealed) using the procedure described in Example 23. The results are summarized in Table 36.

TABLE 36

| | | | T = 1 wk | | T = 1 wk | |
|---|---|---|---|---|---|---|
| | | | (40° C./75% RH | | (40° C./75% RH | |
| | T = 0 | | Exposed) | | Sealed) | |
| | SDD 5 | SDD 6 | SDD 5 | SDD 6 | SDD 5 | SDD 6 |
| Parameter | (1:1) | (1.5:1) | (1:1) | (1.5:1) | (1:1) | (1.5:1) |
| 30 min SGF | 611.68 | 622.80 | 600.49 | 628.89 | 623.46 | 616.29 |
| (μg/mL) | | | | | | |
| Cmax | 245.38 | 261.77 | 240.34 | 291.21 | 272.17 | 269.01 |
| (μg/mL) | | | | | | |
| 4H | 127.07 | 130.55 | 291.28 | 256.72 | 171.49 | 189.91 |
| (μg/mL) | | | | | | |
| Eq Sol | 93.37 | 92.71 | 153.88 | 162.06 | 139.64 | 150.04 |
| (μg/mL) | | | | | | |

### Example 26—Composition and Preparation of a Tablet Dosage Form of Compound 1

### Composition of the Tablet Dosage Form

A tablet dosage form of Compound 1 comprising an SDD made up of Compound 1 and HPMC AS-MG (1:3) compressed into tablets and film coated with compendial excipients was prepared. The tablets were presented as 25 mg (white coated round shaped tablets) and 100 mg (white coated oval shaped tablets) dose strengths. The composition of each dosage strength is summarized in Table 37.

TABLE 37

| Component | | % Formulation | Function |
|---|---|---|---|
| Intra | Compound 1 | 50.00% | Drug Product Intermediate |
| Granular | Spray Dried Dispersion¹ | | |
| Components | Microcrystalline Cellulose | 30.00% | Filler |
| | Crospovidone | 5.00% | Dry binder |
| | Colloidal Silicon Dioxide | 1.00% | Glidant |
| | Magnesium Stearate | 0.25% | Lubricant |
| Extra Granular | Microcrystalline Cellulose | 11.00% | Filler |
| Components | Croscarmellose Sodium | 2.50% | Disintegrant |
| | Magnesium Stearate | 0.25% | Lubricant |
| | Total Common Formulation | 100% | — |
| | Blend per Tablet | | |
| Coating | Sterile Water for Injection | Removed through | Processing aid |
| Components | (SWFI) | processing | |
| | Opadry amb II White | 6.00 | Film Coating Agent |
| | Total weight of coated tablet | | |

¹= Quantity based upon a 25.00% (w/w) potency of Compound 1 drug substance in the spray dried intermediate.

### Preparation of the Tablet Dosage Form

The Compound 1 tablet formulation manufacturing process consists of four steps: 1) spray dry dispersion, 2) intragranular granulation, roller compaction/milling/blending, 3) extragranular granulation/blending, and 4) tableting and coating. The initial step of spray dry dispersion is performed by creating an organic solution containing Compound 1 drug substance, and Hypromellose Acetate Succinate (Hydroxypropyl Methylcellulose Acetate Succinate MG) (HPMCAS-MG). The solution is spray dried to produce an SDD made up of Compound 1 and HPMC AS-MG (1:3), using a method analogous to the method of Example 16. The SDD is blended with intra granular excipients followed by roller compaction/milling and blending. The resulting granulation is then mixed with the extra-granular components to create the final common granulation blend. The final blend is pressed into tablets equivalent to either 25 mg or 100 mg active followed by coating.

### Example 27—Dissolution Assessment of the Tablet Dosage Form

The 100 mg tablets described in Example 26 were tested for dissolution. Dissolution testing parameters are provided in Table 38, and results are summarized in Table 39.

TABLE 38

| Parameter | Condition |
|---|---|
| Apparatus | USP Apparatus 2 (Paddles) |
| Media | SIF without enzyme |
| Vessel Size and Type | 1000 mL Amber |
| Media Volume | 900 mL |
| Temperature | 37.0 ± 0.5° C. |
| Speed | 0-60 min: 50 ± 2 RPM, 60-75 min: 200 ± 8 RPM |
| Time Points | 15, 30, 45, and 60 minutes |
| Filters | 13 mm, 0.2 μm Nylon |
| Theoretical | 111.1111 μg/mL |
| Concentration | |
| Sampling Procedure | Remove 3 mL of sample and filter through a |
| | 13 mm, 0.2 μm Nylon filter, discarding the |
| | first 2 mL to waste and collecting the remaining |
| | 1 mL in an amber UPLC vial for analysis. |

TABLE 39

| Time Point | Min % Diss. | Max % Diss. | Mean % Diss. |
|---|---|---|---|
| 15 min | 58.4 | 89.6 | 79.1 |
| 30 min | 88.0 | 92.9 | 91.2 |
| 45 min | 90.7 | 93.9 | 92.7 |
| 60 min | 90.9 | 94.2 | 93.0 |

### Example 28—Release Testing of the Tablet Dosage Form

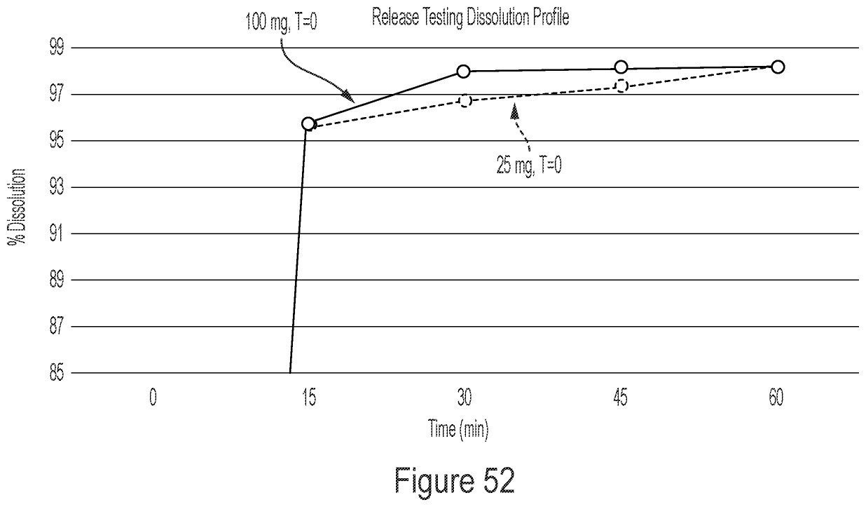

Dissolution testing of 25 mg and 100 mg tablets having the composition specified in Example 26 was performed following the dissolution parameters listed in Table 40. Dissolution was determined by UPLC analysis. The results of the dissolution testing are reported in Table 41 and FIG. 52.

TABLE 40

| Parameter | Condition |
|---|---|
| Apparatus | USP Apparatus 2 (Paddles) |
| Media | SIF without enzyme |
| Vessel Size and Type | 1000 mL Amber |
| Media Volume | 100 mg: 900 mL |
| | 25 mg: 500 mL |
| Temperature | 37.0 ± 0.5° C. |
| Speed | 75 ± 3 RPM |
| Parameter | Condition |
| Time Points | 15, 30, 45, and 60 minutes |
| Filters | 13 mm, 0.2 μm Nylon |
| Theoretical | 100 mg: 111.1111 μg/mL |
| Concentration | 25 mg: 50.0000 μg/mL |
| Sampling Procedure | Remove 3 mL of sample and filter through a 13 mm, 0.2 μm Nylon |
| | filter, discarding the first 2 mL to waste and collecting the remaining |
| | 1 mL in an amber UPLC vial for analysis. |

TABLE 41

| Time | 100 mg Tablet | 25 mg Tablet |
|---|---|---|
| 15 min | Min: 93.7 | Min: 89.6 |
| | Max: 98.3 | Max: 99.8 |
| | Mean: 95.8 | Mean: 95.7 |
| 30 min | Min: 97.2 | Min: 91.6 |
| | Max: 98.6 | Max: 100.4 |
| | Mean: 98.1 | Mean: 96.8 |
| 45 min | Min: 97.4 | Min: 93.1 |
| | Max: 98.8 | Max: 100.6 |
| | Mean: 98.2 | Mean: 97.4 |
| 60 min | Min: 97.4 | Min: 94.4 |
| | Max: 98.9 | Max: 101.1 |
| | Mean: 98.2 | Mean: 98.3 |

### Example 29—Stability Assessment of the Tablet Dosage Form

Stability studies were conducted under the conditions outlined in Table 42 on two distinct lots of 25 mg and 100 mg tablets having the composition specified in Example 26. The results of the stability study for each lot and storage condition are reported in the Tables identified in Table 42.

The tablets were prepared for XRPD analysis (Method D) by crushing a tablet with a mortar and pestle and transferring 5-10 mg of material to a sample pan, slightly overfilling and ensuring that powder is spread evenly to cover the bottom of the plate. Weigh paper was placed atop the powder and pressed down gently to even the powder surface. The XRPD pattern of the tablet was overlaid with the XRPD pattern of a reference standard (Compound 1, Type A). A tablet was deemed to be free of the diffraction peaks that are present in the reference standard only if the peak at ˜15 degrees 2-theta is absent. A small, irregular peak at ˜3 degrees 2-theta is acceptable.

TABLE 42

| Dosage & Lot | | Storage | |
|---|---|---|---|
| No. | Container | Condition | Results Table |
| 25 mg | 30 cc wide mouth | 25 ± 2° C./ | Table 43 |
| Lot 1 | round white HDPE | 60 ± 5% RH | |
| | bottle, capped with | | |
| | 28 mm child | 40 ± 2° C./ | Table 44 |
| | resistant closure and | 75 ± 5% RH | |
| | induction sealed | | |
| 100 mg | 60 cc wide mouth | 25 ± 2° C./ | Table 45 |
| Lot 1 | round white HDPE | 60 ± 5% RH | |
| | bottle, capped with | | |
| | 33 mm child | 40 ± 2° C./ | Table 46 |
| | resistant closure and | 75 ± 5% RH | |
| | induction sealed | | |
| 25 mg | 30 cc wide mouth | 25 ± 2 °C./ | Table 47 |
| Lot 2 | round white HDPE | 60 ± 5% RH | |
| | bottle, capped with | | |
| | 28 mm child | 40 ± 2° C./ | Table 48 |
| | resistant closure and | 75 ± 5% RH | |
| | induction sealed | | |
| 100 mg | 60 cc wide mouth | 25 ± 2° C./ | Table 49 |
| Lot 2 | round white HDPE | 60 ± 5% RH | |
| | bottle, capped with | | |
| | 33 mm child | 40 ± 2° C./ | Table 450 |
| | resistant closure and | 75 ± 5% RH | |
| | induction sealed | | |

TABLE 43
Lot 1 Tablets, 25 mg
Storage Conditions: 25 ± 2° C./60 ± 5% RH

| Test | | Time Point (months) | | | |
|---|---|---|---|---|---|
| Method | | 0 | 1 | 2 | 3 |
| Disso- | 15 | Min: 90% | Min: 90% | Min: 85% | Min: 78% |
| lution | min | Max: 100% | Max: 98% | Max: 98% | Max: 86% |
| | | Mean: 96% | Mean: 94% | Mean: 93% | Mean: 81% |
| | 30 | Min: 92% | Min: 92% | Min: 87% | Min: 81% |
| | min | Max: 100% | Max: 99% | Max: 99% | Max: 89% |
| | | Mean: 97% | Mean: 96% | Mean: 94% | Mean: 84% |
| | 45 | Min: 93% | Min: 93% | Min: 88% | Min: 82% |
| | min | Max: 101% | Max: 99% | Max: 100% | Max: 89% |
| | | Mean: 97% | Mean: 97% | Mean: 95% | Mean: 85% |
| | 60 | Min: 94% | Min: 94% | Min: 90% | Min: 84% |
| | min | Max: 101% | Max: 100% | Max: 100% | Max: 91% |
| | | Mean: 98% | Mean: 98% | Mean: 95% | Mean: 87% |
| Water | | 3.52% | 2.90% | 1.92% | 1.79% |
| Content | | | | | |
| XPRD | | No | No | No | No |
| (Method | | crystalline | crystalline | crystalline | crystalline |
| D) | | peaks | peaks | peaks | peaks |
| | | present | present | present | present |

TABLE 44
Lot 1 Tablets, 25 mg
Storage Conditions: 40 ± 2° C./75 ± 5% RH

| Test | | Time Point (months) | | | |
|---|---|---|---|---|---|
| Method | | 0 | 1 | 2 | 3 |
| Disso- | 15 | Min: 90% | Min: 81% | Min: 78% | Min: 74% |
| lution | min | Max: 100% | Max: 92% | Max: 97% | Max: 85% |
| | | Mean: 96% | Mean: 87% | Mean: 89% | Mean: 80% |
| | 30 | Min: 92% | Min: 83% | Min: 81% | Min: 78% |
| | min | Max: 100% | Max: 99% | Max: 97% | Max: 91% |
| | | Mean: 97% | Mean: 91% | Mean: 91% | Mean: 85% |
| | 45 | Min: 93% | Min: 85% | Min: 83% | Min: 80% |
| | min | Max: 101% | Max: 100% | Max: 98% | Max: 92% |
| | | Mean: 97% | Mean: 92% | Mean: 92% | Mean: 86% |
| | 60 | Min: 94% | Min: 85% | Min: 84% | Min: 81% |
| | min | Max: 101% | Max: 100% | Max: 98% | Max: 93% |
| | | Mean: 98% | Mean: 92% | Mean: 93% | Mean: 88% |
| Water | | 3.52% | 2.66% | 1.71% | 1.95% |
| Content | | | | | |
| XPRD | | No | No | No | No |
| (Method | | crystalline | crystalline | crystalline | crystalline |
| D) | | peaks | peaks | peaks | peaks |
| | | present | present | present | present |

TABLE 45
Lot 1 Tablets, 100 mg
Storage Conditions: 25 ± 2° C./60 ± 5% RH

| Test | | Time Point (months) | | | |
|---|---|---|---|---|---|
| Method | | 0 | 1 | 2 | 3 |
| Disso- | 15 | Min: 94% | Min: 85% | Min: 100% | Min: 96% |
| lution | min | Max: 98% | Max: 87% | Max: 100% | Max: 98% |
| | | Mean: 96% | Mean: 86% | Mean: 100% | Mean: 97% |
| | 30 | Min: 97% | Min: 89% | Min: 100% | Min: 97% |
| | min | Max: 99% | Max: 90% | Max: 100% | Max: 99% |
| | | Mean: 98% | Mean: 90% | Mean: 100% | Mean: 98% |
| | 45 | Min: 97% | Min: 91% | Min: 100% | Min: 98% |
| | min | Max: 99% | Max: 92% | Max: 101% | Max: 99% |
| | | Mean: 98% | Mean: 91% | Mean: 100% | Mean: 99% |
| | 60 | Min: 97% | Min: 92% | Min: 100% | Min: 98% |
| | min | Max: 99% | Max: 92% | Max: 101% | Max: 99% |
| | | Mean: 98% | Mean: 92% | Mean: 100% | Mean: 99% |
| Water | | 3.83% | 2.57% | 2.39% | 2.72% |
| Content | | | | | |
| XPRD | | No | No | No | No |
| (Method | | crystalline | crystalline | crystalline | crystalline |
| D) | | peaks | peaks | peaks | peaks |
| | | present | present | present | present |

TABLE 46
Lot 1 Tablets, 100 mg
Storage Conditions: 40 ± 2° C./75 ± 5% RH

| Test | | Time Point (months) | | | |
|---|---|---|---|---|---|
| Method | | 0 | 1 | 2 | 3 |
| Disso- | 15 | Min: 94% | Min: 86% | Min: 99% | Min: 94% |
| lution | min | Max: 98% | Max: 94% | Max: 99% | Max: 95% |
| | | Mean: 96% | Mean: 89% | Mean: 99% | Mean: 95% |
| | 30 | Min: 97% | Min: 90% | Min: 99% | Min: 95% |
| | min | Max: 99% | Max: 95% | Max: 99% | Max: 96% |
| | | Mean: 98% | Mean: 93% | Mean: 99% | Mean: 95% |
| | 45 | Min: 97% | Min: 92% | Min: 99% | Min: 95% |
| | min | Max: 99% | Max: 95% | Max: 99% | Max: 96% |
| | | Mean: 98% | Mean: 94% | Mean: 99% | Mean: 96% |
| | 60 | Min: 97% | Min: 93% | Min: 99% | Min: 95% |
| | min | Max: 99% | Max: 96% | Max: 99% | Max: 96% |
| | | Mean: 98% | Mean: 95% | Mean: 99% | Mean: 96% |
| Water | | 3.83% | 2.90% | 2.73% | 4.09% |
| Content | | | | | |
| XPRD | | No | No | No | No |
| (Method | | crystalline | crystalline | crystalline | crystalline |
| D) | | peaks | peaks | peaks | peaks |
| | | present | present | present | present |

TABLE 47
Lot 2 Tablets, 25 mg
Storage Conditions: 25 ± 2° C./60 ± 5% RH

| | | Time Point (months) | |
|---|---|---|---|
| Test Method | | 0 | 1 |
| Dissolution | 15 min | Min: 92% Max: 98% | Min: 81% Max: 96% |
| | | Mean: 95% | Mean: 91% |
| | 30 min | Min: 92% Max: 99% | Min: 83% Max: 98% |
| | | Mean: 96% | Mean: 93% |
| | 45 min | Min: 94% Max: 99% | Min: 85% Max: 99% |
| | | Mean: 97% | Mean: 94% |
| | 60 min | Min: 94% Max: 100% | Min: 87% Max: 100% |
| | | Mean: 98% | Mean: 96% |
| Water Content | | 2.18% | 1.62% |
| XRPD | | Free of the | NT |
| (Method D) | | diffraction peaks | |
| | | that are present | |
| | | in the reference standard | |

TABLE 48
Lot 2 Tablets, 25 mg
Storage Conditions: 40 ± 2° C./75 ± 5% RH

| | | Time Point (months) | |
|---|---|---|---|
| Test Method | | 0 | 1 |
| Dissolution | 15 min | Min: 92% Max: 98% | Min: 88% Max: 98% |
| | | Mean: 95% | Mean: 95% |
| | 30 min | Min: 92% Max: 99% | Min: 94% Max: 100% |
| | | Mean: 96% | Mean: 97% |
| | 45 min | Min: 94% Max: 99% | Min: 97% Max: 101% |
| | | Mean: 97% | Mean: 99% |
| | 60 min | Min: 94% Max: 100% | Min: 98% Max: 101% |
| | | Mean: 98% | Mean: 100% |
| Water Content | | 2.18% | 1.65% |
| XRPD | | Free of the diffraction | NT |
| (Method D) | | peaks that are present in | |
| | | the reference standard | |

TABLE 49
Lot 2 Tablets, 100 mg
Storage Conditions: 25 ± 2° C./60 ± 5% RH

| | | Time Point (months) | |
|---|---|---|---|
| Test Method | | 0 | 1 |
| Dissolution | 15 min | Min: 99% Max: 101% | Min: 96% Max: 98% |
| | | Mean: 100% | Mean: 97% |
| | 30 min | Min: 98% Max: 100% | Min: 98% Max: 100% |
| | | Mean: 99% | Mean: 99% |
| | 45 min | Min: 99% Max: 101% | Min: 98% Max: 100% |
| | | Mean: 100% | Mean: 99% |
| | 60 min | Min: 100% Max: 101% | Min: 98% Max: 99% |
| | | Mean: 100% | Mean: 98% |
| Water Content | | 2.74% | 2.94% |
| XRPD | | Free of the diffraction | NT |
| (Method D) | | peaks that are present | |
| | | in the reference standard | |

TABLE 50
Lot 2 Tablets, 100 mg
Storage Conditions: 40 ± 2° C./75 ± 5% RH

| | | Time Point (months) | |
|---|---|---|---|
| Test Method | | 0 | 1 |
| Dissolution | 15 min | Min: 99% Max: 101% | Min: 96% Max: 98% |
| | | Mean: 100% | Mean: 97% |
| | 30 min | Min: 98% Max: 100% | Min: 98% Max: 99% |
| | | Mean: 99% | Mean: 98% |
| | 45 min | Min: 99% Max: 101% | Min: 99% Max: 100% |
| | | Mean: 100% | Mean: 99% |
| | 60 min | Min: 100% Max: 101% | Min: 99% Max: 100% |
| | | Mean: 100% | Mean: 99% |
| Water Content | | 2.74% | 3.04% |
| XRPD | | Free of the diffraction | NT |
| (Method D) | | peaks that are present in | |
| | | the reference standard | |

The results for the tablet batches from Lot 1 at the 3 month time point and Lot 2 at the 1 month time point remained consistent with the T=0 time points.

### Example 30—Composition and Preparation of a Tablet Dosage Form of Compound 1

Tablets comprising a spray dried dispersion (SDD) of Compound 1 and compendial excipients are prepared at 100 mg and 200 mg dosage strengths. The compositions of the tablets are set forth in Tables 51 and 52.

The tablets are prepared by first manufacturing the SDD (spray drying an organic solution of Compound 1 and HPMC-AS (1:1 w/w) (Table 51) or an organic solution of Compound 1 and HPMC-AS (1.5:1 w/w) (Table 52)), followed by roller compaction/milling with intragranular excipients and blending with extragranular excipients. The final blend is pressed into tablets and then film coated.

TABLE 51

| Component | Function | Range |
|---|---|---|
| SDD (1:1) | Active | 50-75% |
| Microcrystalline | Filler | 15-30% |
| Cellulose | | |
| Lactose Monohydrate | Filler | 0-20% |
| Crosslinked | Dry Binder | 2-10% |
| polyvinylpyrrolidone | | |
| Colloidal Silicon | Glidant | <2% |
| Dioxide | | |
| Croscarmellose | Disintegrant | 2-10% |
| Sodium | | |
| Magnesium Stearate | Lubricant | <2% |

TABLE 52

| Component | Function | Range |
|---|---|---|
| SDD (1.5:1) | Active | 50-75% |
| Microcrystalline | Filler | 15-30% |
| Cellulose | | |
| Lactose Monohydrate | Filler | 0-20% |
| Crosslinked | Dry Binder | 2-10% |
| polyvinylpyrrolidone | | |
| Colloidal Silicon | Glidant | <2% |
| Dioxide | | |
| Croscarmellose | Disintegrant | 2-10% |
| Sodium | | |
| Magnesium Stearate | Lubricant | <2% |

### Example 31—Stability Assessment of the Tablet Dosage Form

Tablets having the compositions set forth in Tables 51 and 52 were prepared for XRPD analysis (Method D) by crushing a tablet with a mortar and pestle and transferring 5-10 mg of material to a sample pan, slightly overfilling and ensuring that powder is spread evenly to cover the bottom of the plate. Weigh paper was placed atop the powder and pressed down gently to even the powder surface. The XRPD pattern of the tablet was overlaid with the XRPD pattern of a reference standard (Compound 1, Type A). The XRPD pattern of a tablet was deemed to be free of the diffraction peaks that are present in the reference standard only if the peak at ˜15 degrees 2-theta is absent. A small, irregular peak at ˜3 degrees 2-theta is acceptable. The tablets were determined to be free of crystalline Type A because the XRPD patterns were free of the diffraction peaks that are present in the reference standard.

### Example 32—Composition and Preparation of a Minitablet Dosage Form of Compound 1

Minitablets comprising a spray dried dispersion (SDD) of Compound 1 and compendial excipients are prepared with the compositions set forth in Tables 53 and 54.

For both compositions, the minitablets are prepared by first manufacturing the SDD (spray drying an organic solution of Compound 1 and HPMC-AS (1:1 w/w)). Following preparation of the SDD, the minitablets are prepared by granulation and blending with the excipients, followed by tableting of the granules into 2 mm (˜2 mg strength) and 3 mm (˜3 mg strength) minitablets. The resulting minitablets are enterically coated.

TABLE 53

| Component | Function | Range |
|---|---|---|
| SDD (1:1) | Active | 60-80% |
| Microcrystalline | Filler | 10-20% |
| Cellulose | | |
| Lactose Monohydrate | Filler | 5-10% |
| Crosslinked | Dry Binder | 2-10% |
| polyvinylpyrrolidone | | |
| Colloidal Silicon | Glidant | <2% |
| Dioxide | | |
| Magnesium Stearate | Lubricant | <2% |

TABLE 54

| Component | Function | Range |
|---|---|---|
| SDD (1:1) | Active | 50-75% |
| Microcrystalline | Filler | 15-30% |
| Cellulose | | |
| Lactose Monohydrate | Filler | 0-20% |
| Crosslinked | Dry Binder | 2-10% |
| polyvinylpyrrolidone | | |
| Colloidal Silicon | Glidant | <2.5% |
| Dioxide | | |
| Croscarmellose | Disintegrant | 2-10% |
| Sodium | | |
| Magnesium Stearate | Lubricant | <2% |

### Example 33—Stability Assessment of Minitablet Dosage Form

Coated 2 mm minitablets having the composition set forth in Table 54 were evaluated for stability under open conditions (glass petri dishes) and closed conditions (nitrogen purged glass vials). Minitablets stored for up to 4 weeks at 2-8° C. (closed), 25° C./50% RH (closed), 40° C./75% RH (closed), 40° C./75% RH (open), and 60° C. (closed) showed no changes in amorphicity by XRPD analysis, with all XRPD peaks assignable to the excipients in the formulation.

### Example 34—Determination of Maximum Dose of Compound 2

Good Laboratory Practice (GLP) toxicology testing of a Compound 1 test article comprising a pre-determined amount of Compound 2 was performed in rat and cynomolgus monkeys. A no-observed-adverse-effect level (NOAEL) was determined for each species using standard toxicology techniques, and a resulting dose level for humans was calculated using the FDA Human Equivalent Dose approach based upon dose per body surface area. Based on these experiments and calculations, a maximum recommended starting dose (MRSD) for first-in-human clinical trials was determined based on results from GLP toxicology testing. API compositions of Compound 1 containing less than 5.0% (as determined by percentage area HPLC) of Compound 2 are well within the safe human equivalent dose determined for Compound 2.

1. A solid oral dosage form comprising a stabilized amorphous compound (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one, wherein the stabilized amorphous compound does not show crystallinity by PXRD (Method D) after 2 weeks of storage at 60° C./75% RH (exposed).

2. The solid oral dosage form of claim 1, wherein the stabilized amorphous compound shows a single glass transition temperature (T_{G}) and no melt endotherm by DSC (Method B) after 2 weeks of storage at 60° C./75% RH (exposed).

3. The solid oral dosage form of claim 1, wherein the solid oral dosage form contains a total of 200 mg of (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one.

4. The solid oral dosage form of claim 3, wherein the solid oral dosage form has a total weight of not more than 800 mg.

5. The solid oral dosage form of claim 4, wherein the solid oral dosage form is a tablet or capsule.

6. The solid oral dosage form of claim 4, wherein the stabilized amorphous compound is in a spray dried dispersion with a polymer.

7. The solid oral dosage form of claim 6, wherein the polymer is selected from the group consisting of hydroxypropylmethyl cellulose (HPMC), hydroxypropylmethyl cellulose acetate succinate (HPMC AS), hydroxypropyl methyl cellulose phthalate (HPMCP), hydroxypropyl cellulose (HPC), ethylcellulose, cellulose acetate phthalate, polyvinylpyrrolidone (PVP), and a combination thereof.

8. The solid oral dosage form of claim 6, wherein the polymer is HPMC AS.

9. The solid oral dosage form of claim 8, wherein the (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one is spray dried with HPMC AS in a weight ratio of 1:3 to 2:1.

10. The solid oral dosage form of claim 8, wherein the (S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one is spray dried with HPMC AS in a weight ratio of 1:1.

11-18. (canceled)

19. An amorphous solid dispersion comprising Compound 1: 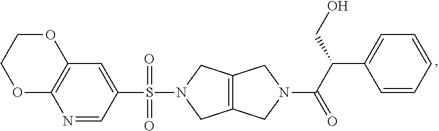 [SMILES: O=C([C@H](CO)c1ccccc1)N1CC2=C(C1)CN(S(=O)(=O)c1cnc3c(c1)OCCO3)C2]
and a polymer.

20. A pharmaceutical composition comprising a therapeutically effective amount of the amorphous solid dispersion of claim 19, and one or more pharmaceutically acceptable excipients.

21. A tablet dosage form comprising a tablet core, the tablet core comprising at least 10 weight % of Compound 1 in amorphous form: 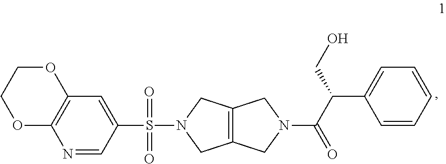 [SMILES: O=C([C@H](CO)c1ccccc1)N1CC2=C(C1)CN(S(=O)(=O)c1cnc3c(c1)OCCO3)C2]
wherein crystalline Compound 1 (Type A) is not observable by XRPD analysis (Method D) of the tablet core.

22. The tablet dosage form of claim 21, wherein the tablet core comprises at least 30 weight % of Compound 1 in amorphous form.

23. The tablet dosage form of claim 21, wherein the tablet core comprises about 200 mg of Compound 1 per tablet and has a total weight of no more than about 1200 mg per tablet.

24. The tablet dosage form of claim 23, wherein the tablet core has a total weight of no more than about 1100 mg, about 1000 mg, about 900 mg, about 800 mg, or about 700 mg per tablet.

25. The tablet dosage form of claim 21, wherein the tablet core is a minitablet comprising about 1-4 mg of Compound 1.

26. The tablet dosage form of claim 25, wherein crystalline Compound 1 (Type A) is not observable by XRPD analysis of the tablet core after storage under closed conditions as described in Example 33 for 4 weeks at 40° C. and 75% relative humidity.

27. The tablet dosage form of claim 21, wherein crystalline Compound 1 (Type A) is not observable by XRPD analysis (Method D) of the tablet core after storage in a sealed container as described in Example 29 for 1 month at 25° C. and 60% relative humidity.

28. The tablet dosage form of claim 21, wherein Compound 1 is present in an amorphous solid dispersion comprising Compound 1 and a polymer.

29. The amorphous solid dispersion of claim 19, wherein the polymer is selected from the group consisting of hydroxypropylmethyl cellulose (HPMC), hydroxypropylmethyl cellulose acetate succinate (HPMC AS), hydroxypropyl methyl cellulose phthalate (HPMCP), hydroxypropyl cellulose (HPC), ethylcellulose, cellulose acetate phthalate, polyvinylpyrrolidone (PVP), and a combination thereof.

30. The amorphous solid dispersion of claim 19, wherein the polymer is HPMC AS.
